(19) United States
(12) Patent Application Publication
(10) Pub. No.: US 2023/0025932 A1
(43) Pub. Date: Jan. 26, 2023

(54) NOVEL FUNCTIONALIZED LACTONES AS MODULATORS OF THE 5-HYDROXYTRYPTAMINE RECEPTOR 7 AND THEIR METHOD OF USE

(71) Applicants: Temple University-Of The Commonwealth System of Higher Education, Philadelphia, PA (US); Praeventix, LLC, Exton, PA (US)

(72) Inventors: Daniel J. Canney, Ambler, PA (US); Benjamin Blass Blass, Eagleville, PA (US); Kevin M. Blattner, Folsom, PA (US); Douglas A. Pippin, Chester Springs, PA (US)

(21) Appl. No.: 17/776,531

(22) PCT Filed: Nov. 12, 2020
(86) PCT No.: PCT/US2020/060271

§ 371 Date: May 12, 2022

Related U.S. Application Data
(60) Provisional application No. 62/934,985, filed on Nov. 13, 2019.

(51) Int. Cl. C07D 491/107; C07D 405/06

(52) U.S. Cl. CPC C07D 491/107; C07D 405/06

(57) ABSTRACT

Described herein are new, selective modulators of the 5-HT₇receptor. These selective compounds can be useful for the treatment of CNS and non-CNS indications. Compounds described herein can be selective in targeting 5-HT₇ receptors as compared to other receptors and/or by selective targeting 5-HT₇ receptors expressed in certain tissues or organs, thereby effective selectivity through a particular partitioning profile of the 5-HT₇ modulator

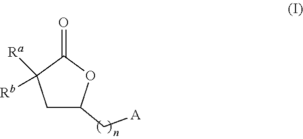

### CROSS-REFERENCE TO RELATED APPLICATIONS

This application claims priority to U.S. Provisional Patent Application 62/934,985, filed Nov. 13, 2019, which is incorporated by reference in its entirety.

### STATEMENT OF FEDERALLY FUNDED RESEARCH

This invention was made with government support under Grant Number 2R44DK115254-02A1 awarded by the National Institute of Diabetes and Digestive and Kidney Disease. The government has certain rights in the invention.

### BACKGROUND

Serotonin was discovered in the late 1940s and is present in both the peripheral and central nervous systems [Physiol. Res, 60 (2011) 15-25; Psychopharmacology 213 (2011) 167-169]. Serotonin or 5-hydroxytryptamine (5-HT) is a monoamine neurotransmitter of the indolalkylamine group that acts at synapses of nerve cells. Seven distinct families of serotonin receptors have been identified and at least 20 subpopulations have been cloned on the basis of sequence similarity, signal transduction coupling and pharmacological characteristics. The seven families of 5-HT receptor are named 5-HT₁, 5-HT₂, 5-HT₃, 5-HT₄, 5-HT₅, 5-HT₆, and 5-HT₇ and each of these receptors in turn has subfamilies or subpopulations. The signal transduction mechanism for all seven families have been studied and it is known that activation of 5-HT₁ and 5-HT₅ receptors causes a decrease in intracellular cAMP whereas activation of 5-HT₂, 5-HT₃, 5-HT₄, 5-HT₆, and 5-HT₇ results in an increase in intracellular IP3 and DAG. The 5-HT pathways in the brain are important targets for drug development in the area of CNS disorders. The neurotransmitter binds to its a G-protein coupled receptor and is involved in a wide variety of actions including cognition, mood, anxiety, attention, appetite, cardiovascular function, vasoconstriction, sleep (ACS Medicinal Chemistry Letters, 2011, 2, 929-932; Physiological Research, 2011, 60, 15-25), inflammatory bowel disease (IBD), and intestinal inflammation (WO 2012058769, Khan, W. I., et al. Journal of Immunology, 2013, 190, 4795-4804), epilepsy, seizure disorders (Epilepsy Research (2007) 75, 39), drug addiction, and alcohol addiction (Hauser, S. R. et al. Frontiers in Neuroscience, 2015, 8, 1-9) among others.

Described herein are new, selective modulators of the 5-HT₇ receptor. These selective compounds can be useful for the treatment of CNS and non-CNS indications. Compounds described herein can be selective in targeting 5-HT₇ receptors as compared to other receptors and/or by selective targeting 5-HT₇ receptors expressed in certain tissues or organs, thereby effective selectivity through a particular partitioning profile of the 5-HT₇ modulator.

### SUMMARY OF THE INVENTION

In one aspect, the invention features a compound having a structure according to Formula (I*):

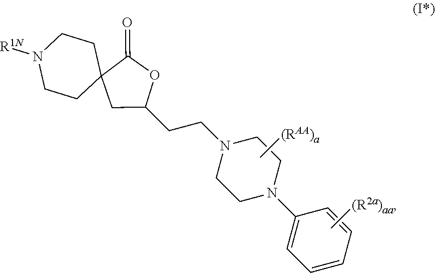

including enantiomers, diastereomers, hydrates, solvates, pharmaceutically acceptable salts, prodrugs and complexes thereof, wherein:
R^{1N} is selected from the group consisting of imidazole, oxazole, isoxazole,

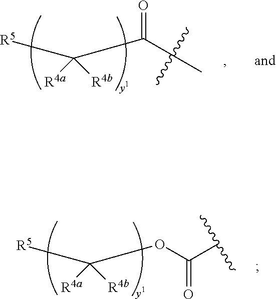

wherein
each R^{4a} and R^{4b} is hydrogen or C₁-C₇ alkyl; or R^{4a} and R^{4b} optionally are taken together with the atoms to which they are bound to form a ring containing 3 to 7 atoms, optionally containing oxygen;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and

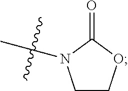 [SMILES: *N1CCOC1=O]

each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
each R^{AA} is independently C₃-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
a is 0, 1, or 2;
aa is 0, 1, or 2;
y¹ is 0, 1 or 2; and
wherein when R⁵ is unsubstituted C₁-C₇ alkyl or unsubstituted C₃-C₇ cycloalkyl, then a is 1 or 2.

In another aspect, the invention features a compound having a structure according to Formula (I*-N):

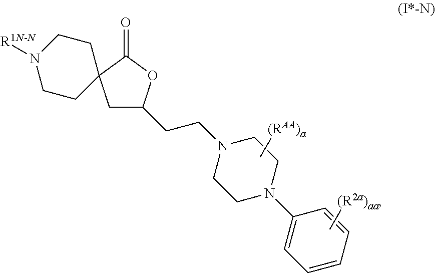

including enantiomers, diastereomers, hydrates, solvates, pharmaceutically acceptable salts, prodrugs and complexes thereof, wherein:
R^{1N-N} is selected from the group consisting of C₆-C₁₀ heteroaryl, five- to ten-membered heteroaryl,

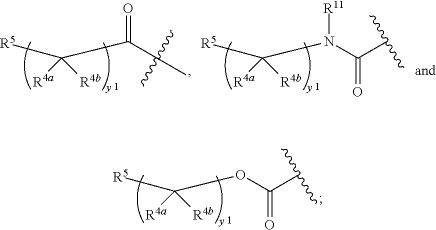

wherein
each R^{4a} and R^{4b} is hydrogen or C₁-C₇ alkyl; or R^{4a} and R^{4b} optionally are taken together with the atoms to which they are bound to form a ring containing 3 to 7 atoms, optionally containing oxygen;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and

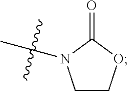 [SMILES: *N1CCOC1=O]

each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
each R^{AA} is independently C₃-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
a is 0, 1, or 2;
aa is 0, 1, or 2;
y¹ is 0, 1 or 2; and
wherein when R⁵ is unsubstituted C₁-C₇ alkyl or unsubstituted C₃-C₇ cycloalkyl, then a is 1 or 2.

In embodiments, a compound of Formula (I*) or (I*-N) has a structure according to Formula (I*-1),

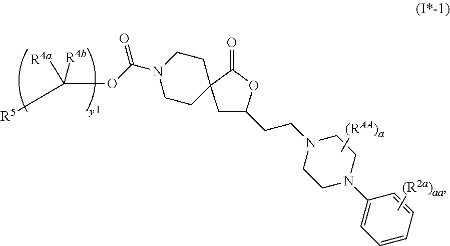

including enantiomers, diastereomers, hydrates, solvates, pharmaceutically acceptable salts, prodrugs and complexes thereof.

In embodiments, a compound of Formula (I*) or (I*-N) has a structure according to Formula (I*-2),

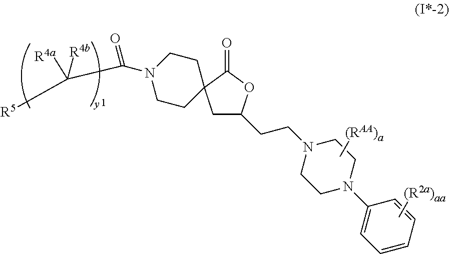

including enantiomers, diastereomers, hydrates, solvates, pharmaceutically acceptable salts, prodrugs and complexes thereof.

In embodiments, a compound of Formula (I*-N) has a structure according to Formula (I*-3),

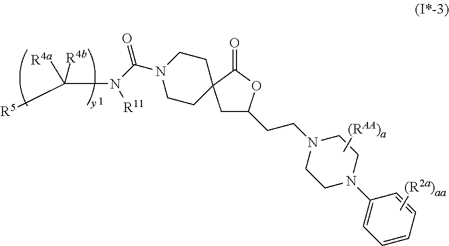

including enantiomers, diastereomers, hydrates, solvates, pharmaceutically acceptable salts, prodrugs and complexes thereof.

In embodiments, R^{1N} is:

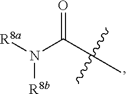

wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl;

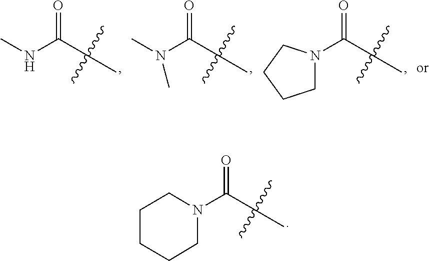

In embodiments, R^{1N} is:

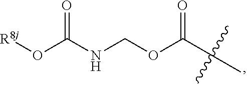

wherein R^{8j} is selected from the group consisting of C₁-C₇ alkyl C₃-C₇ cycloalkyl, C₆-C₁₀ aryl, and 5- to 10-membered heteroaryl;

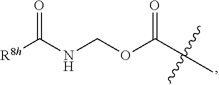

wherein R^{8h} is unsubstituted C₁-C₇ alkyl;

or

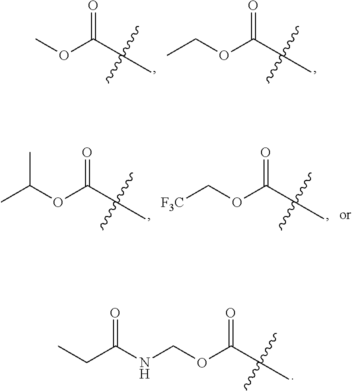

In embodiments R^{1N} is:

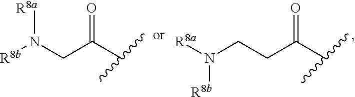

wherein each R^{8a} and R^{8b} is independently H or unsubstituted C₁-C₇ alkyl;

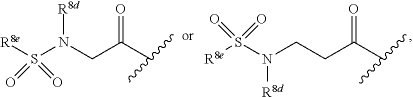

wherein R^{8d} is independently H or unsubstituted C₁-C₇ alkyl, and R^{8e} is unsubstituted C₁-C₇ alkyl;

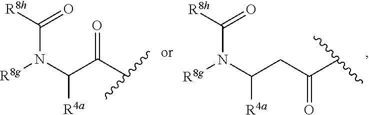

wherein each of R^{4a} and R^{8g} is independently H or unsubstituted C₁-C₇ alkyl; and R^{8h} is unsubstituted C₁-C₇ alkyl;

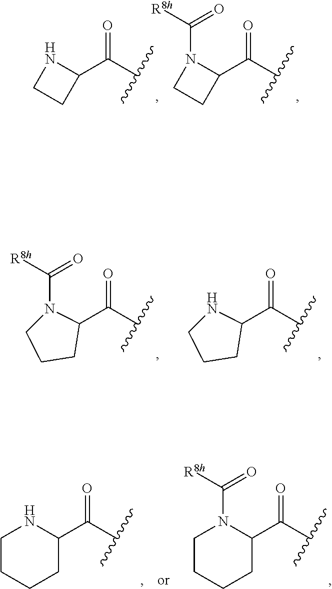

wherein R^{8h} is unsubstituted C₁-C₇ alkyl;

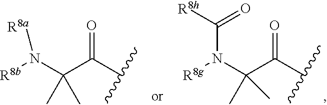

wherein each R^{8a}, R^{8b}, and R^{8g} is independently H or unsubstituted C₁-C₇ alkyl, and R^{8h} is unsubstituted C₁-C₇ alkyl;

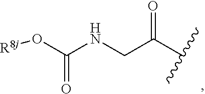

wherein R^{8j} is selected from the group consisting of C₁-C₇ alkyl, C₃-C₇ cycloalkyl, C₆-C₁₀ aryl, and 5- to 10-membered heteroaryl;

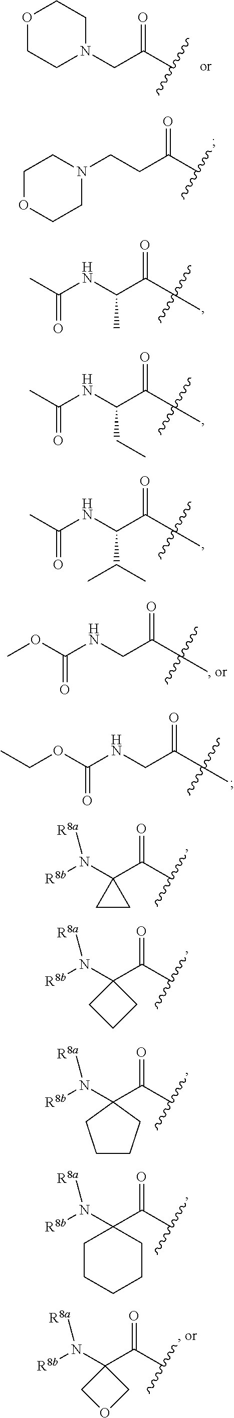

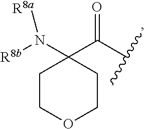

wherein each R^{8a} and R^{8b} is independently H or unsubstituted C₁-C₇ alkyl,

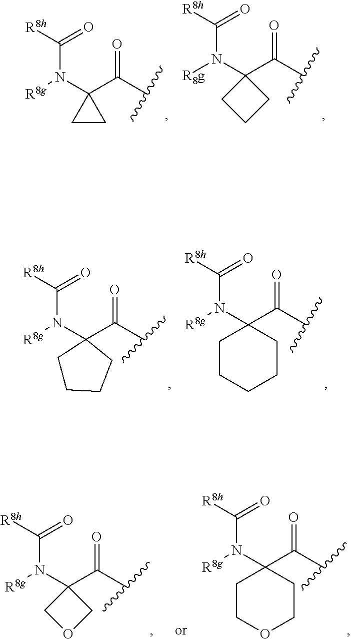

wherein R^{8g} is independently H or unsubstituted C₁-C₇ alkyl, and R^{8h} is independently unsubstituted C₁-C₇ alkyl;

or

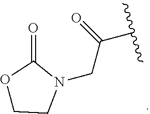 [SMILES: *C(=O)CN1CCOC1=O]

In embodiments, R^{1N} is

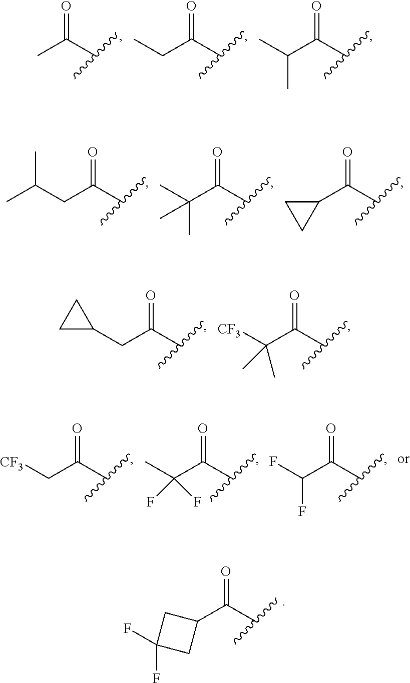

In embodiments, R^{1N} is

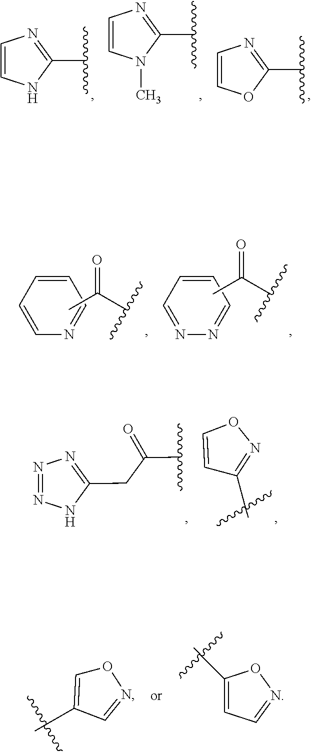

In another aspect, the invention features a compound having a structure according to Formula (I**):

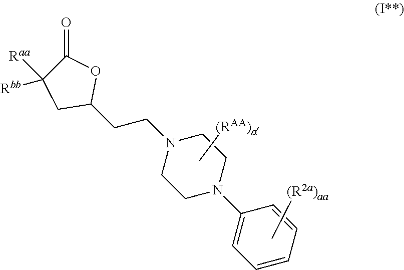

including enantiomers, diastereomers, hydrates, solvates, pharmaceutically acceptable salts, prodrugs and complexes thereof, wherein:
each R^{aa} and R^{bb} is selected from the group consisting of hydrogen, C₁-C₇ alkyl and C₃-C₇ branched alkyl;
each R^{AA} is independently C₁-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
aa is 0, 1, or 2; and
a′ is 1 or 2.

In embodiments, R^{aa} and R^{bb} are each ethyl.

In embodiments, a is 0 or 1.

In embodiments, a is 1 or 2, and each R^{AA} is methyl.

In embodiments, a′ is 1 or 2, and each R^{AA} is methyl.

In embodiments, aa is 0 or 1.

In embodiments, each R^{2a} is independently halogen.

In embodiments, each R^{2a} is independently —F or —Cl.

In embodiments, the C5 carbon of the 2-dihydrofuranone has the (R)-configuration.

In embodiments, the C5 carbon of the 2-dihydrofuranone has the (R)-configuration.

In another aspect, the invention features a compound having a structure according to Formula (I)

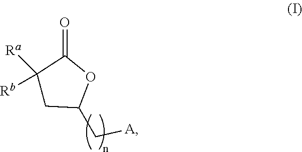

including enantiomers, diastereomers, hydrates, solvates, pharmaceutically acceptable salts, prodrugs and complexes thereof, wherein:
each R^{a} and R^{b} is selected from the group consisting hydrogen, C₁-C₇ alkyl, and C₃-C₇ branched alkyl; or R^{a} and R^{b} are taken together with the atoms to which they are bound to form a carbocyclic ring having from 5 to 7 ring atoms, optionally containing a double bond; or R^{a} and R^{b} are taken together with the atoms to which they are bound to form a ring having from 6 to 8 ring atoms comprising a moiety selected from the group consisting of O, S, SO, SO₂, and NR¹;
A is an N-linked, five- to twelve-membered nitrogen-containing heterocyclyl, wherein said nitrogen-containing heterocyclyl is bicyclic or polycyclic and optionally includes further heteroatoms selected from O, N, and S, and wherein a non-aromatic, nitrogen-containing heterocyclyl further comprises a group R²;
R¹ is a H, C₁-C₇ alkyl, C₃-C₇ cycloalkyl, phenyl, benzyl, five- to six-membered heteroaryl ring, a polar acyl group, or a polar sulfonyl group;
R² is selected from the group consisting of 6- to 10-membered aryl, 5- to 10-membered nitrogen-containing heteroaryl, and

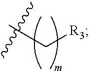

R³ is a 6- to 10-membered aryl or 5- to 10-membered nitrogen-containing heteroaryl;
m is 1, 2, or 3; and
n is 1, 2, 3, or 4; and
wherein when A is 1,2,3,4-tetrahydroquinol-1-yl, 1,2,3,4-tetrahydroisoquinol-2-yl, octahydropyrrolo[3,4-c]pyrrol-1-yl, or 2,6-diazaspiro[3.3]heptan-1-yl, then R^{a} and R^{b} cannot both be methyl, both be ethyl, or both be phenyl nor can R^{a} and R^{b} combine to form unsubstituted C₃-C₆ cycloalkyl.

In embodiments of Formula (I), R^{a} and R^{b} are taken together with the atoms to which they are bound to form a ring having from 6 to 8 ring atoms, wherein one of the ring atoms is a moiety selected from the group consisting of O, S, SO, SO₂, and NR¹.

In embodiments, the compound of Formula (I) has one of the following structures,

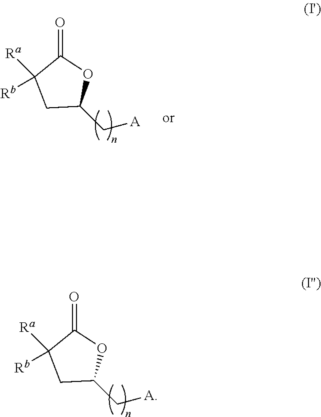

In embodiments of Formula (I), R^{a} and R^{b} are both methyl or ethyl, or R^{a} and R^{b} combine to form unsubstituted cyclopropyl, cyclobutyl, cyclopentyl, or cyclohexyl.

In embodiments, a compound of Formula (I) has one of the following structures:

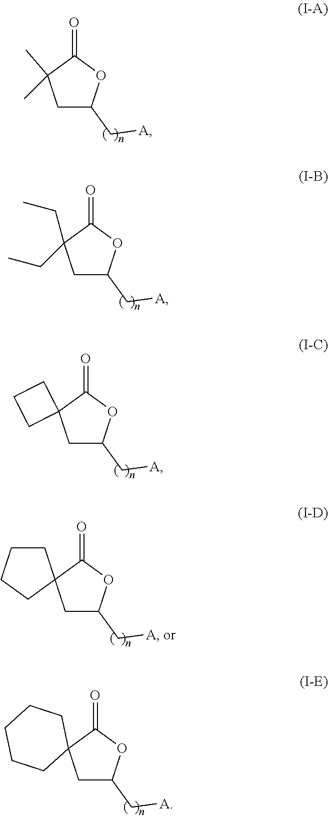

In embodiments, a compound of Formula (I) is according to one of the following structures,

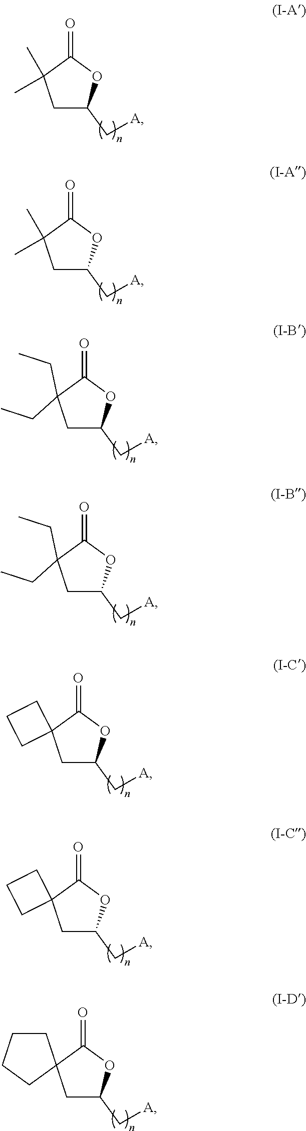

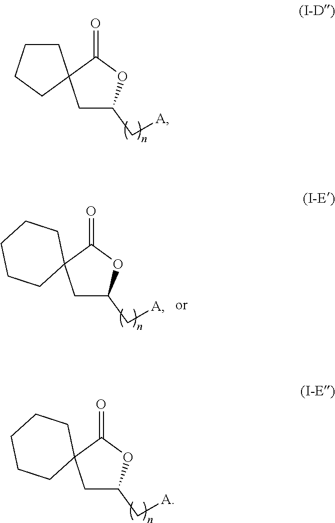

In embodiments of Formula (I), R^{a} and R^{b} are taken together with the atoms to which they are bound to form a ring having from 6 to 8 ring atoms.

In embodiments, a compound of Formula (I) has a structure according to Formula (I-F), (I-F).

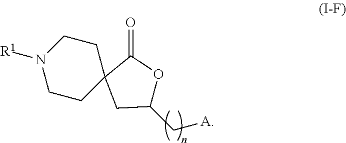

In embodiments, a compound of Formula (I) has a structure according to one of the following formulas,

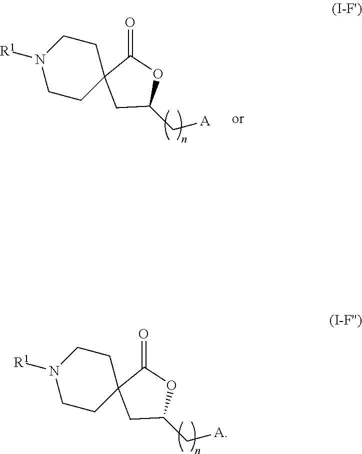

In embodiments of Formula (I), A is selected from the group consisting of:

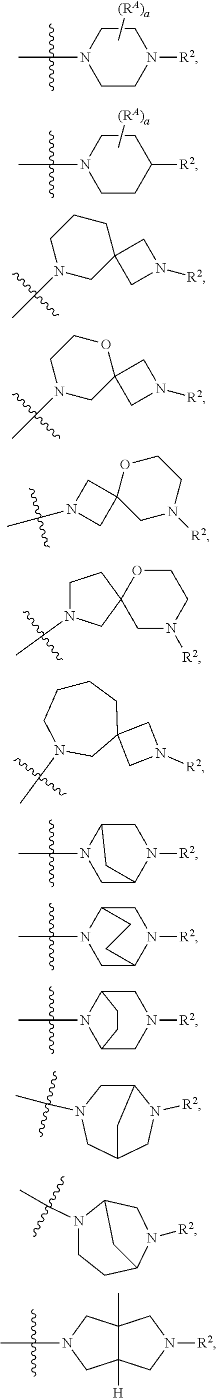

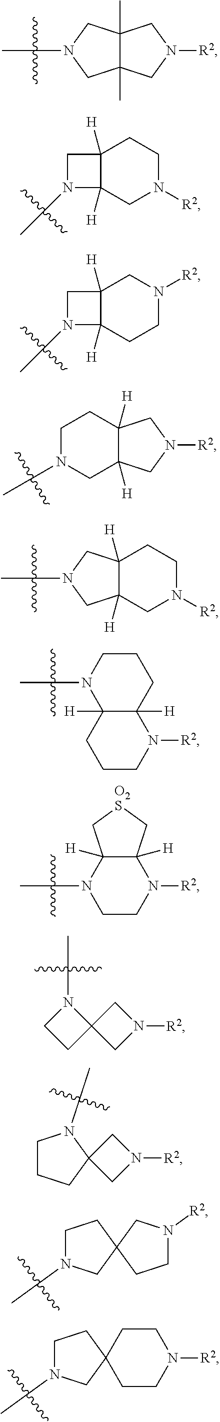

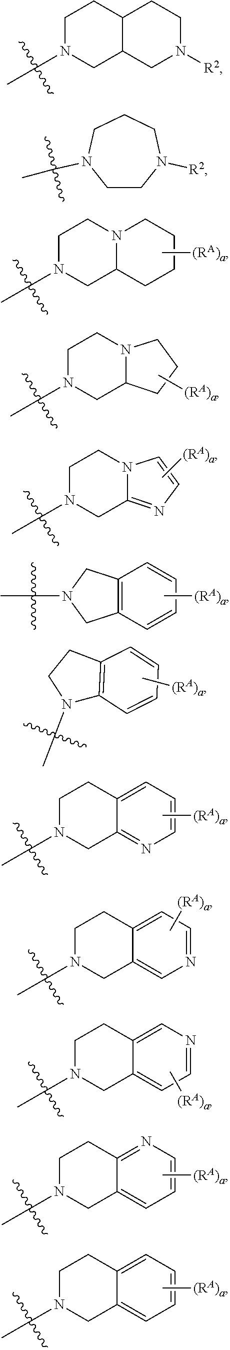

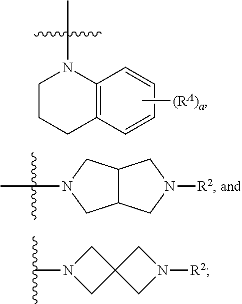

wherein
R² is selected from the group consisting of phenyl, naphthyl, pyridyl, indolyl and;

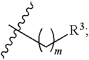

R³ is selected from the group consisting of phenyl, naphthyl, pyridyl and indolyl;
R^{A} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen; and
a is 0, 1, or 2.

In embodiments of Formula (I), A is selected from the group consisting of:

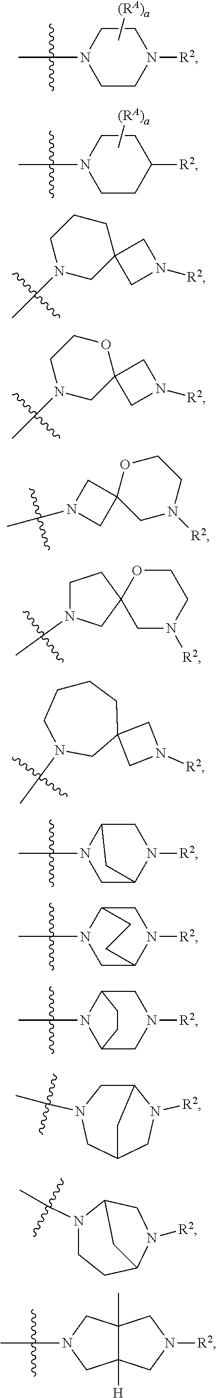

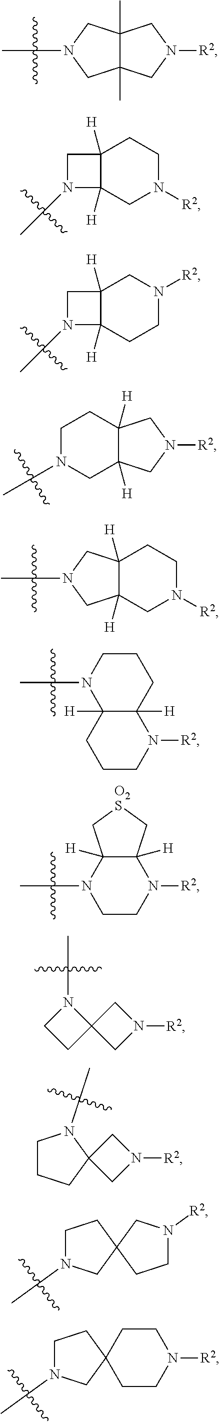

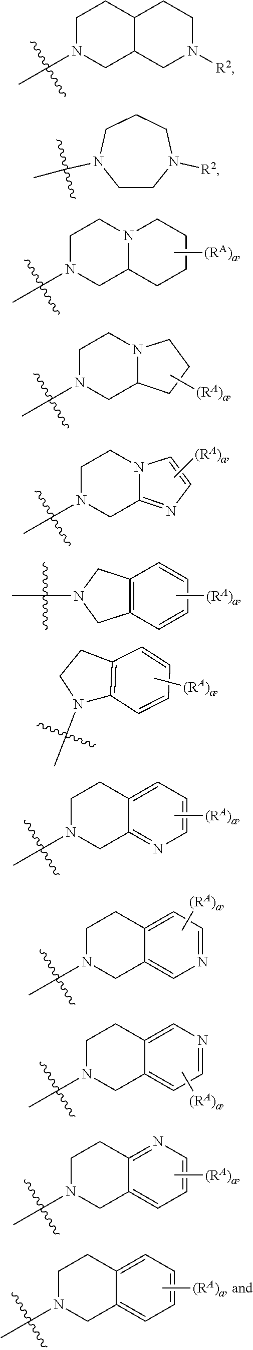

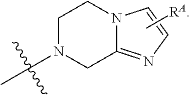

In embodiments of Formula (I), A is selected from the group consisting of:

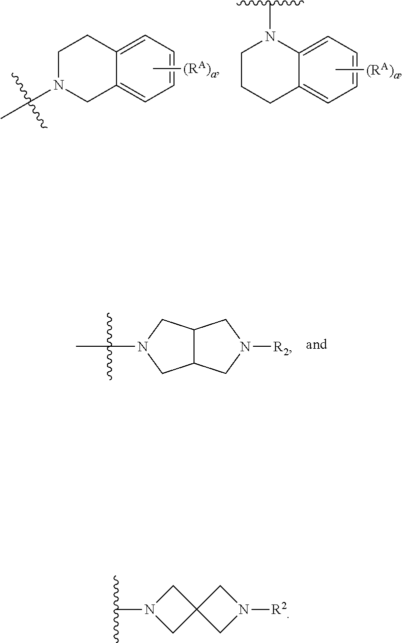

In embodiments of Formula (I), a is 0. In embodiments of Formula (I), a is 1. In embodiments of Formula (I), a is 2.

In embodiments of Formula (I), R¹ is selected from the group consisting of H, C₁-C₇ alkyl, C₃-C₇ cycloalkyl, phenyl, benzyl, imidazole, oxazole, isoxazole,

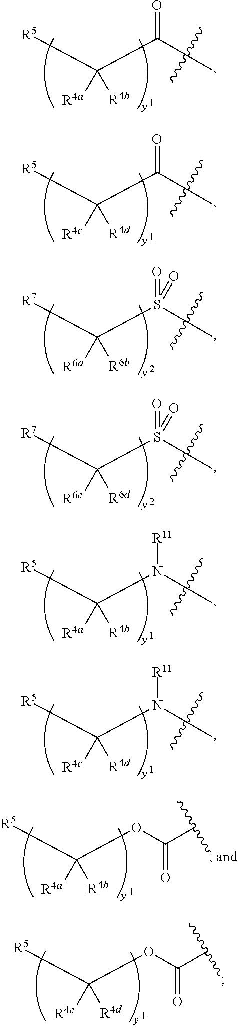

each R^{4a}, R^{4b}, R^{4c}, R^{6a}, R^{6b} and R^{6c} is selected from the group consisting of hydrogen, C₁-C₇ alkyl and C₃-C₇ cycloalkyl;
R^{4a} and R^{4b} optionally are taken together with the atoms to which they are bound to form a ring containing 3 to 7 atoms, optionally containing oxygen;
R^{6a} and R^{6b} optionally are taken together with the atoms to which they are bound to form a ring containing 3 to 7 atoms, optionally containing oxygen;
each R^{4d} and R^{6d} is selected from the group consisting of phenyl, benzyl, pyridyl, —CH₂(pyridyl), imidazole, and —CH₂(imidazole).
R⁵ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8c}, NR⁸ⁱCOOR^{8j}, NHCONR^{8f}, NR^{8g}COR^{8h} and

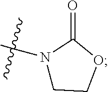 [SMILES: *N1CCOC1=O]

R⁷ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8c}, NHCONR^{8f};
each R^{8a}, R^{8b}, R^{8d}, R⁸, and R^{8i g} is selected from the group consisting of hydrogen, C₁-C₇ alkyl, and C₃-C₇ cycloalkyl;
R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
R¹¹ is selected from the group consisting of hydrogen, C₁-C₇ alkyl, and C₃-C₇ cycloalkyl;
y¹ is 0, 1 or 2; and
y² is 0, 1, or 2.

In embodiments of Formula (I), R¹ is selected from the group consisting of:

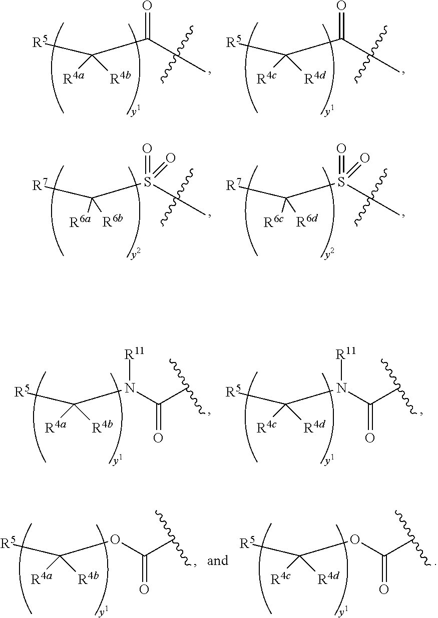

In embodiments of Formula (I), R¹ is selected from the group consisting of: COOR⁵, wherein R⁵ is C₆-C₁₀ aryl or 5- to 10-membered heteroaryl;

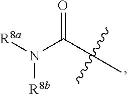

wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl;

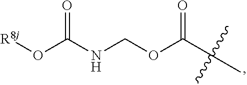

wherein R^{8j} is selected from the group consisting of C₁-C₇ alkyl, C₃-C₇ cycloalkyl, C₆-C₁₀ aryl, and 5- to 10-membered heteroaryl;

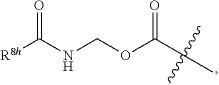

wherein R^{8h} is unsubstituted C₁-C₇alkyl;

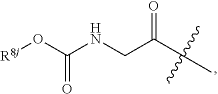

wherein R^{8j} is selected from the group consisting of C₁-C₇ alkyl, C₃-C₇ cycloalkyl, C₆-C₁₀ aryl, and 5- to 10-membered heteroaryl;

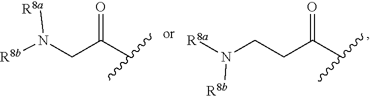

wherein each R^{8a} and R^{8b} is independently H or unsubstituted C₁-C₇ alkyl;

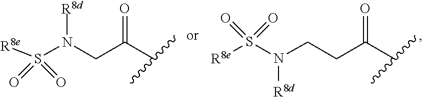

wherein R^{8d} is independently H or unsubstituted C₁-C₇ alkyl, and R⁸¹ is unsubstituted C₁-C₇ alkyl

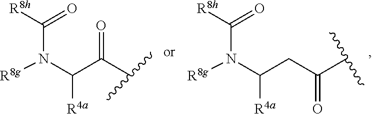

wherein each of R^{4a} and R^{8g} is independently H or unsubstituted C₁-C₇ alkyl and R^{8h} is unsubstituted C₁-C₇ alkyl

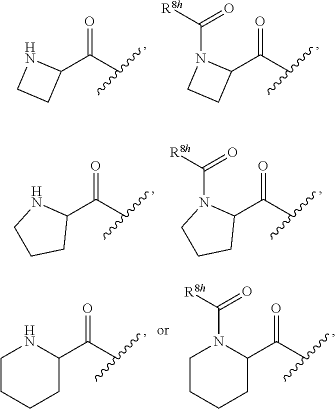

wherein R^{8h} is unsubstituted C₁-C₇ alkyl;

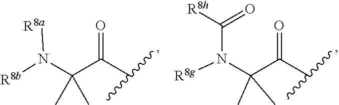

wherein each R^{8a}, R^{8b}, and R^{8g} is independently H or unsubstituted C₁-C₇ alkyl, and R^{8e} is unsubstituted C₁-C₇ alkyl;

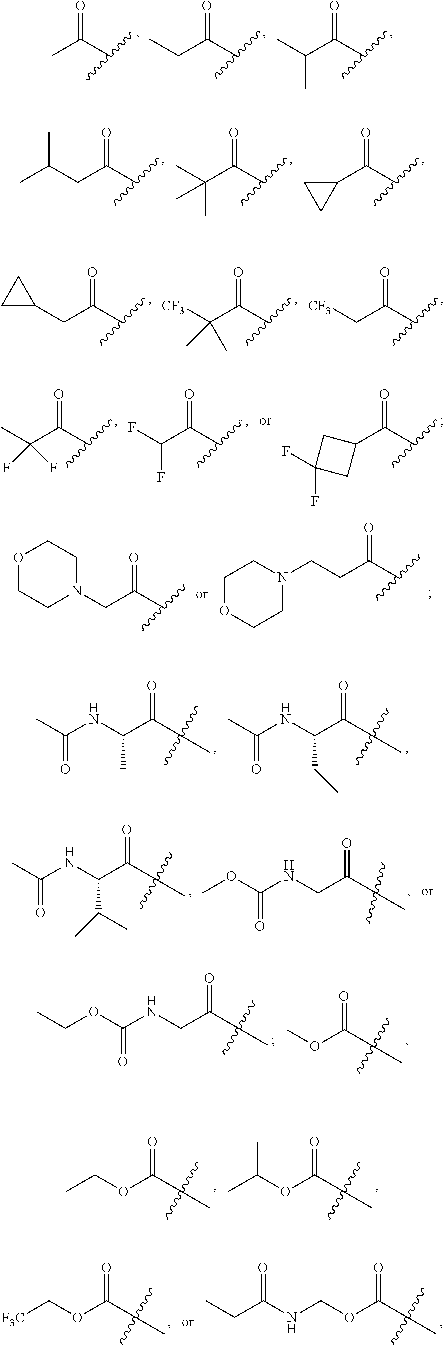

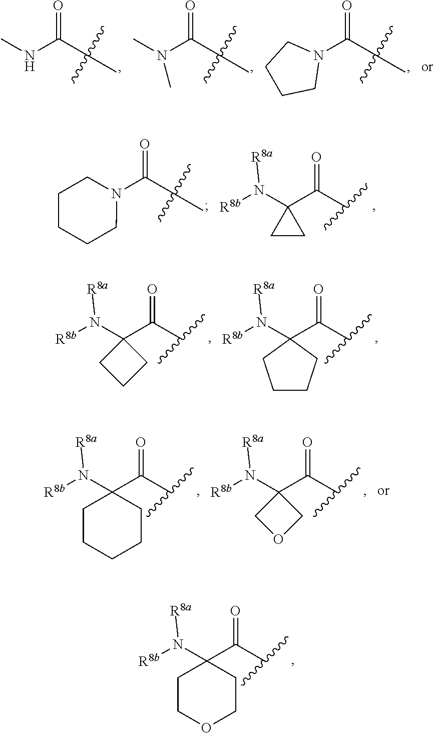

wherein each R^{8a} and R^{8b} is independently H or unsubstituted C₁-C₇ alkyl;

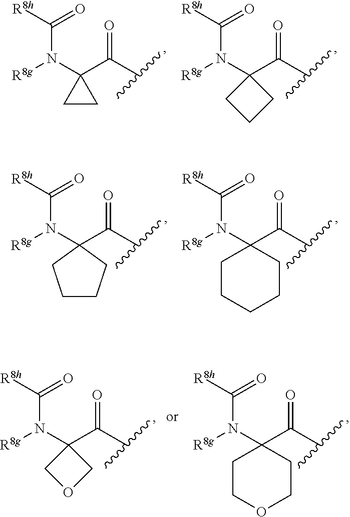

wherein R^{8g} is independently H or unsubstituted C₁-C₇ alkyl, and R^{8h} is independently unsubstituted C₁-C₇ alkyl; or

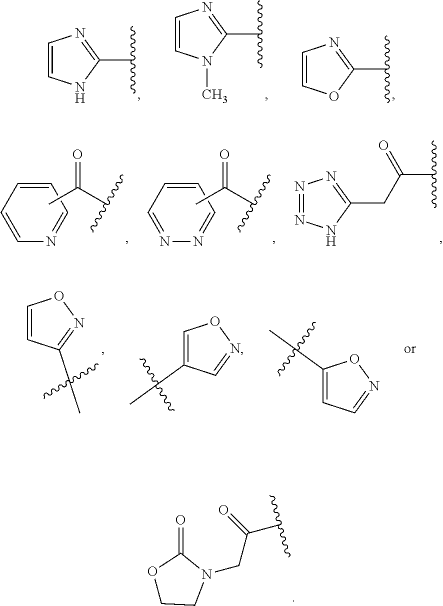

In another aspect, the invention features a compound according to Formula (II),

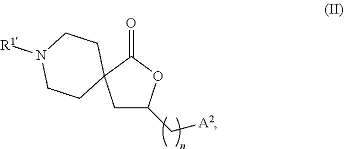

including enantiomers, diastereomers, hydrates, solvates, pharmaceutically acceptable salts, prodrugs and complexes thereof, wherein:

A² is

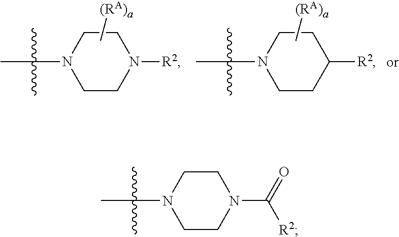

R² is selected from the group consisting of 6- to 10-membered aryl, 5- to 10-membered nitrogen-containing heteroaryl, and

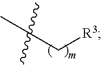

R³ is a 6- to 10-membered aryl or 5- to 10-membered nitrogen-containing heteroaryl;
R^{A} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen;
a is 0, 1, or 2;
m is 1, 2, or 3;
n is 1, 2, 3, or 4;
R^{1′} is selected from the group consisting of a C₆-C₁₀ aryl, a five- to six-membered heteroaryl ring,

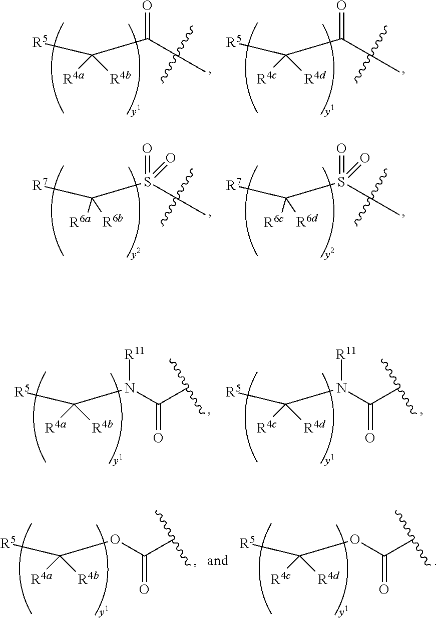

each R^{4a}, R^{4b}, R^{4c}, R^{6a}, R^{6b} and R^{6c} is selected from the group consisting of hydrogen, C₁-C₇ alkyl and C₃-C₇ cycloalkyl;
R^{4a} and R^{4b} optionally are taken together with the atoms to which they are bound to form a ring containing 3 to 7 atoms, optionally containing oxygen;
R^{6a} and R^{6b} optionally are taken together with the atoms to which they are bound to form a ring containing 3 to 7 atoms, optionally containing oxygen;
each R^{4d} and R^{6d} is selected from the group consisting of phenyl, benzyl, pyridyl, —CH₂(pyridyl), imidazole, and —CH₂(imidazole).
R⁵ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}CR^{8h} and

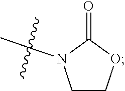 [SMILES: *N1CCOC1=O]

R⁷ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NHCONR^{8f};
each R^{8a}, R^{8b}, R^{8d}, R^{8g}, and R⁸ⁱ is selected from the group consisting of hydrogen, C₁-C₇ alkyl, and C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
R¹¹ is selected from the group consisting of hydrogen, C₁-C₇ alkyl, and C₃-C₇ cycloalkyl;
y¹ is 0, 1 or 2; and
y² is 0, 1, or 2; and
wherein when A² is

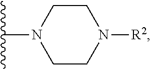

R² is phenyl, R^{1′} is

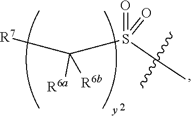

y² is 0, and n is 2, then R⁷ is not methyl, CH₂SO₂CH₃, CH₂CN, tetrahydropyranyl, phenyl, 4-substituted phenyl, or 5- to 8-membered heteroaryl.

In embodiments, a compound according to Formula (II) has one of the following structures,

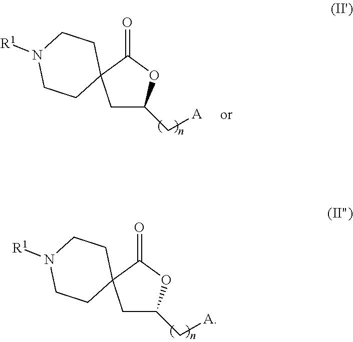

In embodiments of Formula (II), R^{1′} is:
COOR⁵, wherein R⁵ is C₆-C₁₀ aryl or 5- to 10-membered heteroaryl;

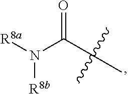

wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl;

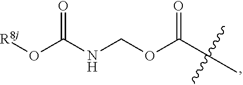

wherein R^{8j} is selected from the group consisting of C₁-C₇ alkyl, C₃-C₇ cycloalkyl, C₆-C₁₀ aryl, and 5- to 10-membered heteroaryl;

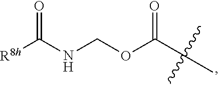

wherein R^{8f} is unsubstituted C₁-C₇ alkyl;

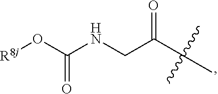

wherein R^{8j} is selected from the group consisting of C₁-C₇ alkyl, C₃-C₇ cycloalkyl, C₆-C₁₀ aryl, and 5- to 10-membered heteroaryl;

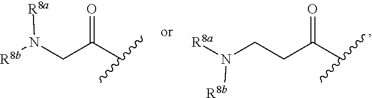

wherein each R^{8a} and R^{8b} is independently H or unsubstituted C₁-C₇ alkyl;

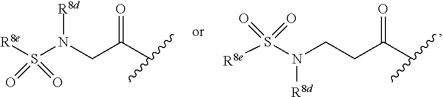

wherein R^{8d} is independently H or unsubstituted C₁-C₇ alkyl, and R^{8e} is unsubstituted C₁-C₇ alkyl

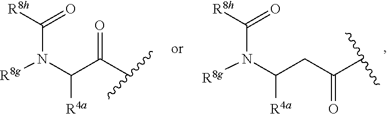

wherein each of R^{4a} and R^{8g} is independently H or unsubstituted C₁-C₇ alkyl; and R^{8h} is unsubstituted C₁-C₇ alkyl;

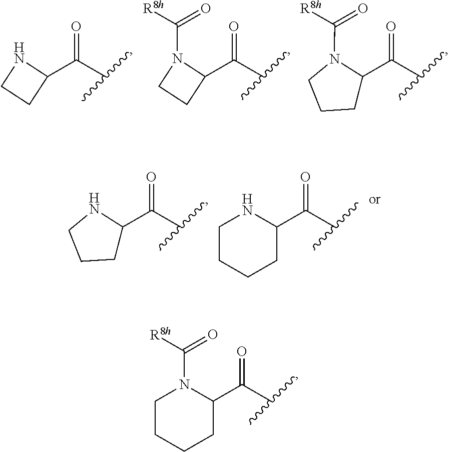

wherein R^{8h} is unsubstituted C₁-C₇ alkyl;

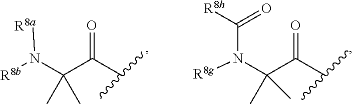

wherein each R^{8a}, R^{8b}, and R^{8g} is independently H or unsubstituted C₁-C₇ alkyl, and R^{8h} is unsubstituted C₁-C₇ alkyl;

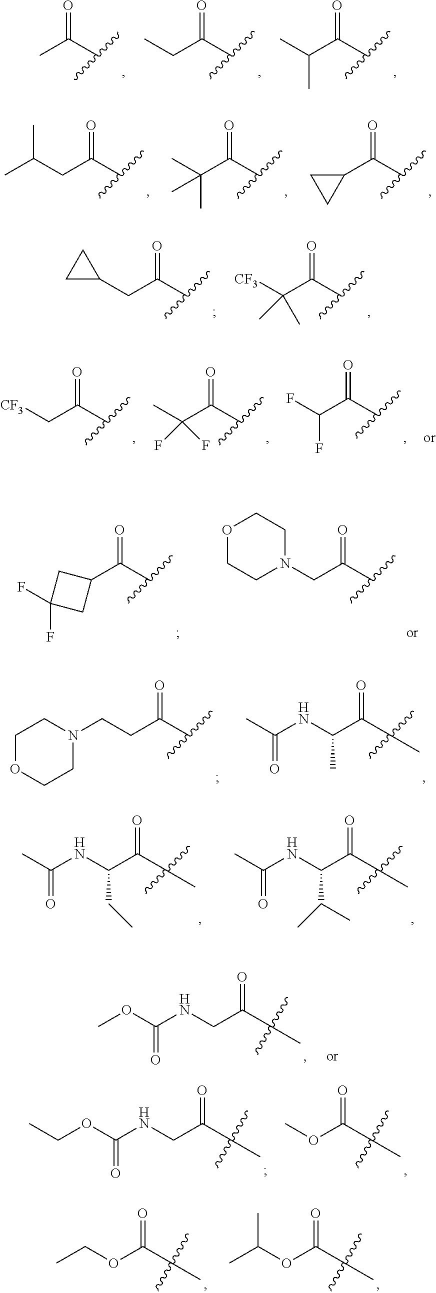

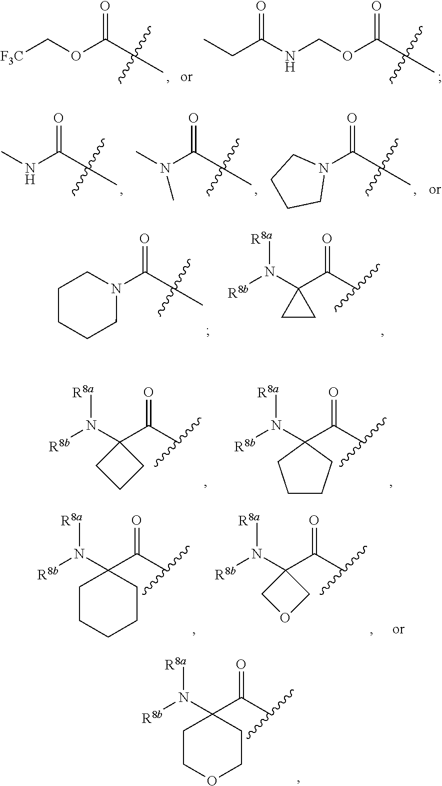

wherein each R^{8a} and R^{8b} is independently H or unsubstituted C₁-C₇ alkyl;

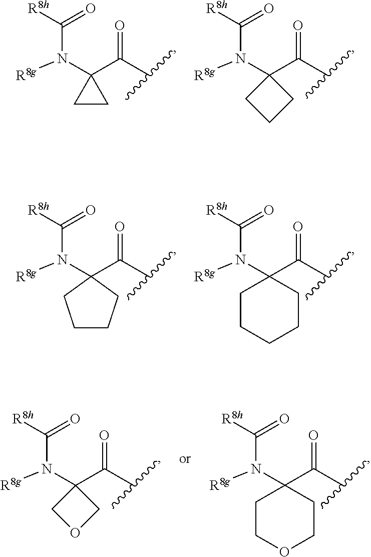

wherein R^{a}d is independently H or unsubstituted C₁-C₇ alkyl, and R^{8h} is independently unsubstituted C₁-C₇ alkyl; or

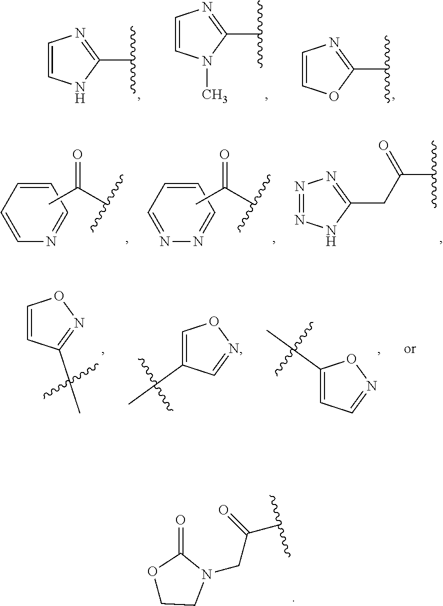

In embodiments of Formula (II), A² is

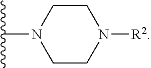

In embodiments of Formula (II), A² is

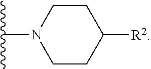

In embodiments of Formula (II), A² is

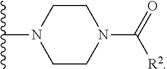

In embodiments, a compound of Formula (I), (I*), (I**), or (II) is any one of Compounds 1-145, including enantiomers, diastereomers, hydrates, solvates, pharmaceutically acceptable salts, prodrugs and complexes thereof.

In another aspect, the invention features a pharmaceutical composition comprising any compound described herein (e.g., a compound according to Formula (I), (I*), (I**), or (II)), or a pharmaceutically acceptable salt thereof.

In embodiments, a pharmaceutical composition further comprises at least one pharmaceutically acceptable excipient.

In another aspect, the invention features a method of treating a disease associated with dysregulation of 5-hydroxytryptamine receptor 7 activity, said method comprising administering to a subject an effective amount of at least one compound as described herein (e.g., a compound according to Formula (I), (I*), (I**), or (II)), or a pharmaceutically acceptable salt thereof.

In embodiments, the at least one compound, or a pharmaceutically acceptable salt thereof, is administered in a composition further comprising at least one excipient.

In embodiments, the disease associated with dysregulation of 5-hydroxytryptamine receptor 7 activity is selected from the group consisting of peripherally selective diseases, nervous system diseases, circadian rhythm disorder, depression, schizophrenia, neurogenic inflammation, hypertension, peripheral, vascular diseases, migraine, neuropathic pain, peripheral pain, allodynia, thermoregulation disorder, learning disorder, memory disorder, hippocampal signaling disorder, sleep disorder, attention deficit/hyperactivity disorder, anxiety, avoidant personality disorder, premature ejaculation, eating disorder, premenstrual syndrome, premenstrual dysphonic disorder, seasonal affective disorder, bipolar disorder, inflammatory bowel disease (IBD), intestinal inflammation, epilepsy, seizure disorders, drug addiction, alcohol addiction, breast cancer, liver fibrosis, chronic liver injury, hepatocellular carcinoma, small intestine neuroendocrine tumors, and lung injury.

In embodiments, the disease associated with dysregulation of 5-hydroxytryptamine receptor 7 activity is inflammatory bowel disease (IBD) or intestinal inflammation.

### DETAILED DESCRIPTION

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, exemplary methods, devices, and materials are now described. All technical and patent publications cited herein are incorporated herein by reference in their entirety. Nothing herein is to be construed as an admission that the invention is not entitled to antedate such disclosure by virtue of prior invention.

As used throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

As used throughout the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g., C₁-C₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be unsubstituted or substituted, including with any substitutents and combination of substitutents described herein. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁-C₆ alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be unsubstituted or substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be unsubstituted or substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “haloalkoxy” refers to the group —O-haloalkyl, wherein the haloalkyl group is as defined above. Examples of haloalkoxy groups include, but are not limited to, fluoromethoxy, difluoromethoxy, trifluoromethoxy, and pentafluoroethoxyl.

The term “aryl,” wherein used alone or as part of another group, is defined herein as an unsaturated, aromatic monocyclic ring of 6 carbon members or to an unsaturated, aromatic polycyclic ring of from 6 to 14 carbon members. Aryl groups can be unsubstituted or substituted. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocyclyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be unsubstituted or substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be unsubstituted or substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, 1H-benzo[d]imidazol-2(3H)-onyl, 1H-benzo[d]imidazolyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

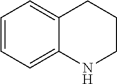

is, for the purposes of the present invention, considered a heterocyclic unit. 6,7-Dihydro-5H-cyclopentapyrimidine having the formula:

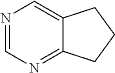

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

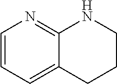

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated. The following are non-limiting examples of substituents which can substitute for hydrogen atoms on a moiety: halogen (chlorine (Cl), bromine (Br), fluorine (F) and iodine(I)), —CN, —NO₂, oxo (═O), —OR′, —SR′, —N(R′)₂, —NR′C(O)R′, —SO₂R′, —SO₂OR′, —SO₂N(R′)₂, —C(O)R′, —C(O)OR′, —C(O)N(R′)₂, C₁-C₆ alkyl, C₁-C₆ haloalkyl, C₁-C₆ alkoxy, C₂-C₈ alkenyl, C₂-C₈ alkynyl, C₃-C₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R′; wherein R′, at each occurrence, independently is hydrogen, —OR″, —SR″, —C(O)R″, —C(O)OR″, —C(O)N(R″)₂, —SO₂R″, —S(O)₂OR″, —N(R″)₂, —NR″C(O)R″, C₁-C₆ alkyl, C₁-C₆ haloalkyl, C₂-C₈ alkenyl, C₂-C₈ alkynyl, cycloalkyl (e.g., C₃-C₆ cycloalkyl), aryl, heterocycle, or heteroaryl, or two R′ units taken together with the atom(s) to which they are bound form an optionally substituted carbocycle or heterocycle wherein said carbocycle or heterocycle has 3 to 7 ring atoms; wherein R″, at each occurrence, independently is hydrogen, C₁-C₆ alkyl, C₁-C₆ haloalkyl, C₂-C₈ alkenyl, C₂-C₈ alkynyl, cycloalkyl (e.g., C₃-C₆ cycloalkyl), aryl, heterocycle, or heteroaryl, or two R″ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR′″; for example, —OH, —OCH₃, —OCH₂CH₃, —OCH₂CH₂CH₃;
ii) —C(O)R′″; for example, —COCH₃, —COCH₂CH₃, —COCH₂CH₂CH₃;
iii) —C(O)OR′″; for example, —CO₂CH₃, —CO₂CH₂CH₃, —CO₂CH₂CH₂CH₃;
iv) —C(O)N(R′)₂; for example, —CONH₂, —CONHCH₃, —CON(CH₃)₂;
v) —N(R′″)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R′″; for example, —SO₂H; —SO₂CH₃; —SO₂C₆H₅;
ix) C₁-C₆ linear, branched, or cyclic alkyl;
x) Cyano
xi) Nitro;
xii) N(R′)C(O)R′;
xiii) Oxo (═O);
xiv) Heterocycle; and
xv) Heteroaryl.

wherein each R′″ is independently hydrogen, optionally substituted C₁-C₆ linear or branched alkyl (e.g., optionally substituted C₁-C₄ linear or branched alkyl), or optionally substituted C₃-C₆ cycloalkyl (e.g optionally substituted C₃-C₄ cycloalkyl); or two R′″ units can be taken together to form a ring comprising 3-7 ring atoms. In certain aspects, each R′″ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₈, C₂-C₄, C₂-C₃, C₃-C₆, C₃-C₅, C₃-C₄, C₄-C₆, C₄-C₅, and C₅-C₆ alkyl.

For the purposes of the present invention the terms “compound,” “analog,” and “composition of matter” stand equally well for the 5-hydroxytryptamine receptor 7 activity modulators described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. For example, described herein are certain gammabutyrolactones comprising a substituent at the C5 carbon of the heterocycle. In embodiments of any compound or formula described herein, the C5 carbon has the (S)-configuration. In embodiments of any compound or formula described herein, the C5 carbon has the (R)-configuration. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di-, or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R⁹)₂, each R⁹ may be the same or different than the other). Combinations of substituents and/or variables are permissible only if such combinations result in stable compounds.

The terms “treat,” “treating,” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

### 5-Hydroxytryptamine Receptor 7 Activity Modulators

Modulators of 5-HT7 Activity

Described herein are lactone compounds that can modulate 5-hydroxy receptor 7 (5-HT₇) activity. In particular, compounds described herein can be selective modulators of 5-HT₇ receptors. In embodiments, selective modulation of 5-HT₇ encompasses selective modulation of 5-HT₇ as compared to other receptors. In embodiments, selective modulation of 5-HT₇ encompasses selective modulation of 5-HT₇ expressed in, e.g., a particular organ or tissue. Accordingly, the compounds described herein can be useful for the treatment of various diseases and conditions (e.g., as described herein).

In embodiments of any formula described herein, a C₁-C₇ alkyl is C₁-C₇ linear alkyl. In embodiments, a C₁-C₇ alkyl is unsubstituted C₁-C₇ linear alkyl. In embodiments, a C₁-C₇ alkyl is substituted C₁-C₇ linear alkyl (e.g., substituted with 1, 2, 3, or more substituent groups as described herein). In embodiments, a substituted C₁-C₇ linear alkyl is a C₁-C₇ linear perhaloalkyl (e.g., perfluoroalkyl). In embodiments, a substituted C₁-C₇ linear alkyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of C₁-C₇ alkyl are described herein.

In embodiments of any formula described herein, a C₁-C₇ alkyl is C₃-C₇ branched alkyl. In embodiments, a C₃-C₇ branched is unsubstituted C₃-C₇ branched alkyl. In embodiments, a C₃-C₇ branched alkyl is substituted C₃-C₇ branched alkyl (e.g., substituted with 1, 2, 3, or more substituent groups as described herein). In embodiments, a substituted C₃-C₇ branched alkyl is a C₃-C₇ branched perhaloalkyl (e.g., perfluoroalkyl). In embodiments, a substituted C₃-C₇ branched alkyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of C₃-C₇ branched alkyl are described herein.

In embodiments of any formula described herein, a cycloalkyl is a C₃-C₇ or C₃-C₈ cycloalkyl. In embodiments a cycloalkyl is cyclopropyl. In embodiments a cycloalkyl is cyclobutyl. In embodiments a cycloalkyl is cyclopentyl. In embodiments a cycloalkyl is cyclohexyl. In embodiments, a cycloalkyl is unsubstituted cycloalkyl (e.g., unsubstituted cyclopropyl, cyclobutyl, cyclopentyl, or cyclohexyl). In embodiments, a cycloalkyl is substituted cycloalkyl (e.g., a cyclopropyl, cyclobutyl, cyclopentyl, or cyclohexyl comprising 1, 2, 3, 4, or 5 substituent groups including exemplary substituent groups described herein). In embodiments, a substituted cycloalkyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of cycloalkyl are described herein.

In embodiments of any formula described herein, a C₆-C₁₀ aryl is phenyl. In embodiments, a phenyl is unsubstituted phenyl. In embodiments, a phenyl is substituted phenyl (e.g., a phenyl comprising 1, 2, 3, 4, or 5 substituent groups including exemplary substituent groups described herein). A substituted phenyl group can be attached via any available carbon of the ring, including as described herein. For example, a phenyl can have a substituent as described herein (e.g., OH, OCH₃, NH₂, CN, CH₃, CF₃, CH₂CH₃, isopropyl, F, Cl, Br, morpholino, CO₂H, CO₂CH₃, and CO₂NH₂)para to the point of attachment to a molecule (e.g., a 4-substituted phenyl group). In embodiments, a phenyl can have a substituent as described herein (e.g., OH, OCH₃, NH₂, CN, CH₃, CF₃, CH₂CH₃, isopropyl, F, Cl, Br, morpholino, CO₂H, CO₂CH₃, and CO₂NH₂) meta to the point of attachment to a molecule (e.g., a 3-substituted phenyl group). In embodiments, a phenyl can have a substituent as described herein (e.g., OH, OCH₃, NH₂, CN, CH₃, CF₃, CH₂CH₃, isopropyl, F, Cl, Br, morpholino, CO₂H, CO₂CH₃, and CO₂NH₂) ortho to the point of attachment to a molecule (a 2-substituted phenyl group). A phenyl group may have two or more (e.g., a 2,3-disubstituted, 2,4-disubstituted, 2,5-disubstituted, 2,6-disubstituted, 3,4-disubstituted, or 3,5-disubstituted phenyl) or three or more substituents (e.g., 2,3,4-trisubstituted 2,3,5-trisubstituted, 2,3,6-trisubstituted, 2,4,5-trisubstituted, 2,4,6-trisubstituted, 3,4,5-trisubstituted, or 3,4,6-trisubstituted). In embodiments, a substituted phenyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of phenyl are described herein. In embodiments, a phenyl is unsubstituted phenyl, 4-OH-phenyl, 3-OH-phenyl, 2-OH-phenyl, 4-OMe-phenyl, 3-OMe-phenyl, 2-OMe-phenyl, 4-CN-phenyl, 3-CN-phenyl, 2-CN-phenyl, 4-Me-phenyl, 3-Me-phenyl, 2-Me-phenyl, 4-Et-phenyl, 3-Et-phenyl, 2-Et-phenyl, 4-ⁱPr-phenyl, 3-ⁱPr-phenyl, 2-ⁱPr-phenyl, 4-F-phenyl, 3-F-phenyl, 2-F-phenyl, 4-Cl-phenyl, 3-Cl-phenyl, 2-Cl-phenyl, 4-Br-phenyl, 3-Br-phenyl, 2-Br-phenyl, 4-NH₂-phenyl, 3-NH₂-phenyl, 2-NH₂-phenyl, 4-CF₃-phenyl, 3-CF₃-phenyl, 2-CF₃-phenyl, 2,3-di-Me-phenyl, 2,4-di-Me-phenyl, 2,5-di-Me-phenyl, 2,6-di-Me-phenyl, 4-morpholino-phenyl, 3-morpholino-phenyl, 2-morpholino-phenyl, 4-CN-2-morpholino-phenyl, 4-CH₃-2-morpholino-phenyl, or 4-OH-2-morpholino-phenyl.

In embodiments of any formula described herein, a C₆-C₁₀ aryl is napthyl. In embodiments, a napthyl is unsubstituted napthyl. In embodiments, a napthyl is substituted napthyl (e.g., a napthyl comprising 1, 2, 3, 4, or 5 substituent groups including exemplary substituent groups described herein). In embodiments, a naphthyl is attached to a molecule at the C1-position (a 1-naphthyl). In embodiments, a naphthyl is attached to a molecule at the C2-position (a 2-naphthyl). In embodiments, a naphthyl is attached to a molecule at the C3-position (a 3-naphthyl). In embodiments, a naphthyl is attached to a molecule at the C4-position (a 4-naphthyl). In embodiments, a naphthyl is attached to a molecule at the C5-position (a 5-naphthyl). In embodiments, a naphthyl is attached to a molecule at the C6-position (a 6-naphthyl). In embodiments, a naphthyl is attached to a molecule at the C7-position (a 7-naphthyl). In embodiments, a naphthyl is attached to a molecule at the C8-position (an 8-naphthyl). In embodiments, a substituted naphthyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of napthyl are described herein.

In embodiments of any formula described herein, a 5- to 10-membered heteroaryl is imidazolyl. In embodiments, an imidazolyl is unsubstituted imidazolyl. In embodiments, an imidazolyl is substituted imidazolyl (e.g., an imidazolyl comprising 1, 2, or 3 substituent groups including exemplary substituent groups described herein). In embodiments, an imidazolyl is an N-linked imdazolyl and is attached to a molecule via the N1 position of the imidazolyl (a 1-imidazolyl). In embodiments, an imidazolyl is an C-linked imdazolyl. In embodiments, an imidazolyl is attached to a molecule via the C2 position of the imidazolyl group (a 2-imidazolyl). In embodiments, an imidazolyl is attached to a molecule via the C4 position of the imidazolyl group (a 4-imidazolyl). In embodiments, an imidazolyl is attached to a molecule via the C5 position of the imidazolyl group (a 5-imidazolyl). In embodiments, a substituted imidazolyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. In embodiments, an substituted imidazolyl is N-methylimidazolyl. Still other exemplary embodiments of imidazolyl are described herein.

In embodiments of any formula described herein, a 5- to 10-membered heteroaryl is pyrrolyl. In embodiments, a pyrrolyl is unsubstituted pyrrolyl. In embodiments, a pyrrolyl is an N-linked pyrrolyl and is attached to a molecule via the N1 position of the pyrrolyl (a 1-pyrrolyl). In embodiments, a pyrrolyl is an C-linked pyrrolyl. In embodiments, a pyrrolyl is attached to a molecule via the C2 position of the pyrrolyl (a 2-pyrrolyl). In embodiments, a pyrrolyl is attached to a molecule via the C3 position of the pyrrolyl (a 3-pyrrolyl). In embodiments, a pyrrolyl is attached to a molecule via the C4 position of the pyrrolyl (a 4-pyrrolyl). In embodiments, a pyrrolyl is attached to a molecule via the C5 position of the pyrrolyl (a 5-pyrrolyl). In embodiments, a pyrrolyl is substituted pyrrolyl (e.g., a pyrrolyl comprising 1, 2, or 3 substituent groups including exemplary substituent groups described herein). In embodiments, a substituted pyrrolyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of pyrrolyl are described herein.

In embodiments of any formula described herein, a 5- to 10-membered heteroaryl is oxazolyl. In embodiments, an oxazolyl is unsubstituted oxazolyl. In embodiments, an oxazolyl is attached to a molecule via the C2 position of the oxazolyl (a 2-oxazolyl). In embodiments, an oxazolyl is attached to a molecule via the C3 position of the oxazolyl (a 3-oxazolyl). In embodiments, an oxazolyl is attached to a molecule via the C4 position of the oxazolyl (a 4-oxazolyl). In embodiments, an oxazolyl is substituted oxazolyl (e.g., an oxazolyl comprising 1 or 2 substituent groups including exemplary substituent groups described herein). In embodiments, a substituted oxazolyl comprises 1 or 2 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of imidazolyl are described herein.

In embodiments of any formula described herein, a 5- to 10-membered heteroaryl is tetrazolyl. In embodiments, a tetrazolyl is unsubstituted tetrazolyl. In embodiments, a tetrazolyl is substituted tetrazolyl (e.g., an N-substituted tetrazolyl including exemplary substituent groups described herein). Still other exemplary embodiments of tetrazolyl are described herein.

In embodiments of any formula described herein, a 5- to 10-membered heteroaryl is pyridyl. In embodiments, a pyridyl is unsubstituted pyridyl. In embodiments, a pyridyl is attached to a molecule via the C2 position (a 2-pyridyl). In embodiments, a pyridyl is attached to a molecule via the C3 position (a 3-pyridyl). In embodiments, a pyridyl is attached to a molecule via the C4 position (a 4-pyridyl). In embodiments, a pyridyl is attached to a molecule via the C2 position (a 5-pyridyl). In embodiments, a pyridyl is attached to a molecule via the C2 position (a 6-pyridyl). In embodiments, a pyridyl is substituted pyridyl (e.g., a pyridyl comprising 1, 2, 3, or 4 substituent groups including exemplary substituent groups described herein). In embodiments, a substituted pyridyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of pyridyl are described herein.

In embodiments of any formula described herein, a 5- to 10-membered heteroaryl is pyrazinyl. In embodiments, a pyrazinyl is unsubstituted pyrazinyl. In embodiments, a pyrazinyl is a 2-pyrazinyl. In embodiments, a pyrazinyl is a 3-pyrazinyl. In embodiments, a pyrazinyl is a 5-pyrazinyl. In embodiments, a pyrazinyl is a 6-pyrazinyl. In embodiments, a pyrazinyl is substituted pyrazinyl (e.g., a pyrazinyl comprising 1, 2, 3, or 4 substituent groups including exemplary substituent groups described herein). In embodiments, a substituted pyrazinyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of pyrazinyl are described herein.

In embodiments of any formula described herein, a 5- to 10-membered heteroaryl is indolyl. In embodiments, an indolyl is unsubstituted indolyl. In embodiments, an indolyl is an N-linked indolyl and is attached to a molecule via the N1 position of the indolyl (a 1-indolyl). In embodiments, an indolyl is an C-linked indolyl. In embodiments, an indolyl is attached to a molecule via the C2 position (a 2-indolyl). In embodiments, an indolyl is attached to a molecule via the C3 position (a 3-indolyl). In embodiments, an indolyl is attached to a molecule via the C4 position (a 4-indolyl). In embodiments, an indolyl is attached to a molecule via the C5 position (a 5-indolyl). In embodiments, an indolyl is attached to a molecule via the C6 position (a 6-indolyl). In embodiments, an indolyl is attached to a molecule via the C7 position (a 7-indolyl). In embodiments, an indolyl is substituted indolyl (e.g., an indolyl comprising 1, 2, 3, or 4 substituent groups including exemplary substituent groups described herein). In embodiments, a substituted indolyl comprises 1, 2, or 3 substituents selected from the group consisting of OH, OCH₃, NH₂, CN, CH₃, CF₃, CH₂CH₃, isopropyl, F, Cl, Br, morpholino, CO₂H, CO₂CH₃, and CO₂NH₂. Still other exemplary embodiments of indolyl are described herein.

In embodiments of any formula described herein, substituents groups are selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen. In embodiments, a substituent group is itself unsubstituted. In embodiments, substituent groups are selected from the group consisting of OH, OCH₃, NH₂, CN, CH₃, CF₃, CH₂CH₃, isopropyl, F, Cl, Br, morpholino, CO₂H, CO₂CH₃, and CO₂NH₂.

In embodiments of any formula described herein, the C5 carbon of the 2-dihydrofuranone core has the (R)-configuration.

In embodiments of any formula described herein, the C5 carbon of the 2-dihydrofuranone core has the (S)-configuration.

In embodiments of any formula described herein, the carbon substituted by R^{A} or R^{AA} has the (R)-configuration.

In embodiments of any formula described herein, the carbon substituted by R^{A} or R^{AA} has the (S)-configuration.

Compounds of Formula (I*) and (I**)

Described herein are compounds of Formula (I*) along with exemplary embodiments of Formula (I*).

The exemplary formulas and compounds described herein can also encompass hydrates, solvates, enantiomers, diastereomers, pharmaceutically acceptable salts, and complexes thereof.

In one aspect, the present invention features a compound having a structure according to Formula (I*)

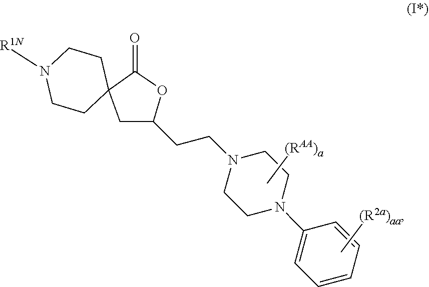

including enantiomers, diastereomers, hydrates, solvates, pharmaceutically acceptable salts, prodrugs and complexes thereof, wherein:
R^{1N} is selected from the group consisting of imidazole, oxazole, isoxazole,

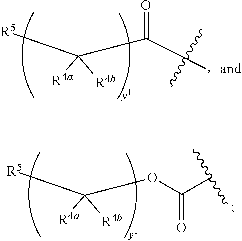

herein
each R^{4a} and R^{4b} is hydrogen or C₁-C₇ alkyl; or R^{4a} and R^{4b} optionally are taken together with the atoms to which they are bound to form a ring containing 3 to 7 atoms, optionally containing oxygen;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and

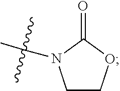 [SMILES: *N1CCOC1=O]

each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
each R^{AA} is independently C₁-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
a is 0, 1, or 2;
aa is 0, 1, or 2; and
y¹ is 0, 1 or 2.

In another aspect, the present invention features a compound having a structure according to Formula (I*-N)

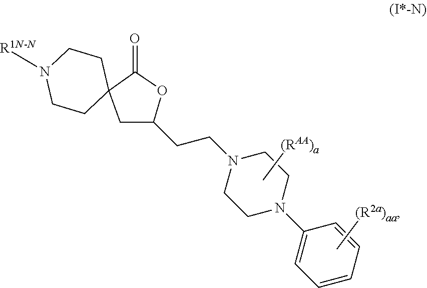

including enantiomers, diastereomers, hydrates, solvates, pharmaceutically acceptable salts, prodrugs and complexes thereof, wherein:
R^{1N-N} is selected from the group consisting of C₆-C₁₀ heteroaryl five- to ten-membered heteroaryl,

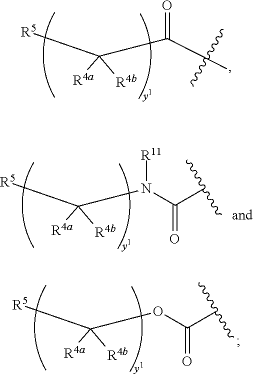

wherein
each R^{4a} and R^{4b} is hydrogen or C₁-C₇ alkyl; or R^{4a} and R^{4b} optionally are taken together with the atoms to which they are bound to form a ring containing 3 to 7 atoms, optionally containing oxygen;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and

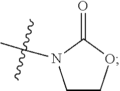 [SMILES: *N1CCOC1=O]

each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
each R^{AA} is independently C₁-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
a is 0, 1, or 2;
aa is 0, 1, or 2;
y¹ is 0, 1 or 2; and
wherein when R⁵ is unsubstituted C₁-C₇ alkyl or unsubstituted C₃-C₇ cycloalkyl, then a is 1 or 2.

In embodiments, R⁵ is unsubstituted C₁-C₇ alkyl or unsubstituted C₃-C₇ cycloalkyl, then a is 1 or 2.

In embodiments, a compound according to Formula (I*) has a structure according to the following formula,

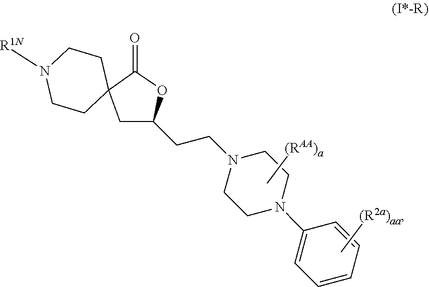

where R^{1N}, R^{AA}, R², aa, and a are according to any aspect or embodiment as described herein.

In embodiments, a compound according to Formula (I*) has a structure according to the following formula,

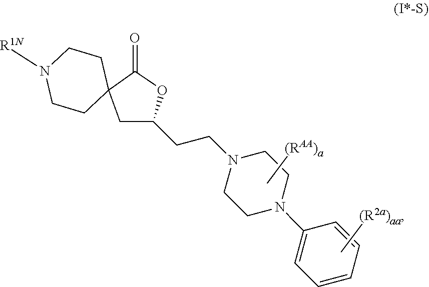

where R^{1N}, R^{AA}, R², aa, and a are according to any aspect or embodiment as described herein.

In embodiments, a compound according to Formula (I*-N) has a structure according to the following formula,

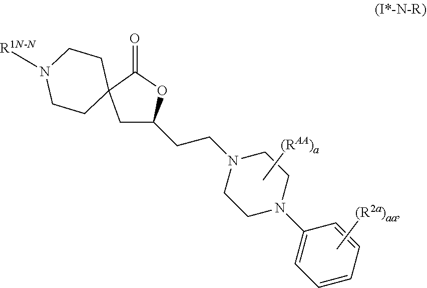

where R^{1N-N}, R^{AA}, R^{2a}, aa, and a are according to any aspect or embodiment as described herein.

In embodiments, a compound according to Formula (I*-N) has a structure according to the following formula,

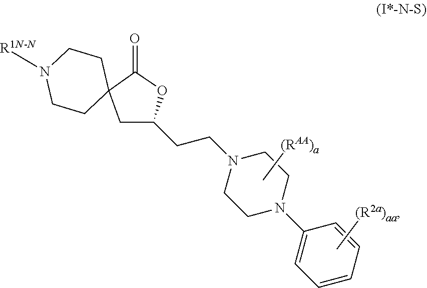

where R^{1N-N}, R^{AA}, R^{2a}, aa, and a are according to any aspect or embodiment as described herein.

In embodiments, each R^{AA} is independently C₁-C₇ linear alkyl. In embodiments, each R^{AA} is independently methyl.

In embodiments, a is 0. In embodiments, a is 1. In embodiments, a is 2. In embodiments, a is not 0. In embodiments, a excludes 0. In embodiments, a is 0 or 1. In embodiments, a is 1 or 2.

In embodiments, each R^{2a} is independently halogen. In embodiments, each R^{2a} is independently F. In embodiments, each R^{2a} is independently Cl.

In embodiments, aa is 0. In embodiments, aa is 1. In embodiments, aa is 2. In embodiments, aa is 1 or 2.

In embodiments, R^{1N} is selected from the group consisting of imidazole, oxazole, isoxazole,

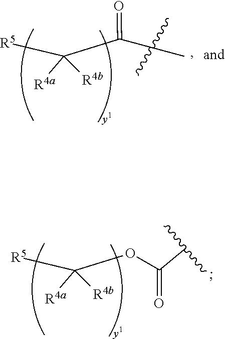

wherein
each R^{4a} and R^{4b} is hydrogen or C₁-C₇ alkyl; or R^{4a} and R^{4b} optionally are taken together with the atoms to which they are bound to form a ring containing 3 to 7 atoms, optionally containing oxygen;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and

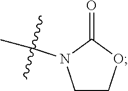 [SMILES: *N1CCOC1=O]

each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl; and
y¹ is 0, 1 or 2.

In embodiments, R^{1N-N} is selected from the group consisting of C₆-C₁₀ heteroaryl, five- to ten-membered heteroaryl,

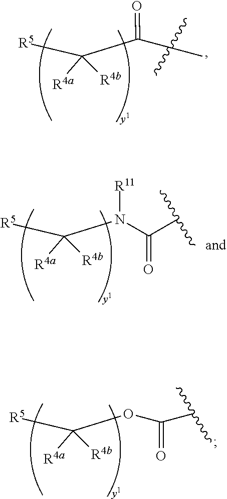

wherein
each R^{4a} and R^{4b} is hydrogen or C₁-C₇ alkyl; or R^{4a} and R^{4b} optionally are taken together with the atoms to which they are bound to form a ring containing 3 to 7 atoms, optionally containing oxygen;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and

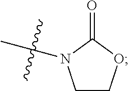 [SMILES: *N1CCOC1=O]

each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
R¹¹ is selected from the group consisting of hydrogen, C₁-C₇ alkyl, and C₃-C₇ cycloalkyl; and
y¹ is 0, 1 or 2.

In embodiments, R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8e}, NR^{8d}SO₂R^{8e}, NR⁸ⁱCOOR^{8j}, NHCONR^{8f}, NR^{8g}COR^{8h} and

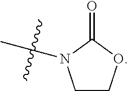 [SMILES: *N1CCOC1=O]

In embodiments, R⁵ excludes unsubstituted C₁-C₇ alkyl. In embodiments, R⁵ excludes unsubstituted C₃-C₇ cycloalkyl.

In embodiments, R^{1N} is

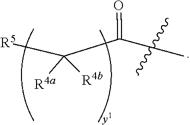

In embodiments, R^{1N-N} is

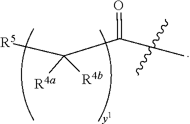

In embodiments, y¹ is 0. In embodiments, y¹ is 1. In embodiments, y¹ is 2.

In embodiments, R^{1N} is

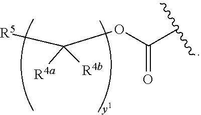

In embodiments, R^{1N-N} is

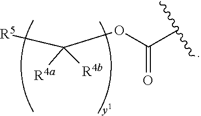

In embodiments, y¹ is 0. In embodiments, y¹ is 1. In embodiments, y¹ is 2.

In embodiments, R^{1N-N} is

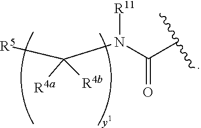

In embodiments, y¹ is 0. In embodiments, y¹ is 1. In embodiments, y¹ is 2.

In embodiments, y¹ is 0, and R¹ is COR⁵. In embodiments, R⁵ is pyridyl. In embodiments, R⁵ is pyridazine. In embodiments, R⁵ is C₁-C₇ alkyl. In embodiments, R⁵ is C₃-C₇ cycloalkyl. In embodiments, R⁵ is C₁-C₇ haloalkyl. In embodiments, R⁵ is C₃-C₇ cyclohaloalkyl. In embodiments, R⁵ is C₁-C₇ fluoroalkyl. In embodiments, R⁵ is C₃-C₇ cyclofluoroalkyl.

In embodiments, y¹ is 0. In embodiments, y¹ is 1. In embodiments, y¹ is 2.

In embodiments, R^{4a} is H. In embodiments, R^{4b} is H. In embodiments, R^{4a} and R^{4b} are both H. In embodiments, y¹ is 0. In embodiments, y¹ is 1. In embodiments, y¹ is 2.

In embodiments, R⁵ is pyridyl. In embodiments, R⁵ is pyridazine. In embodiments, R⁵ is C₁-C₇ alkyl. In embodiments, R⁵ is C₃-C₇ cycloalkyl. In embodiments, R⁵ is C₁-C₇ haloalkyl. In embodiments, R⁵ is C₃-C₇ cyclohaloalkyl. In embodiments, R⁵ is C₁-C₇ fluoroalkyl. In embodiments, R⁵ is C₃-C₇ cyclofluoroalkyl. In embodiments, R⁵ is unsubstituted C₁-C₇ alkyl. In embodiments, R⁵ is substituted C₁-C₇ alkyl (e.g., comprising an amino substituent such as —NH₂, —NHCH₃, or —N(CH₃)₂). In embodiments, R⁵ is phenyl. In embodiments, R⁵ is unsubstituted phenyl. In embodiments, R⁵ is substituted phenyl. In embodiments, R⁵ is NR^{8a}R^{8b}. In embodiments, R⁵ is SO₂R^{8c}. In embodiments, R⁵ is NR^{8d}SO₂R^{8e}. In embodiments, R⁵ is NR⁸ⁱCOOR^{8j}. In embodiments, R⁵ is NHCONR^{8f}. In embodiments, R⁵ is NR^{8g}COR^{8h}. In embodiments, R⁵ is not unsubstituted C₁-C₇ alkyl.

In embodiments, R^{1′} is hydrogen. In embodiments, R^{1′} is C₁-C₇ alkyl (e.g. methyl). In embodiments, R^{1′} is C₃-C₇ cycloalkyl.

In embodiments, R^{1N} or R^{1N-N} is

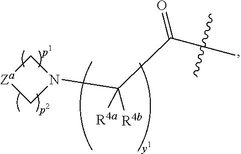

wherein

R^{4a}, R^{4b}, and y¹ are according to any aspect or embodiment described herein;

Z^{a} is CH₂ or 0;

when Z^{a} is CH₂, p¹+p² is 1, 2, 3, or 4; and

when Z^{a} is O, p¹+p² is 1, 2, 3, or 4; and both p¹ and p² are not 0.

In embodiments, R^{4a} and R^{4b} are taken together with the atoms to which they are bound to form a carbocyclic ring containing 3 to 7 atoms. In embodiments, R^{4a} and R^{4b} are taken together with the atoms to which they are bound to form a oxygen-containing ring containing 3 to 7 atoms.

In embodiments, R^{1N} or R^{1N-N} is

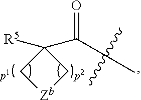

wherein
Z^{b} is CH₂ or 0;
when Z^{b} is CH₂, p¹+p² is 1, 2, 3, or 4;
when Z^{b} is O, p¹+p² is 1, 2, 3, or 4; and both p¹ and p² are not 0;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and

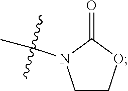 [SMILES: *N1CCOC1=O]

each R^{8a}, R^{8b}, R^{8d}, R^{8g}, R⁸ⁱ and R⁹ is selected from the group consisting of hydrogen, C₁-C₇ alkyl, and C₃-C₇ cycloalkyl;
R^{8a} and R^{8b} optionally are taken together with the atoms to which they are bound to form a heterocyle containing 3 to 7 atoms, optionally containing a group selected from oxygen, sulfur, and NR⁹;
R^{8j} is selected from the group consisting of C₁-C₇ alkyl, C₃-C₇ cycloalkyl, C₆-C₁₀ aryl, and 5- to 10-membered heteroaryl;
each R^{8c}, R^{8e}, R^{8f} and R^{8h} is C₃-C₇ alkyl or C₃-C₇ cycloalkyl.

In embodiments, R^{1N} or R^{1N-N} is

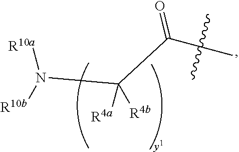

wherein
R^{4a}, R^{4b}, and y¹ are according to any aspect or embodiment described herein;
R^{10a} and R^{10b} is independently selected from the group consisting of H, C₁-C₇ linear alkyl, C₃-C₇ branched alkyl, C₃-C₇ cycloalkyl, SO₂R^{8e}, COOR^{8j}, CONR^{8f}, and COR^{8h}; and
at least one of R^{10a} and R^{10b} is selected from the group consisting of H, C₁-C₇ linear alkyl, C₃-C₇ branched alkyl, and C₃-C₇ cycloalkyl;
each R^{8e}, R^{8f} and R^{8h} is selected from the group consisting of H, C₁-C₇ linear alkyl, C₃-C₇ branched alkyl, C₃-C₇ cycloalkyl.

In embodiments, R^{1N-N} is a urea group

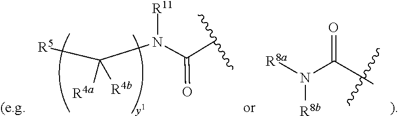

In embodiments, R^{1N} or R^{1N-N} is a carbamate group

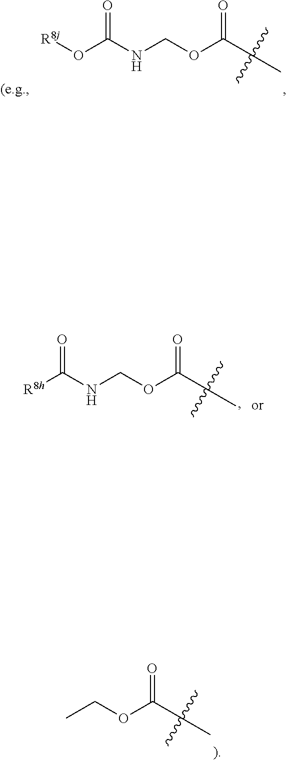

In embodiments, R^{1N} is an aminoacyl group

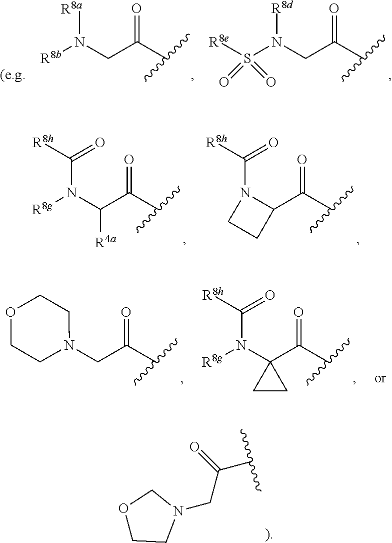

In embodiments, R^{1N} is an alkylacyl group

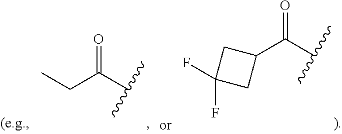

In embodiments, R^{1N} or R^{1N-N} is an aryl. In embodiments, R^{1N} is a heteroaryl

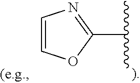 [SMILES: *c1ncco1]

In embodiments, R^{1N} is a heteroaryl containing acyl group

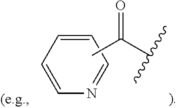

In embodiments, R^{1N} or R^{1N-N} is

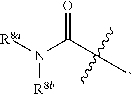

wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl.

In embodiments, R^{1N} or R^{1N-N} is

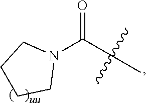

wherein uu is 1 or 2.

In embodiments, R^{1N} or R^{1N-N} is

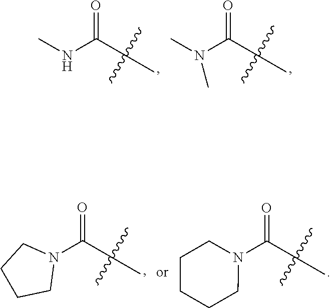

In embodiments, R^{1N} or R^{1N-N} is

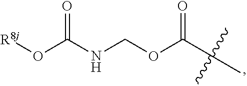

wherein R^{8j} is selected from the group consisting of C₁-C₇ alkyl, C₃-C₇ cycloalkyl, C₆-C₁₀ aryl, and 5- to 10-membered heteroaryl.

In embodiments, R^{1N} or R^{1N-N} is

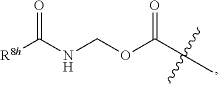

wherein R^{8h} is unsubstituted C₁-C₇ alkyl.

In embodiments, R^{1N} or R^{1N-N} is

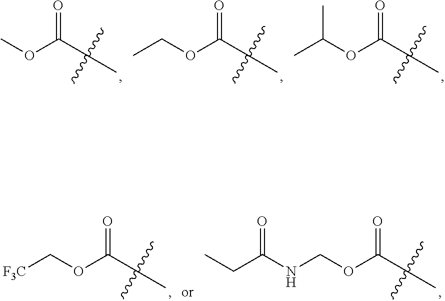

In embodiments, R^{1N} or R^{1N-N} is

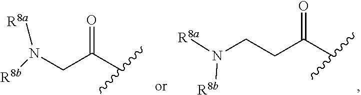

wherein each R^{8a} and R^{8b} is independently H or unsubstituted C₁-C₇ alkyl.

In embodiments, R^{1N} or R^{1N-N} is

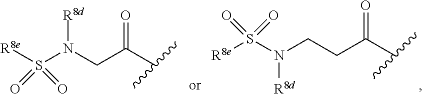

wherein R^{8d} is independently H or unsubstituted C₁-C₇ alkyl, and R^{8e} is unsubstituted C₁-C₇ alkyl.

In embodiments, R^{1N} or R^{1N-N} is

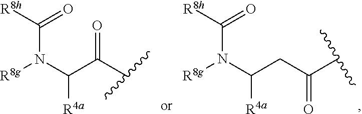

wherein each of R^{4a} and R^{8g} is independently H or unsubstituted C₁-C₇ alkyl; and R^{8h} is unsubstituted C₁-C₇ alkyl.

In embodiments, R^{1N} or R^{1N-N} is

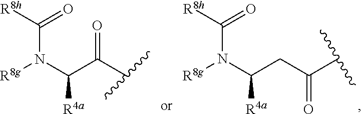

wherein each of R^{4a} and R^{8g} is independently H or unsubstituted C₁-C₇ alkyl; and R^{8h} is unsubstituted C₁-C₇ alkyl.

In embodiments, R^{1N} or R^{1N-N} is

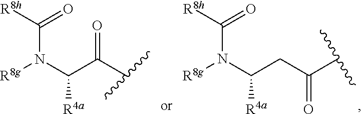

wherein each of R^{4a} and R^{8g} is independently H or unsubstituted C₁-C₇ alkyl; and R^{8h} is unsubstituted C₁-C₇ alkyl.

In embodiments, R^{1N} or R^{1N-N} is

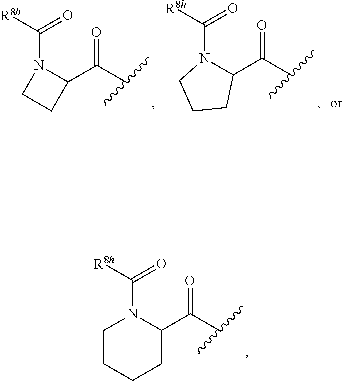

wherein R^{8h} is unsubstituted C₁-C₇ alkyl.

In embodiments, R^{1N} or R^{1N-N} is

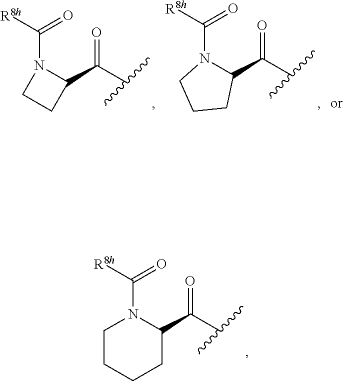

wherein R^{8h} is unsubstituted C₁-C₇ alkyl.

In embodiments, R^{1N} or R^{1N-N} is

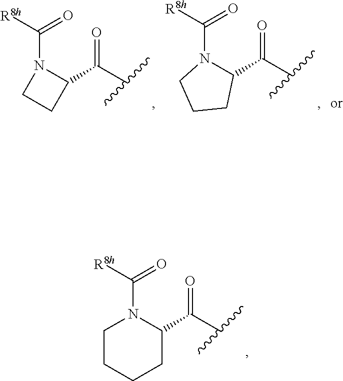

wherein R^{8h} is unsubstituted C₁-C₇ alkyl.

In embodiments, R^{1N} or R^{1N-N} is

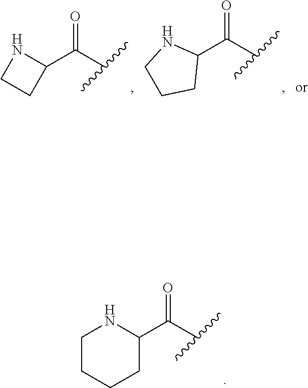

In embodiments, R^{1N} or R^{1N-N} is or

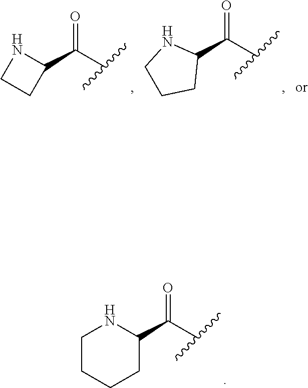

In embodiments, R^{1N} or R^{1N-N} is

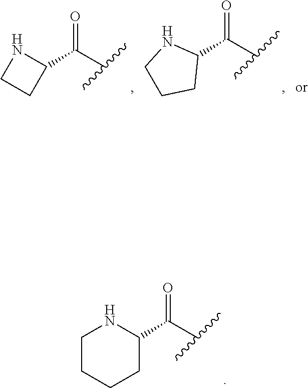

In embodiments, R^{1N} or R^{1N-N} is

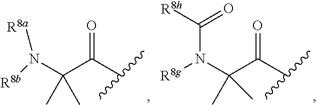

wherein each R^{8a}, R^{8b}, and R^{8g} is independently H or unsubstituted C₁-C₇ alkyl, and R^{8h} is unsubstituted C₃-C₇ alkyl.

In embodiments, R^{1N} or R^{1N-N} is

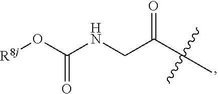

wherein R^{8j} is selected from the group consisting of C₁-C₇ alkyl, C₃-C₇ cycloalkyl, C₆-C₁₀ aryl, and 5- to 10-membered heteroaryl.

In embodiments, R^{1N} or R^{1N-N} is

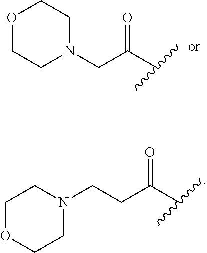 [SMILES: *C(=O)CN1CCOCC1 ; *C(=O)CCN1CCOCC1]

In embodiments, R^{1N} or R^{1N-N} is

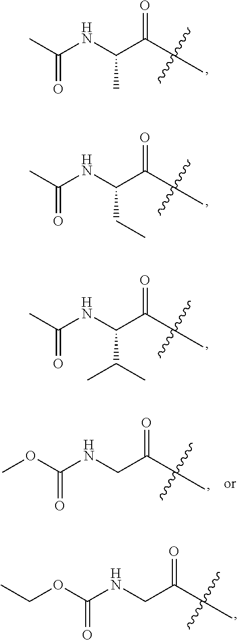

In embodiments, R^{1N} or R^{1N-N} is

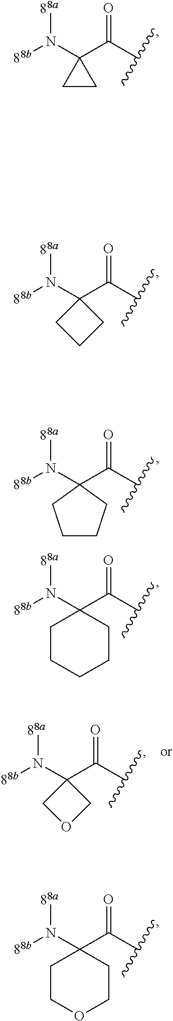

wherein each R^{8a} and R^{8b} is independently H or unsubstituted C₁-C₇ alkyl.

In embodiments, R^{1N} or R^{1N-N} is

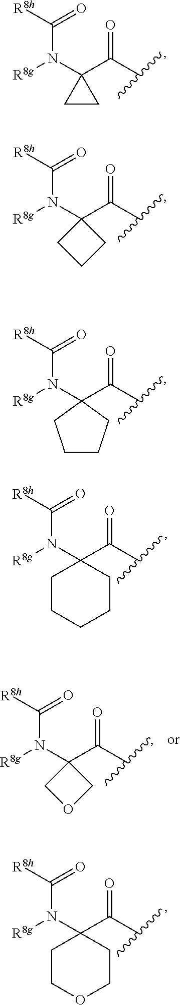

wherein R^{8g} is independently H or unsubstituted C₁-C₇ alkyl, and R^{8h} is independently unsubstituted C₁-C₇ alkyl.

In embodiments, R^{1N} or R^{1N-N} is

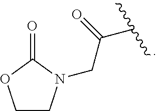 [SMILES: *C(=O)CN1CCOC1=O]

In embodiments, R^{1N} or R^{1N-N} is

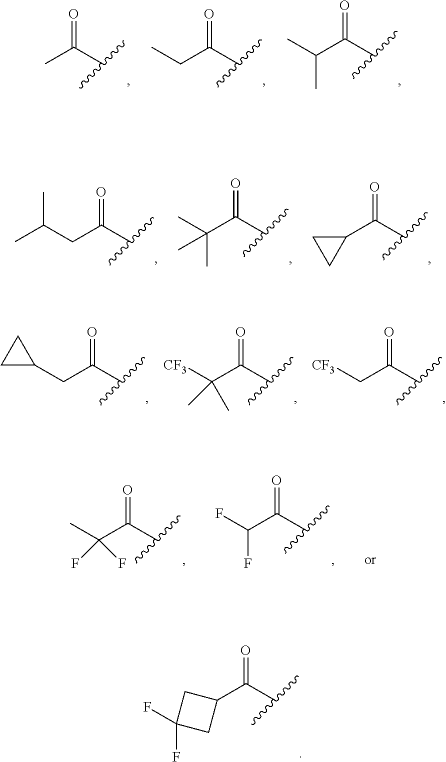

In embodiments, R^{1N} or R^{1N-N} is

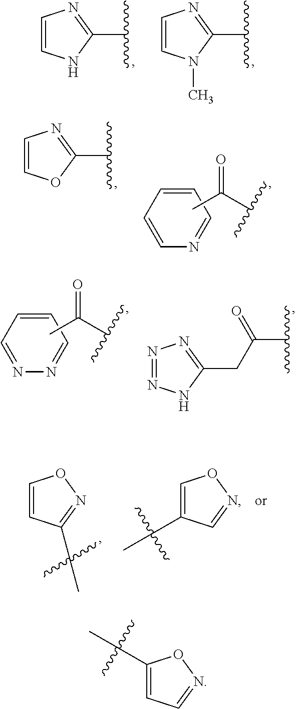

In embodiments, a compound according to Formula (I*) or (I*-N) has the following structure,

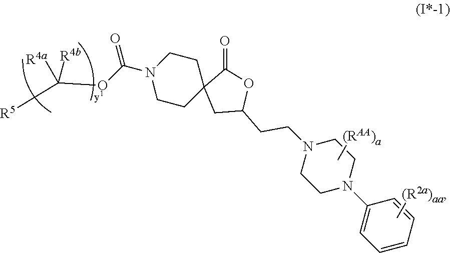

wherein each R⁵, R^{4a}, R^{4b}, R^{2a}, R^{AA}, y¹, aa, and a is according to any aspect or embodiment as described herein.

In embodiments, a compound according to Formula (I*) or (I*-N) has the following structure,

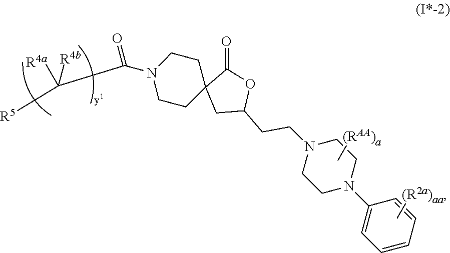

wherein each R⁵, R^{4a}, R^{4b}, R^{2a}, R^{AA}, y¹, aa, and a is according to any aspect or embodiment as described herein.

In embodiments, a compound according to Formula (I*-N) has the following structure,

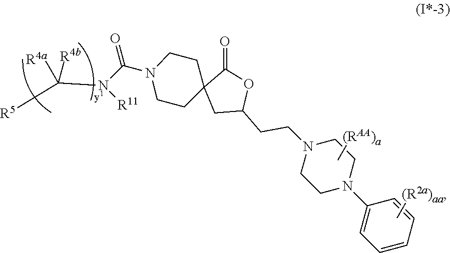

(I*-3), wherein each R⁵, R¹¹, R^{4a}, R^{4b}, R^{2a}, R^{AA}, y¹, aa, and a is according to any aspect or embodiment as described herein.

In embodiments, a compound according to Formula (I*-1), (I*-2) or (I*-3) has the one of the following structures,

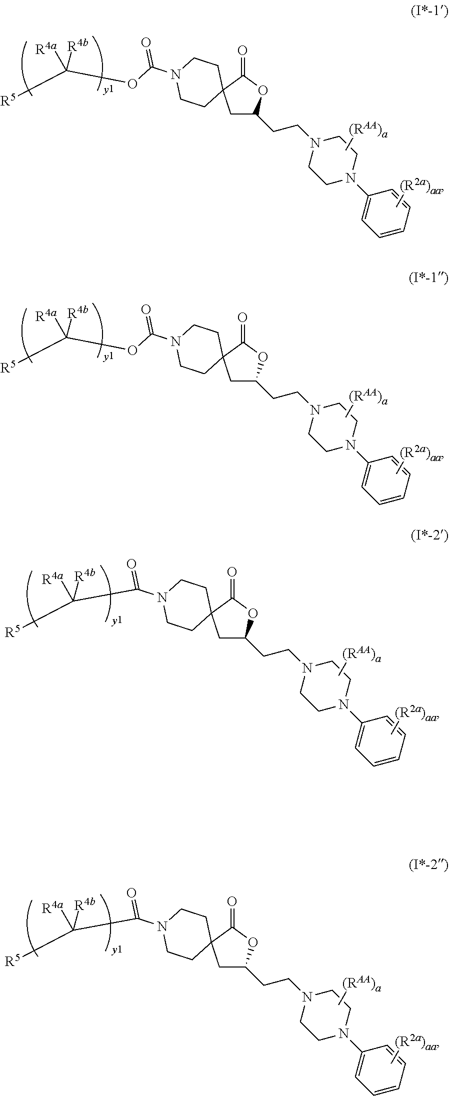

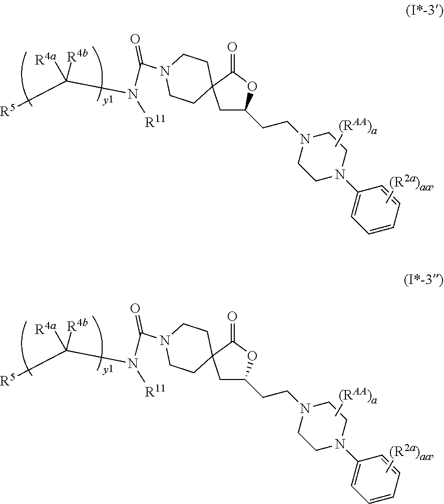

wherein
each R⁵, R¹¹, R^{4a}, R^{4b}, R^{2a}, R^{AA}, y¹, aa, and a is according to any aspect or embodiment as described herein.

In another aspect, the present invention features a compound having a structure according to Formula (I**)

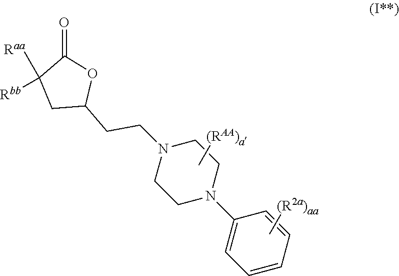

including enantiomers, diastereomers, hydrates, solvates, pharmaceutically acceptable salts, prodrugs and complexes thereof, wherein:
each R^{aa} and R^{bb} is selected from the group consisting of hydrogen, C₁-C₇ alkyl and C₃-C₇ branched alkyl;
each R^{AA} is independently C₁-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
a′ is 1, or 2; and
aa is 0, 1, or 2.

In embodiments, a compound according to Formula (I**) has a structure according to the following formula,

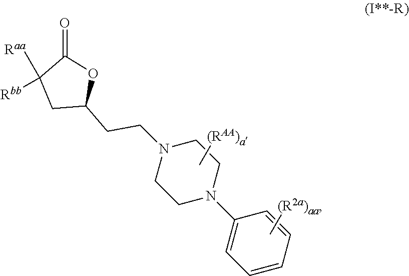

where R^{aa}, R^{bb}, R^{AA}, R^{2a}, aa, and a′ are according to any aspect or embodiment as described herein.

In embodiments, a compound according to Formula (I**) has a structure according to the following formula,

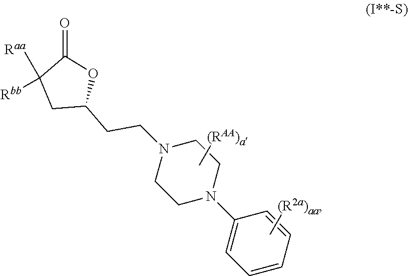

where R^{aa}, R^{bb}, R^{AA}, R^{2a}, aa, and a′ are according to any aspect or embodiment as described herein.

In embodiments, each R^{AA} is independently C₁-C₇ linear alkyl. In embodiments,
each R^{AA} is independently methyl.

In embodiments, a′ is 1. In embodiments, a′ is 2. In embodiments, a′ is 1 or 2.

In embodiments, each R^{2a} is independently halogen. In embodiments, each R^{2a} is independently F. In embodiments, each R^{2a} is independently Cl.

In embodiments, aa is 0. In embodiments, aa is 1. In embodiments, aa is 2. In embodiments, aa is 1 or 2.

In embodiments, R^{aa} is C₁-C₇ linear alkyl. In embodiments, R^{aa} is C₃-C₇ branched alkyl. In embodiments, R^{aa} is ethyl. In embodiments, R^{bb} is C₁-C₇ linear alkyl. In embodiments, R^{bb} is C₃-C₇ branched alkyl. In embodiments, R^{bb} is ethyl. In embodiments R^{aa} and R^{bb} are each ethyl.

Compounds of Formula (I)

In one aspect, the present invention features a compound having a structure according to Formula (I)

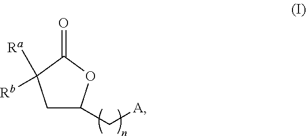

including enantiomers, diastereomers, hydrates, solvates, pharmaceutically acceptable salts, prodrugs and complexes thereof, wherein:

each R^{a} and R^{b} is selected from the group consisting hydrogen, C₁-C₇ alkyl, and C₃-C₇ branched alkyl; or R^{a} and R^{b} are taken together with the atoms to which they are bound to form a carbocyclic ring having from 5 to 7 ring atoms, optionally containing a double bond;

or R^{a} and R^{b} are taken together with the atoms to which they are bound to form a ring having from 6 to 8 ring atoms comprising a moiety selected from the group consisting of O, S, SO, SO₂, and NR¹;

A is an N-linked, five- to twelve-membered nitrogen-containing heterocyclyl, wherein said nitrogen-containing heterocyclyl is bicyclic or polycyclic and optionally includes further heteroatoms selected from O, N, and S, and wherein a non-aromatic, nitrogen-containing heterocyclyl further comprises a group R²;

R¹ is a H, C₁-C₇ alkyl, C₃-C₇ cycloalkyl, phenyl, benzyl, five- to six-membered heteroaryl ring, a polar acyl group, or a polar sulfonyl group;

R² is selected from the group consisting of 6- to 10-membered aryl, 5- to 10-membered nitrogen-containing heteroaryl, and

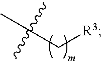

R³ is a 6- to 10-membered aryl or 5- to 10-membered nitrogen containing heteroaryl;

m is 1, 2, or 3; and

n is 1, 2, 3, or 4.

In embodiments, when A is 1,2,3,4-tetrahydroquinol-1-yl, 1,2,3,4-tetrahydroisoquinol-2-yl, octahydropyrrolo[3,4-c]pyrrol-1-yl, or 2,6-diazaspiro[3.3]heptan-1-yl, then R^{a} and R^{b} cannot both be methyl, both be ethyl, or both be phenyl nor can R^{a} and R^{b} combine to form unsubstituted C₃-C₆ cycloalkyl.

In embodiments, A is not 1,2,3,4-tetrahydroquinol-1-yl, 1,2,3,4-tetrahydroisoquinol-2-yl, octahydropyrrolo[3,4-c]pyrrol-1-yl, or 2,6-diazaspiro[3.3]heptan-1-yl. In embodiments, A excludes 1,2,3,4-tetrahydroquinol-1-yl, 1,2,3,4-tetrahydroisoquinol-2-yl, octahydropyrrolo[3,4-c]pyrrol-1-yl, or 2,6-diazaspiro[3.3]heptan-1-yl.

In embodiments, R^{a} and R^{b} are taken together with the atoms to which they are bound to form a ring having from 6 to 8 ring atoms, wherein one of the ring atoms is a moiety selected from the group consisting of O, S, SO, SO₂, and NR¹.

In embodiments, a compound according to Formula (I) has a structure according to the following formula,

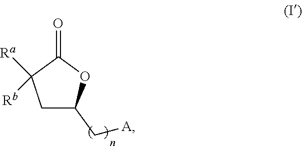

where R^{a}, R^{b}, A, and n are according to any aspect or embodiment as described herein.

In embodiments, a compound according to Formula (I) has a structure according to the following formula,

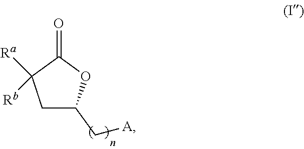

where R^{a}, R^{b}, A, and n are according to any aspect or embodiment as described herein.

In embodiments, each R^{a} and R^{b} is methyl.

In embodiments, each R^{a} and R^{b} is ethyl.

In embodiments, R^{a} and R^{b} combine to form unsubstituted cyclopropyl, cyclobutyl, cyclopentyl, or cyclohexyl. In embodiments, R^{a} and R^{b} combine to form unsubstituted cyclopropyl. In embodiments, R^{a} and R^{b} combine to form unsubstituted cyclobutyl. In embodiments, R^{a} and R^{b} combine to form unsubstituted cyclopentyl. In embodiments, R^{a} and R^{b} combine to form unsubstituted cyclohexyl.

In embodiments, R^{a} and R^{b} are taken together with the atoms to which they are bound to form a ring having from 6 to 8 ring atoms comprising a moiety that is NR¹.

In embodiments, R^{a} and R^{b} combine to form a group that is

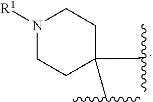

In embodiments, a compound according to Formula (I) has a structure according to the following formula,

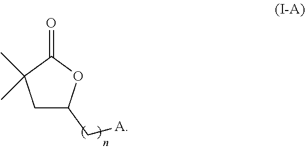

In embodiments, n is 1. In embodiments, n is 2. In embodiments, n is 3.

In embodiments, a compound according to Formula (I) has a structure according to the following formula,

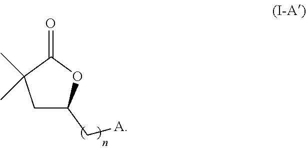

In embodiments, n is 1. In embodiments, n is 2. In embodiments, n is 3.

In embodiments, a compound according to Formula (I) has a structure according to the following formula,

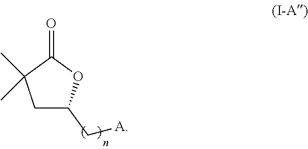

In embodiments, n is 1. In embodiments, n is 2. In embodiments, n is 3.

In embodiments, a compound according to Formula (I) has a structure according to the following formula,

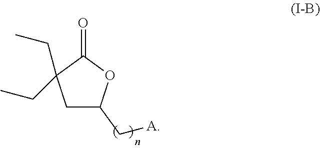

In embodiments, n is 1. In embodiments, n is 2. In embodiments, n is 3.

In embodiments, a compound according to Formula (I) has a structure according to the following formula,

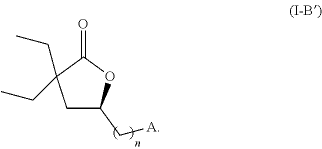

In embodiments, n is 1. In embodiments, n is 2. In embodiments, n is 3.

In embodiments, a compound according to Formula (I) has a structure according to the following formula,

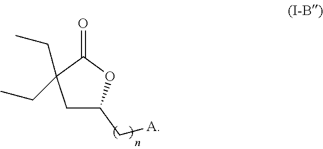

In embodiments, n is 1. In embodiments, n is 2. In embodiments, n is 3.

In embodiments, a compound according to Formula (I) has a structure according to the following formula.

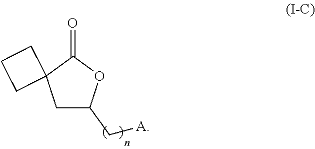

In embodiments, n is 1. In embodiments, n is 2. In embodiments, n is 3.

In embodiments, a compound according to Formula (I) has a structure according to the following formula,

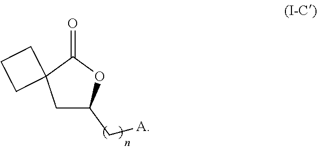

In embodiments, n is 1. In embodiments, n is 2. In embodiments, n is 3.

In embodiments, a compound according to Formula (I) has a structure according to the following formula,

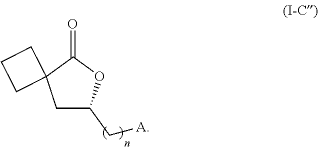

In embodiments, n is 1. In embodiments, n is 2. In embodiments, n is 3.

In embodiments, a compound according to Formula (I) has a structure according to the following formula,

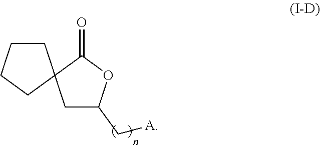

In embodiments, n is 1. In embodiments, n is 2. In embodiments, n is 3.

In embodiments, a compound according to Formula (I) has a structure according to the following formula,

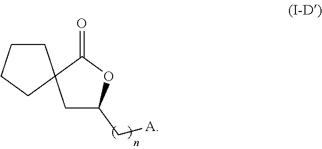

In embodiments, n is 1. In embodiments, n is 2. In embodiments, n is 3.

In embodiments, a compound according to Formula (I) has a structure according to the following formula,

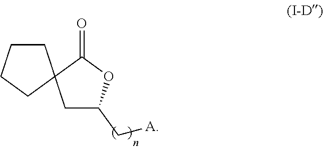

In embodiments, n is 1. In embodiments, n is 2. In embodiments, n is 3.

In embodiments, a compound according to Formula (I) has a structure according to the following formula,

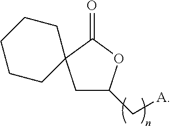

In embodiments, n is 1. In embodiments, n is 2. In embodiments, n is 3.

In embodiments, a compound according to Formula (I) has a structure according to the following formula,

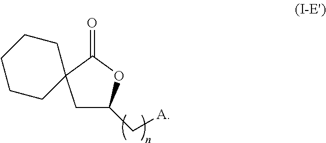

In embodiments, n is 1. In embodiments, n is 2. In embodiments, n is 3.

In embodiments, a compound according to Formula (I) has a structure according to the following formula,

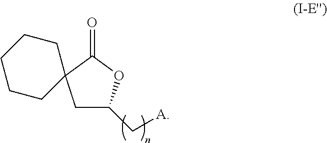

In embodiments, n is 1. In embodiments, n is 2. In embodiments, n is 3.

In embodiments, a compound according to Formula (I) has a structure according to the following formula,

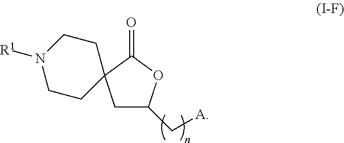

In embodiments, n is 1. In embodiments, n is 2. In embodiments, n is 3.

In embodiments, a compound according to Formula (I) has a structure according to the following formula,

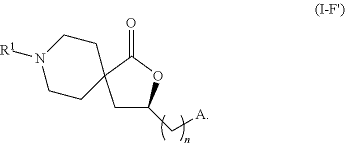

In embodiments, n is 1. In embodiments, n is 2. In embodiments, n is 3.

In embodiments, a compound according to Formula (I) has a structure according to the following formula,

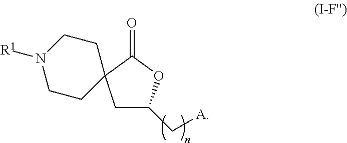

In embodiments, n is 1. In embodiments, n is 2. In embodiments, n is 3.

In embodiments, A is selected from the group consisting of

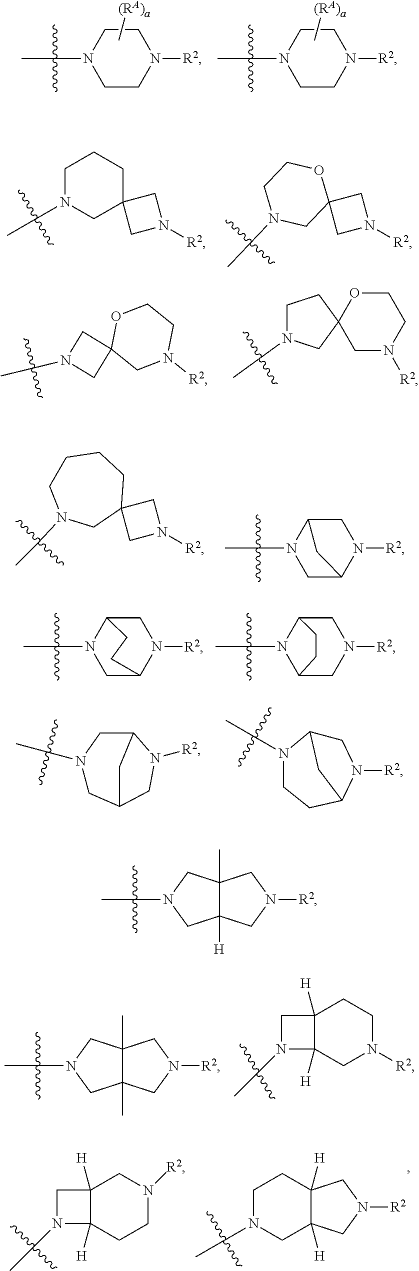

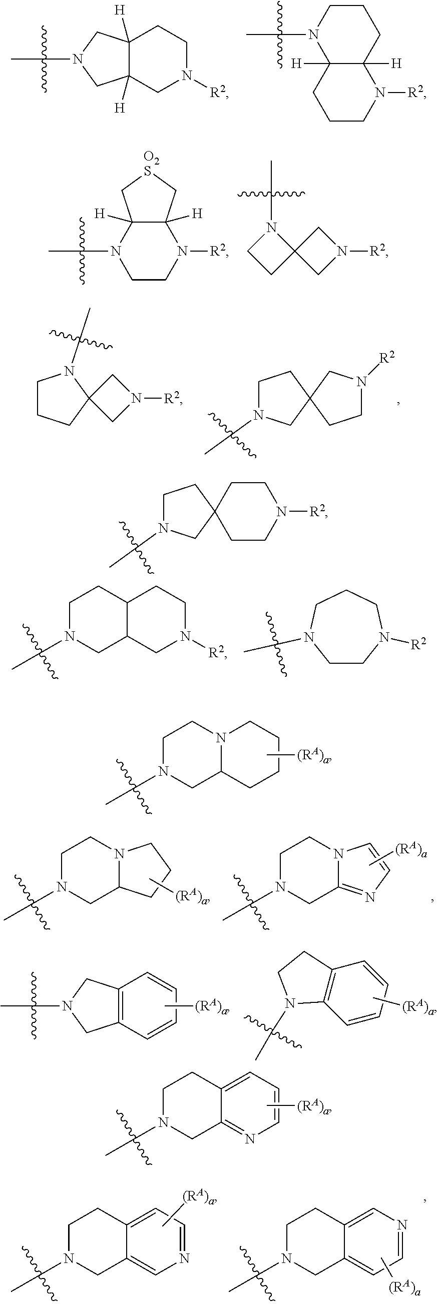

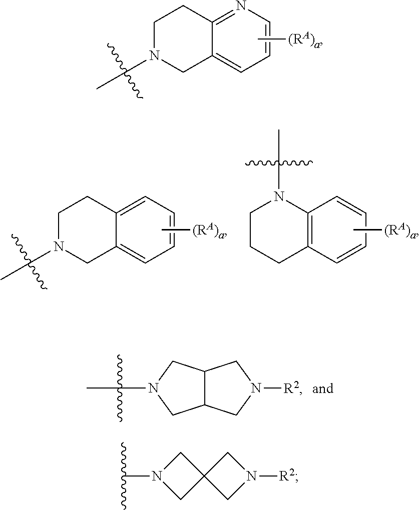

wherein

R² is selected from the group consisting of phenyl, naphthyl, pyridyl, indolyl and

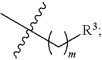

R³ is selected from the group consisting of phenyl, naphthyl, pyridyl and indolyl;

R^{A} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen; and

a is 0, 1, or 2.

In embodiments, a is 0.

In embodiments, a is 1.

In embodiments, a is 2.

In embodiments, A is selected from the group consisting of:

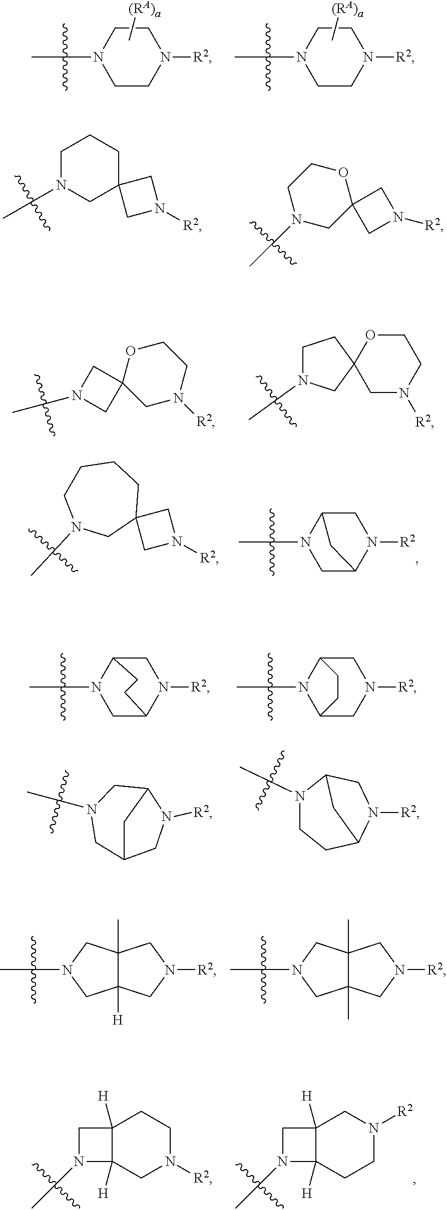

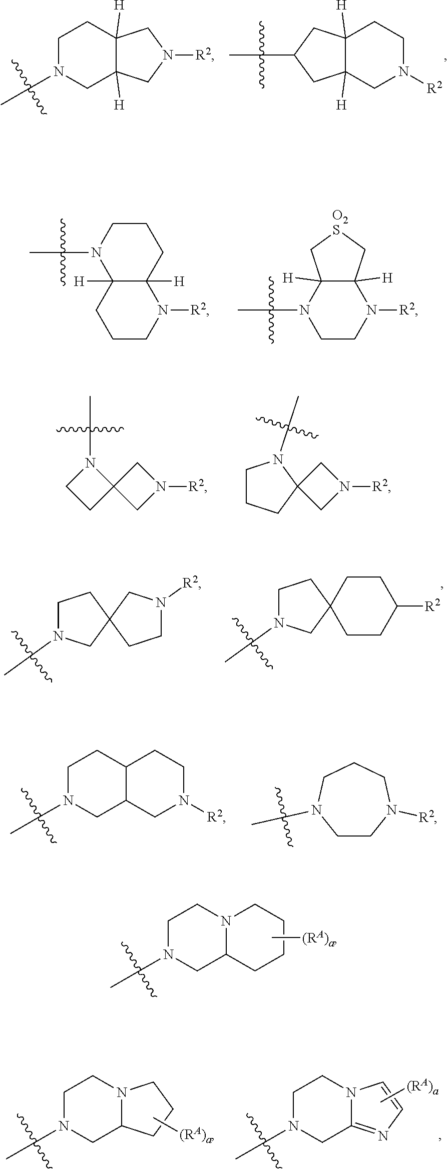

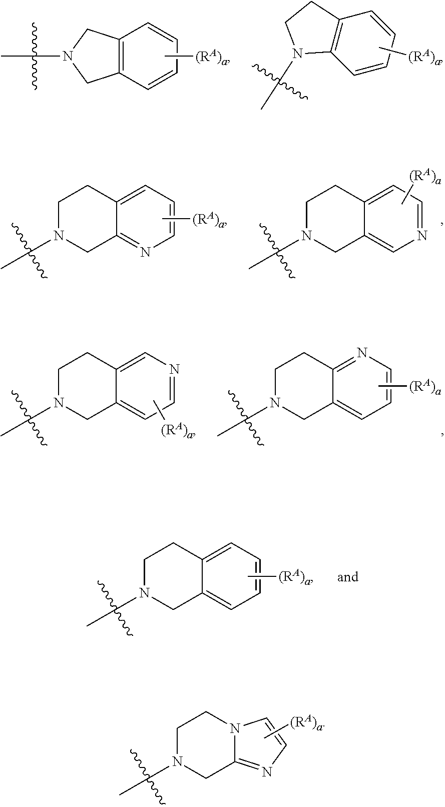

In embodiments, A is

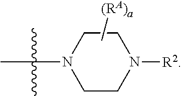

In embodiments, A is

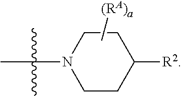

In embodiments, A is

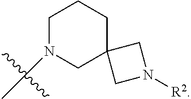

In embodiments, A is

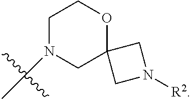

In embodiments, A is

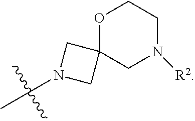

In embodiments, A is

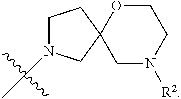

In embodiments, A is

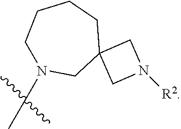

In embodiments, A is R²

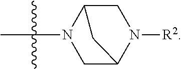

In embodiments, A is

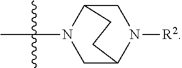

In embodiments, A is

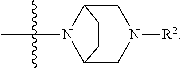

In embodiments, A is

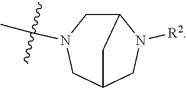

In embodiments, A is

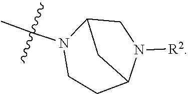

In embodiments, A is

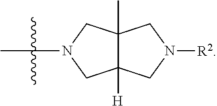

In embodiments, A is

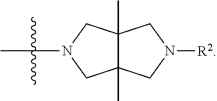

In embodiments, A is

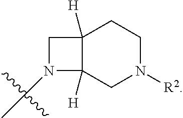

In embodiments, A is

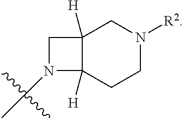

In embodiments, A is H

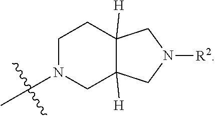

In embodiments, A is H

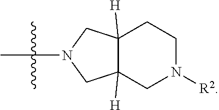

In embodiments, A is

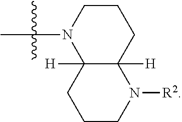

In embodiments, A is

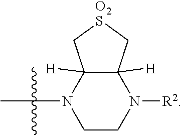

In embodiments, A is

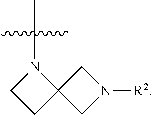

In embodiments, A is

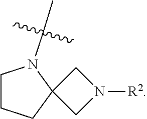

In embodiments, A is

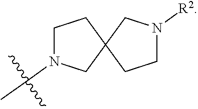

In embodiments, A is

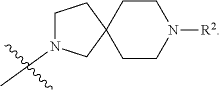

In embodiments, A is

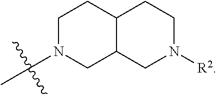

In embodiments, A is

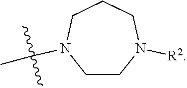

In embodiments, A is

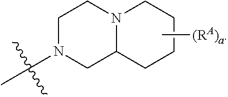

In embodiments, A is

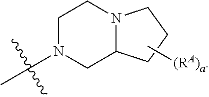

In embodiments, A is

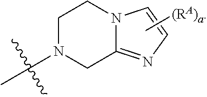

In embodiments, A is

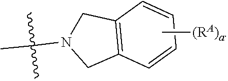

In embodiments, A is R

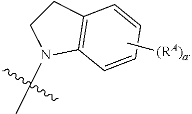

In embodiments, A is

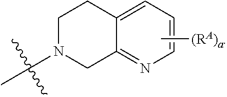

In embodiments, A is

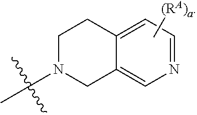

In embodiments, A is

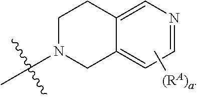

In embodiments, A is

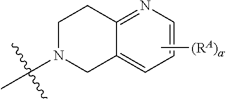

In embodiments of Formula (I), A is selected from the group consisting of:

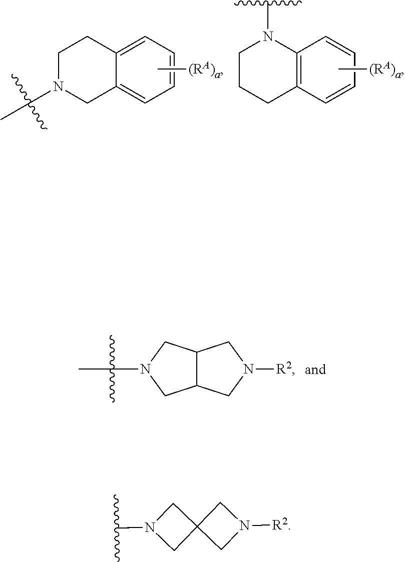

In embodiments, a is 0.

In embodiments, a is 1.

In embodiments, a is 2.

In embodiments, A is

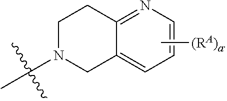

In embodiments, A is

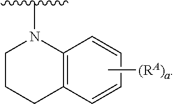

In embodiments, A is

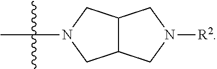

In embodiments, A is

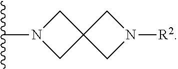

In embodiments, R² is phenyl. In embodiments, R² is unsubstituted phenyl. In embodiments, R² is phenyl comprising at least one halogen substitutent (e.g., at least one substituent that is chloro or fluoro. In embodiments, R² is fluorophenyl (e.g., 2-, 3-, or 4-fluorophenyl), difluorophenyl, chlorophenyl (e.g., 2-, 3-, or 4-chlorophenyl), dichlorophenyl, chlorofluorophenyl. In embodiments, R² is phenyl substituted by 1, 2, or 3 groups (e.g., one or two groups) selected from OH, OCH₃, NH₂, CN, CH₃, CF₃, CH₂CH₃, isopropyl, F, Cl, Br, morpholino, CO₂H, CO₂CH₃, and CO₂NH₂.

In embodiments, R² is naphthyl. In embodiments, R² is unsubstituted naphthyl. In embodiments, R² is naphthyl comprising at least one halogen substitutent (e.g., at least one substituent that is chloro or fluoro. In embodiments, R² is naphthyl substituted by 1, 2, or 3 groups (e.g., one or two groups) selected from OH, OCH₃, NH₂, CN, CH₃, CF₃, CH₂CH₃, isopropyl, F, Cl, Br, morpholino, CO₂H, CO₂CH₃, and CO₂NH₂.

In embodiments, R² is pyridyl. In embodiments, R² is 2-pyridyl. In embodiments, R² is 3-pyridyl. In embodiments, R² is 4-pyridyl. In embodiments, R² is unsubstituted pyridyl. In embodiments, R² is pyridyl comprising at least one halogen substitutent (e.g., at least one substituent that is chloro or fluoro. In embodiments, R² is pyridyl substituted by 1, 2, or 3 groups (e.g., one or two groups) selected from OH, OCH₃, NH₂, CN, CH₃, CF₃, CH₂CH₃, isopropyl, F, Cl, Br, morpholino, CO₂H, CO₂CH₃, and CO₂NH₂.

In embodiments, R² is indolyl. In embodiments, R² is unsubstituted indolyl. In embodiments, R² is indolyl comprising at least one halogen substitutent (e.g., at least one substituent that is chloro or fluoro. In embodiments, R² is indolyl substituted by 1, 2, or 3 groups (e.g., one or two groups) selected from OH, OCH₃, NH₂, CN, CH₃, CF₃, CH₂CH₃, isopropyl, F, Cl, Br, morpholino, CO₂H, CO₂CH₃, and CO₂NH₂.

In embodiments, R² is phenyl, naphthyl, pyridyl, or

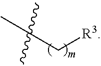

In embodiments, R² is

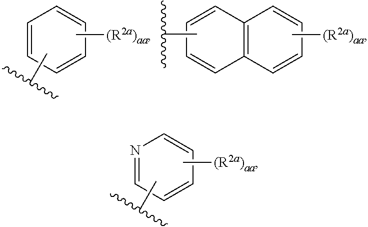

wherein aa is 0, 1, 2, or 3, and each R^{2a} is independently any substituent group described herein. In embodiments, each R^{2a} is independently selected from OH, OCH₃, NH₂, CN, CH₃, CF₃, CH₂CH₃, isopropyl, F, Cl, Br, morpholino, CO₂H, CO₂CH₃, and CO₂NH₂. In embodiments, each R^{2a} is independently selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen.

In embodiments, R² is

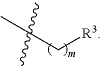

In embodiments, m is 1. In embodiments, m is 2. In embodiments, m is 3.

In embodiments, R³ is phenyl. In embodiments, R³ is unsubstituted phenyl. In embodiments, R³ is phenyl comprising at least one halogen substitutent (e.g., at least one substituent that is chloro or fluoro. In embodiments, R³ is fluorophenyl (e.g., 2-, 3-, or 4-fluorophenyl), difluorophenyl, chlorophenyl (e.g., 2-, 3-, or 4-chlorophenyl), dichlorophenyl, chlorofluorophenyl. In embodiments, R³ is phenyl substituted by 1, 2, or 3 groups (e.g., one or two groups) selected from OH, OCH₃, NH₂, CN, CH₃, CF₃, CH₂CH₃, isopropyl, F, Cl, Br, morpholino, CO₂H, CO₂CH₃, and CO₂NH₂.

In embodiments, R³ is naphthyl. In embodiments, R³ is unsubstituted naphthyl. In embodiments, R³ is naphthyl comprising at least one halogen substitutent (e.g., at least one substituent that is chloro or fluoro. In embodiments, R³ is naphthyl substituted by 1, 2, or 3 groups (e.g., one or two groups) selected from OH, OCH₃, NH₂, CN, CH₃, CF₃, CH₂CH₃, isopropyl, F, Cl, Br, morpholino, CO₂H, CO₂CH₃, and CO₂NH₂.

In embodiments, R³ is pyridyl. In embodiments, R³ is 2-pyridyl. In embodiments, R³ is 3-pyridyl. In embodiments, R³ is 4-pyridyl. In embodiments, R³ is unsubstituted pyridyl. In embodiments, R³ is pyridyl comprising at least one halogen substitutent (e.g., at least one substituent that is chloro or fluoro. In embodiments, R³ is pyridyl substituted by 1, 2, or 3 groups (e.g., one or two groups) selected from OH, OCH₃, NH₂, CN, CH₃, CF₃, CH₂CH₃, isopropyl, F, Cl, Br, morpholino, CO₂H, CO₂CH₃, and CO₂NH₂.

In embodiments, R³ is indolyl. In embodiments, R³ is unsubstituted indolyl. In embodiments, R³ is indolyl comprising at least one halogen substitutent (e.g., at least one substituent that is chloro or fluoro. In embodiments, R³ is indolyl substituted by 1, 2, or 3 groups (e.g., one or two groups) selected from OH, OCH₃, NH₂, CN, CH₃, CF₃, CH₂CH₃, isopropyl, F, Cl, Br, morpholino, CO₂H, CO₂CH₃, and CO₂NH₂.

In embodiments, R³ is phenyl, naphthyl, or pyridyl.

In embodiments, R³ is

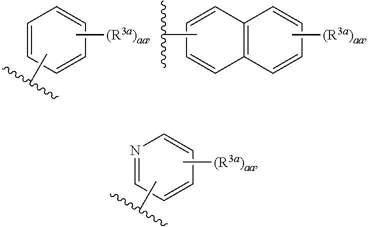

wherein aa is 0, 1, 2, or 3, and each R^{3a} is independently any substituent group described herein. In embodiments, each R^{3a} is independently selected from OH, OCH₃, NH₂, CN, CH₃, CF₃, CH₂CH₃, isopropyl, F, Cl, Br, morpholino, CO₂H, CO₂CH₃, and CO₂NH₂. In embodiments, each R^{3a} is independently selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen.

In a specific embodiments, A is

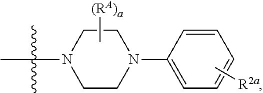

wherein R^{2a} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen; R^{A} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen; and a is independently 0, 1, or 2.

In embodiments, R¹ is a C₆-C₁₀ aryl.

In embodiments, R¹ is a five- to six-membered heteroaryl ring. In embodiments, R¹ is imidazolyl (e.g., unsubstituted imidazolyl or N-methylimidazolyl). In embodiments, R¹ is oxazolyl (e.g., unsubstituted oxazolyl). In embodiments, R¹ is isoxazolyl (e.g., unsubstituted oxazolyl).

In embodiments, R¹ is

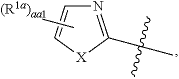

wherein X is 0, NH, or NCH₃, aa1 is 0, 1, or 2, and R^{1a} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen.

In embodiments, R¹ is selected from the group consisting of

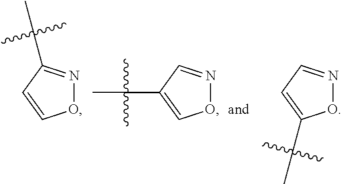 [SMILES: *c1ccon1 ; *c1cnoc1 ; *c1ccno1]

In embodiments, R¹ is

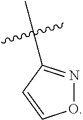 [SMILES: *c1ccon1]

In embodiments, R¹ is a polar acyl group (e.g., substructures

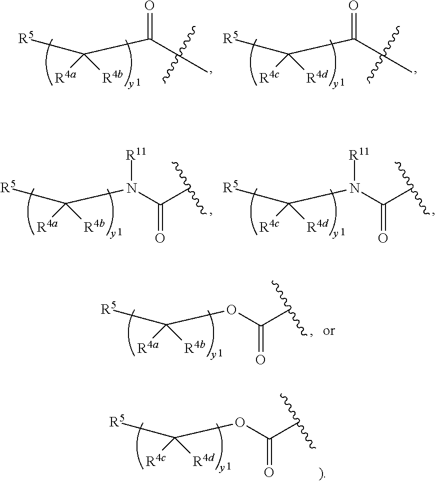

In embodiments, R¹ is an acyl moiety comprising a C₁-C₇ alkyl group, a C₃-C₇ cycloalkyl group (e.g., cyclopropyl, cyclobutyl, cyclopentyl, or cyclohexyl), a C₁-C₇ haloalkyl group, a C₃-C₇ cyclohaloalkyl group (e.g., cyclohalopropyl, cyclohalobutyl, cyclohalopentyl, or cyclohalohexyl), a 4-6-membered oxygen containing heterocyclyl (e.g., oxetanyl, tetrahydrofuranyl, tetrahydropyranyl, or oxazalidonone) or a 4-6-membered nitrogen containing heterocyclyl (e.g., azetidinyl, pyrrolidinyl, or piperidinyl), wherein said group comprises a substituent that is an amino group (e.g., —NH₂, monoalkylamino (e.g., —NHMe), or dialkylamino (e.g., —NMe₂)), an acetamido group (e.g., —NHCOMe or NMeCOMe), an carbamate group (e.g., —NHCO₂Me or —NMeCO₂Me), an alkylsulfonamido group (e.g., —NHSO₂Me or —NMeSO₂Me), or a 5-10-membered nitrogen-containing heterocycyl (e.g., tetrazolyl, imidazolyl, N-methylimidazolyl, pyridyl or pyridazinyl). In embodiments, R¹ is alkylacyl groups (e.g., —C(O)(C₁-C₇ alkyl) or —C(O)(C₃-C₇ cycloalkyl)). In embodiments, R¹ excludes unsubstituted alkylacyl groups (e.g., —C(O)(C₁-C₇ alkyl) or —C(O)(C₃-C₇ cycloalkyl)). In embodiments, R¹ is a polar sulfonyl group (e.g., substructures

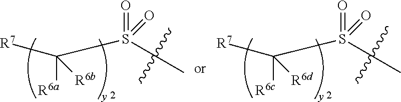

as further described herein). In embodiments, R¹ is a sulfonyl moiety comprising a C₁-C₇ alkyl group, a C₃-C₇ cycloalkyl group (e.g., cyclopropyl, cyclobutyl, cyclopentyl, or cyclohexyl), a C₁-C₇ haloalkyl group, a C₃-C₇ cyclohaloalkyl group (e.g., cyclohalopropyl, cyclohalobutyl, cyclohalopentyl, or cyclohalohexyl), a 4-6-membered oxygen containing heterocyclyl (e.g., oxetanyl, tetrahydrofuranyl, tetrahydropyranyl, or oxazalidonone) or a 4-6-membered nitrogen containing heterocyclyl (e.g., azetidinyl, pyrrolidinyl, or piperidinyl), wherein said group comprises a substituent that is an amino group (e.g., —NH₂, monoalkylamino (e.g., —NHMe), or dialkylamino (e.g., —NMe₂)), an acetamido group (e.g., —NHCOMe or NMeCOMe), an alkylsulfonamido group (e.g., —NHSO₂Me or —NMeSO₂Me), or a 5-10-membered nitrogen-containing heterocycyl (e.g., tetrazolyl, imidazolyl, N-methylimidazolyl, pyridyl or pyridazinyl).

In embodiments, R¹ is selected from the group consisting of

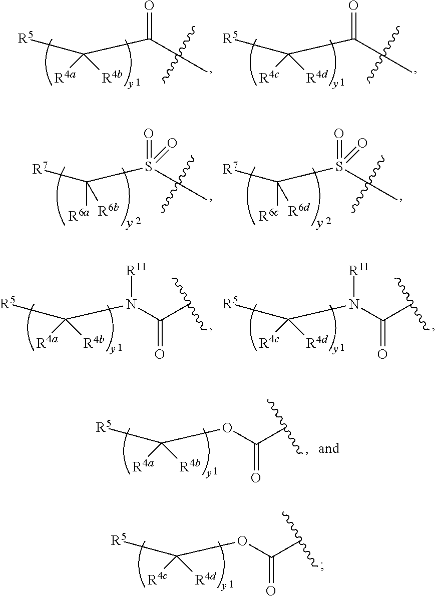

each R^{4a}, R^{4b}, R^{4c}, R^{6a}, R^{6b} and R^{6c} is selected from the group consisting of hydrogen, C₁-C₇ alkyl and C₃-C₇ cycloalkyl; or R^{4a} and R^{4b} optionally are taken together with the atoms to which they are bound to form a ring containing 3 to 7 atoms, optionally containing oxygen; or R^{6a} and R^{6b} optionally are taken together with the atoms to which they are bound to form a ring containing 3 to 7 atoms, optionally containing oxygen;

each R^{4d} and R^{6d} is selected from the group consisting of phenyl, benzyl, pyridyl, —CH₂(pyridyl), imidazole, and —CH₂(imidazole).

R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and

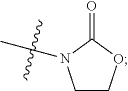 [SMILES: *N1CCOC1=O]

R⁷ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NHCONR^{8f};

each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;

each R^{8c}, R^{8e}, R^{8f} and R^{8h} is C₃-C₇ alkyl or C₃-C₇ cycloalkyl;

R^{8j} is selected from the group consisting of C₁-C₇ alkyl, C₃-C₇ cycloalkyl, C₆-C₁₀ aryl, and 5- to 10-membered heteroaryl; or

when R^{4a} and R^{8a} both present, or R^{4a} and R^{8g} both present, these groups are optionally taken together with the atoms to which they are bound to form a ring containing 4 to 7 atoms;

R⁹ is selected from the group consisting of hydrogen, C₁-C₇ alkyl, and C₃-C₇ cycloalkyl;

R¹¹ is selected from the group consisting of hydrogen, C₁-C₇ alkyl, and C₃-C₇ cycloalkyl;

y¹ is 0, 1 or 2; and

y² is 0, 1, or 2.

In embodiments, y¹ is 0. In embodiments, y¹ is 1. In embodiments, y¹ is 2.

In embodiments, y² is 0. In embodiments, y² is 1. In embodiments, y² is 2.

In embodiments, R^{4a} is H. In embodiments, R^{4b} is H. In embodiments, R^{4a} and R^{4b} are both H. In embodiments, y¹ is 0. In embodiments, y¹ is 1. In embodiments, y¹ is 2.

In embodiments, R^{4a} and R^{4b} are taken together with the atoms to which they are bound to form a carbocyclic ring containing 3 to 7 atoms. In embodiments, R^{4a} and R^{4b} are taken together with the atoms to which they are bound to form an oxygen-containing ring containing 3 to 7 atoms.

In embodiments, R^{4e} is H. In embodiments, R^{4d} is phenyl. In embodiments, R^{4d} is benzyl. In embodiments, R^{4d} is pyridyl. In embodiments, R^{4d} is —CH₂(pyridyl). In embodiments, R^{4d} is imidazole. In embodiments, R^{4d} is —CH₂(imidazole). In embodiments, y¹ is 0. In embodiments, y¹ is 1. In embodiments, y¹ is 2.

In embodiments, R^{6a} is H. In embodiments, R^{6b} is H. In embodiments, R^{6a} and R^{6b} are both H. In embodiments, y² is 0. In embodiments, y² is 1. In embodiments, y² is 2.

In embodiments, R⁴⁶ and R^{6b} are taken together with the atoms to which they are bound to form a carbocyclic ring containing 3 to 7 atoms. In embodiments, R^{6a} and R^{bb} are taken together with the atoms to which they are bound to form a oxygen-containing ring containing 3 to 7 atoms.

In embodiments, R^{6c} is H. In embodiments, R^{6d} is phenyl. In embodiments, R^{6d} is benzyl. In embodiments, R^{6d} is pyridyl. In embodiments, R^{6d} is —CH₂(pyridyl). In embodiments, R^{6d} is imidazole. In embodiments, R^{6d} is —CH₂(imidazole). In embodiments, y² is 0. In embodiments, y² is 1. In embodiments, y² is 2.

In embodiments, R⁵ is pyridyl. In embodiments, R⁵ is pyridazine. In embodiments, R⁵ is C₁-C₇ alkyl. In embodiments, R⁵ is C₃-C₇ cycloalkyl. In embodiments, R⁵ is C₁-C₇ haloalkyl. In embodiments, R⁵ is C₃-C₇ cyclohaloalkyl. In embodiments, R⁵ is C₁-C₇ fluoroalkyl. In embodiments, R⁵ is C₃-C₇ cyclofluoroalkyl. In embodiments, R⁵ is unsubstituted C₁-C₇ alkyl. In embodiments, R⁵ is substituted C₁-C₇ alkyl (e.g., comprising an amino substituent such as —NH₂, —NHCH₃, or —N(CH₃)₂). In embodiments, R⁵ is phenyl. In embodiments, R⁵ is phenyl. In embodiments, R⁵ is unsubstituted phenyl. In embodiments, R⁵ is substituted phenyl. In embodiments, R⁵ is NR^{8a}R^{8b}. In embodiments, R⁵ is SO₂R^{8c}. In embodiments, R⁵ is NR^{8d}SO₂R^{8e}. In embodiments, R⁵ is NR⁸ⁱCOOR^{8j}. In embodiments, R⁵ is NHCONR^{8f}. In embodiments, R⁵ is NR^{8g}COR^{8h}. In embodiments, R⁵ is not unsubstituted C₁-C₇ alkyl.

In embodiments, R⁷ is pyridyl. In embodiments, R⁷ is pyridazine. In embodiments, R⁷ is C₁-C₇ alkyl. In embodiments, R⁷ is C₃-C₇ cycloalkyl. In embodiments, R⁷ is C₁-C₇ haloalkyl. In embodiments, R⁷ is C₃-C₇ cyclohaloalkyl. In embodiments, R⁷ is C₁-C₇ fluoroalkyl. In embodiments, R⁷ is C₃-C₇ cyclofluoroalkyl. In embodiments, R⁷ is unsubstituted C₁-C₇ alkyl. In embodiments, R⁷ is substituted C₁-C₇ alkyl. In embodiments, R⁷ is phenyl. In embodiments, R⁷ is phenyl. In embodiments, R⁷ is unsubstituted phenyl. In embodiments, R⁷ is substituted phenyl. In embodiments, R⁷ is NR^{8a}R^{8b}. In embodiments, R⁷ is SO₂R^{8c}. In embodiments, R⁷ is NR^{8d}SO₂R^{8e}. In embodiments, R⁷ is NHCONR^{8f}. In embodiments, R⁷ is not unsubstituted C₁-C₇ alkyl.

In embodiments, R^{4d} is selected from the group consisting of

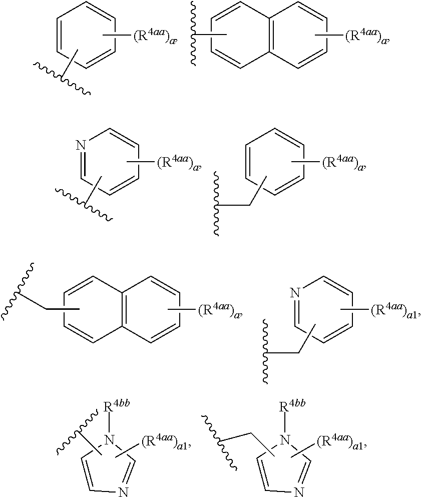

wherein R^{4bb} is H or CH₃, a1 is 1 or 2, and each R^{4aa} is independently any substituent group described herein. In embodiments, each R^{4aa} is independently selected from OH, OCH₃, NH₂, CN, CH₃, CF₃, CH₂CH₃, isopropyl, F, Cl, Br, morpholino, CO₂H, CO₂CH₃, and CO₂NH₂. In embodiments, each R^{4aa} is independently selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen.

In embodiments, R^{6d} is selected from the group consisting of

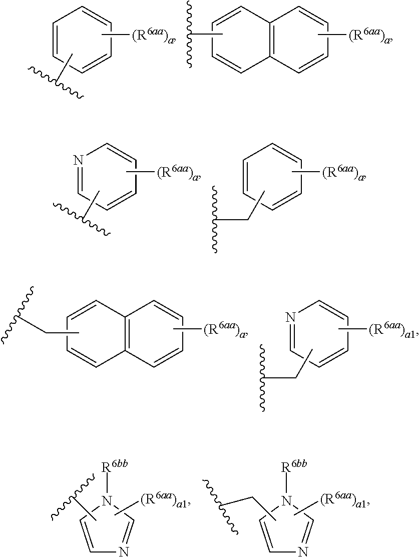

wherein R^{6bb} is H or CH₃, a1 is 1 or 2, and each R^{6aa} is independently any substituent group described herein. In embodiments, each R^{6aa} is independently selected from OH, OCH₃, NH₂, CN, CH₃, CF₃, CH₂CH₃, isopropyl, F, Cl, Br, morpholino, CO₂H, CO₂CH₃, and CO₂NH₂. In embodiments, each R^{6aa} is independently selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen.

In embodiments, R¹ is

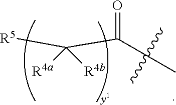

In embodiments, y¹ is 0. In embodiments, y¹ is 1. In embodiments, y¹ is 2.

In embodiments, R¹ is

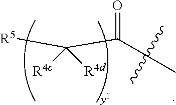

In embodiments, y¹ is 0. In embodiments, y¹ is 1. In embodiments, y¹ is 2.

In embodiments, R¹ is

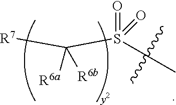

In embodiments, y² is 0. In embodiments, y² is 1. In embodiments, y² is 2.

In embodiments, R¹ is

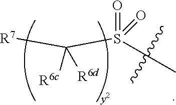

In embodiments, y² is 0. In embodiments, y² is 1. In embodiments, y² is 2.

In embodiments, R¹ is

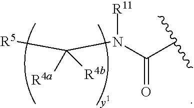

In embodiments, y² is 0. In embodiments, y² is 1. In embodiments, y² is 2.

In embodiments, R¹ is

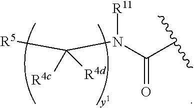

In embodiments, y² is 0. In embodiments, y² is 1. In embodiments, y² is 2.

In embodiments, R¹ is

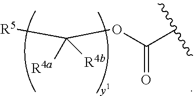

In embodiments, y¹ is 0. In embodiments, y¹ is 1. In embodiments, y¹ is 2.

In embodiments, R¹ is

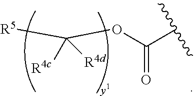

In embodiments, y¹ is 0. In embodiments, y¹ is 1. In embodiments, y¹ is 2.

In embodiments, y¹ is 0, and R¹ is COR⁵. In embodiments, R⁵ is pyridyl. In embodiments, R⁵ is pyridazine. In embodiments, R⁵ is C₁-C₇ alkyl. In embodiments, R⁵ is C₃-C₇ cycloalkyl. In embodiments, R⁵ is C₁-C₇ haloalkyl. In embodiments, R⁵ is C₃-C₇ cyclohaloalkyl. In embodiments, R⁵ is C₁-C₇ fluoroalkyl. In embodiments, R⁵ is C₃-C₇ cyclofluoroalkyl.

In embodiments, R^{1′} is hydrogen. In embodiments, R^{1′} is C₁-C₇ alkyl (e.g. methyl). In embodiments, R^{1′} is C₃-C₇ cycloalkyl.

In embodiments, R¹ is

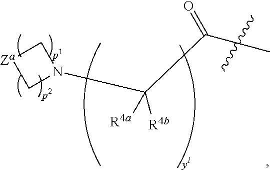

wherein

R^{4a}, R^{4b}, and y¹ are according to any aspect or embodiment described herein;

Z^{a} is CH₂ or 0;

when Z^{a} is CH₂, p¹+p² is 1, 2, 3, or 4; and

when Z^{a} is 0, p¹+p² is 1, 2, 3, or 4; and both p¹ and p² are not 0.

In embodiments, R¹ is

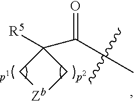

wherein

Z^{b} is CH₂ or 0;

when Z^{b} is CH₂, p¹+p² is 1, 2, 3, or 4;

when Z^{b} is 0, p¹+p² is 1, 2, 3, or 4; and both p¹ and p² are not 0;

R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NHCONR^{8f}, NR^{8g}COR^{8h} and

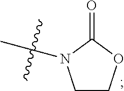 [SMILES: *N1CCOC1=O]

each R^{a}, R^{8b}, R^{8d}, R^{8g} and R⁹ is selected from the group consisting of hydrogen, C₁-C₇ alkyl, and C₃-C₇ cycloalkyl;

R^{8a} and R^{8b} optionally are taken together with the atoms to which they are bound to form a heterocyle containing 3 to 7 atoms, optionally containing a group selected from oxygen, sulfur, and NR⁹;

each R^{8c}, R^{8e}, R^{8f} and R^{8h} is C₃-C₇ alkyl or C₃-C₇ cycloalkyl.

In embodiments, R¹ is

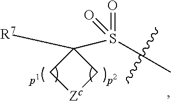

wherein

Z^{c} is CH₂ or 0;

when Z^{c} is CH₂, p¹+p² is 1, 2, 3, or 4;

when Z^{c} is 0, p¹+p² is 1, 2, 3, or 4; and both p¹ and p² are not 0;

R⁷ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NHCONR^{8f};

each R^{8a}, R^{8b}, R^{8d}, R^{8g} and R⁹ is selected from the group consisting of hydrogen, C₁-C₇ alkyl, and C₃-C₇ cycloalkyl;

R^{8a} and R^{8b} optionally are taken together with the atoms to which they are bound to form a heterocyle containing 3 to 7 atoms, optionally containing a group selected from oxygen, sulfur, and NR⁹;

each R^{8c}, R^{8e}, R^{8f} and R^{8h} is C₃-C₇ alkyl or C₃-C₇ cycloalkyl.

In embodiments, R¹ is

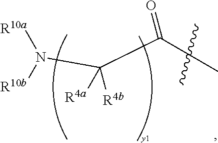

wherein

R^{10a} and R^{10b} is independently selected from the group consisting of H, C₁-C₇ linear alkyl, C₃-C₇ branched alkyl, C₃-C₇ cycloalkyl, SO₂R^{8e}, COOR^{8j}, CONR^{8f}, and COR^{8h}; and

at least one of R^{10a} and R^{10b} is selected from the group consisting of H, C₁-C₇ linear alkyl, C₃-C₇ branched alkyl, and C₃-C₇ cycloalkyl;

each R^{8e}, R^{8f} and R^{8h} is selected from the group consisting of H, C₁-C₇ linear alkyl, C₃-C₇ branched alkyl, C₃-C₇ cycloalkyl; and

R^{8j} is selected from the group consisting of C₁-C₇ linear alkyl, C₃-C₇ branched alkyl, C₃-C₇ cycloalkyl, C₆-C₁₀ aryl, and 5- to 10-membered heteroaryl.

In embodiments, R¹ is COOR⁵, wherein R⁵ is C₆-C₁₀ aryl or 5- to 10-membered heteroaryl.

In embodiments, R¹ is

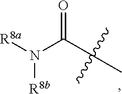

wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl.

In embodiments, R¹ is

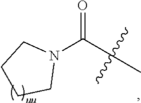

wherein uu is 1 or 2.

In embodiments, R¹ is

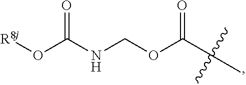

wherein R^{8j} is selected from the group consisting of C₁-C₇ alkyl, C₃-C₇ cycloalkyl, C₆-C₁₀ aryl, and 5- to 10-membered heteroaryl.

In embodiments, R¹ is

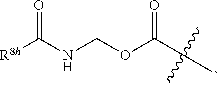

wherein R^{8h} is unsubstituted C₁-C₇ alkyl.

In embodiments, R¹ is

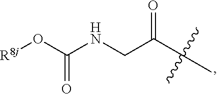

wherein R^{8g} is selected from the group consisting of C₁-C₇ alkyl, C₃-C₇ cycloalkyl, C₆-C₁₀ aryl, and 5- to 10-membered heteroaryl.

In embodiments, R¹ is

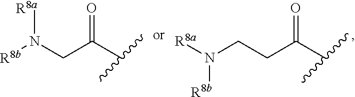

wherein each R^{8a} and R⁸⁸ is independently H or unsubstituted C₁-C₇ alkyl.

In embodiments, R¹ is

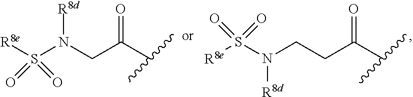

wherein R^{8d} is independently H or unsubstituted C₁-C₇ alkyl, and R^{8f} is unsubstituted C₁-C₇ alkyl.

In embodiments, R¹ is

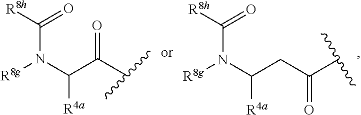

wherein each of R and R^{8g} is independently H or unsubstituted C₁-C₇ alkyl; and R^{8h} is unsubstituted C₃-C₇ alkyl.

In embodiments, R¹ is

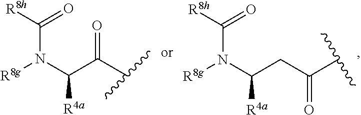

wherein each of R and R^{8g} is independently H or unsubstituted C₁-C₇ alkyl; and R^{8h} is unsubstituted C₃-C₇ alkyl.

In embodiments, R¹ is

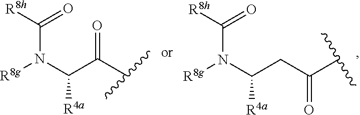

wherein each of R and R^{8g} is independently H or unsubstituted C₁-C₇ alkyl; and R^{8h} is unsubstituted C₁-C₇ alkyl.

In embodiments, R¹ is

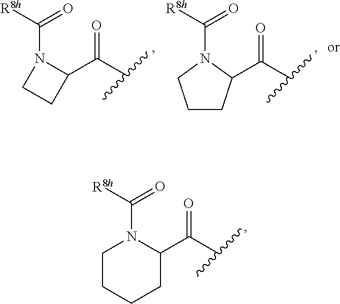

wherein R^{8h} is unsubstituted C₁-C₇ alkyl.

In embodiments, R¹ is

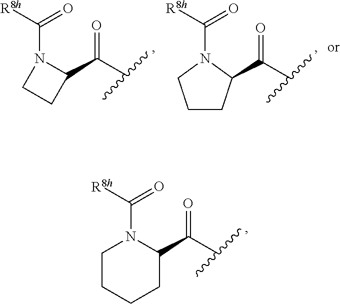

wherein R^{8h} is unsubstituted C₁-C₇ alkyl.

In embodiments, R¹ is

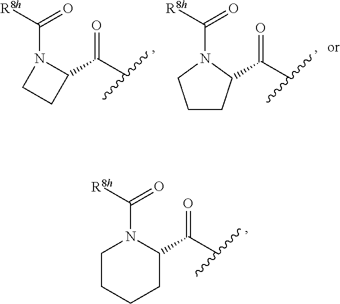

wherein R^{8h} is unsubstituted C₁-C₇ alkyl.

In embodiments, R¹ is or

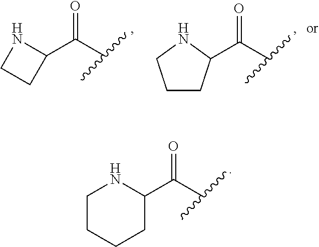 [SMILES: *C(=O)C1CCN1 ; *C(=O)C1CCCN1 ; *C(=O)C1CCCCN1]

In embodiments, R¹ is

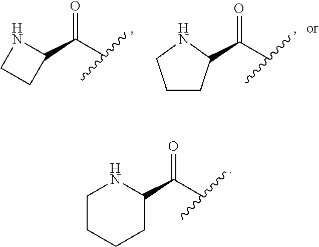

In embodiments, R¹ is or

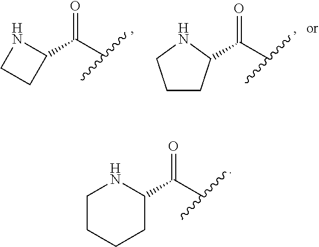

In embodiments, R¹ is

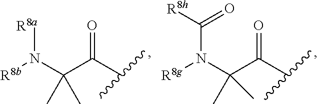

wherein each R^{a}, R^{8b}, and R^{8g} is independently H or unsubstituted C₁-C₇ alkyl, and R^{8h} is unsubstituted C₁-C₇ alkyl.

In embodiments, R¹ is

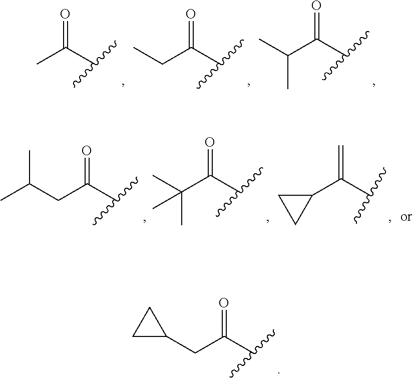

In embodiments, R¹ is

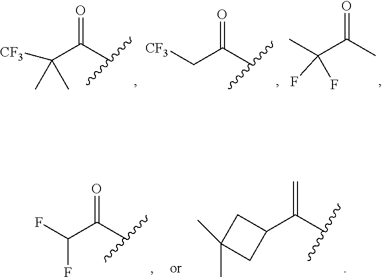

In embodiments, R¹ is

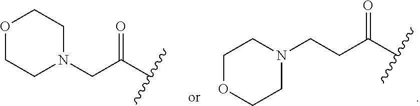 [SMILES: *C(=O)CN1CCOCC1 ; *C(=O)CCN1CCOCC1]

In embodiments, R¹ is

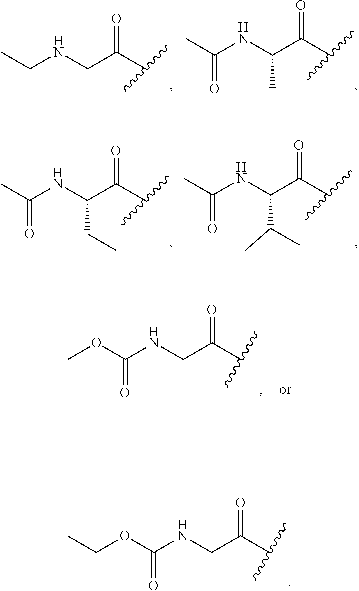

In embodiments, R¹ is

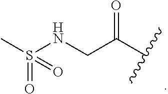 [SMILES: *C(=O)CNS(C)(=O)=O]

In embodiments, R¹ is

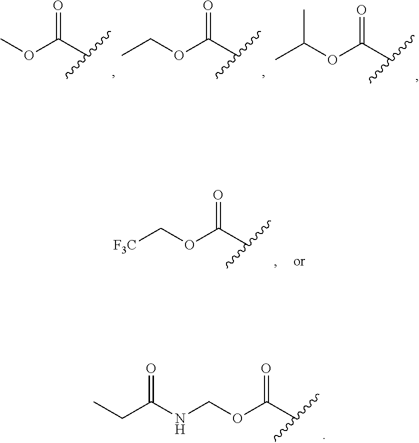

In embodiments, R¹ is

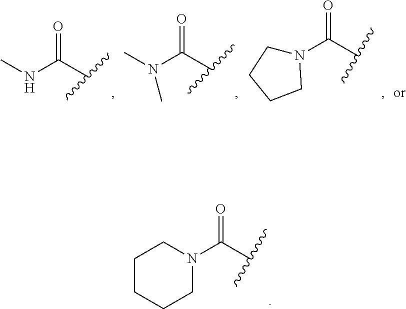

In embodiments, R¹ is

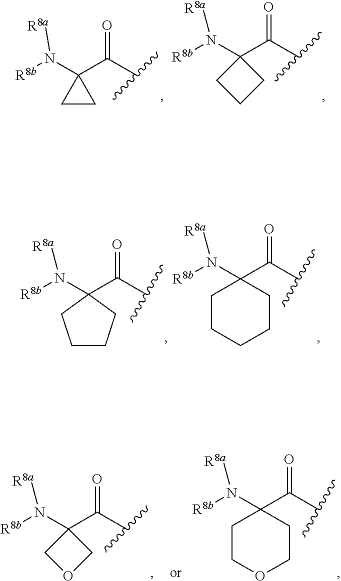

wherein each R^{8a} and R^{8b} is independently H or unsubstituted C₁-C₇ alkyl.

In embodiments, R¹ is

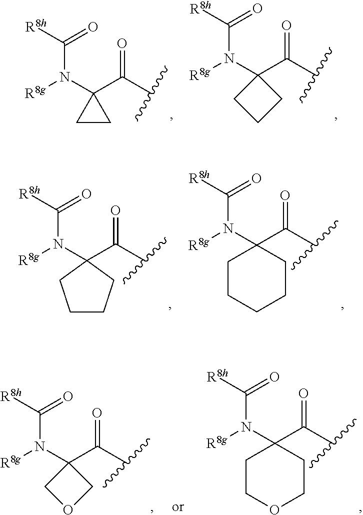

wherein R^{8g} is independently H or unsubstituted C₁-C₇ alkyl, and R^{8h} is independently unsubstituted C₁-C₇ alkyl.

In embodiments, R¹ is

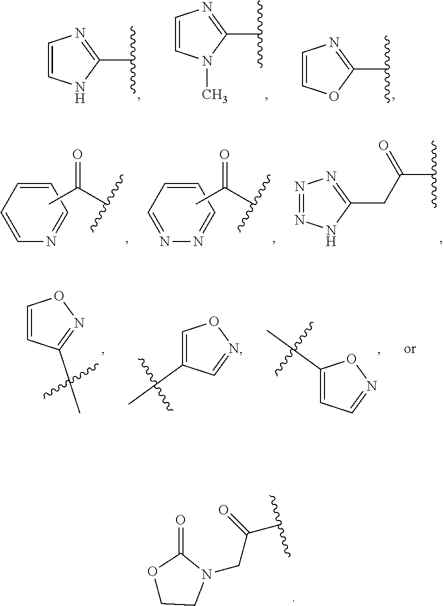

Compounds of Formula (II)

In one aspect, the present invention features a compound having a structure according to Formula (II)

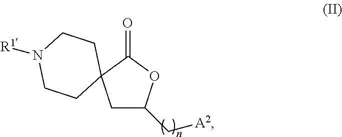

including enantiomers, diastereomers, hydrates, solvates, pharmaceutically acceptable salts, prodrugs and complexes thereof, wherein:
A² is

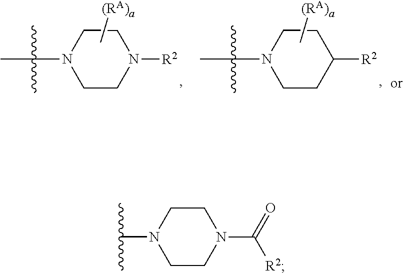

R² is selected from the group consisting of 6- to 10-membered aryl, 5- to 10-membered nitrogen-containing heteroaryl, and

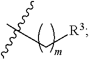

R³ is a 6- to 10-membered aryl or 5- to 10-membered nitrogen-containing heteroaryl;
m is 1, 2, or 3;
n is 1, 2, 3, or 4;
R^{1′} is selected from the group consisting of a C₆-C₁₀ aryl, a five- to six-membered heteroaryl ring,

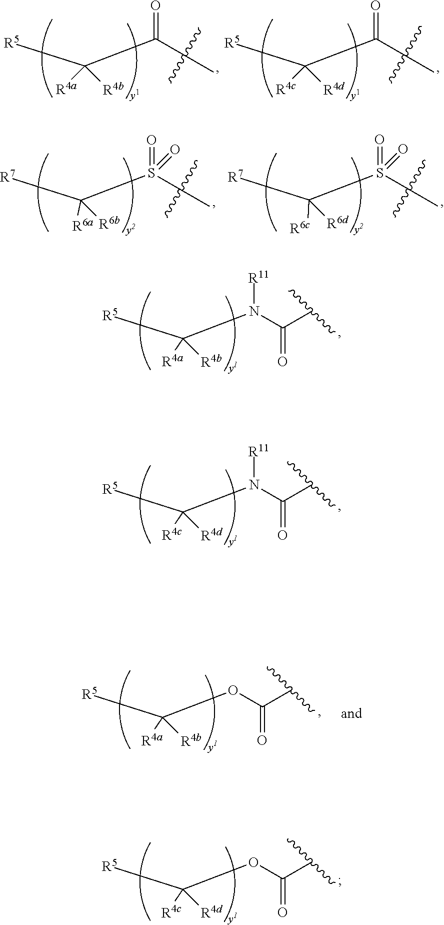

each R^{4a}, R^{4b}, R^{4c}, R^{6a}, R^{6b} and R^{6c} is selected from the group consisting of hydrogen, C₁-C₇ alkyl and C₃-C₇ cycloalkyl;
R^{4a} and R^{4b} optionally are taken together with the atoms to which they are bound to form a ring containing 3 to 7 atoms, optionally containing oxygen;
R^{6a} and R^{6b} optionally are taken together with the atoms to which they are bound to form a ring containing 3 to 7 atoms, optionally containing oxygen;
each R^{4d} and R^{6d} is selected from the group consisting of phenyl, benzyl, pyridyl, —CH₂(pyridyl), imidazole, and —CH₂(imidazole).
R⁵ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR″COOR^{8j}, NHCONR^{8f}, NR^{8g}CR^{8h} and

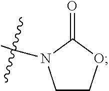 [SMILES: *N1CCOC1=O]

R⁷ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NHCONR^{8f};
each R^{8a}, R^{8b}, R^{8d}, R^{8g}, and R⁸ⁱ is selected from the group consisting of hydrogen, C₁-C₇ alkyl, and C₃-C₇ cycloalkyl; or
R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
R¹¹ is selected from the group consisting of hydrogen, C₁-C₇ alkyl, and C₃-C₇ cycloalkyl;
R^{A} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen;
a is 0, 1 or 2;
y¹ is 0, 1 or 2; and
y² is 0, 1, or 2.

In embodiments, when A² is

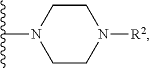

R² is phenyl, R^{1′} is

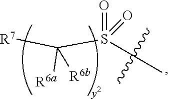

y² is 0, and n is 2, then R⁷ is not methyl, CH₂SO₂CH₃, CH₂CN, tetrahydropyranyl, phenyl, 4-substituted phenyl, or 5- to 8-membered heteroaryl.

In embodiments, the compound according to Formula (II) has a structure according to Formula (II′) or Formula (II″).

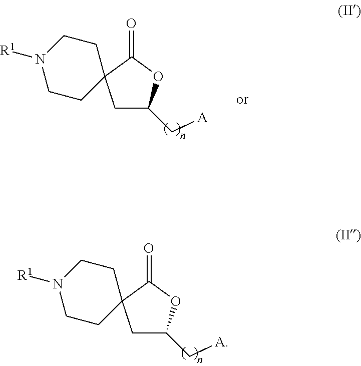

In embodiments, A² is

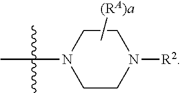

In embodiments, A² is

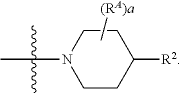

In embodiments, A² is

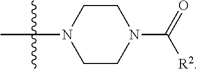

In embodiments, R^{A} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen. In embodiments, R^{A} is unsubstituted C₁-C₇ alkyl. In embodiments, R^{A} is methyl.

In embodiments, a is 0. In embodiments, a is 1. In embodiments, a is 2. In embodiments, a is not 0. In embodiments, a excludes 0. In embodiments, a is 0 or 1. In embodiments, a is 1 or 2.

In embodiments, y¹ is 0. In embodiments, y¹ is 1. In embodiments, y¹ is 2.

In embodiments, y² is 0. In embodiments, y² is 1. In embodiments, y² is 2.

In embodiments, R^{4a} is H. In embodiments, R^{4b} is H. In embodiments, R^{4a} and R^{4b} are both H. In embodiments, y¹ is 0. In embodiments, y¹ is 1. In embodiments, y¹ is 2.

In embodiments, R^{4a} and R^{4b} are taken together with the atoms to which they are bound to form a carbocyclic ring containing 3 to 7 atoms. In embodiments, R^{4a} and R^{4b} are taken together with the atoms to which they are bound to form a oxygen-containing ring containing 3 to 7 atoms.

In embodiments, R⁴¹ is H. In embodiments, R^{4d} is phenyl. In embodiments, R^{4d} is benzyl. In embodiments, R^{4d} is pyridyl. In embodiments, R^{4d} is —CH₂(pyridyl). In embodiments, R^{4d} is imidazole. In embodiments, R^{4d} is —CH₂(imidazole). In embodiments, y¹ is 0. In embodiments, y¹ is 1. In embodiments, y¹ is 2.

In embodiments, R^{6a} is H. In embodiments, R^{6b} is H. In embodiments, R^{6a} and R^{6b} are both H. In embodiments, y² is 0. In embodiments, y² is 1. In embodiments, y² is 2.

In embodiments, R^{6a} and R^{6b} are taken together with the atoms to which they are bound to form a carbocyclic ring containing 3 to 7 atoms. In embodiments, R^{6a} and R^{6b} are taken together with the atoms to which they are bound to form a oxygen-containing ring containing 3 to 7 atoms.

In embodiments, R^{6c} is H. In embodiments, R^{6d} is phenyl. In embodiments, R^{6d} is benzyl. In embodiments, R^{6d} is pyridyl. In embodiments, R^{6d} is —CH₂(pyridyl). In embodiments, R^{6d} is imidazole. In embodiments, R^{6d} is —CH₂(imidazole). In embodiments, y² is 0. In embodiments, y² is 1. In embodiments, y² is 2.

In embodiments, R⁵ is pyridyl. In embodiments, R⁵ is pyridazine. In embodiments, R⁵ is C₁-C₇ alkyl. In embodiments, R⁵ is C₃-C₇ cycloalkyl. In embodiments, R⁵ is C₁-C₇ haloalkyl. In embodiments, R⁵ is C₃-C₇ cyclohaloalkyl. In embodiments, R⁵ is C₁-C₇ fluoroalkyl. In embodiments, R⁵ is C₃-C₇ cyclofluoroalkyl. In embodiments, R⁵ is unsubstituted C₁-C₇ alkyl. In embodiments, R⁵ is substituted C₁-C₇ alkyl (e.g., comprising an amino substituent such as —NH₂, —NHCH₃, or —N(CH₃)₂). In embodiments, R⁵ is phenyl. In embodiments, R⁵ is phenyl. In embodiments, R⁵ is unsubstituted phenyl. In embodiments, R⁵ is substituted phenyl. In embodiments, R⁵ is NR^{8a}R^{8b}. In embodiments, R⁵ is SO₂R^{8c}. In embodiments, R⁵ is NR^{8d}SO₂R^{8e}. In embodiments, R⁵ is NR⁸ⁱCOOR^{8j}. In embodiments, R⁵ is NHCONR^{8f}. In embodiments, R⁵ is NR^{8g}COR^{8h}. In embodiments, R⁵ is not unsubstituted C₁-C₇ alkyl.

In embodiments, R⁷ is pyridyl. In embodiments, R⁷ is pyridazine. In embodiments, R⁷ is C₁-C₇ alkyl. In embodiments, R⁷ is C₃-C₇ cycloalkyl. In embodiments, R⁷ is C₁-C₇ haloalkyl. In embodiments, R⁷ is C₃-C₇ cyclohaloalkyl. In embodiments, R⁷ is C₁-C₇ fluoroalkyl. In embodiments, R⁷ is C₃-C₇ cyclofluoroalkyl. In embodiments, R⁷ is unsubstituted C₁-C₇ alkyl. In embodiments, R⁷ is substituted C₁-C₇ alkyl. In embodiments, R⁷ is phenyl. In embodiments, R⁷ is phenyl. In embodiments, R⁷ is unsubstituted phenyl. In embodiments, R⁷ is substituted phenyl. In embodiments, R⁷ is NR^{8a}R^{8b}. In embodiments, R⁷ is SO₂R^{8c}. In embodiments, R⁷ is NR^{8d}SO₂R^{8e}. In embodiments, R⁷ is NHCONR^{8f}. In embodiments, R⁷ is not unsubstituted C₁-C₇ alkyl.

In embodiments, R^{1′} is a five- to six-membered heteroaryl ring. In embodiments, R^{1′} is imidazolyl (e.g., unsubstituted imidazolyl or N-methylimidazolyl). In embodiments, R¹ is oxazolyl (e.g., unsubstituted oxazolyl). In embodiments, R¹ is isoxazolyl (e.g., unsubstituted oxazolyl).

In embodiments, R^{1′} is

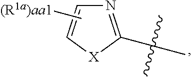

wherein X is O, NH, or NCH₃, aa1 is 0, 1, or 2, and R^{1a} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen.

In embodiments, R^{1′} is selected from the group consisting of

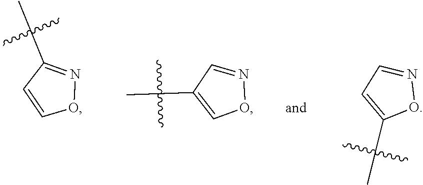 [SMILES: *c1ccon1 ; *c1cnoc1 ; "and" ; *c1ccno1]

In embodiments, R^{1′} is

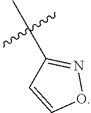 [SMILES: *c1ccon1]

In embodiments, R^{1′} is

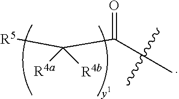

In embodiments, y¹ is 0. In embodiments, y¹ is 1. In embodiments, y¹ is 2.

In embodiments, R^{1′} is

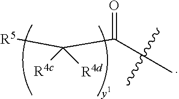

In embodiments, y¹ is 0. In embodiments, y¹ is 1. In embodiments, y¹ is 2.

In embodiments, R^{1′} is

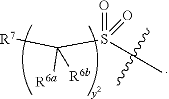

In embodiments, y² is 0. In embodiments, y² is 1. In embodiments, y² is 2.

In embodiments, R is

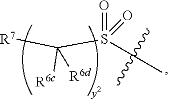

In embodiments, y² is 0. In embodiments, y² is 1. In embodiments, y² is 2.

In embodiments, R^{1′} is

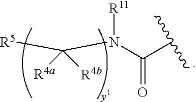

In embodiments, y² is 0. In embodiments, y² is 1. In embodiments, y² is 2.

In embodiments, R^{1′} is

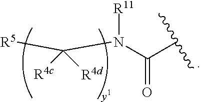

In embodiments, y² is 0. In embodiments, y² is 1. In embodiments, y² is 2.

embodiments, R^{1′} is

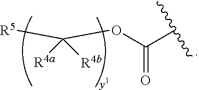

In embodiments, y¹ is 0. In embodiments, y¹ is 1. In embodiments, y¹ is 2.

In embodiments, R^{1′} is

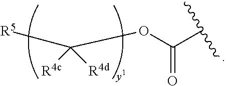

In embodiments, y¹ is 0. In embodiments, y¹ is 1. In embodiments, y¹ is 2.

In embodiments, y¹ is 0, and R^{1′} is COR⁵. In embodiments, R⁵ is pyridyl. In embodiments, R⁵ is pyridazine. In embodiments, R⁵ is C₁-C₇ alkyl. In embodiments, R⁵ is C₃-C₇ cycloalkyl. In embodiments, R⁵ is C₁-C₇ haloalkyl. In embodiments, R⁵ is C₃-C₇ cyclohaloalkyl. In embodiments, R⁵ is C₁-C₇ fluoroalkyl. In embodiments, R⁵ is C₃-C₇ cyclofluoroalkyl.

In embodiments, R^{1′} is hydrogen. In embodiments, R^{1′} is C₁-C₇ alkyl (e.g. methyl). In embodiments, R^{1′} is C₃-C₇ cycloalkyl.

In embodiments, R^{1′} is

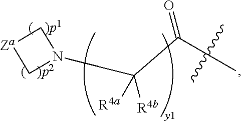

wherein

R^{4a}, R^{4b}, and y¹ are according to any aspect or embodiment described herein;

Z^{a} is CH₂ or 0;

when Z^{a} is CH₂, p¹+p² is 1, 2, 3, or 4; and

when Z^{a} is 0, p¹+p² is 1, 2, 3, or 4; and both p¹ and p² are not 0.

In embodiments, R^{1′} is

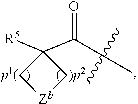

wherein

Z^{b} is CH₂ or 0;

when Z^{b} is CH₂, p¹+p² is 1, 2, 3, or 4;

when Z^{b} is 0, p¹+p² is 1, 2, 3, or 4; and both p¹ and p² are not 0;

R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NHCONR^{8f}, NR^{8g}COR^{8h} and

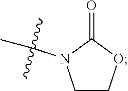 [SMILES: *N1CCOC1=O]

each R^{8a}, R^{8b}, R^{8d}, R^{8g} and R⁹ is selected from the group consisting of hydrogen, C₁-C₇ alkyl, and C₃-C₇ cycloalkyl;

R^{8a} and R^{8b} optionally are taken together with the atoms to which they are bound to form a heterocyle containing 3 to 7 atoms, optionally containing a group selected from oxygen, sulfur, and NR⁹;

each R^{8c}, R^{8e}, R^{8f} and R^{8h} is C₁-C₇ alkyl or C₃-C₇ cycloalkyl.

In embodiments, R^{1′} is

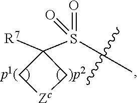

wherein

Z^{c} is CH₂ or 0;

when Z^{c} is CH₂, p¹+p² is 1, 2, 3, or 4;

when Z^{c} is 0, p¹+p² is 1, 2, 3, or 4; and both p¹ and p² are not 0;

R⁷ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NHCONR^{8f};

each R^{8a}, R^{8b}, R^{8d}, R^{8g} and R⁹ is selected from the group consisting of hydrogen, C₁-C₇ alkyl, and C₃-C₇ cycloalkyl;

R^{8a} and R^{8b} optionally are taken together with the atoms to which they are bound to form a heterocyle containing 3 to 7 atoms, optionally containing a group selected from oxygen, sulfur, and NR⁹;

each R^{8c}, R^{8e}, R^{8f} and R^{8h} is C₃-C₇ alkyl or C₃-C₇ cycloalkyl.

In embodiments, R^{1′} is

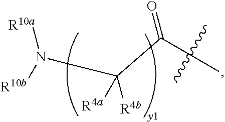

wherein

R^{10a} and R^{10b} is independently selected from the group consisting of H, C₁-C₇ linear alkyl, C₃-C₇ branched alkyl, C₃-C₇ cycloalkyl, SO₂R^{8c}, COOR⁸3, CONR^{8g}, and COR^{8h}; and

at least one of R^{10a} and R^{10b} is selected from the group consisting of H, C₁-C₇ linear alkyl, C₃-C₇ branched alkyl, and C₃-C₇ cycloalkyl;

each R^{8e}, R^{8f} and R^{8h} is selected from the group consisting of H, C₁-C₇ linear alkyl, C₃-C₇ branched alkyl, C₃-C₇ cycloalkyl;

R^{8j} is selected from the group consisting of C₁-C₇ linear alkyl, C₃-C₇ branched alkyl, C₃-C₇ cycloalkyl, C₆-C₁₀ aryl, and 5- to 10-membered heteroaryl.

In embodiments, R^{1′} is COOR⁵, wherein R⁵ is C₆-C₁₀ aryl or 5- to 10-membered heteroaryl.

In embodiments, R^{1′} is

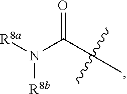

wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl.

In embodiments, R^{1′} is

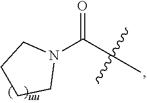

wherein uu is 1 or 2.

In embodiments, R^{1′} is

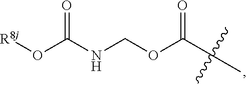

wherein R^{8j} is selected from the group consisting of C₁-C₇ alkyl, C₃-C₇ cycloalkyl, C₆-C₁₀ aryl, and 5- to 10-membered heteroaryl.

In embodiments, R^{1′} is

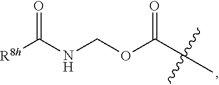

wherein R^{8h} is unsubstituted C₁-C₇ alkyl.

In embodiments, R^{1′} is

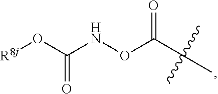

wherein R^{8j} is selected from the group consisting of C₁-C₇ alkyl, C₃-C₇ cycloalkyl, C₆-C₁₀ aryl, and 5- to 10-membered heteroaryl.

In embodiments, R^{1′} is

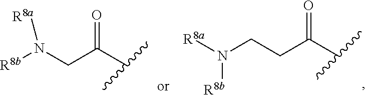

wherein each R^{8a} and R^{8b} is independently H or unsubstituted C₁-C₇ alkyl.

In embodiments, R^{1′} is

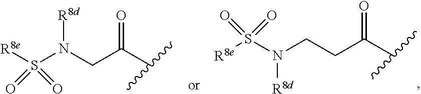

wherein R^{8d} is independently H or unsubstituted C₁-C₇ alkyl, and R⁸¹ is unsubstituted C₁-C₇ alkyl.

In embodiments, R^{1′} is

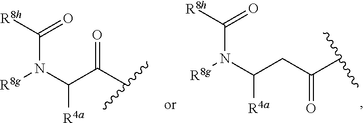

wherein each of R^{4a} and R^{8g} is independently H or unsubstituted C₁-C₇ alkyl; and R^{8h} is unsubstituted C₁-C₇ alkyl.

In embodiments, R^{1′} is

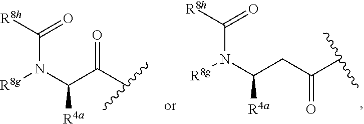

wherein each of R^{4a} and R^{8g} is independently H or unsubstituted C₁-C₇ alkyl; and R^{8h} is unsubstituted C₁-C₇ alkyl.

In embodiments, R^{1′} is

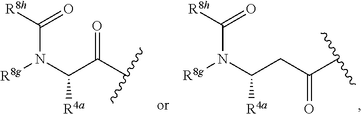

wherein each of R^{4a} and R^{8g} is independently H or unsubstituted C₁-C₇ alkyl; and R^{8h} is unsubstituted C₁-C₇ alkyl.

In embodiments, R^{1′} is

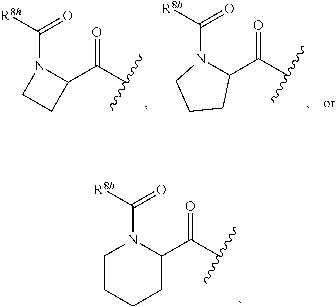

wherein R^{8h} is unsubstituted C₁-C₇ alkyl.

In embodiments, R^{1′} is

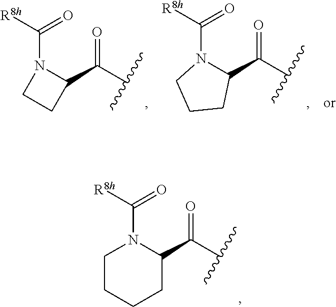

wherein R^{8h} is unsubstituted C₁-C₇ alkyl.

In embodiments, R^{1′} is

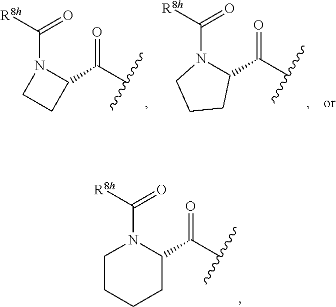

wherein R^{8h} is unsubstituted C₁-C₇ alkyl.

In embodiments, R^{1′} is

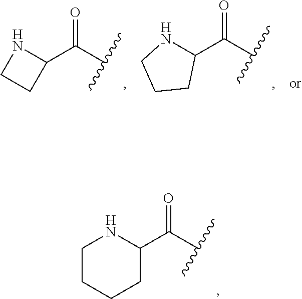

In embodiments, R^{1′} is

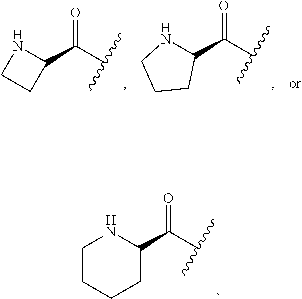

In embodiments, R^{1′} is

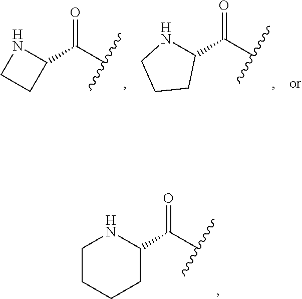

In embodiments, R^{1′} is

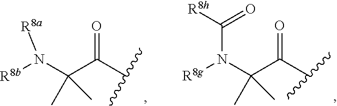

wherein each R^{8a}, R^{8b}, and R^{8g} is independently H or unsubstituted C₁-C₇ alkyl, and R^{8h} is unsubstituted C₁-C₇ alkyl.

In embodiments, R^{1′} is

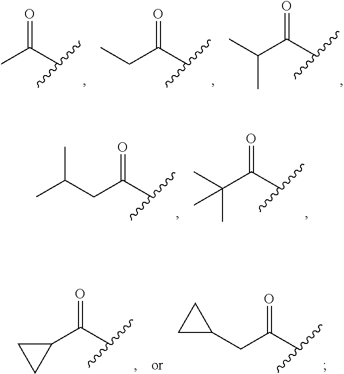

In embodiments, R^{1′} is

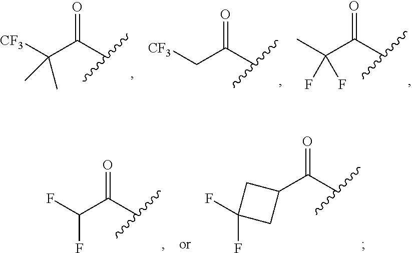

In embodiments, R^{1′} is

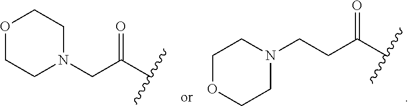 [SMILES: *C(=O)CN1CCOCC1 ; *C(=O)CCN1CCOCC1]

In embodiments, R^{1′} is

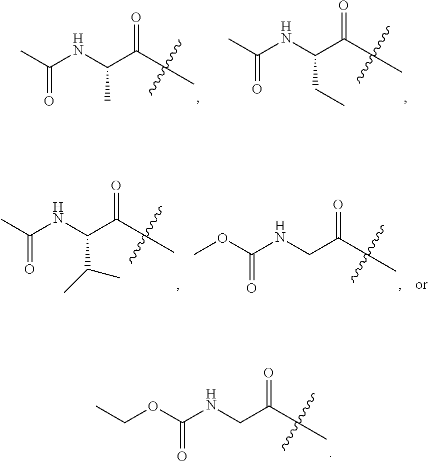

In embodiments, R^{1′} is

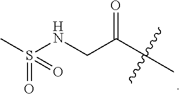 [SMILES: *C(=O)CNS(C)(=O)=O]

In embodiments, R^{1′} is

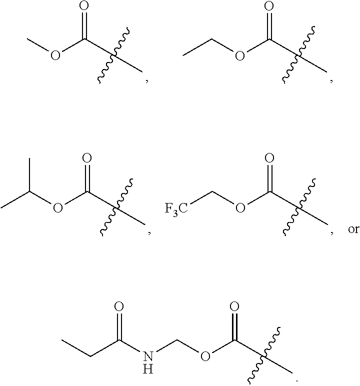

In embodiments, R^{1′} is

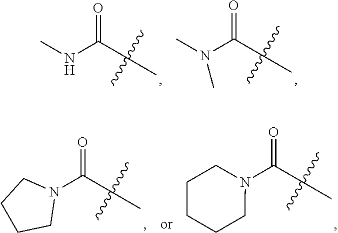

In embodiments, R^{1′} is

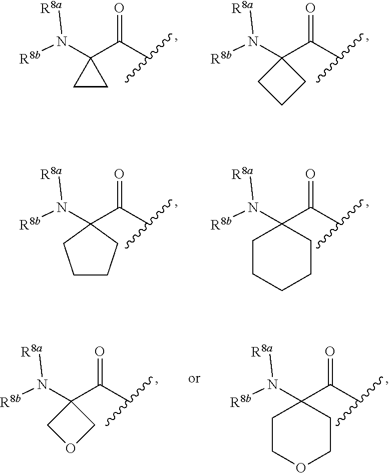

wherein each R^{8a} and R^{8b} is independently H or unsubstituted C₁-C₇ alkyl.

In embodiments, R^{1′} is

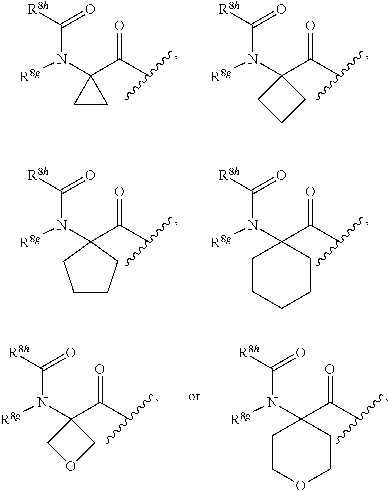

wherein R^{8g} is independently H or unsubstituted C₁-C₇ alkyl, and R^{8h} is independently unsubstituted C₁-C₇ alkyl.

In embodiments, R^{1′} is

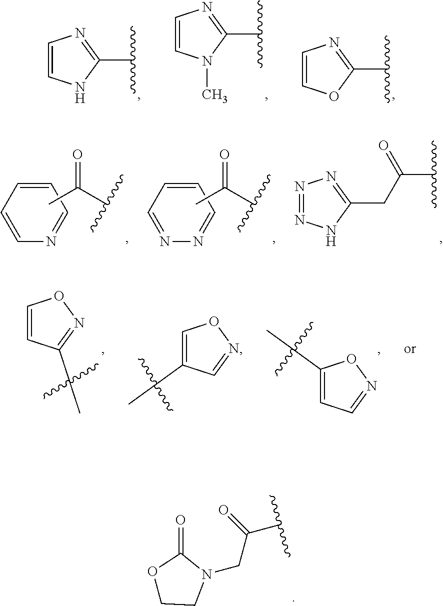

Compounds of Formulas (A)-(AAA)

Still further compounds of Formula (I), (I*), (I**), or (II) include compound of any one of Formulas (A)-(AAA) as described herein, wherein any variable can be according to any aspect or embodiment as described herein.

The further exemplary formulas and compounds described herein can also encompass hydrates, solvates, enantiomers, diastereomers, pharmaceutically acceptable salts, and complexes thereof.

For example, in any of the formulas described herein (e.g., any of Formula (I), (I*), (I**), (II) and any of Formulas (A)-(AAA), the C5 carbon of the 2-dihydrofuranone has the (R)-configuration.

Alternatively, in any of the formulas described herein (e.g., any of Formula (I), (I*), (I**), (II) and any of Formulas (A)-(AAA), the C5 carbon of the 2-dihydrofuranone has the (S)-configuration.

In embodiments, provided herein are compounds having a structure according to Formula (A):

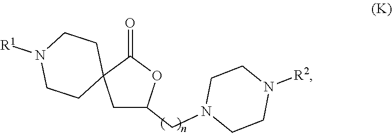

wherein R^{a}, R^{b}, A, n, and aryl are as according to any aspects and embodiments described herein.

In embodiments, provided herein are compounds having a structure according to Formula (B):

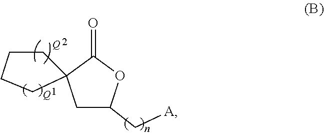

wherein Q¹ is 1 or 2, Q² is 1 or 2, and A and n are as according to any aspects and embodiments described herein.

In embodiments, provided herein are compounds having a structure according to Formula (C):

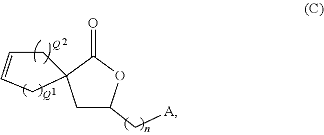

wherein Q¹ is 1 or 2, Q² is 1 or 2, and A and n are as according to any aspects and embodiments described herein.

In embodiments, provided herein are compounds having a structure according to Formula (D):

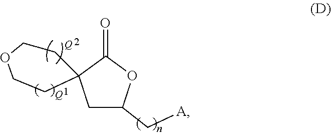

wherein Q¹ is 1 or 2, Q² is 1 or 2, A and n are as according to any aspects and embodiments described herein.

In embodiments, provided herein are compounds having a structure according to Formula (E):

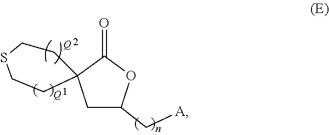

wherein Q¹ is 1 or 2, Q² is 1 or 2, and A and n are as according to any aspects and embodiments described herein.

In embodiments, provided herein are compounds having a structure according to Formula (F):

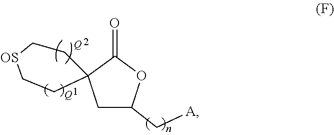

wherein Q¹ is 1 or 2, Q² is 1 or 2, and A and n are as according to any aspects and embodiments described herein.

In embodiments, provided herein are compounds having a structure according to Formula (G):

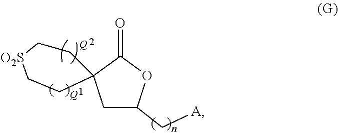

wherein Q¹ is 1 or 2, Q² is 1 or 2, and A and n are as according to any aspects and embodiments described herein.

In embodiments, provided herein are compounds having a structure according to Formula (H):

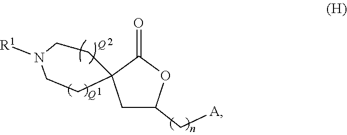

wherein Q¹ is 1 or 2, Q² is 1 or 2, and R¹, A, and n are as according to any aspects and embodiments described herein.

In specific embodiments, provided herein are compounds having a structure according to Formula (J):

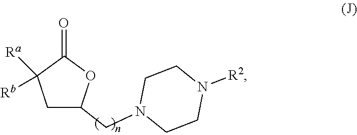

wherein each R^{a} and R^{b} is selected from the group consisting of hydrogen, C₁-C₇ alkyl, and C₃-C₇ branched alkyl;

R² is selected from the group consisting of phenyl, naphthyl, pyridyl, indolyl; and

R³ is selected from the group consisting of phenyl, naphthyl, pyridyl and indolyl.

In specific embodiments, provided herein are compounds having a structure according to Formula (K):

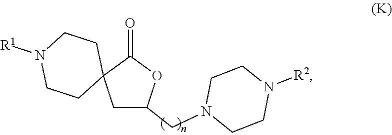

wherein

R² is selected from the group consisting of phenyl, naphthyl, pyridyl, indolyl; and

R³ is selected from the group consisting of phenyl, naphthyl, pyridyl and indolyl.

In embodiments, provided herein are compounds comprising a

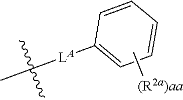

unit, wherein L^{A} is any group for A described herein. In embodiments, L^{A} is selected from the group consisting of

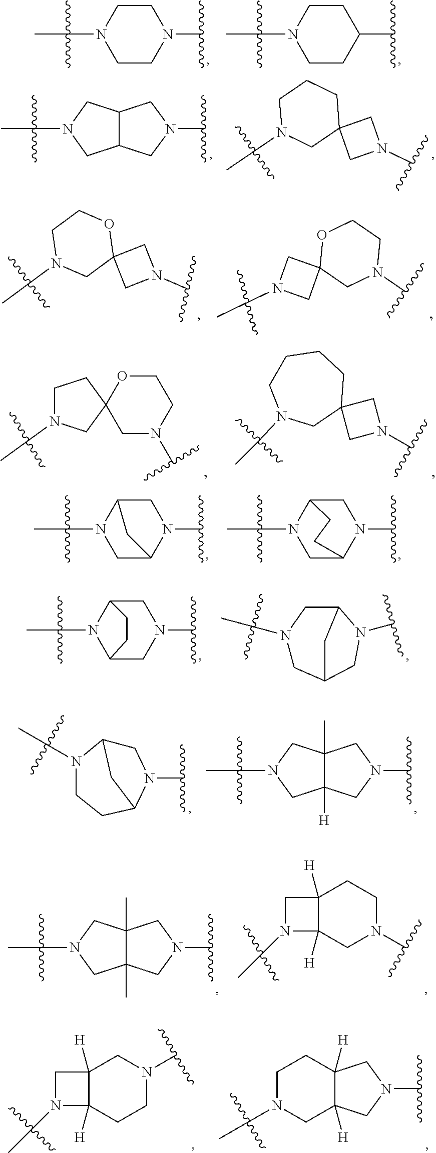

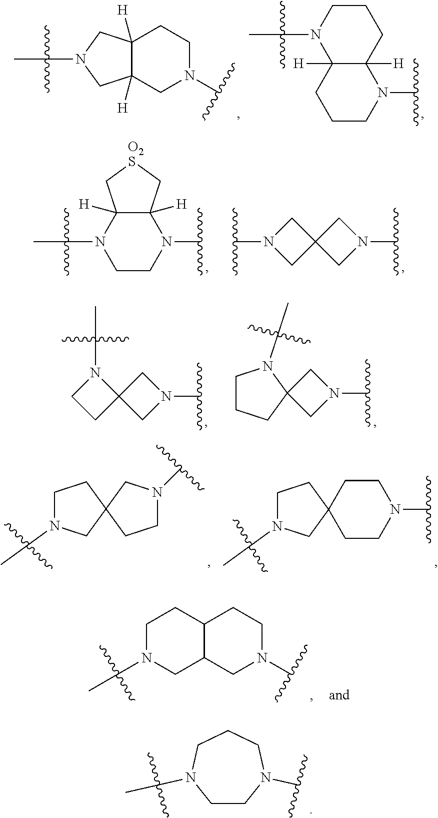

In embodiments, provided herein are compounds having a structure according to Formula (L):

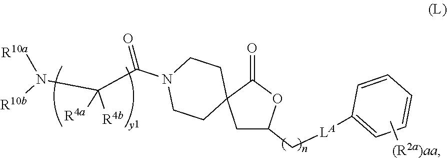

wherein R^{2a}, aa, L^{A}, n, R^{4a}, R^{4b}, y¹, R^{10a}, and R^{10b} are as according to any aspects and embodiments described herein.

In embodiments, provided herein are compounds having a structure according to Formula (M):

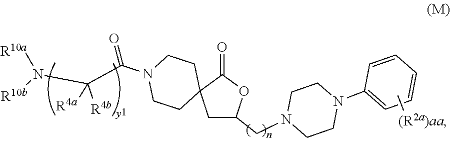

wherein R^{2a}, aa, n, R^{4a}, R^{4b}, y¹, R^{10a}, and R^{10b} are as according to any aspects and embodiments described herein.

In embodiments, provided herein are compounds having a structure according to formula (N):

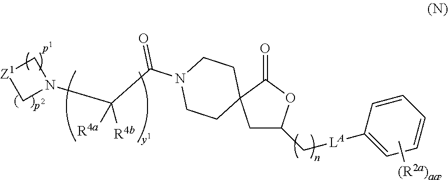

wherein R^{2a}, aa, L^{A}, n, R^{4a}, R^{4b}, y¹, Z¹, p¹, and p² are as according to any aspects and embodiments described herein.

In embodiments, provided herein are compounds having a structure according to formula (O):

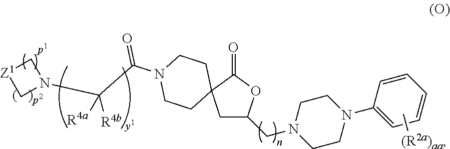

wherein R^{2a}, aa n R^{4a}, R^{4b}, y¹, Z¹, p¹, and p² are as according to any aspects and embodiments described herein.

In embodiments, provided herein are compounds having a structure according to formula (P):

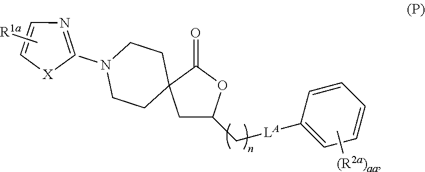

wherein R^{2a}, aa, L^{A}, n, X, and R^{1a} are as according to any aspects and embodiments described herein.

In embodiments, provided herein are compounds having a structure according to formula (Q):

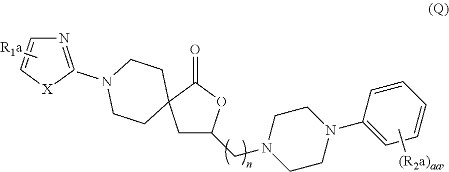

wherein R^{2a}, aa, n, X, and R^{1a} are as according to any aspects and embodiments described herein.

In embodiments, provided herein are compounds having a structure according to formula (R):

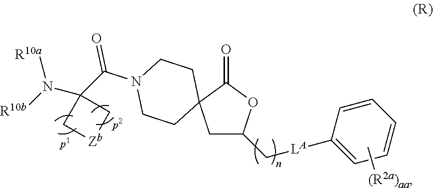

wherein R^{2a}, aa, L^{A}, n Z^{b}, p¹, p², and R^{10a}, and R^{10b} are as according to any aspects and embodiments described herein.

In embodiments, provided herein are compounds having a structure according to formula (S):

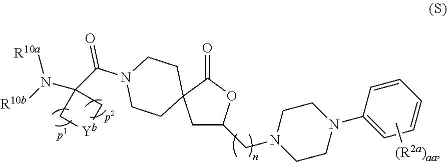

wherein R^{2a}, aa, n, Z^{b}, p¹, p², and R^{10a}, and R^{10b} are as according to any aspects and embodiments described herein.

In embodiments, provided herein are compounds having a structure according to formula (T):

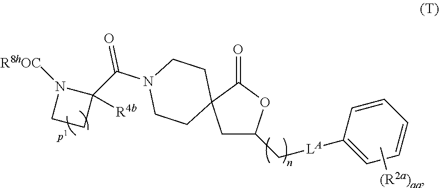

wherein p¹ is 1, 2, 3, or 4, and R^{2a}, aa, L^{A}, n, R^{4b}, and R^{8h} are as according to any aspects and embodiments described herein.

In embodiments, provided herein are compounds having a structure according to formula (U):

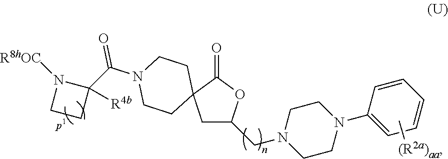

wherein p¹ is 1, 2, 3, or 4, and R^{2a}, aa, n, R^{4b}, and R^{8h} are as according to any aspects and embodiments described herein.

In embodiments, provided herein are compounds having a structure according to formula (V):

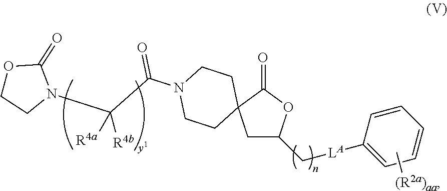

wherein R^{2a}, aa, L^{A}, n, R^{4a}, R^{4b}, and y¹ are as according to any aspects and embodiments described herein.

In embodiments, provided herein are compounds having a structure according to formula (W):

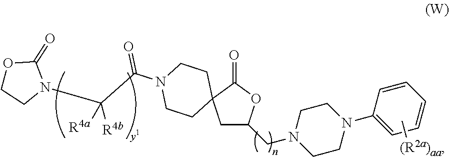

wherein and R^{2a}, aa, n, R^{4a}, R^{4b}, and y¹ are as according to any aspects and embodiments described herein.

In embodiments, provided herein are compounds having a structure according to formula (X):

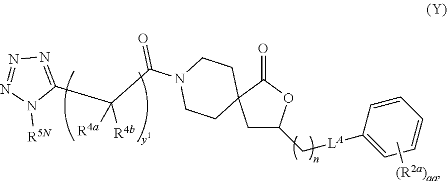

wherein R^{5N} is selected from H, C₁-C₇ linear alkyl, and C₃-C₇ branched alkyl, and R^{2a}, aa, L^{A}, n, R^{4a}, R^{4b}, and y¹ are as according to any aspects and embodiments described herein.

In embodiments, provided herein are compounds having a structure according to formula (Y):

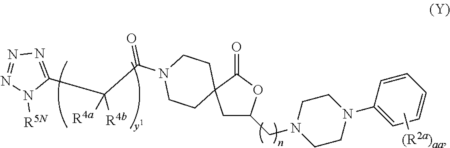

wherein R^{5N} is selected from H, C₁-C₇ linear alkyl, and C₃-C₇ branched alkyl, and R^{2a}, aa, n, R^{4a}, R^{4b}, and y¹ are as according to any aspects and embodiments described herein.

In embodiments, provided herein are compounds having a structure according to formula (Z):

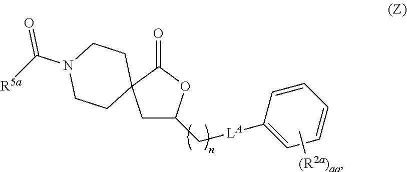

wherein R^{5a} is pyridyl or pyridazine, and R^{2a}, aa, L^{A} and n are as according to any aspects and embodiments described herein.

In embodiments, provided herein are compounds having a structure according to formula (AA):

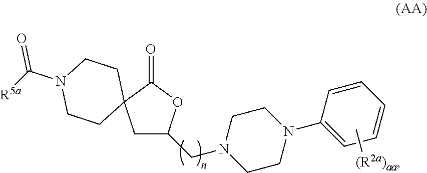

wherein R^{5a} is pyridyl or pyridazine, and R^{2a}, aa, and n are as according to any aspects and embodiments described herein.

In embodiments, provided herein are compounds having a structure according to formula (BB):

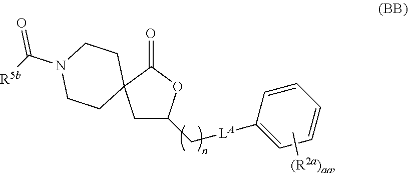

wherein R^{5b} is C₁-C₇ alkyl, C₃-C₇ cycloalkyl, C₁-C₇ fluoroalkyl, or C₃-C₇ cyclofluoroalkyl, and R^{2a}, aa, L^{A}, and n are as according to any aspects and embodiments described herein.

In embodiments, provided herein are compounds having a structure according to formula (CC):

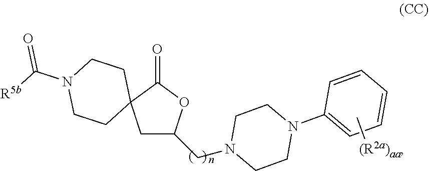

wherein R^{5b} is C₁-C₇ alkyl, C₃-C₇ cycloalkyl, C₁-C₇ fluoroalkyl, or C₃-C₇ cyclofluoroalkyl, and R^{2a}, aa, and n are as according to any aspects and embodiments described herein.

In embodiments, provided herein are compounds having a structure according to formula (DD):

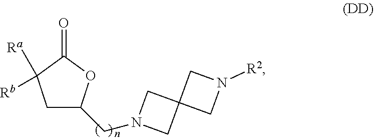

wherein R², n, R^{a}, and R^{b} are as according to any aspects and embodiments described herein.

In embodiments, provided herein are compounds having a structure according to formula (EE):

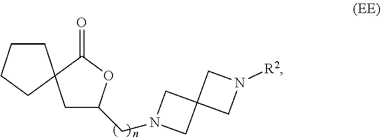

wherein R² and n are as according to any aspects and embodiments described herein.

In embodiments, provided herein are compounds having a structure according to formula (FF):

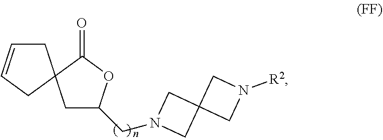

wherein R² and n are as according to any aspects and embodiments described herein.

In embodiments, provided herein are compounds having a structure according to formula (GG):

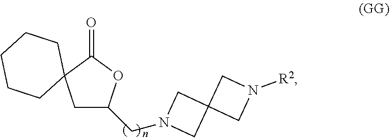

wherein R² and n are as according to any aspects and embodiments described herein.

In embodiments, provided herein are compounds having a structure according to formula (HH):

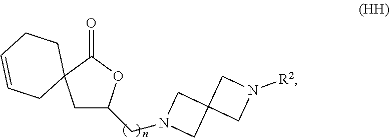

wherein R² and n are as according to any aspects and embodiments described herein.

In embodiments, provided herein are compounds having a structure according to formula (JJ):

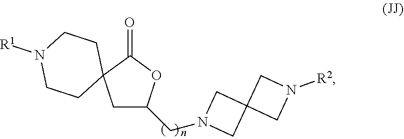

wherein R¹, R² and n are as according to any aspects and embodiments described herein.

In embodiments, provided herein are compounds having a structure according to formula (KK):

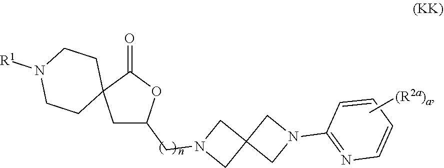

wherein R¹, R^{2a}, a, and n are as according to any aspects and embodiments described herein.

In embodiments, provided herein are compounds having a structure according to formula (LL):

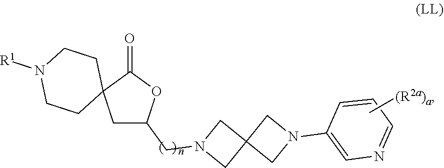

wherein R¹, R^{2a}, a, and n are as according to any aspects and embodiments described herein.

In embodiments, provided herein are compounds having a structure according to formula (MM):

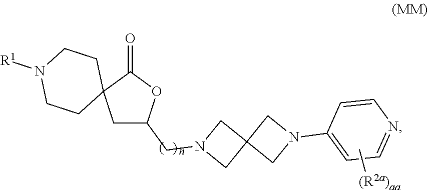

wherein R¹, R^{2a}, aa, and n are as according to any aspects and embodiments described herein.

In embodiments, provided herein are compounds having a structure according to formula (NN):

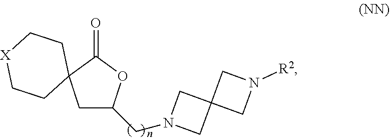

wherein X, R^{2a}, n are as according to any aspects and embodiments described herein.

In embodiments, provided herein are compounds having a structure according to formula (OO):

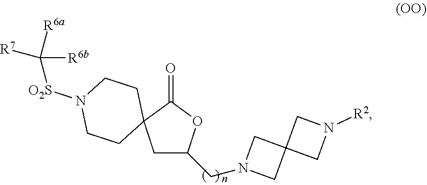

wherein R², n, R^{6a}, R^{6b}, and R⁷ are as according to any aspects and embodiments described herein.

In embodiments, provided herein are compounds having a structure according to formula (PP):

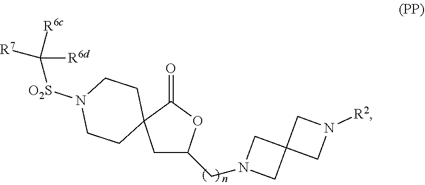

wherein R², n, R^{6c}, R^{6d}, and R⁷ are as according to any aspects and embodiments described herein.

In embodiments, provided herein are compounds having a structure according to formula (QQ):

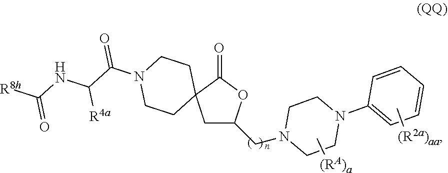

wherein R^{8h}, R^{2a}, R^{4a}, R^{A}, a, aa, and n are as described for any formula, aspect, or embodiment described herein. In embodiments, R^{8h} is unsubstituted C₁-C₇ alkyl (e.g., methyl). In embodiments, R^{4a} is hydrogen or unsubstituted C₁-C₇ alkyl (e.g., R^{4a} is hydrogen or methyl, ethyl, or isopropyl). In embodiments, n is 2. In embodiments, aa is 1 or 2. In embodiments, aa is 1. In embodiments, each R^{2a} is halogen (e.g., —F and/or —Cl). In embodiments, a is 0 or 1. In embodiments, R^{A}, when present, is unsubstituted C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{4a} has the (R)-configuration. In embodiments, the carbon substituted by R^{4a} has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (RR):

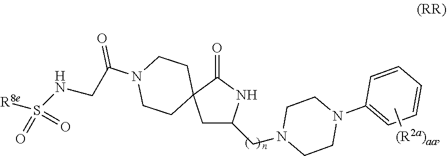

wherein R^{8e}, R^{2a}, a, and n are as described for any formula, aspect, or embodiment described herein. In embodiments, R^{8e} is unsubstituted C₁-C₇ alkyl (e.g., methyl). In embodiments, n is 2. In embodiments, aa is 1 or 2. In embodiments, aa is 1. In embodiments, each R^{2a} is halogen (e.g., —F and/or —Cl). In embodiments, the C5 carbon of the 2-dihydrofuranone core has the (R)-configuration. In embodiments, the C5 carbon of the 2-dihydrofuranone core has the (S)-configuration.

In embodiments, provided herein are compounds having a structure according to formula (SS):

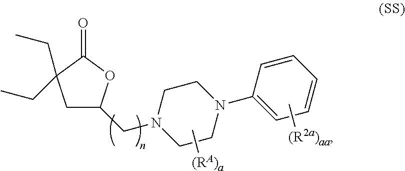

wherein R^{2a}, R^{A}, a, aa, and n are as described for any formula, aspect, or embodiment described herein. In embodiments, n is 2. In embodiments, aa is 0, 1, or 2. In embodiments, aa is 0. In embodiments, aa is 1. In embodiments, each R^{2a} is halogen (e.g., —F and/or —Cl). In embodiments, a is 1. In embodiments, R^{A} is C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (TT):

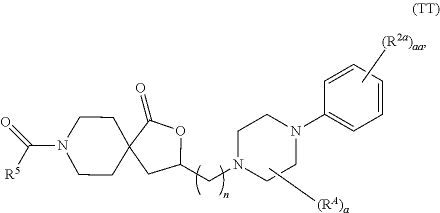

wherein R^{2a}, R^{A}, R⁵, a, aa, and n are as described for any formula, aspect, or embodiment described herein. In embodiments, R⁵ is unsubstituted C₁-C₇ alkyl (e.g., methyl or ethyl). In embodiments, n is 2. In embodiments, a is 0 or 1. In embodiments, R^{A} is C₁-C₇ alkyl (e.g., methyl). In embodiments, aa is 1. In embodiments, aa is 0, 1, or 2. In embodiments, aa is 0. In embodiments, aa is 1. In embodiments, each R^{2a} is halogen (e.g., —F and/or —Cl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (UU):

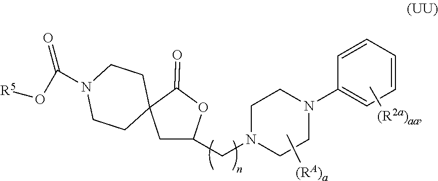

wherein R⁵, R^{A}, R^{2a}, a, aa, and n are as described for any formula, aspect, or embodiment described herein. In embodiments, R⁵ is unsubstituted C₁-C₇ alkyl (e.g., methyl, ethyl, isopropyl). In embodiments, R⁵ is C₁-C₇ haloalkyl (e.g., CH₂CF₃). In embodiments, n is 2. In embodiments, aa is 1 or 2. In embodiments, aa is 1. In embodiments, each R^{2a} is halogen (e.g., —F and/or —Cl). In embodiments, a is 0 or 1. In embodiments, R^{A}, when present, is unsubstituted C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (VV):

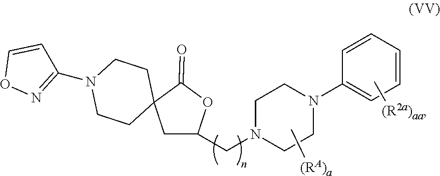

wherein R^{A}, R^{2a}, a, aa, and n are as described for any formula, aspect, or embodiment described herein. In embodiments, n is 2. In embodiments, aa is 1 or 2. In embodiments, aa is 1. In embodiments, each R^{2a} is halogen (e.g., —F and/or —Cl). In embodiments, a is 0 or 1. In embodiments, R^{A}, when present, is unsubstituted C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (WW):

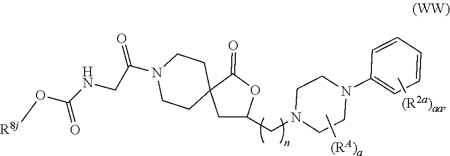

wherein R^{8j}, R^{2a}, R^{A}, a, aa, and n are as described for any formula, aspect, or embodiment described herein. In embodiments, R^{8j} is unsubstituted C₁-C₇ alkyl (e.g., methyl or ethyl). In embodiments, n is 2. In embodiments, aa is 1 or 2. In embodiments, aa is 1. In embodiments, each R^{2a} is halogen (e.g., —F and/or —Cl). In embodiments, a is 0 or 1. In embodiments, R^{A}, when present, is unsubstituted C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (XX):

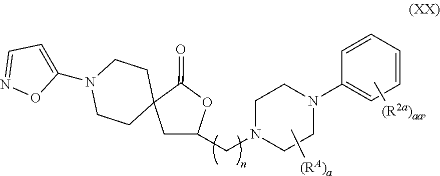

wherein R^{A}, R^{2a}, a, aa, and n are as described for any formula, aspect, or embodiment described herein. In embodiments, n is 2. In embodiments, aa is 1 or 2. In embodiments, aa is 1. In embodiments, each R^{2a} is halogen (e.g., —F and/or —Cl). In embodiments, a is 0 or 1. In embodiments, R^{A}, when present, is unsubstituted C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (YY):

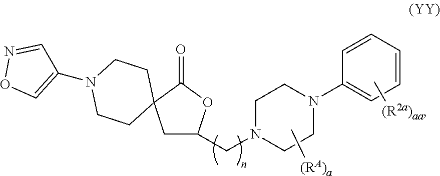

wherein R^{A}, R^{2a}, a, aa, and n are as described for any formula, aspect, or embodiment described herein. In embodiments, n is 2. In embodiments, aa is 1 or 2. In embodiments, aa is 1. In embodiments, each R^{2a} is halogen (e.g., —F and/or —Cl). In embodiments, a is 0 or 1. In embodiments, R^{A}, when present, is unsubstituted C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (ZZ):

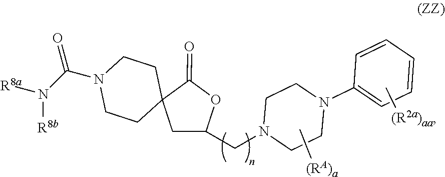

wherein R^{A}, R^{2a}, R^{8a}, R^{8b}, a, aa, and n are as described for any formula, aspect, or embodiment described herein. In embodiments, R^{8a} is hydrogen or unsubstituted C₁-C₇ alkyl (e.g., methyl). In embodiments, R^{8b} is hydrogen or unsubstituted C₁-C₇ alkyl (e.g., methyl). In embodiments, n is 2. In embodiments, aa is 1 or 2. In embodiments, aa is 1. In embodiments, each R^{2a} is halogen (e.g., —F and/or —Cl). In embodiments, a is 0 or 1. In embodiments, R^{A}, when present, is unsubstituted C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

In embodiments, provided herein are compounds having a structure according to formula (AAA):

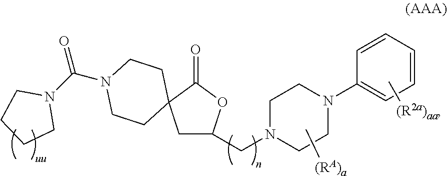

wherein R^{A}, R^{2a}, R^{8a}, R^{8b}, a, aa, and n are as described for any formula, aspect, or embodiment described herein. In embodiments, uu is 1 or 2. In embodiments, n is 2. In embodiments, aa is 1 or 2. In embodiments, aa is 1. In embodiments, each R^{2a} is halogen (e.g., —F and/or —Cl). In embodiments, a is 0 or 1. In embodiments, R^{A}, when present, is unsubstituted C₁-C₇ alkyl (e.g., methyl). In embodiments, the C5 carbon of the 2-dihydrofuranone core has the (R)-configuration. In embodiments, the C5 carbon of the 2-dihydrofuranone core has the (S)-configuration. In embodiments, the carbon substituted by R^{A} has the (R)-configuration. In embodiments, the carbon substituted by R^{A} has the (S)-configuration.

Exemplary Compounds

Exemplary compounds according to formulas described (e.g., according to Formula (I), (I*), (I**), or (II), such as any of Formulas (A)-(AAA)) herein include Compounds 1-145 as described in Table 1.

TABLE 1
Exemplary Compounds

| | | | | 5-HT7 | |
|---|---|---|---|---|---|
| Compound | | | | Ki | Kb |
| Number | Structure | TPSA | clogP | (nM) | (nM) |
| 1. | 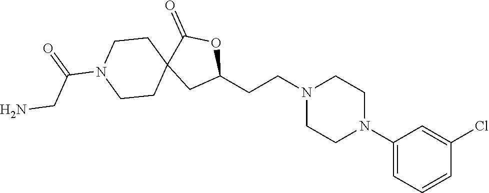 [SMILES: NCC(=O)N1CCC2(CC1)C[C@H](CCN1CCN(c3cccc(Cl)c3)CC1)OC2=O] | 79.1 | 0.89 | — | — |
| 2. | 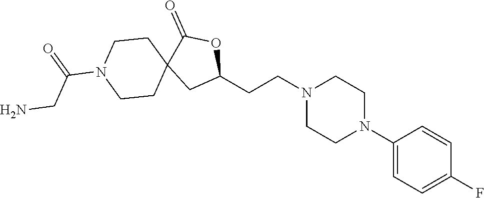 [SMILES: NCC(=O)N1CCC2(CC1)C[C@H](CCN1CCN(c3ccc(F)cc3)CC1)OC2=O] | 79.1 | 0.32 | — | — |
| 3. | 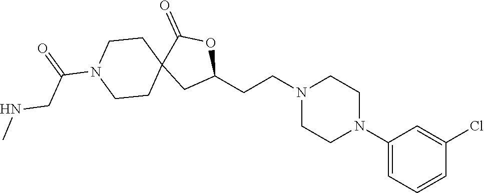 [SMILES: CNCC(=O)N1CCC2(CC1)C[C@H](CCN1CCN(c3cccc(Cl)c3)CC1)OC2=O] | 65.1 | 1.24 | — | — |
| 4. | 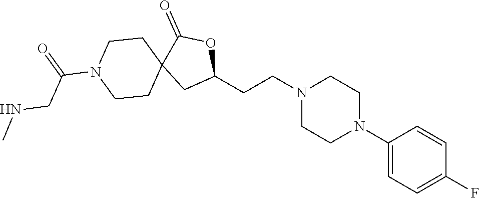 [SMILES: CNCC(=O)N1CCC2(CC1)C[C@H](CCN1CCN(c3ccc(F)cc3)CC1)OC2=O] | 65.1 | 0.67 | — | — |
| 5. | 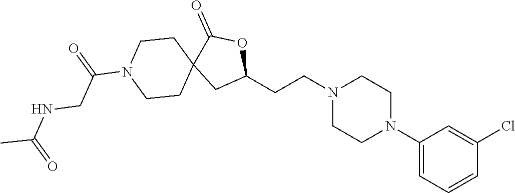 [SMILES: CC(=O)NCC(=O)N1CCC2(CC1)C[C@H](CCN1CCN(c3cccc(Cl)c3)CC1)OC2=O] | 82.1 | 0.85 | — | — |
| 6. | 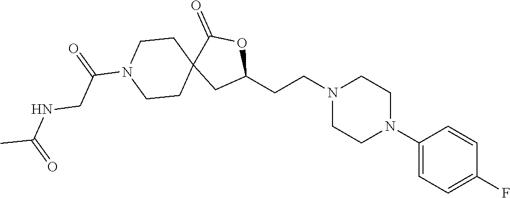 [SMILES: CC(=O)NCC(=O)N1CCC2(CC1)C[C@H](CCN1CCN(c3ccc(F)cc3)CC1)OC2=O] | 82.1 | 0.28 | — | B |
| 7. | 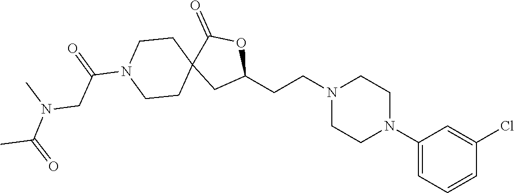 [SMILES: CC(=O)N(C)CC(=O)N1CCC2(CC1)C[C@H](CCN1CCN(c3cccc(Cl)c3)CC1)OC2=O] | 73.4 | 1.28 | — | — |
| 8. | 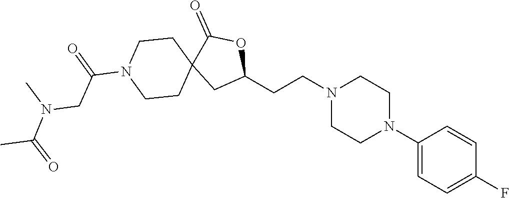 [SMILES: CC(=O)N(C)CC(=O)N1CCC2(CC1)C[C@H](CCN1CCN(c3ccc(F)cc3)CC1)OC2=O] | 73.4 | 0.71 | — | — |
| 9. | 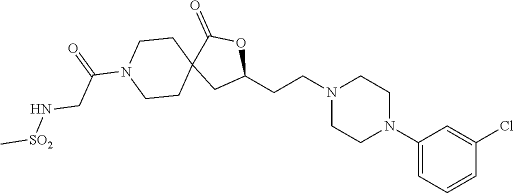 [SMILES: CS(=O)(=O)NCC(=O)N1CCC2(CC1)C[C@H](CCN1CCN(c3cccc(Cl)c3)CC1)OC2=O] | 99.2 | 1.00 | B | B |
| 10. | 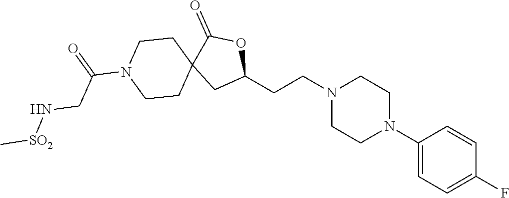 [SMILES: CS(=O)(=O)NCC(=O)N1CCC2(CC1)C[C@H](CCN1CCN(c3ccc(F)cc3)CC1)OC2=O] | 99.2 | 0.43 | — | — |
| 11. | 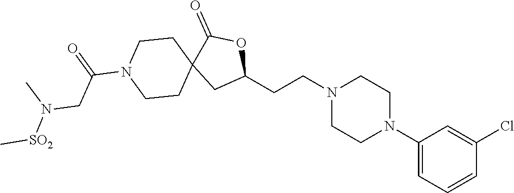 [SMILES: CN(CC(=O)N1CCC2(CC1)C[C@H](CCN1CCN(c3cccc(Cl)c3)CC1)OC2=O)S(C)(=O)=O] | 90.4 | 1.41 | — | — |
| 12. | 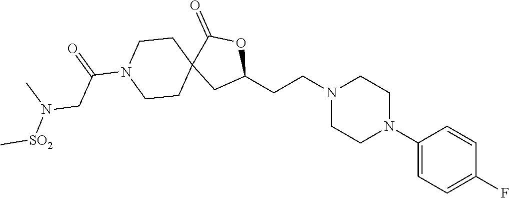 [SMILES: CN(CC(=O)N1CCC2(CC1)C[C@H](CCN1CCN(c3ccc(F)cc3)CC1)OC2=O)S(C)(=O)=O] | 90.4 | 0.84 | — | — |
| 13. | 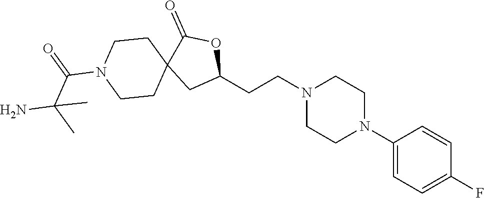 [SMILES: CC(C)(N)C(=O)N1CCC2(CC1)C[C@H](CCN1CCN(c3ccc(F)cc3)CC1)OC2=O] | 79.1 | 0.94 | — | — |
| 14. | 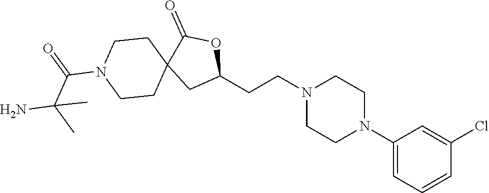 [SMILES: CC(C)(N)C(=O)N1CCC2(CC1)C[C@H](CCN1CCN(c3cccc(Cl)c3)CC1)OC2=O] | 79.1 | 1.51 | — | — |
| 15. | 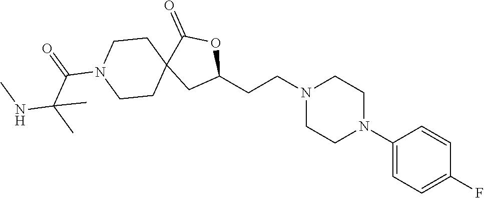 [SMILES: CNC(C)(C)C(=O)N1CCC2(CC1)C[C@H](CCN1CCN(c3ccc(F)cc3)CC1)OC2=O] | 65.1 | 1.28 | — | — |
| 16. | 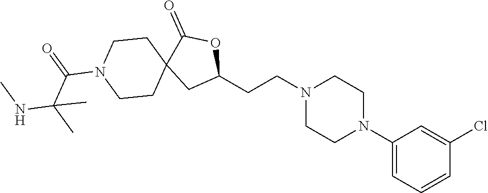 [SMILES: CNC(C)(C)C(=O)N1CCC2(CC1)C[C@H](CCN1CCN(c3cccc(Cl)c3)CC1)OC2=O] | 65.1 | 1.85 | — | — |
| 17. | 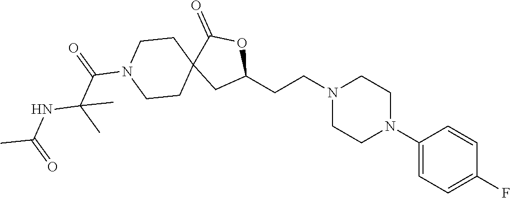 [SMILES: CC(=O)NC(C)(C)C(=O)N1CCC2(CC1)C[C@H](CCN1CCN(c3ccc(F)cc3)CC1)OC2=O] | 82.1 | 0.90 | — | — |
| 18. | 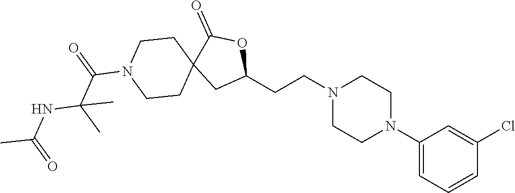 [SMILES: CC(=O)NC(C)(C)C(=O)N1CCC2(CC1)C[C@H](CCN1CCN(c3cccc(Cl)c3)CC1)OC2=O] | 82.1 | 1.47 | — | — |
| 19. | 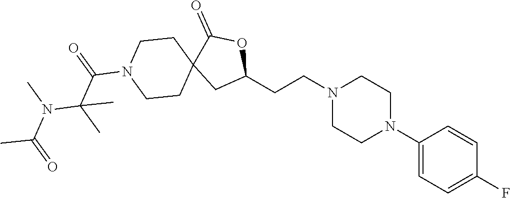 [SMILES: CC(=O)N(C)C(C)(C)C(=O)N1CCC2(CC1)C[C@H](CCN1CCN(c3ccc(F)cc3)CC1)OC2=O] | 73.4 | 1.33 | — | — |
| 20. | 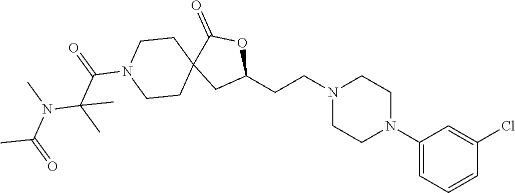 [SMILES: CC(=O)N(C)C(C)(C)C(=O)N1CCC2(CC1)C[C@H](CCN1CCN(c3cccc(Cl)c3)CC1)OC2=O] | 73.4 | 1.90 | — | — |
| 21. | 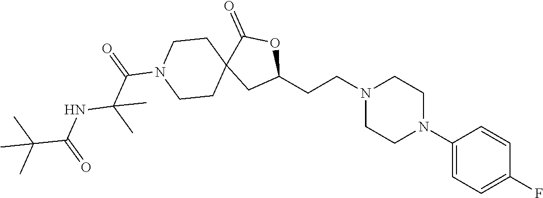 [SMILES: CC(C)(C)C(=O)NC(C)(C)C(=O)N1CCC2(CC1)C[C@H](CCN1CCN(c3ccc(F)cc3)CC1)OC2=O] | 82.1 | 2.14 | — | — |
| 22. | 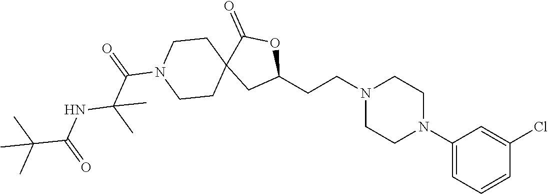 [SMILES: CC(C)(C)C(=O)NC(C)(C)C(=O)N1CCC2(CC1)C[C@H](CCN1CCN(c3cccc(Cl)c3)CC1)OC2=O] | 82.1 | 2.71 | — | — |
| 23. | 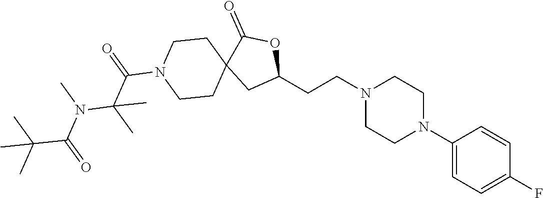 [SMILES: CN(C(=O)C(C)(C)C)C(C)(C)C(=O)N1CCC2(CC1)C[C@H](CCN1CCN(c3ccc(F)cc3)CC1)OC2=O] | 73.4 | 2.57 | — | — |
| 24. | 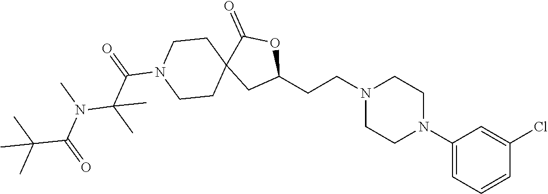 [SMILES: CN(C(=O)C(C)(C)C)C(C)(C)C(=O)N1CCC2(CC1)C[C@H](CCN1CCN(c3cccc(Cl)c3)CC1)OC2=O] | 73.4 | 3.14 | — | — |
| 25. | 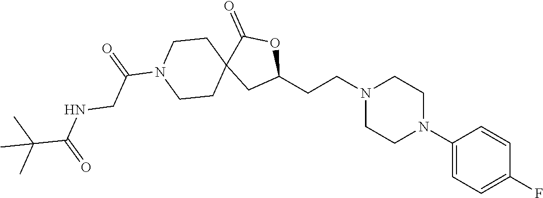 [SMILES: CC(C)(C)C(=O)NCC(=O)N1CCC2(CC1)C[C@H](CCN1CCN(c3ccc(F)cc3)CC1)OC2=O] | 82.1 | 1.52 | — | — |
| 26. | 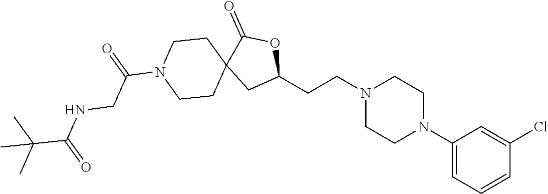 [SMILES: CC(C)(C)C(=O)NCC(=O)N1CCC2(CC1)C[C@H](CCN1CCN(c3cccc(Cl)c3)CC1)OC2=O] | 82.1 | 2.09 | — | — |
| 27. | 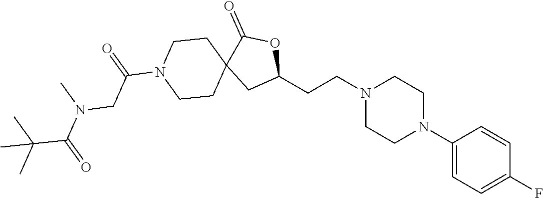 [SMILES: CN(CC(=O)N1CCC2(CC1)C[C@H](CCN1CCN(c3ccc(F)cc3)CC1)OC2=O)C(=O)C(C)(C)C] | 73.4 | 1.95 | — | — |
| 28. | 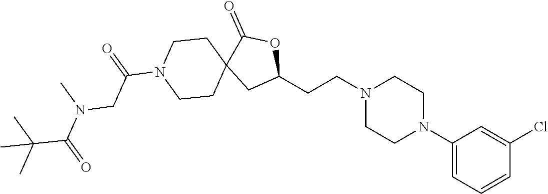 [SMILES: CN(CC(=O)N1CCC2(CC1)C[C@H](CCN1CCN(c3cccc(Cl)c3)CC1)OC2=O)C(=O)C(C)(C)C] | 73.4 | 2.52 | — | — |
| 29. | 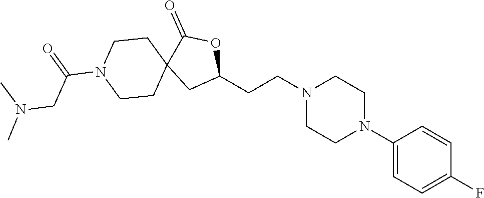 [SMILES: CN(C)CC(=O)N1CCC2(CC1)C[C@H](CCN1CCN(c3ccc(F)cc3)CC1)OC2=O] | 56.3 | 1.27 | — | — |
| 30. | 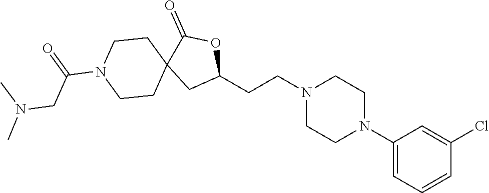 [SMILES: CN(C)CC(=O)N1CCC2(CC1)C[C@H](CCN1CCN(c3cccc(Cl)c3)CC1)OC2=O] | 56.3 | 1.84 | — | — |
| 31. | 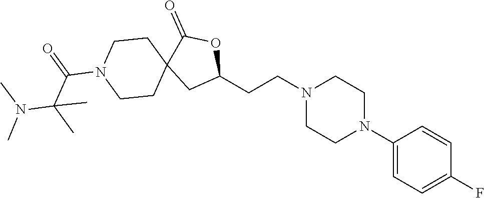 [SMILES: CN(C)C(C)(C)C(=O)N1CCC2(CC1)C[C@H](CCN1CCN(c3ccc(F)cc3)CC1)OC2=O] | 56.3 | 1.89 | — | — |
| 32. | 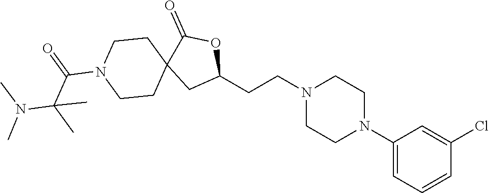 [SMILES: CN(C)C(C)(C)C(=O)N1CCC2(CC1)C[C@H](CCN1CCN(c3cccc(Cl)c3)CC1)OC2=O] | 56.3 | 2.46 | — | — |
| 33. | 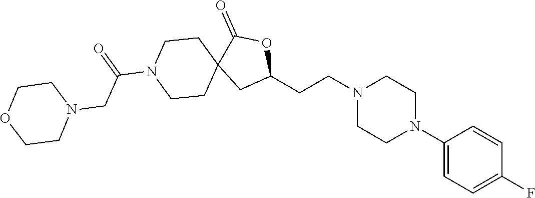 [SMILES: O=C(CN1CCOCC1)N1CCC2(CC1)C[C@H](CCN1CCN(c3ccc(F)cc3)CC1)OC2=O] | 65.5 | 1.26 | — | — |
| 34. | 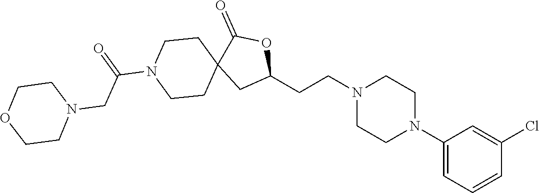 [SMILES: O=C(CN1CCOCC1)N1CCC2(CC1)C[C@H](CCN1CCN(c3cccc(Cl)c3)CC1)OC2=O] | 65.5 | 1.83 | — | — |
| 35. | 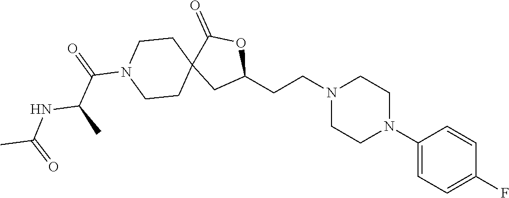 [SMILES: CC(=O)N[C@H](C)C(=O)N1CCC2(CC1)C[C@H](CCN1CCN(c3ccc(F)cc3)CC1)OC2=O] | 82.1 | 0.59 | — | — |
| 36. | 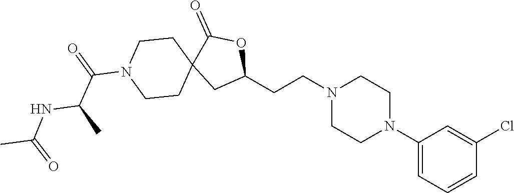 [SMILES: CC(=O)N[C@H](C)C(=O)N1CCC2(CC1)C[C@H](CCN1CCN(c3cccc(Cl)c3)CC1)OC2=O] | 82.1 | 1.16 | — | — |
| 37. | 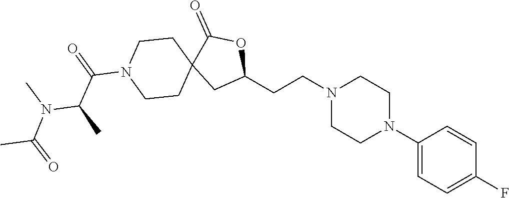 [SMILES: CC(=O)N(C)[C@H](C)C(=O)N1CCC2(CC1)C[C@H](CCN1CCN(c3ccc(F)cc3)CC1)OC2=O] | 73.4 | 1.02 | — | — |
| 38. | 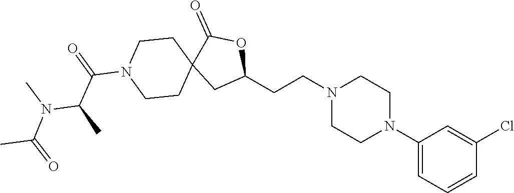 [SMILES: CC(=O)N(C)[C@H](C)C(=O)N1CCC2(CC1)C[C@H](CCN1CCN(c3cccc(Cl)c3)CC1)OC2=O] | 73.4 | 1.59 | — | — |
| 39. | 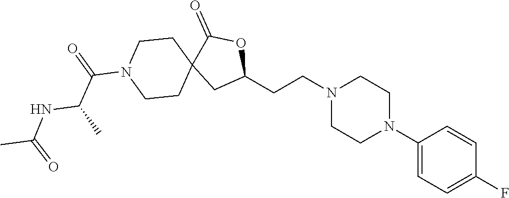 [SMILES: CC(=O)N[C@@H](C)C(=O)N1CCC2(CC1)C[C@H](CCN1CCN(c3ccc(F)cc3)CC1)OC2=O] | 82.1 | 0.59 | — | — |
| 40. | 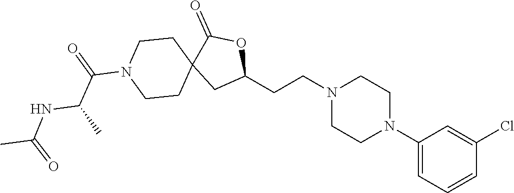 [SMILES: CC(=O)N[C@@H](C)C(=O)N1CCC2(CC1)C[C@H](CCN1CCN(c3cccc(Cl)c3)CC1)OC2=O] | 82.1 | 1.16 | — | — |
| 41. | 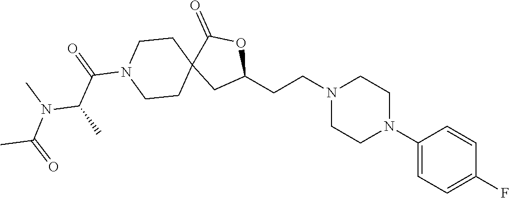 [SMILES: CC(=O)N(C)[C@@H](C)C(=O)N1CCC2(CC1)C[C@H](CCN1CCN(c3ccc(F)cc3)CC1)OC2=O] | 73.4 | 1.02 | — | — |
| 42. | 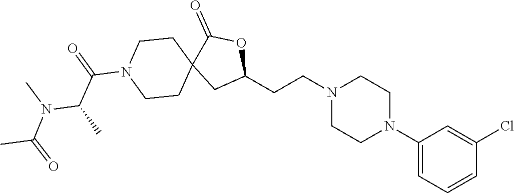 [SMILES: CC(=O)N(C)[C@@H](C)C(=O)N1CCC2(CC1)C[C@H](CCN1CCN(c3cccc(Cl)c3)CC1)OC2=O] | 73.4 | 1.59 | — | — |
| 43. | 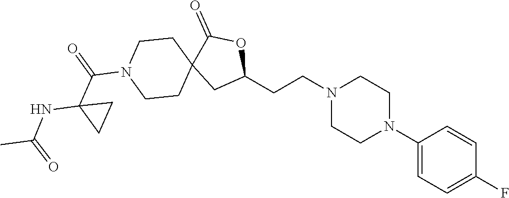 [SMILES: CC(=O)NC1(C(=O)N2CCC3(CC2)C[C@H](CCN2CCN(c4ccc(F)cc4)CC2)OC3=O)CC1] | 82.1 | 0.54 | — | — |
| 44. | 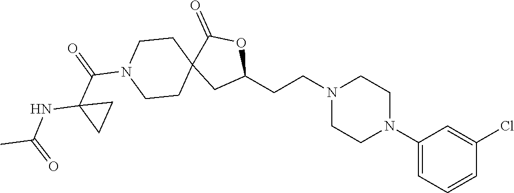 [SMILES: CC(=O)NC1(C(=O)N2CCC3(CC2)C[C@H](CCN2CCN(c4cccc(Cl)c4)CC2)OC3=O)CC1] | 82.1 | 1.14 | — | — |
| 45. | 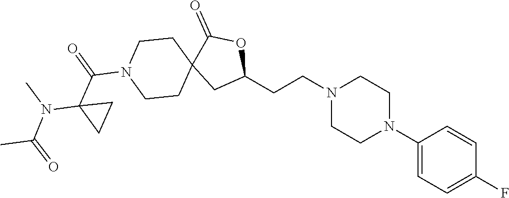 [SMILES: CC(=O)N(C)C1(C(=O)N2CCC3(CC2)C[C@H](CCN2CCN(c4ccc(F)cc4)CC2)OC3=O)CC1] | 73.4 | 0.97 | — | — |
| 46. | 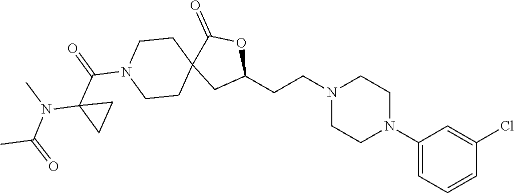 [SMILES: CC(=O)N(C)C1(C(=O)N2CCC3(CC2)C[C@H](CCN2CCN(c4cccc(Cl)c4)CC2)OC3=O)CC1] | 73.4 | 1.54 | — | — |
| 47. | 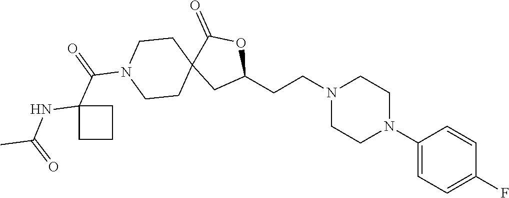 [SMILES: CC(=O)NC1(C(=O)N2CCC3(CC2)C[C@H](CCN2CCN(c4ccc(F)cc4)CC2)OC3=O)CCC1] | 82.1 | 1.10 | — | — |
| 48. | 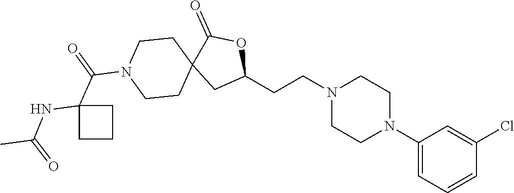 [SMILES: CC(=O)NC1(C(=O)N2CCC3(CC2)C[C@H](CCN2CCN(c4cccc(Cl)c4)CC2)OC3=O)CCC1] | 82.1 | 1.67 | — | — |
| 49. | 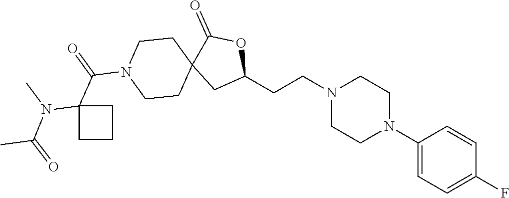 [SMILES: CC(=O)N(C)C1(C(=O)N2CCC3(CC2)C[C@H](CCN2CCN(c4ccc(F)cc4)CC2)OC3=O)CCC1] | 73.4 | 1.53 | — | — |
| 50. | 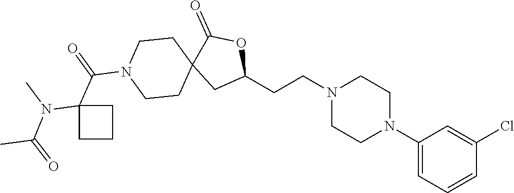 [SMILES: CC(=O)N(C)C1(C(=O)N2CCC3(CC2)C[C@H](CCN2CCN(c4cccc(Cl)c4)CC2)OC3=O)CCC1] | 73.4 | 2.10 | — | — |
| 51. | 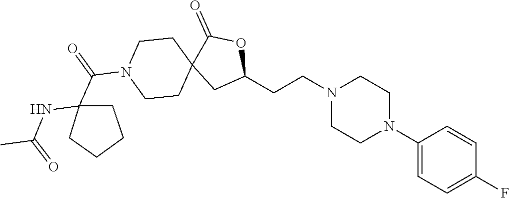 [SMILES: CC(=O)NC1(C(=O)N2CCC3(CC2)C[C@H](CCN2CCN(c4ccc(F)cc4)CC2)OC3=O)CCCC1] | 82.1 | 1.66 | — | — |
| 52. | 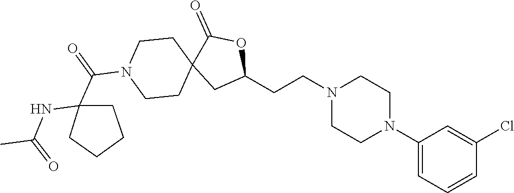 [SMILES: CC(=O)NC1(C(=O)N2CCC3(CC2)C[C@H](CCN2CCN(c4cccc(Cl)c4)CC2)OC3=O)CCCC1] | 82.1 | 2.23 | — | — |
| 53. | 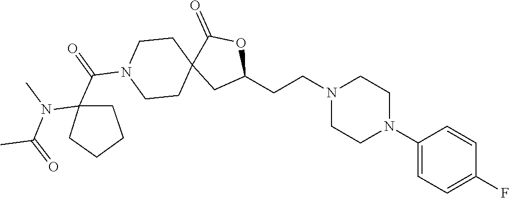 [SMILES: CC(=O)N(C)C1(C(=O)N2CCC3(CC2)C[C@H](CCN2CCN(c4ccc(F)cc4)CC2)OC3=O)CCCC1] | 73.4 | 2.09 | — | — |
| 54. | 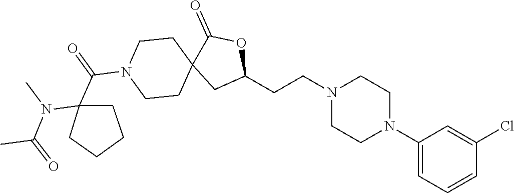 [SMILES: CC(=O)N(C)C1(C(=O)N2CCC3(CC2)C[C@H](CCN2CCN(c4cccc(Cl)c4)CC2)OC3=O)CCCC1] | 73.4 | 2.66 | — | — |
| 55. | 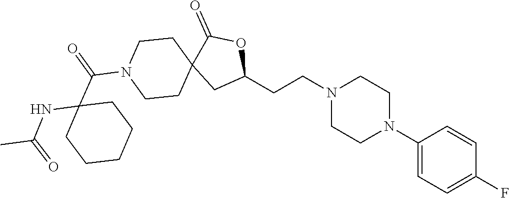 [SMILES: CC(=O)NC1(C(=O)N2CCC3(CC2)C[C@H](CCN2CCN(c4ccc(F)cc4)CC2)OC3=O)CCCCC1] | 82.1 | 2.22 | — | — |
| 56. | 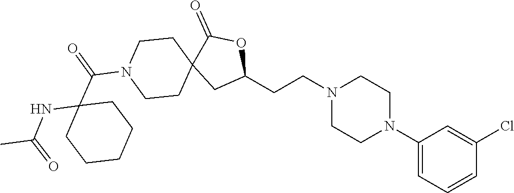 [SMILES: CC(=O)NC1(C(=O)N2CCC3(CC2)C[C@H](CCN2CCN(c4cccc(Cl)c4)CC2)OC3=O)CCCCC1] | 82.1 | 2.79 | — | — |
| 57. | 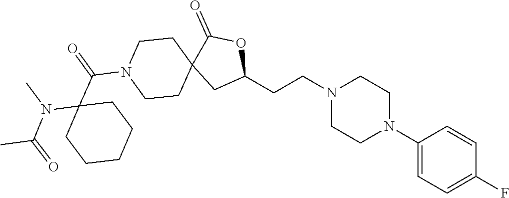 [SMILES: CC(=O)N(C)C1(C(=O)N2CCC3(CC2)C[C@H](CCN2CCN(c4ccc(F)cc4)CC2)OC3=O)CCCCC1] | 73.4 | 2.65 | — | — |
| 58. | 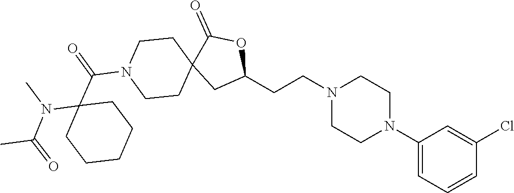 [SMILES: CC(=O)N(C)C1(C(=O)N2CCC3(CC2)C[C@H](CCN2CCN(c4cccc(Cl)c4)CC2)OC3=O)CCCCC1] | 73.4 | 3.22 | — | — |
| 59. | 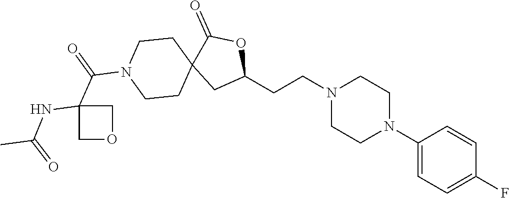 [SMILES: CC(=O)NC1(C(=O)N2CCC3(CC2)C[C@H](CCN2CCN(c4ccc(F)cc4)CC2)OC3=O)COC1] | 91.4 | 0.96 | — | — |
| 60. | 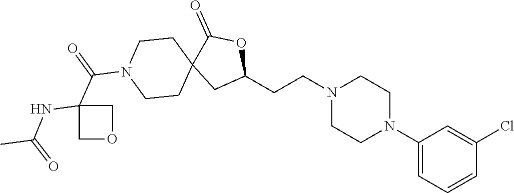 [SMILES: CC(=O)NC1(C(=O)N2CCC3(CC2)C[C@H](CCN2CCN(c4cccc(Cl)c4)CC2)OC3=O)COC1] | 91.4 | 1.53 | — | — |
| 61. | 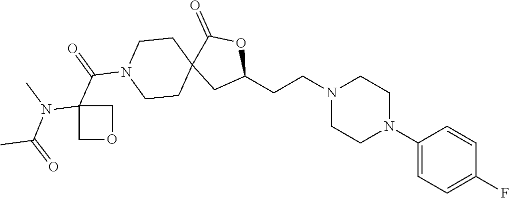 [SMILES: CC(=O)N(C)C1(C(=O)N2CCC3(CC2)C[C@H](CCN2CCN(c4ccc(F)cc4)CC2)OC3=O)COC1] | 82.6 | 1.50 | — | — |
| 62. | 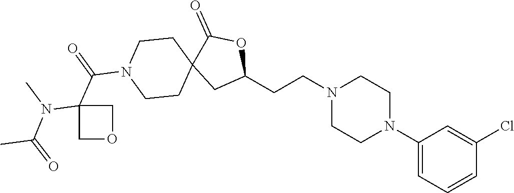 [SMILES: CC(=O)N(C)C1(C(=O)N2CCC3(CC2)C[C@H](CCN2CCN(c4cccc(Cl)c4)CC2)OC3=O)COC1] | 82.6 | 2.07 | — | — |
| 63. | 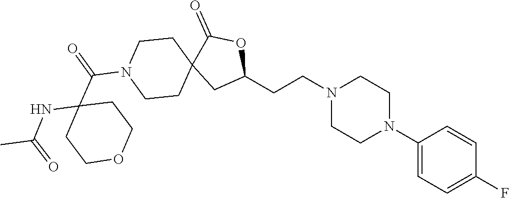 [SMILES: CC(=O)NC1(C(=O)N2CCC3(CC2)C[C@H](CCN2CCN(c4ccc(F)cc4)CC2)OC3=O)CCOCC1] | 91.4 | −0.17 | — | — |
| 64. | 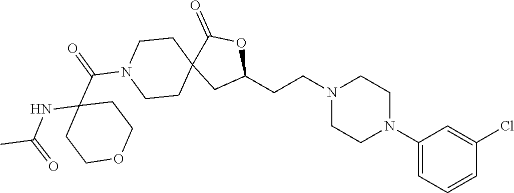 [SMILES: CC(=O)NC1(C(=O)N2CCC3(CC2)C[C@H](CCN2CCN(c4cccc(Cl)c4)CC2)OC3=O)CCOCC1] | 91.4 | 0.39 | — | — |
| 65. | 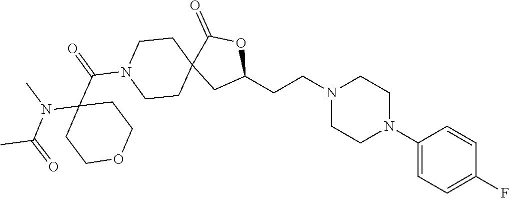 [SMILES: CC(=O)N(C)C1(C(=O)N2CCC3(CC2)C[C@H](CCN2CCN(c4ccc(F)cc4)CC2)OC3=O)CCOCC1] | 82.6 | 0.25 | — | — |
| 66. | 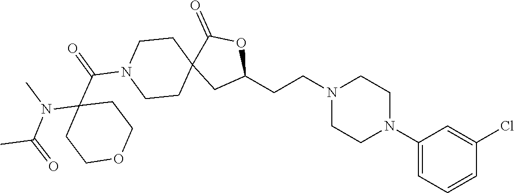 [SMILES: CC(=O)N(C)C1(C(=O)N2CCC3(CC2)C[C@H](CCN2CCN(c4cccc(Cl)c4)CC2)OC3=O)CCOCC1] | 82.6 | 0.82 | — | — |
| 67. | 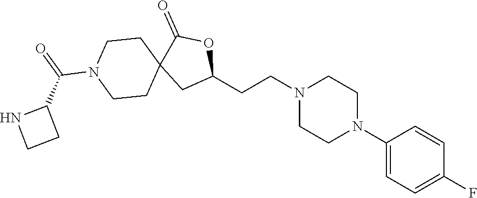 | 65.1 | 0.68 | — | — |
| 68. | 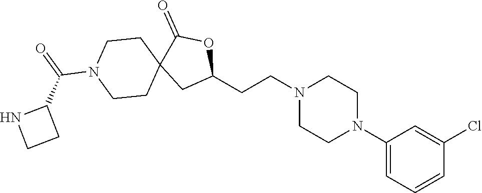 | 65.1 | 1.25 | — | — |
| 69. | 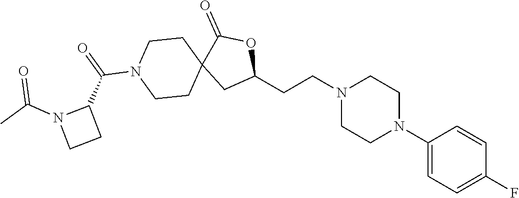 | 73.4 | 0.41 | — | — |
| 70. | 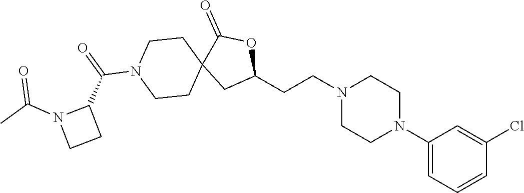 | 73.4 | 0.98 | — | — |
| 71. | 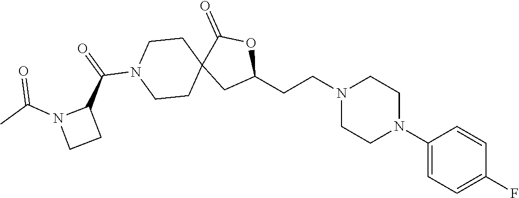 | 73.4 | 0.41 | — | — |
| 72. | 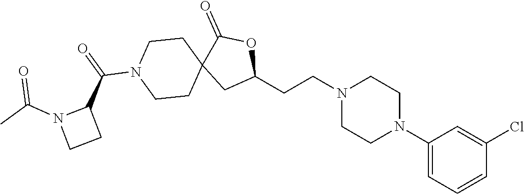 | 73.4 | 0.98 | — | — |
| 73. | 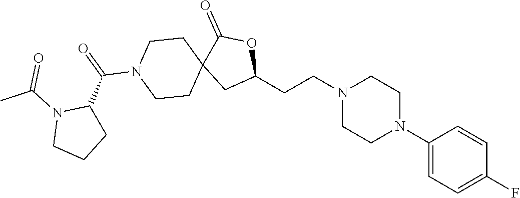 | 73.4 | 0.97 | — | — |
| 74. | 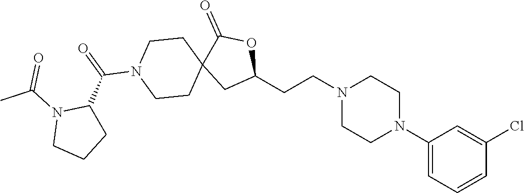 | 73.4 | 1.54 | — | — |
| 75. | 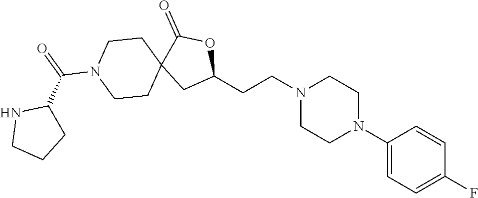 | 65.1 | 1.24 | — | — |
| 76. | 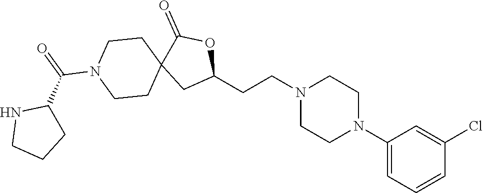 | 65.1 | 1.81 | — | — |
| 77. | 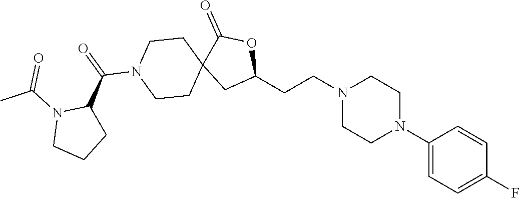 | 73.4 | 0.97 | — | — |
| 78. | 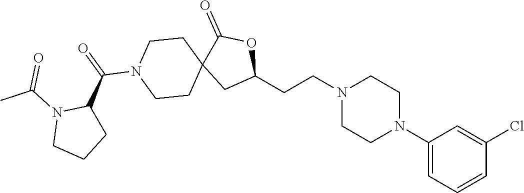 | 73.4 | 1.54 | — | — |
| 79. | 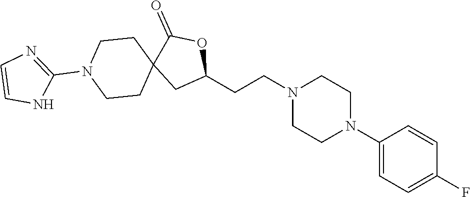 [SMILES: O=C1O[C@@H](CCN2CCN(c3ccc(F)cc3)CC2)CC12CCN(c1ncc[nH]1)CC2] | 60.4 | 1.84 | — | — |
| 80. | 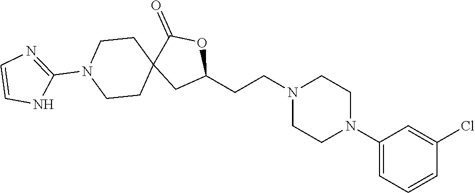 [SMILES: O=C1O[C@@H](CCN2CCN(c3cccc(Cl)c3)CC2)CC12CCN(c1ncc[nH]1)CC2] | 60.4 | 2.41 | — | — |
| 81. | 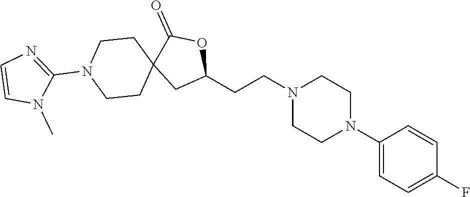 [SMILES: Cn1ccnc1N1CCC2(CC1)C[C@H](CCN1CCN(c3ccc(F)cc3)CC1)OC2=O] | 51.6 | 1.96 | — | — |
| 82. | 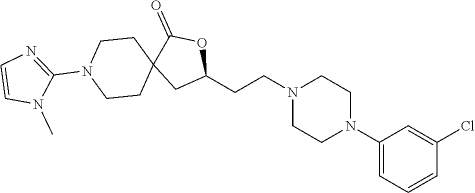 [SMILES: Cn1ccnc1N1CCC2(CC1)C[C@H](CCN1CCN(c3cccc(Cl)c3)CC1)OC2=O] | 51.6 | 2.53 | — | — |
| 83. | 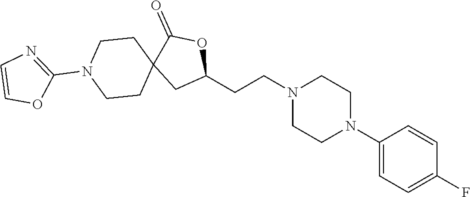 [SMILES: O=C1O[C@@H](CCN2CCN(c3ccc(F)cc3)CC2)CC12CCN(c1ncco1)CC2] | 57.6 | 1.69 | — | — |
| 84. | 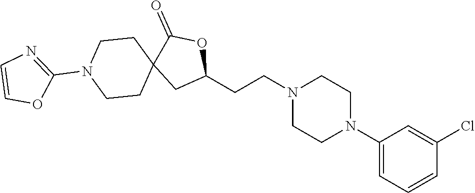 [SMILES: O=C1O[C@@H](CCN2CCN(c3cccc(Cl)c3)CC2)CC12CCN(c1ncco1)CC2] | 57.6 | 2.26 | — | — |
| 85. | 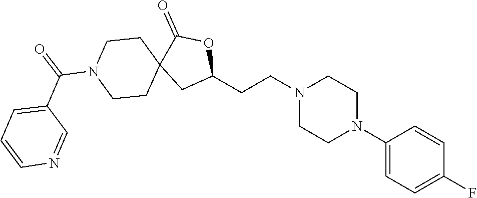 [SMILES: O=C(c1cccnc1)N1CCC2(CC1)C[C@H](CCN1CCN(c3ccc(F)cc3)CC1)OC2=O] | 65.4 | 1.18 | — | — |
| 86. | 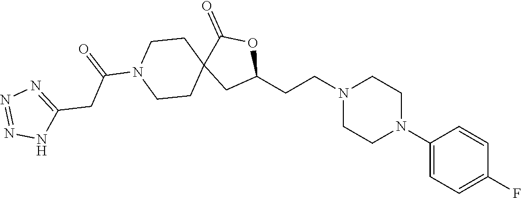 [SMILES: O=C(Cc1nnn[nH]1)N1CCC2(CC1)C[C@H](CCN1CCN(c3ccc(F)cc3)CC1)OC2=O] | 102 | 0.13 | — | — |
| 87. | 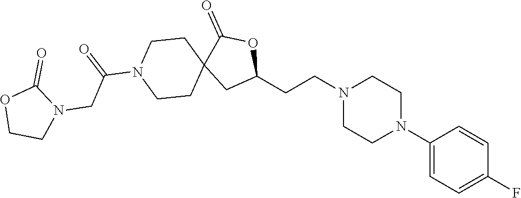 [SMILES: O=C(CN1CCOC1=O)N1CCC2(CC1)C[C@H](CCN1CCN(c3ccc(F)cc3)CC1)OC2=O] | 82.6 | 0.97 | — | — |
| 88. | 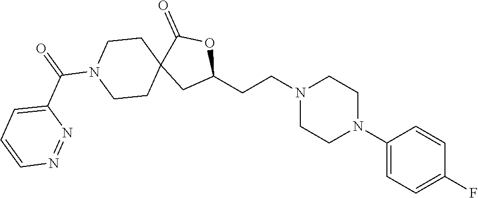 [SMILES: O=C(c1cccnn1)N1CCC2(CC1)C[C@H](CCN1CCN(c3ccc(F)cc3)CC1)OC2=O] | 77.8 | 0.19 | — | — |
| 89. | 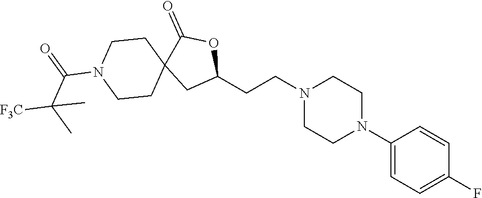 [SMILES: CC(C)(C(=O)N1CCC2(CC1)C[C@H](CCN1CCN(c3ccc(F)cc3)CC1)OC2=O)C(F)(F)F] | 53.0 | 1.98 | — | — |
| 90. | 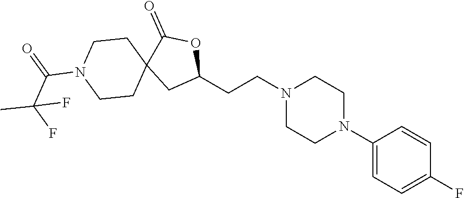 [SMILES: CC(F)(F)C(=O)N1CCC2(CC1)C[C@H](CCN1CCN(c3ccc(F)cc3)CC1)OC2=O] | 53.0 | 1.54 | — | — |
| 91. | 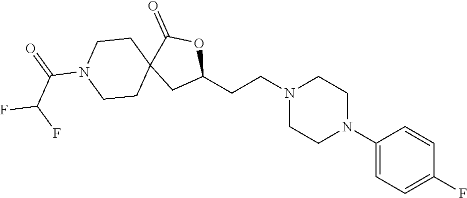 [SMILES: O=C(C(F)F)N1CCC2(CC1)C[C@H](CCN1CCN(c3ccc(F)cc3)CC1)OC2=O] | 53.0 | 1.01 | — | — |
| 92. | 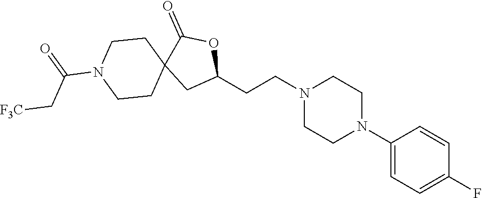 [SMILES: O=C(CC(F)(F)F)N1CCC2(CC1)C[C@H](CCN1CCN(c3ccc(F)cc3)CC1)OC2=O] | 53.0 | 1.26 | — | — |
| 93. | 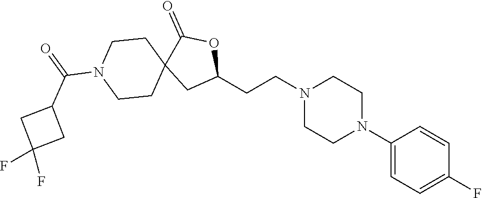 [SMILES: O=C(C1CC(F)(F)C1)N1CCC2(CC1)C[C@H](CCN1CCN(c3ccc(F)cc3)CC1)OC2=O] | 53.0 | 0.76 | — | — |
| 94. | 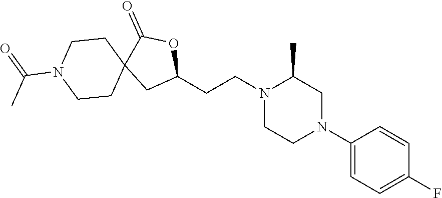 [SMILES: CC(=O)N1CCC2(CC1)C[C@H](CCN1CCN(c3ccc(F)cc3)C[C@@H]1C)OC2=O] | 53 | 0.98 | — | — |
| 95. | 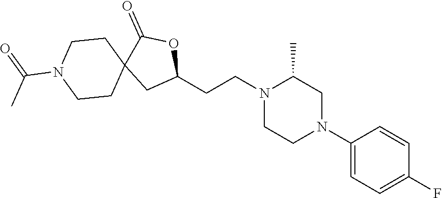 [SMILES: CC(=O)N1CCC2(CC1)C[C@H](CCN1CCN(c3ccc(F)cc3)C[C@H]1C)OC2=O] | 53 | 0.98 | — | — |
| 96. | 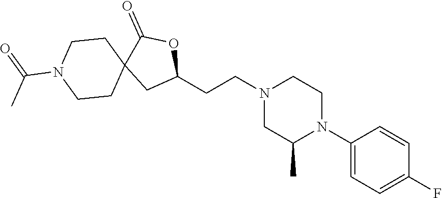 [SMILES: CC(=O)N1CCC2(CC1)C[C@H](CCN1CCN(c3ccc(F)cc3)[C@@H](C)C1)OC2=O] | 53 | 0.98 | — | — |
| 97. | 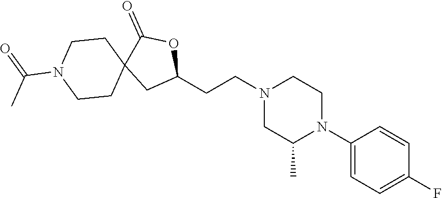 [SMILES: CC(=O)N1CCC2(CC1)C[C@H](CCN1CCN(c3ccc(F)cc3)[C@H](C)C1)OC2=O] | 53 | 0.98 | — | — |
| 98. | 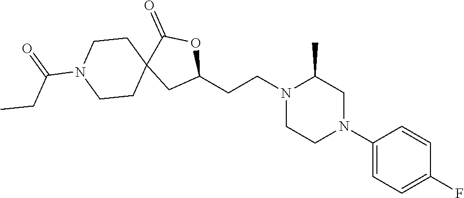 [SMILES: CCC(=O)N1CCC2(CC1)C[C@H](CCN1CCN(c3ccc(F)cc3)C[C@@H]1C)OC2=O] | 53 | 1.61 | — | — |
| 99. | 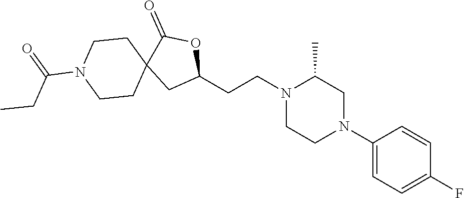 [SMILES: CCC(=O)N1CCC2(CC1)C[C@H](CCN1CCN(c3ccc(F)cc3)C[C@H]1C)OC2=O] | 53 | 1.61 | — | — |
| 100. | 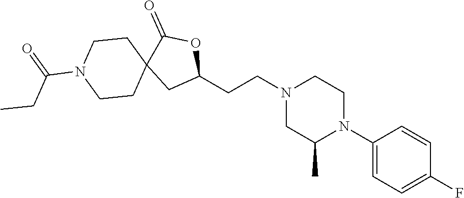 [SMILES: CCC(=O)N1CCC2(CC1)C[C@H](CCN1CCN(c3ccc(F)cc3)[C@@H](C)C1)OC2=O] | 53 | 1.61 | — | — |
| 101. | 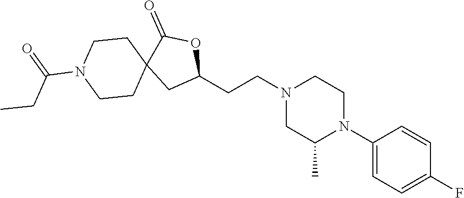 [SMILES: CCC(=O)N1CCC2(CC1)C[C@H](CCN1CCN(c3ccc(F)cc3)[C@H](C)C1)OC2=O] | 53 | 1.61 | — | — |
| 102. | 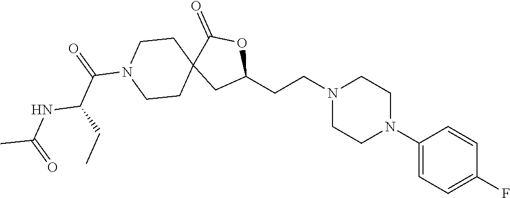 [SMILES: CC[C@H](NC(C)=O)C(=O)N1CCC2(CC1)C[C@H](CCN1CCN(c3ccc(F)cc3)CC1)OC2=O] | 82.1 | 1.12 | — | — |
| 103. | 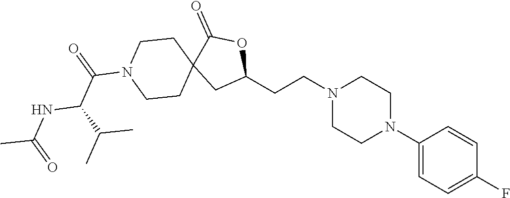 [SMILES: CC(=O)N[C@H](C(=O)N1CCC2(CC1)C[C@H](CCN1CCN(c3ccc(F)cc3)CC1)OC2=O)C(C)C] | 82.1 | 1.52 | — | — |
| 104. | 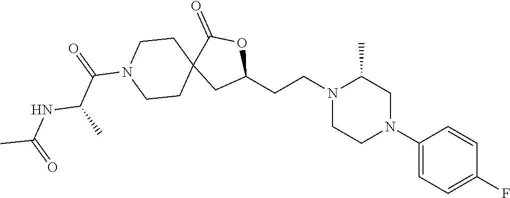 [SMILES: CC(=O)N[C@@H](C)C(=O)N1CCC2(CC1)C[C@H](CCN1CCN(c3ccc(F)cc3)C[C@H]1C)OC2=O] | 82.1 | 1.01 | — | — |
| 105. | 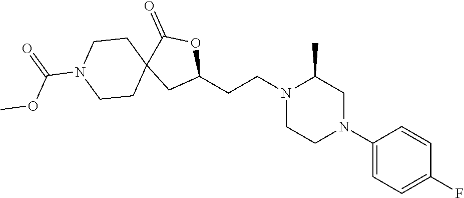 [SMILES: COC(=O)N1CCC2(CC1)C[C@H](CCN1CCN(c3ccc(F)cc3)C[C@@H]1C)OC2=O] | 62.3 | 2.74 | — | — |
| 106. | 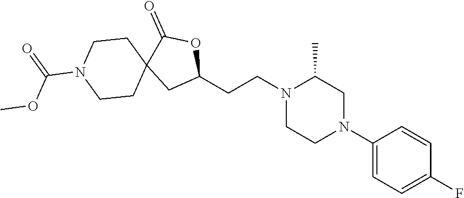 [SMILES: COC(=O)N1CCC2(CC1)C[C@H](CCN1CCN(c3ccc(F)cc3)C[C@H]1C)OC2=O] | 62.3 | 2.74 | — | — |
| 107. | 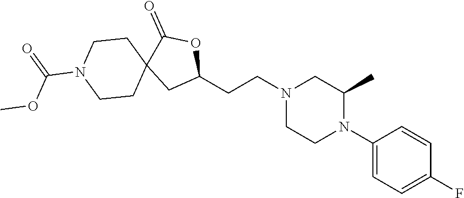 [SMILES: COC(=O)N1CCC2(CC1)C[C@H](CCN1CCN(c3ccc(F)cc3)[C@H](C)C1)OC2=O] | 62.3 | 2.74 | — | — |
| 108. | 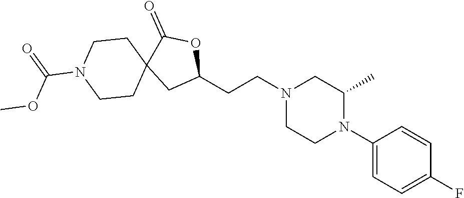 [SMILES: COC(=O)N1CCC2(CC1)C[C@H](CCN1CCN(c3ccc(F)cc3)[C@@H](C)C1)OC2=O] | 62.3 | 2.74 | — | — |
| 109. | 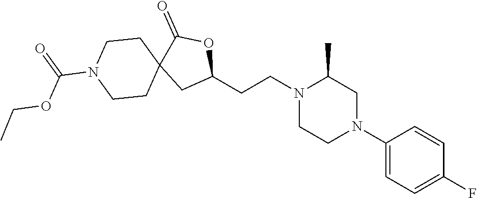 [SMILES: CCOC(=O)N1CCC2(CC1)C[C@H](CCN1CCN(c3ccc(F)cc3)C[C@@H]1C)OC2=O] | 62.3 | 3.26 | — | — |
| 110. | 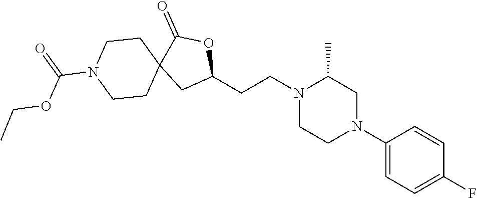 [SMILES: CCOC(=O)N1CCC2(CC1)C[C@H](CCN1CCN(c3ccc(F)cc3)C[C@H]1C)OC2=O] | 62.3 | 3.26 | — | — |
| 111. | 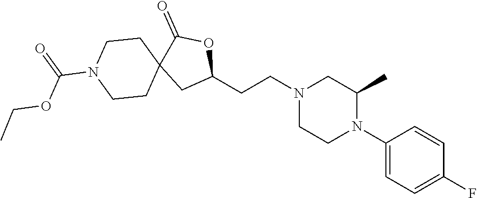 [SMILES: CCOC(=O)N1CCC2(CC1)C[C@H](CCN1CCN(c3ccc(F)cc3)[C@H](C)C1)OC2=O] | 62.3 | 3.26 | — | — |
| 112. | 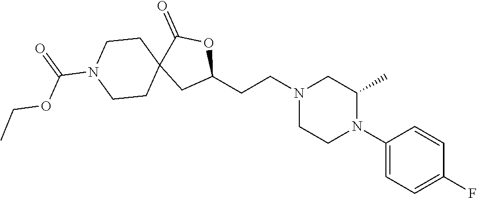 [SMILES: CCOC(=O)N1CCC2(CC1)C[C@H](CCN1CCN(c3ccc(F)cc3)[C@@H](C)C1)OC2=O] | 62.3 | 3.26 | — | — |
| 113. | 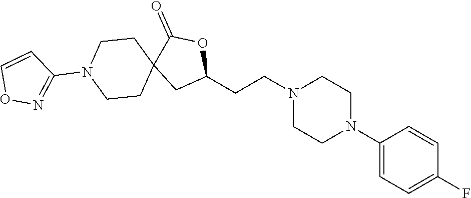 [SMILES: O=C1O[C@@H](CCN2CCN(c3ccc(F)cc3)CC2)CC12CCN(c1ccon1)CC2] | 57.6 | 1.98 | — | — |
| 114. | 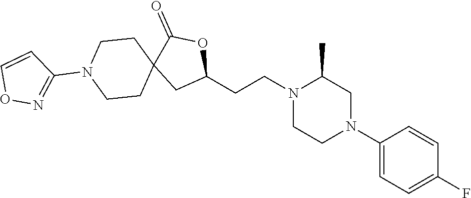 [SMILES: C[C@H]1CN(c2ccc(F)cc2)CCN1CC[C@H]1CC2(CCN(c3ccon3)CC2)C(=O)O1] | 57.6 | 2.5 | — | — |
| 115. | 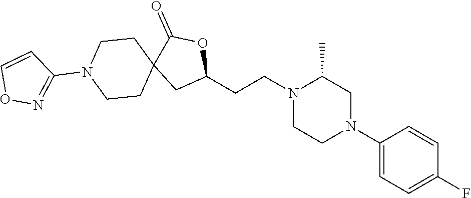 [SMILES: C[C@@H]1CN(c2ccc(F)cc2)CCN1CC[C@H]1CC2(CCN(c3ccon3)CC2)C(=O)O1] | 57.6 | 2.5 | — | — |
| 116. | 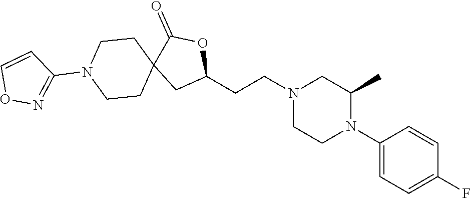 [SMILES: C[C@@H]1CN(CC[C@H]2CC3(CCN(c4ccon4)CC3)C(=O)O2)CCN1c1ccc(F)cc1] | 57.6 | 2.5 | — | — |
| 117. | 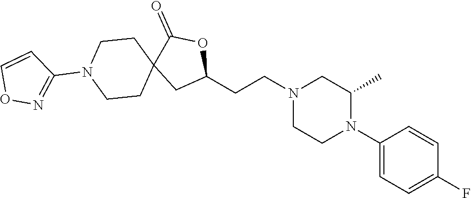 [SMILES: C[C@H]1CN(CC[C@H]2CC3(CCN(c4ccon4)CC3)C(=O)O2)CCN1c1ccc(F)cc1] | 57.6 | 2.5 | — | — |
| 118. | 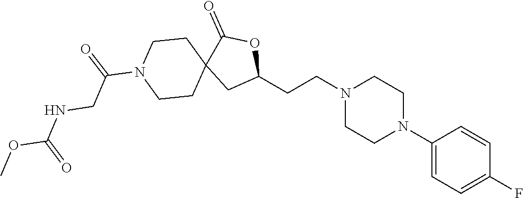 [SMILES: COC(=O)NCC(=O)N1CCC2(CC1)C[C@H](CCN1CCN(c3ccc(F)cc3)CC1)OC2=O] | 91.4 | 0.91 | — | — |
| 119. | 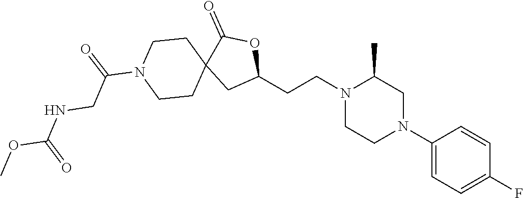 [SMILES: COC(=O)NCC(=O)N1CCC2(CC1)C[C@H](CCN1CCN(c3ccc(F)cc3)C[C@@H]1C)OC2=O] | 91.4 | 1.43 | — | — |
| 120. | 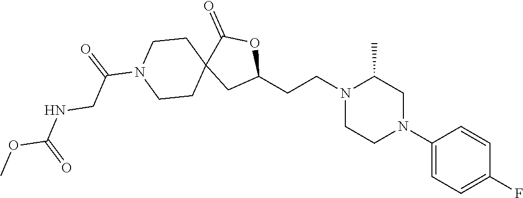 [SMILES: COC(=O)NCC(=O)N1CCC2(CC1)C[C@H](CCN1CCN(c3ccc(F)cc3)C[C@H]1C)OC2=O] | 91.4 | 1.43 | — | — |
| 121. | 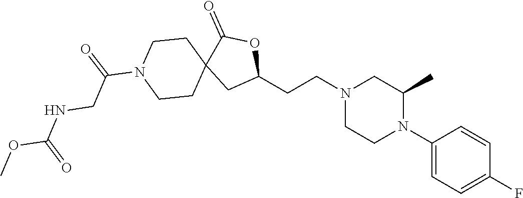 [SMILES: COC(=O)NCC(=O)N1CCC2(CC1)C[C@H](CCN1CCN(c3ccc(F)cc3)[C@H](C)C1)OC2=O] | 91.4 | 1.43 | — | — |
| 122. | 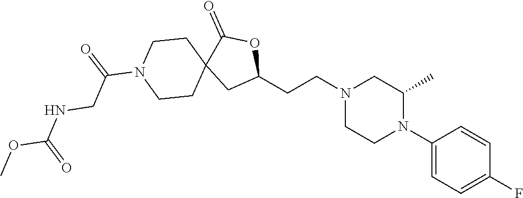 [SMILES: COC(=O)NCC(=O)N1CCC2(CC1)C[C@H](CCN1CCN(c3ccc(F)cc3)[C@@H](C)C1)OC2=O] | 91.4 | 1.43 | — | — |
| 123. | 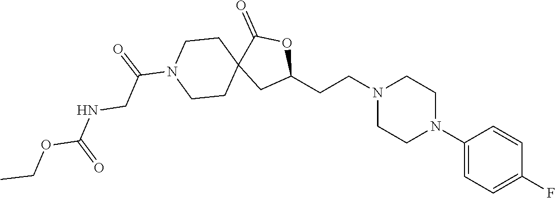 [SMILES: CCOC(=O)NCC(=O)N1CCC2(CC1)C[C@H](CCN1CCN(c3ccc(F)cc3)CC1)OC2=O] | 91.4 | 1.44 | — | — |
| 124. | 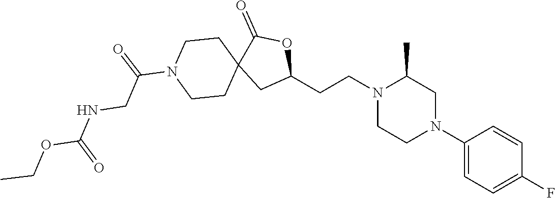 [SMILES: CCOC(=O)NCC(=O)N1CCC2(CC1)C[C@H](CCN1CCN(c3ccc(F)cc3)C[C@@H]1C)OC2=O] | 91.4 | 1.96 | — | — |
| 125. | 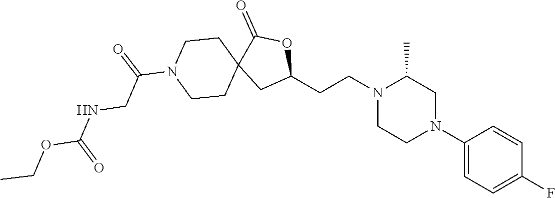 [SMILES: CCOC(=O)NCC(=O)N1CCC2(CC1)C[C@H](CCN1CCN(c3ccc(F)cc3)C[C@H]1C)OC2=O] | 91.4 | 1.96 | — | — |
| 126. | 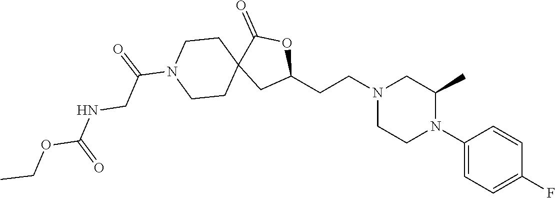 [SMILES: CCOC(=O)NCC(=O)N1CCC2(CC1)C[C@H](CCN1CCN(c3ccc(F)cc3)[C@H](C)C1)OC2=O] | 91.4 | 1.96 | — | — |
| 127. | 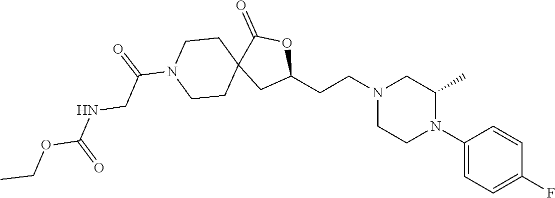 [SMILES: CCOC(=O)NCC(=O)N1CCC2(CC1)C[C@H](CCN1CCN(c3ccc(F)cc3)[C@@H](C)C1)OC2=O] | 91.4 | 1.96 | — | — |
| 128. | 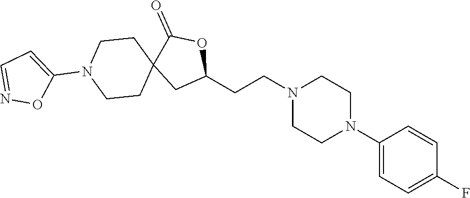 [SMILES: O=C1O[C@@H](CCN2CCN(c3ccc(F)cc3)CC2)CC12CCN(c1ccno1)CC2] | 57.6 | 1.98 | — | — |
| 129. | 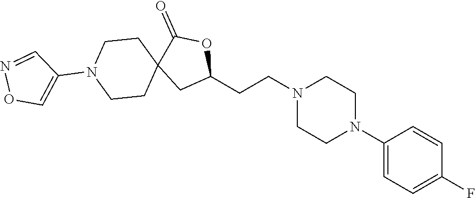 [SMILES: O=C1O[C@@H](CCN2CCN(c3ccc(F)cc3)CC2)CC12CCN(c1cnoc1)CC2] | 57.6 | 1.98 | — | — |
| 130. | 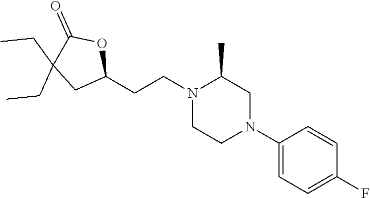 [SMILES: CCC1(CC)C[C@H](CCN2CCN(c3ccc(F)cc3)C[C@@H]2C)OC1=O] | 32.7 | 4.56 | — | — |
| 131. | 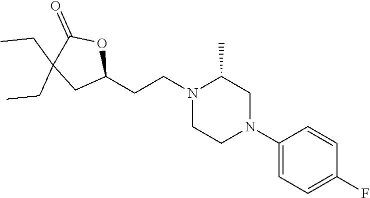 [SMILES: CCC1(CC)C[C@H](CCN2CCN(c3ccc(F)cc3)C[C@H]2C)OC1=O] | 32.7 | 4.56 | — | — |
| 132. | 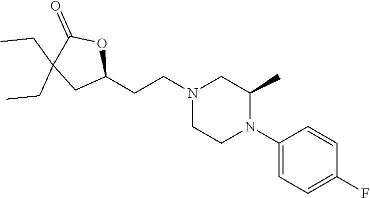 [SMILES: CCC1(CC)C[C@H](CCN2CCN(c3ccc(F)cc3)[C@H](C)C2)OC1=O] | 32.7 | 4.56 | — | — |
| 133. | 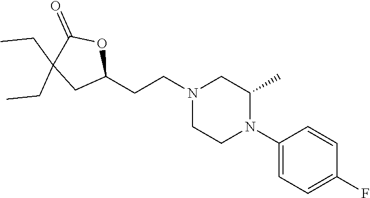 [SMILES: CCC1(CC)C[C@H](CCN2CCN(c3ccc(F)cc3)[C@@H](C)C2)OC1=O] | 32.7 | 4.56 | — | — |
| 134. | 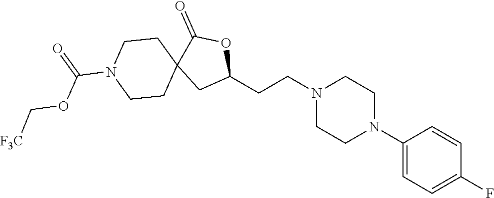 [SMILES: O=C(OCC(F)(F)F)N1CCC2(CC1)C[C@H](CCN1CCN(c3ccc(F)cc3)CC1)OC2=O] | 62.3 | 2.91 | — | — |
| 135. | 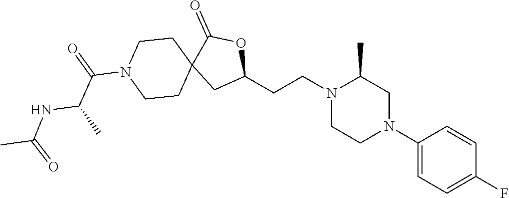 [SMILES: CC(=O)N[C@@H](C)C(=O)N1CCC2(CC1)C[C@H](CCN1CCN(c3ccc(F)cc3)C[C@@H]1C)OC2=O] | — | — | — | — |
| 136. | 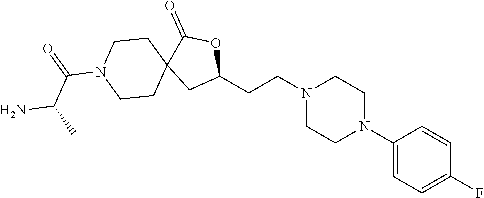 [SMILES: C[C@H](N)C(=O)N1CCC2(CC1)C[C@H](CCN1CCN(c3ccc(F)cc3)CC1)OC2=O] | — | — | — | — |
| 137. | 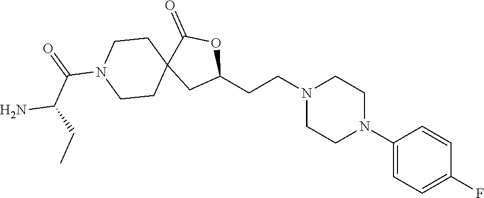 [SMILES: CC[C@H](N)C(=O)N1CCC2(CC1)C[C@H](CCN1CCN(c3ccc(F)cc3)CC1)OC2=O] | — | — | — | — |
| 138. | 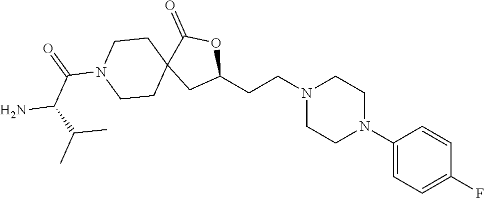 [SMILES: CC(C)[C@H](N)C(=O)N1CCC2(CC1)C[C@H](CCN1CCN(c3ccc(F)cc3)CC1)OC2=O] | — | — | — | — |
| 139. | 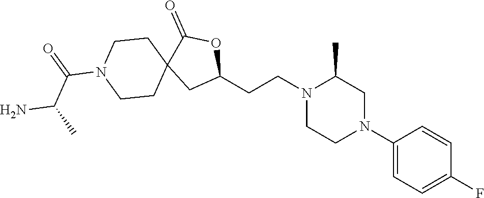 [SMILES: C[C@H](N)C(=O)N1CCC2(CC1)C[C@H](CCN1CCN(c3ccc(F)cc3)C[C@@H]1C)OC2=O] | — | — | — | — |
| 140. | 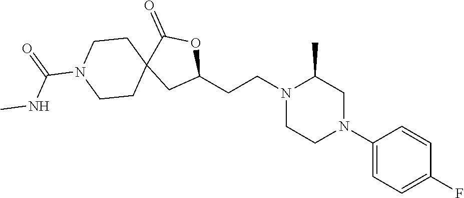 [SMILES: CNC(=O)N1CCC2(CC1)C[C@H](CCN1CCN(c3ccc(F)cc3)C[C@@H]1C)OC2=O] | 65.1 | 1.89 | — | — |
| 141. | 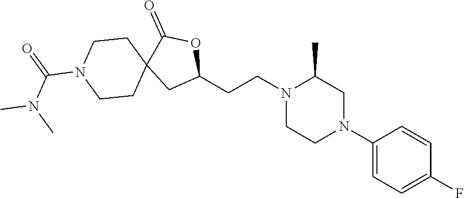 [SMILES: C[C@H]1CN(c2ccc(F)cc2)CCN1CC[C@H]1CC2(CCN(C(=O)N(C)C)CC2)C(=O)O1] | 56.3 | 2.25 | — | — |
| 142. | 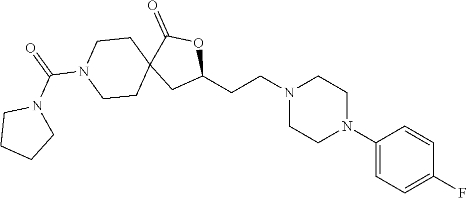 [SMILES: O=C(N1CCCC1)N1CCC2(CC1)C[C@H](CCN1CCN(c3ccc(F)cc3)CC1)OC2=O] | 56.3 | 2.32 | — | — |
| 143. | 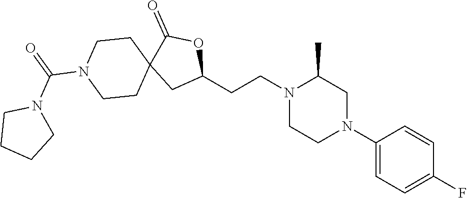 [SMILES: C[C@H]1CN(c2ccc(F)cc2)CCN1CC[C@H]1CC2(CCN(C(=O)N3CCCC3)CC2)C(=O)O1] | 56.3 | 2.84 | — | — |
| 144. | 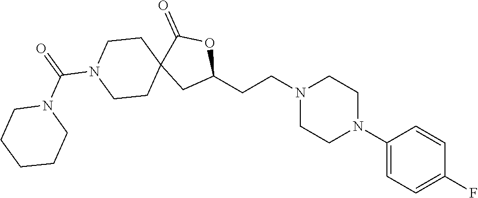 [SMILES: O=C(N1CCCCC1)N1CCC2(CC1)C[C@H](CCN1CCN(c3ccc(F)cc3)CC1)OC2=O] | 56.3 | 2.88 | — | — |
| 145. | 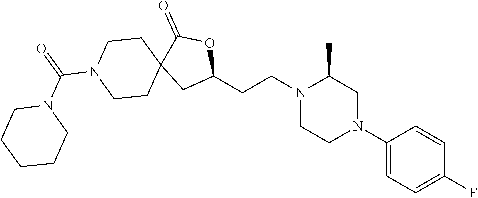 [SMILES: C[C@H]1CN(c2ccc(F)cc2)CCN1CC[C@H]1CC2(CCN(C(=O)N3CCCCC3)CC2)C(=O)O1] | 56.3 | 3.40 | — | — |

Legend:
A = Kb < 10 nM, or Ki < 50 nM
B = 10 nM < Kb < 100 nM, or 50 nM < Ki < 200 nM
C = 100 nM < Kb < 500 nM, or 200 nM < Ki < 500 nM
D = Kb > 500 nM, or Ki > 500 nM

General Synthetic Methods for Preparation of the 5-HT7 Modulators

The reagents used in the preparation of the compounds of this invention can be either commercially obtained or can be prepared by standard procedures described in the literature. In accordance with this invention, compounds in the genus may be produced by one of the following reaction schemes.

The compounds disclosed herein can be prepared by those methods described described in International Application No. PCT/US2017/061677, filed Nov. 15, 2017; International Application No. PCT/US2013/071926, filed Nov. 26, 2013; International Application No. PCT/US2014/023400, filed Mar. 11, 2014; International Application No. PCT/US2015/049303, filed Sep. 10, 2015; International Application No. PCT/US2016/031780, filed May 11, 2016; International Application No. PCT/US2018/022581, filed Mar. 15, 2018; and International Application No. PCT/US2018/022574, filed Mar. 15, 2018, the entireties of each of which is incorporated by reference herein.

In addition, certain exemplary methods are described in Schemes 1-4. In these schemes, the variables in any structure can be according to any aspect or embodiment as described herein.

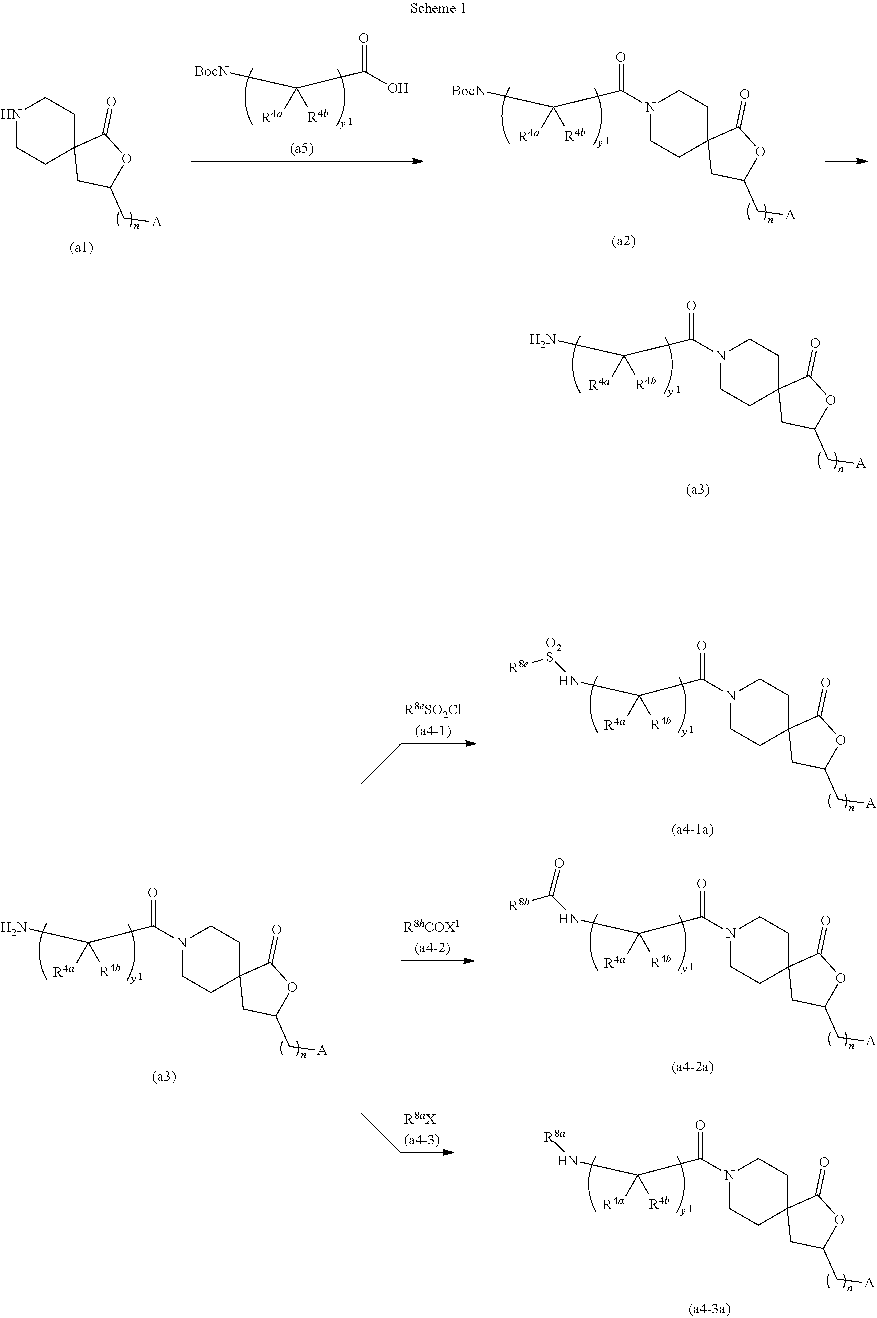

A compound of the formula (a1) is reacted with a compound of the formula (a5), a known compound or a compound prepared by known methods, in the presence of a coupling agent such as 1-[bis(dimethylamino)methylene]-1H-1,2,3-triazolo[4,5-b]pyridinium 3-oxid hexafluorophosphate, O-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide. and the like, optionally in the presence of hydroxybenzotriazole, optionally in the presence of 1-hydroxy-7-azabenzotriazole, and optionally in the presence of a base such as sodium carbonate, potassium carbonate, lithium carbonate, sodium bicarbonate, potassium bicarbonate, lithium bicarbonate, triethylamine, N, N-diisopropylethylamine, pyridine, and the like, in the presence of a solvent such as such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (a2). A compound according to formula (a2) is reacted with an acid such as trifluoroacetic acid, hydrochloric acid, sulphuric acid, and the like in a solvent such as tetrahydrofuran, 1,4-dioxane, methylene chloride, 1,2-dichloroethane, methanol, ethanol, 1,2-dimethoxyethane, N,N-dimethylformamide, N,N-dimethylacetamide, and the like, optionally with heating, optionally with microwave irradiation to provide a compound of the formula (a3).

A compound of the formula (a3) is reacted with a compound of the formula (a4-1), a known compound or a compound prepared by known methods, in the presence of a base such as triethylamine, diisopropylethylamine, pyridine, and the like, in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, and the like, optionally with heating, optionally with microwave irradiation to provide a compound of the formula (a4-1a). Alternatively, a compound of the formula (a3) is reacted with a compound of the formula (a4-2), a known compound or a compound prepared by known methods wherein X¹ is chlorine, in the presence of a base such as triethylamine, diisopropylethylamine, pyridine, and the like, in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, and the like, optionally with heating, optionally with microwave irradiation to provide a compound of the formula (a4-2a). Alternatively, a compound of the formula (a3) is reacted with a compound of the formula (a4-2), a known compound or a compound prepared by known methods wherein X¹ is OH, in the presence of a coupling agent such as 1-[bis(dimethylamino)methylene]-1H-1,2,3-triazolo[4,5-b]pyridinium 3-oxid hexafluorophosphate, O-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide. and the like, optionally in the presence of hydroxybenzotriazole, optionally in the presence of 1-hydroxy-7-azabenzotriazole, and the like, optionally in the presence of a base such as triethylamine, N,N-diisopropylethylamine, pyridine, 2,6-dimethylpyridine, and the like, in the presence of a solvent such as such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (a4-2a).

Alternatively, a compound of the formula (a3) is reacted with a compound of the formula (a4-3), a known compound or a compound prepared by known methods wherein X is selected from the group consisting of iodide, bromine, chlorine, methansulfonate, and para-tolylsufonate, in the presence of a base such as triethylamine, diisopropylethylamine, pyridine, and the like, in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, and the like, optionally with heating, optionally with microwave irradiation to provide a compound of the formula (a4-3a).

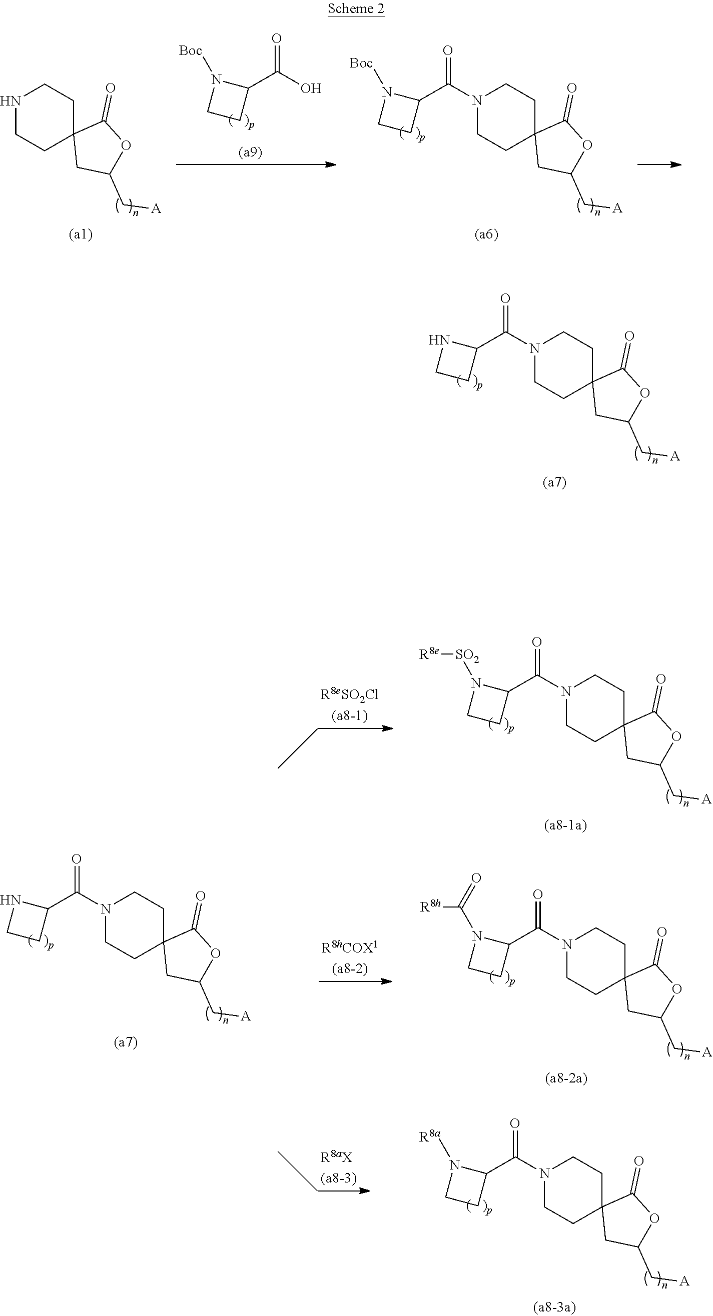

A compound of the formula (a1) is reacted with a compound of the formula (09), a known compound or a compound prepared by known methods, in the presence of a coupling agent such as 1-[bis(dimethylamino)methylene]-1H-1,2,3-triazolo[4,5-b]pyridinium 3-oxid hexafluorophosphate, O-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide. and the like, optionally in the presence of hydroxybenzotriazole, optionally in the presence of 1-hydroxy-7-azabenzotriazole, optionally in the presence of a base such as sodium carbonate, potassium carbonate, lithium carbonate, sodium bicarbonate, potassium bicarbonate, lithium bicarbonate, triethylamine, N,N-diisopropylethylamine, pyridine, and the like, in the presence of a solvent such as such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (a6). A compound according to formula (a6) is reacted with an acid such as trifluoroacetic acid, hydrochloric acid, sulphuric acid, and the like in a solvent such as tetrahydrofuran, 1,4-dioxane, methylene chloride, 1,2-dichloroethane, methanol, ethanol, 1,2-dimethoxyethane, N,N-dimethylformamide, N,N-dimethylacetamide, and the like, optionally with heating, optionally with microwave irradiation to provide a compound of the formula (a7). A compound of the formula (a7) is reacted with a compound of the formula (a8-1), a known compound or a compound prepared by known methods, in the presence of a base such as triethylamine, diisopropylethylamine, pyridine, and the like, in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, and the like, optionally with heating, optionally with microwave irradiation to provide a compound of the formula (a8-1a). Alternatively, a compound of the formula (a7) is reacted with a compound of the formula (a8-2), a known compound or a compound prepared by known methods wherein X¹ is chlorine, in the presence of a base such as triethylamine, diisopropylethylamine, pyridine, and the like, in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, and the like, optionally with heating, optionally with microwave irradiation to provide a compound of the formula (a8-2a). Alternatively, a compound of the formula (a7) is reacted with a compound of the formula (a8-2), a known compound or a compound prepared by known methods wherein X¹ is OH, in the presence of a coupling agent such as 1-[bis(dimethylamino)methylene]-1H-1,2,3-triazolo[4,5-b]pyridinium 3-oxid hexafluorophosphate, O-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide. and the like, optionally in the presence of hydroxybenzotriazole, optionally in the presence of 1-hydroxy-7-azabenzotriazole, and the like, optionally in the presence of a base such as triethylamine, N,N-diisopropylethylamine, pyridine, 2,6-dimethylpyridine, and the like, in the presence of a solvent such as such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (a4-8a).

Alternatively, a compound of the formula (a7) is reacted with a compound of the formula (a8-3), a known compound or a compound prepared by known methods wherein X is selected from the group consisting of iodide, bromine, chlorine, methansulfonate, and para-tolylsufonate, in the presence of a base such as triethylamine, diisopropylethylamine, pyridine, and the like, in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, and the like, optionally with heating, optionally with microwave irradiation to provide a compound of the formula (a8-3a).

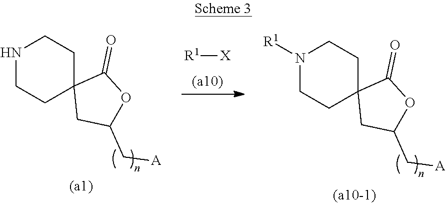

A compound of the formula (a1) is reacted with a compound of the formula (a10), a known compound of a compound prepared by known methods wherein X is selected from the group consisting of iodide, bromine, chlorine, methansulfonate, and para-tolylsufonate, in the presence of a palladium catalyst such as palladium (II) acetate, tetrakis(triphenylphosphine)palladium(O), dichlorobis (triphenylphosphine) palladium(II), palladium on carbon, bis(acetonitrile)dichloropalladium(II), and the like, in the presence of an organophosphine such as 2-dicyclohexylphosphino-2′,6′-dimethoxybiphenyl, 2-dicyclohexylphosphino-2′-(N,N-dimethylamino)biphenyl, 2-dicyclohexylphosphino-2′,4′,6′-triiso propylbiphenyl, 2-di-tert-butylphosphino-2′,4′,6′-triisopropylbiphenyl, (2-biphenyl)dicyclohexyl phosphine, (2-biphenyl)di-tert-butylphosphine, 2-dicyclohexylphosphino-2′,6′-diisopropoxybiphenyl, 2-di-tert-butylphosphino-3,4,5,6-tetramethyl-2′,4′,6′-triisopropyl-1,1′-biphenyl, Sodium 2′-dicyclo hexylphosphino-2,6-dimethoxy-1,1′-biphenyl-3-sulfonate, 2-di-tert-butylphosphino-2′-methyl biphenyl, 2-dicyclohexylphosphino-2′-methylbiphenyl, 2′-(di-tert-butylphosphino)-N,N-dimethyl biphenyl-2-amine, 2′-(diphenylphosphino)-N,N′-dimethyl-(1,1′-biphenyl)-2-amine, and the like, optionally in the presence of a base such as sodium carbonate, lithium carbonate, potassium carbonate, caesium carbonate, sodium hydroxide, lithium hydroxide, potassium hydroxide, triethylamine, N,N-diisopropylethylamine, pyridine, 2,6-dimethylpyridine, and the like, in a solvent such as tetrahydrofuran, 1,4-dioxane, acetonitrile, methylene chloride, chloroform, 1,2-dichloroethane, 1,2-dimethoxyethane, and the like, optionally with heating, optionally with microwave irradiation to provide a compound of the formula (a10-1).

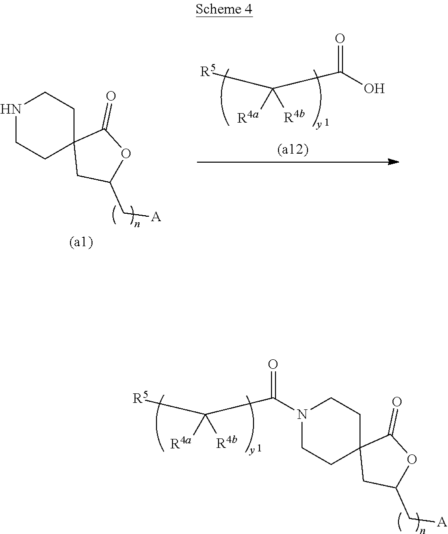

A compound of the formula (a1) is reacted with a compound of the formula (a12), a known compound or a compound prepared by known methods, in the presence of a coupling agent such as 1-[bis(dimethylamino)methylene]-1H-1,2,3-triazolo[4,5-b]pyridinium 3-oxid hexafluorophosphate, 0-(benzotriazol-1-yl)-N,N,N′,N′-tetramethyluronium hexafluorophosphate, N,N′-dicyclohexylcarbodiimide, 1-ethyl-3-(3-dimethylaminopropyl) carbodiimide. and the like, optionally in the presence of hydroxybenzotriazole, optionally in the presence of 1-hydroxy-7-azabenzotriazole, and the like, optionally in the presence of a base such as sodium carbonate, potassium carbonate, lithium carbonate, sodium bicarbonate, potassium bicarbonate, lithium bicarbonate, triethylamine, N, N-diisopropylethylamine, pyridine, and the like, in the presence of a solvent such as such as N-methyl-2-pyrrolidone, N,N-dimethylformamide, dimethylsulfoxide, N,N-dimethylacetamide, methylene chloride, chloroform, 1,2-dichloroethane, tetrahydrofuran, 1,4-dioxane, acetonitrile, 1,2-dimethoxyethane, and the like, optionally with heating, optionally with microwave irradiation to provide a compound of the formula (all).

### Methods of Treatment

In embodiments, compounds described herein are selective modulators of the serotonin 5-HT₇ receptor. In embodiments, compounds described herein can more potently bind a serotonin 5-HT₇ receptor as compared to other targets (e.g., other serotonin receptors). In embodiments, the compounds described herein may selectively bind a serotonin 5-HT₇ receptor in a particular tissue or organ.

For example, the compounds described herein may selectively bind serotonin 5-HT₇ receptors in the intestine of a subject. Accordingly, the compounds described herein may be used to treat or prevent inflammatory bowel disease (IBD) or intestinal inflammation.

In other embodiments, compounds described herein may have particularly favorable properties for effective therapy (e.g., of any of the diseases or conditions described herein). For example, in the treatment of CNS or mental disorders, the compounds described herein may exhibit favorably effective blood-brain barrier permeability. Alternatively, in the treatment of non-CNS or -mental disorders, a compound described herein will not have high blood-brain barrier permeability (e.g., off-target effects will be reduced). Without being bound by theory, molecular elements of a compound may be an effective strategy for obtaining the desired biological targeting.

There is evidence that suggests a role for the 5-HT₇ receptor in a number of medical disorders. 5-HT₇ receptor activity modulators are likely to have a beneficial effect on patients suffering from these disorders. The disorders in which 5-HT₇ dysregulation plays a role and modulation of 5-HT₇ receptor activity by a therapeutic agent may be a viable approach to therapeutic relief include, but are not limited to, circadian rhythm disorder, depression, schizophrenia, neurogenic inflammation, hypertension, peripheral, vascular diseases, migraine (Vanhoenacker, P. et al. Trends in Pharmacological Sciences, 2000, 21, 2, 70-77), neuropathic pain, peripheral pain, allodynia (EP1875899), thermoregulation disorder, learning disorder, memory disorder, hippocampal signaling disorder, sleep disorder (WO20100197700) attention deficit/hyperactivity disorder (ADHD) (WO20100069390), anxiety, avoidant personality disorder, premature ejaculation, eating disorder, premenstrual syndrome, premenstrual dysphonic disorder, seasonal affective disorder, bipolar disorder (WO20040229874), inflammatory bowel disease (IBD), intestinal inflammation (WO 2012058769, Khan, W. I., et al. Journal of Immunology, 2013, 190, 4795-4804), epilepsy, seizure disorders (Epilepsy Research (2007) 75, 39), drug addiction, alcohol addiction (Hauser, S. R. et al. Frontiers in Neuroscience, 2015, 8, 1-9), breast cancer (Gautam, J. Molecular Cancer, 2016, 15, 75, 1-14, Gautam, J. Breast Cancer Research and Treatment, 2017, 161, 29-40), liver fibrosis, chronic liver injury (Halici, Z. International Immunopharmacology, 2017, 43, 227-235), hepatocellular carcinoma (Bian, Z. X. Molecular Oncology, 2016, 10, 195-212), small intestine neuroendocrine tumors (Modlin, I. M. Cancer Science, 2013, 104, 7, 844-855), and lung injury (Halici, Z. Immunology, 2013, 1271-1283.).

There is a long felt need for new 5-HT₇ modulators that will provide therapeutic relief from patients suffering from diseases associated with dysregulation of 5-hydroxytryptamine receptor 7 activity. The invention addresses the need to identify novel 5-HT₇ modulators capable of treating disease associated with dysregulation of 5-hydroxytryptamine receptor 7 activity. The present invention addresses the need to develop new therapeutic agents for the treatment and prevention of circadian rhythm disorder, depression, schizophrenia, neurogenic inflammation, hypertension, peripheral, vascular diseases, migraine, neuropathic pain, peripheral pain, allodynia, thermoregulation disorder, learning disorder, memory disorder, hippocampal signaling disorder, sleep disorder, attention deficit/hyperactivity disorder, anxiety, avoidant personality disorder, premature ejaculation, eating disorder, premenstrual syndrome, premenstrual dysphonic disorder, seasonal affective disorder, bipolar disorder, inflammatory bowel disease (IBD), intestinal inflammation epilepsy, seizure disorders, drug addiction, alcohol addiction, breast cancer, liver fibrosis, chronic liver injury, hepatocellular carcinoma, small intestine neuroendocrine tumors, and lung injury.

The 5-hydroxytryptamine receptor 7 activity modulators of the present invention are capable of treating and preventing diseases associated with dysregulation of 5-hydroxytryptamine receptor 7 activity, for example circadian rhythm disorder, depression, schizophrenia, neurogenic inflammation, hypertension, peripheral, vascular diseases, migraine, neuropathic pain, peripheral pain, allodynia, thermoregulation disorder, learning disorder, memory disorder, hippocampal signaling disorder, sleep disorder, attention deficit/hyperactivity disorder, anxiety, avoidant personality disorder, premature ejaculation, eating disorder, premenstrual syndrome, premenstrual dysphonic disorder, seasonal affective disorder, bipolar disorder, inflammatory bowel disease (IBD), intestinal inflammation, epilepsy, seizure disorders, drug addiction, alcohol addiction, breast cancer, liver fibrosis, chronic liver injury, hepatocellular carcinoma, small intestine neuroendocrine tumors, and lung injury. It has been discovered that the 5-hydroxytryptamine receptor 7 play a role in a number of medical disorders, and therefore, 5-HT₇ receptor activity modulators are likely to have a beneficial effect on patients suffering from these disorders. The disorders in which 5-HT₇ dysregulation plays a role and modulation of 5-HT₇ receptor activity by a therapeutic agent may be a viable approach to therapeutic relief include, but are not limited to, circadian rhythm disorder, depression, schizophrenia, neurogenic inflammation, hypertension, peripheral, vascular diseases, migraine (Vanhoenacker, P. et al. Trends in Pharmacological Sciences, 2000, 21, 2, 70-77), neuropathic pain, peripheral pain, allodynia (EP1875899), thermoregulation disorder, learning disorder, memory disorder, hippocampal signaling disorder, sleep disorder (WO20100197700) attention deficit/hyperactivity disorder (ADHD) (WO20100069390), anxiety, avoidant personality disorder, premature ejaculation, eating disorder, premenstrual syndrome, premenstrual dysphonic disorder, seasonal affective disorder, bipolar disorder (WO20040229874), inflammatory bowel disease (IBD), intestinal inflammation (WO 2012058769) epilepsy, seizure disorders (Epilepsy Research (2007) 75, 39), drug addiction, alcohol addiction (Hauser, S. R. et al. Frontiers in Neuroscience, 2015, 8, 1-9), breast cancer (Gautam, J. Molecular Cancer, 2016, 15, 75, 1-14, Gautam, J. Breast Cancer Research and Treatment, 2017, 161, 29-40), liver fibrosis, chronic liver injury (Halici, Z. International Immunopharmacology, 2017, 43, 227-235), hepatocellular carcinoma (Bian, Z. X. Molecular Oncology, 2016, 10, 195-212), small intestine neuroendocrine tumors (Modlin, I. M. Cancer Science, 2013, 104, 7, 844-855), and lung injury (Halici, Z. Immunology, 2013, 1271-1283.).

Without wishing to be limited by theory, it is believed that 5-hydroxytryptamine receptor 7 receptor activity modulators of the present invention can ameliorate, abate, otherwise cause to be controlled, diseases associated with dysregulation of 5-hydroxytryptamine receptor 7 activity. The diseases include, but are not limited to circadian rhythm disorder, depression, schizophrenia, neurogenic inflammation, hypertension, peripheral, vascular diseases, migraine, neuropathic pain, peripheral pain, allodynia, thermoregulation disorder, learning disorder, memory disorder, hippocampal signaling disorder, sleep disorder, attention deficit/hyperactivity disorder, anxiety, avoidant personality disorder, premature ejaculation, eating disorder, premenstrual syndrome, premenstrual dysphonic disorder, seasonal affective disorder, bipolar disorder, inflammatory bowel disease (IBD), intestinal inflammation, epilepsy, seizure disorders, drug addiction, alcohol addiction, breast cancer, liver fibrosis, chronic liver injury, hepatocellular carcinoma, small intestine neuroendocrine tumors, and lung injury.

In embodiments, a disease is depression, schizophrenia, anxiety, or bipolar disorder. In embodiments, a disease is depression. In embodiments, a disease is schizophrenia. In embodiments, a disease is anxiety. In embodiments, a disease is bipolar disorder.

In embodiments, a disease is attention deficit/hyperactivity disorder.

In embodiments, a disease is avoidant personality disorder.

In embodiments, a disease is seasonal affective disorder.

In embodiments, a disease is circadian rhythm disorder or hippocampal signaling disorder. In embodiments, a disease is circadian rhythm disorder. In embodiments, a disease is hippocampal signaling disorder.

In embodiments, a disease is neurogenic inflammation.

In embodiments, a disease is neuropathic pain, peripheral pain, or allodynia. In embodiments, a disease is neuropathic pain. In embodiments, a disease is peripheral pain. In embodiments, a disease is allodynia.

In embodiments, a disease is migraine.

In embodiments, a disease is epilepsy or a seizure disorder. In embodiments, a disease is epilepsy. In embodiments, a disease is a seizure disorder.

In embodiments, a disease is a learning disorder or a memory disorder. In embodiments, a disease is a learning disorder. In embodiments, a disease is a memory disorder.

In embodiments, a disease is an eating disorder.

In embodiments, a disease is drug addiction or alcohol addiction.

In embodiments, a disease is a sleep disorder.

In embodiments, a disease is hypertension or peripheral vascular disease. In embodiments, a disease is hypertension. In embodiments, a disease is peripheral vascular disease.

In embodiments, a disease is thermoregulation disorder.

In embodiments, a disease is premature ejaculation.

In embodiments, a disease is premenstrual syndrome or premenstrual dysphonic disorder. In embodiments, a disease is premenstrual syndrome. In embodiments, a disease is premenstrual dysphonic disorder.

In embodiments, a disease is inflammatory bowel disease (IBD) or intestinal inflammation. In embodiments, a disease is inflammatory bowel disease (IBD). In embodiments, a disease is intestinal inflammation.

In embodiments, a disease is breast cancer.

In embodiments, a disease is liver fibrosis, chronic liver injury, or hepatocellular carcinoma. In embodiments, a disease is liver fibrosis. In embodiments, a disease is chronic liver injury. In embodiments, a disease is hepatocellular carcinoma.

In embodiments, a disease is a small intestine neuroendocrine tumor.

In embodiments, a disease is lung injury.

In embodiments, a disease is inflammatory bowel disease (IBD).

### Formulations (Pharmaceutical Compositions) of the 5-HT7 Modulators

The present invention also relates to compositions or formulations which comprise the 5-hydroxytryptamine receptor 7 activity modulators according to the present invention. In general, the compositions of the present invention comprise an effective amount of one or more compounds of the disclosure and salts thereof according to the present invention which are effective for providing modulation of 5-hydroxytryptamine receptor 7 activity; and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17^{th} edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known 5-hydroxytryptamine receptor 7 activity modulators. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.]

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.001 mg to about 1000 mg of one or more compounds of the disclosure according to the present invention and one or more excipients; from about 0.01 mg to about 100 mg of one or more compounds of the disclosure according to the present invention and one or more excipients; and from about 0.1 mg to about 10 mg of one or more compounds of the disclosure according to the present invention; and one or more excipients.

### EXEMPLIFICATION

The practice of the invention is illustrated by the following non-limiting examples.

### Synthesis of 5-HT7 Modulators

Compounds of the invention may be prepared by methods known in the art. Exemplary methods are disclosed in International Application No. PCT/US2017/061677, filed Nov. 15, 2017; International Application No. PCT/US2013/071926, filed Nov. 26, 2013; International Application No. PCT/US2014/023400, filed Mar. 11, 2014; International Application No. PCT/US2015/049303, filed Sep. 10, 2015; International Application No. PCT/US2016/031780, filed May 11, 2016; International Application No. PCT/US2018/022581, filed Mar. 15, 2018; and International Application No. PCT/US2018/022574, filed Mar. 15, 2018.

The Examples provided below provide representative methods for preparing exemplary compounds of the present invention. The skilled practitioner will know how to substitute the appropriate reagents, starting materials and purification methods known to those skilled in the art, in order to prepare the compounds of the present invention.

### Synthesis and Characterization of the Intermediates

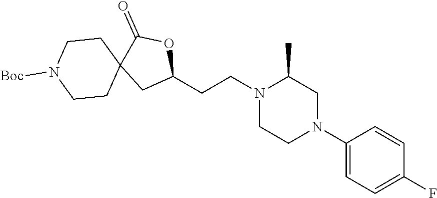 [SMILES: C[C@H]1CN(c2ccc(F)cc2)CCN1CC[C@H]1CC2(CCN(C(=O)OC(C)(C)C)CC2)C(=O)O1]

Preparation of tert-butyl (R)-3-(2-((S)-4-(4-fluorophenyl)-2-methylpiperazin-1-yl)ethyl)-1-oxo-2-oxa-8-azaspiro[4.5]decane-8-carboxylate: To a small vial was added tert-butyl (R)-1-oxo-3-(2-(tosyloxy)ethyl)-2-oxa-8-azaspiro[4.5]decane-8-carboxylate (1.5 g, 3.31 mmol, 1 equiv.) and (S)-1-(4-fluorophenyl)-3-methylpiperazine (1.35 g, 6.95 mmol, 2.1 equiv.) then both were dissolved in acetonitrile (33 mL). Then K₂CO₃ (1.14 g, 8.2 mmol, 2.5 equiv.) was added, the reaction was allowed to stir at 80° C. overnight, and then cooled to 23° C. The mixture was filtered, washed with acetonitrile and filtrate was concentrated in vacuo to give a crude product which was by further purified by column chromatography (MeOH/methylene chloride, 0%˜10%). LC/MS [M+H]=m/z 476.2

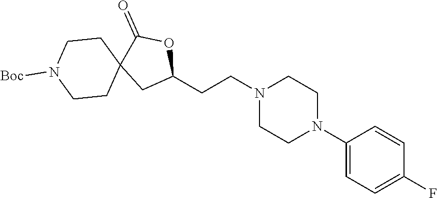 [SMILES: CC(C)(C)OC(=O)N1CCC2(CC1)C[C@H](CCN1CCN(c3ccc(F)cc3)CC1)OC2=O]

Preparation of tert-butyl (R)-3-(2-(4-(4-fluorophenyl)piperazin-1-yl)ethyl)-1-oxo-2-oxa-8-azaspiro[4.5]decane-8-carboxylate: The title compound was prepared according to the procedure for tert-butyl (R)-3-(2-((S)-4-(4-fluorophenyl)-2-methylpiperazin-1-yl)ethyl)-1-oxo-2-oxa-8-azaspiro[4.5]decane-8-carboxylate, except 1-(4-fluorophenyl)piperazine was substituted for (S)-1-(4-fluorophenyl)-3-methylpiperazine. LC/MS [M+H]=m/z 462.2

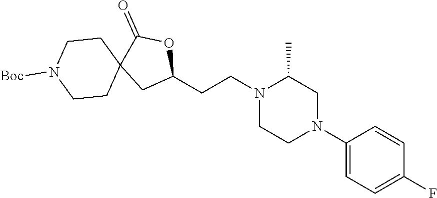 [SMILES: C[C@@H]1CN(c2ccc(F)cc2)CCN1CC[C@H]1CC2(CCN(C(=O)OC(C)(C)C)CC2)C(=O)O1]

Preparation of tert-butyl (R)-3-(2-((R)-4-(4-fluorophenyl)-2-methylpiperazin-1-yl)ethyl)-1-oxo-2-oxa-8-azaspiro[4.5]decane-8-carboxylate: The title compound was prepared according to the procedure for tert-butyl (R)-3-(2-((S)-4-(4-fluorophenyl)-2-methylpiperazin-1-yl)ethyl)-1-oxo-2-oxa-8-azaspiro[4.5]decane-8-carboxylate, except (R)-1-(4-fluorophenyl)-3-methylpiperazine was substituted for (S)-1-(4-fluorophenyl)-3-methylpiperazine. LG/MS [M+H]=m/z 476.2

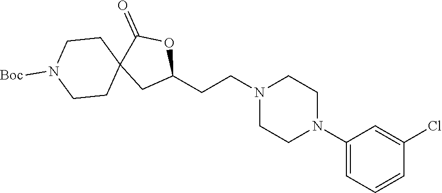 [SMILES: CC(C)(C)OC(=O)N1CCC2(CC1)C[C@H](CCN1CCN(c3cccc(Cl)c3)CC1)OC2=O]

Preparation of tert-butyl (R)-3-(2-(4-(3-chlorophenyl)piperazin-1-yl)ethyl)-1-oxo-2-oxa-8-azaspiro[4.5]decane-8-carboxylate: The title compound was prepared according to the procedure for tert-butyl (R)-3-(2-((S)-4-(4-fluorophenyl)-2-methylpiperazin-1-yl)ethyl)-1-oxo-2-oxa-8-azaspiro[4.5]decane-8-carboxylate, except 1-(3-chlorophenyl)piperazine was substituted for (S)-1-(4-fluorophenyl)-3-methylpiperazine. LC/MS [M+H]=m/z 478.2

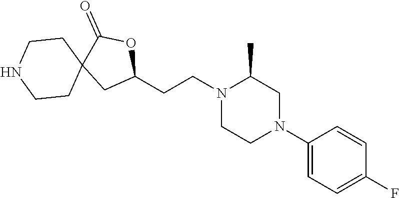 [SMILES: C[C@H]1CN(c2ccc(F)cc2)CCN1CC[C@H]1CC2(CCNCC2)C(=O)O1]

Preparation of (R)-3-(2-((S)-4-(4-fluorophenyl)-2-methylpiperazin-1-yl)ethyl)-2-oxa-8-azaspiro[4.5]decan-1-one: A 6M HCl in methanol solution was prepared via the addition of acetyl chloride (1.2 mL) to methanol (3 mL). tert-butyl (R)-3-(2-((S)-4-(4-fluorophenyl)-2-methylpiperazin-1-yl)ethyl)-1-oxo-2-oxa-8-azaspiro[4.5]decane-8-carboxylate (0.127 g, 0.267 mmol, 1.0 equiv.) was dissolved in the prepared 6M methanolic HCl solution (3 mL) and let stir at 23° C. for 30 minutes before being diluted with methanol and concentrated in vacuo to produce a crude product as an diHCl salt. The product was free based by stirring with Amberlite IRN-78 base resin in methanol for 15 minutes followed by filtration and concentrated in vacuo to produce a crude product LC/MS [M+H]=m/z 376.2

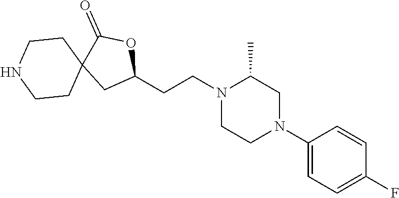 [SMILES: C[C@@H]1CN(c2ccc(F)cc2)CCN1CC[C@H]1CC2(CCNCC2)C(=O)O1]

Preparation of (R)-3-(2-((R)-4-(4-fluorophenyl)-2-methylpiperazin-1-yl)ethyl)-2-oxa-8-azaspiro[4.5]decan-1-one: The title compound was prepared according to the procedure for (R)-3-(2-((S)-4-(4-fluorophenyl)-2-methylpiperazin-1-yl)ethyl)-2-oxa-8-azaspiro[4.5]decan-1-one, except tert-butyl (R)-3-(2-((R)-4-(4-fluorophenyl)-2-methylpiperazin-1-yl)ethyl)-1-oxo-2-oxa-8-azaspiro[4.5]decane-8-carboxylate was substituted for tert-butyl (R)-3-(2-((S)-4-(4-fluorophenyl)-2-methylpiperazin-1-yl)ethyl)-1-oxo-2-oxa-8-azaspiro[4.5]decane-8-carboxylate. LC/MS [M+H]=m/z 376.2

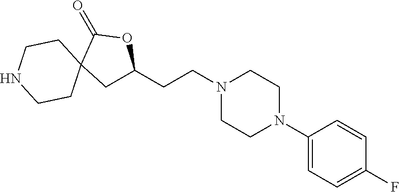 [SMILES: O=C1O[C@@H](CCN2CCN(c3ccc(F)cc3)CC2)CC12CCNCC2]

Preparation of (R)-3-(2-(4-(4-fluorophenyl)piperazin-1-yl)ethyl)-2-oxa-8-azaspiro[4.5]decan-1-one: The title compound was prepared according to the procedure for (R)-3-(2-((S)-4-(4-fluorophenyl)-2-methylpiperazin-1-yl)ethyl)-2-oxa-8-azaspiro[4.5]decan-1-one, except tert-butyl (R)-3-(2-(4-(4-fluorophenyl)piperazin-1-yl)ethyl)-1-oxo-2-oxa-8-azaspiro[4.5]decane-8-carboxylate was substituted for tert-butyl (R)-3-(2-((S)-4-(4-fluorophenyl)-2-methylpiperazin-1-yl)ethyl)-1-oxo-2-oxa-8-azaspiro[4.5]decane-8-carboxylate. LC/MS [M+H]=m/z 362.2

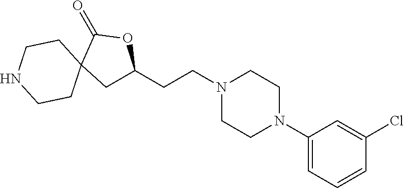 [SMILES: O=C1O[C@@H](CCN2CCN(c3cccc(Cl)c3)CC2)CC12CCNCC2]

Preparation of (R)-3-(2-(4-(3-chlorophenyl)piperazin-1-yl)ethyl)-2-oxa-8-azaspiro[4.5]decan-1-one: The title compound was prepared according to the procedure for (R)-3-(2-((S)-4-(4-fluorophenyl)-2-methylpiperazin-1-yl)ethyl)-2-oxa-8-azaspiro[4.5]decan-1-one, except tert-butyl (R)-3-(2-(4-(3-chlorophenyl)piperazin-1-yl)ethyl)-1-oxo-2-oxa-8-azaspiro[4.5]decane-8-carboxylate was substituted for tert-butyl (R)-3-(2-((S)-4-(4-fluorophenyl)-2-methylpiperazin-1-yl)ethyl)-1-oxo-2-oxa-8-azaspiro[4.5]decane-8-carboxylate. LC/MS [M+H]=m/z 378.2

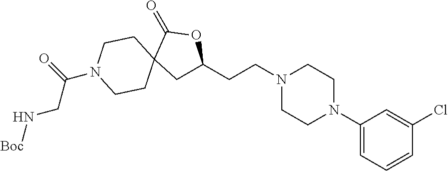 [SMILES: CC(C)(C)OC(=O)NCC(=O)N1CCC2(CC1)C[C@H](CCN1CCN(c3cccc(Cl)c3)CC1)OC2=O]

Preparation of tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate: This reaction was performed in oven-dried glassware under a nitrogen atmosphere. To a small vial was added N-(tert-Butoxycarbonyl)glycine (13.8 mg, 0.078 mmol, 1.05 eq.) and dissolved in dimethylformamide (700 μL). Following, 1-[Bis(dimethylamino)methylene]-1H-1,2,3-triazolo[4,5-b]pyridinium 3-oxid hexafluorophosphate (31.3 mg, 0.082 mmol, 1.1 eq.) and N,N-diisopropylethylamine (39 mg, 0.3 mmol, 4 eq.) were added and the resulting solution was allowed to stir at 23° C. for 15 minutes. In a separate vial, a solution of (R)-3-(2-(4-(3-chlorophenyl)piperazin-1-yl)ethyl)-2-oxa-8-azaspiro[4.5]decan-1-one dihydrochloride (33.3 mg, 0.074 mmol, 1.0 eq.), dimethylformamide (300 μL) and N,N-diisopropylethylamine (17.3 mg, 0.13 mmol, 2.2 eq.) was prepared. Both solutions were combined and allowed to stir at 23° C. for 30 minutes before being diluted with methanol (5 mL) and concentration in vacuo. The resulting residue was suspending in sat. NaHCO₃ (5 mL) and extracted with dichloromethane (3×10 mL). The combined organic layers were dried over Na₂SO₄ and concentrated in vacuo to give a crude product which was further purified by column chromatography (methanol/dichloromethane, 0%˜10%). LC/MS [M+H]=m/z 535.3

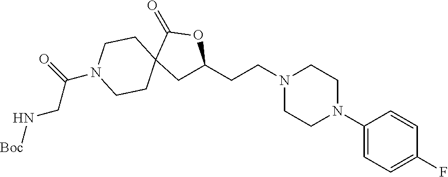 [SMILES: CC(C)(C)OC(=O)NCC(=O)N1CCC2(CC1)C[C@H](CCN1CCN(c3ccc(F)cc3)CC1)OC2=O]

Preparation of tert-butyl (R)-(2-(3-(2-(4-(4-fluorophenyl)piperazin-1-yl)ethyl)-1-oxo-2-oxa-8-azaspiro[4.5]decan-8-yl)-2-oxoethyl)carbamate: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one dihydrochloride was substituted for (R)-3-(2-(4-(3-chlorophenyl)piperazin-1-yl)ethyl)-2-oxa-8-azaspiro[4.5]decan-1-one dihydrochloride. LC/MS [M+H]=m/z 519.3

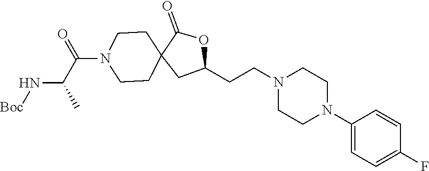 [SMILES: C[C@H](NC(=O)OC(C)(C)C)C(=O)N1CCC2(CC1)C[C@H](CCN1CCN(c3ccc(F)cc3)CC1)OC2=O]

Preparation of tert-butyl ((S)-1-((R)-3-(2-(4-(4-fluorophenyl)piperazin-1-yl)ethyl)-1-oxo-2-oxa-8-azaspiro[4.5]decan-8-yl)-1-oxopropan-2-yl)carbamate: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one dihydrochloride was substituted for (R)-3-(2-(4-(3-chlorophenyl)piperazin-1-yl)ethyl)-2-oxa-8-azaspiro[4.5]decan-1-one dihydrochloride and (tert-butoxycarbonyl)-L-alanine was substituted for N-(tert-Butoxycarbonyl)glycine. LC/MS [M+H]=m/z 533.3

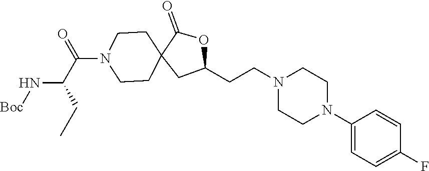 [SMILES: CC[C@H](NC(=O)OC(C)(C)C)C(=O)N1CCC2(CC1)C[C@H](CCN1CCN(c3ccc(F)cc3)CC1)OC2=O]

Preparation of tert-butyl ((S)-1-((R)-3-(2-(4-(4-fluorophenyl)piperazin-1-yl)ethyl)-1-oxo-2-oxa-8-azaspiro[4.5]decan-8-yl)-1-oxobutan-2-yl)carbamate: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one dihydrochloride was substituted for (R)-3-(2-(4-(3-chlorophenyl)piperazin-1-yl)ethyl)-2-oxa-8-azaspiro[4.5]decan-1-one dihydrochloride and (S)-2-((tert-butoxycarbonyl)amino)butanoic acid was substituted for N-(tert-Butoxycarbonyl)glycine. LC/MS [M+H]=m/z 547.3

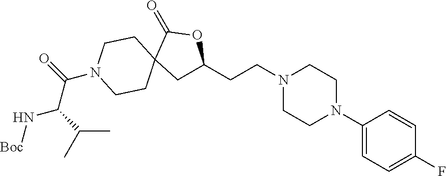 [SMILES: CC(C)[C@H](NC(=O)OC(C)(C)C)C(=O)N1CCC2(CC1)C[C@H](CCN1CCN(c3ccc(F)cc3)CC1)OC2=O]

Preparation of tert-butyl ((S)-1-((R)-3-(2-(4-(4-fluorophenyl)piperazin-1-yl)ethyl)-1-oxo-2-oxa-8-azaspiro[4.5]decan-8-yl)-3-methyl-1-oxobutan-2-yl)carbamate: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one dihydrochloride was substituted for (R)-3-(2-(4-(3-chlorophenyl)piperazin-1-yl)ethyl)-2-oxa-8-azaspiro[4.5]decan-1-one dihydrochloride and (tert-butoxycarbonyl)-L-valine was substituted for N-(tert-Butoxycarbonyl)glycine. LC/MS [M+H]=m/z 561.3

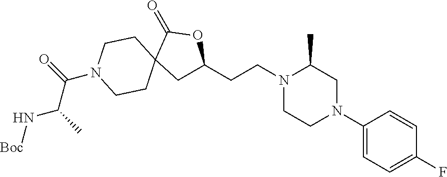 [SMILES: C[C@H](NC(=O)OC(C)(C)C)C(=O)N1CCC2(CC1)C[C@H](CCN1CCN(c3ccc(F)cc3)C[C@@H]1C)OC2=O]

Preparation of tert-butyl ((S)-1-((R)-3-(2-((S)-4-(4-fluorophenyl)-2-methylpiperazin-1-yl)ethyl)-1-oxo-2-oxa-8-azaspiro[4.5]decan-8-yl)-1-oxopropan-2-yl)carbamate: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-((S)-4-(4-fluorophenyl)-2-methylpiperazin-1-yl)ethyl)-2-oxa-8-azaspiro[4.5]decan-1-one was substituted for (R)-3-(2-(4-(3-chlorophenyl)piperazin-1-yl)ethyl)-2-oxa-8-azaspiro[4.5]decan-1-one dihydrochloride and (tert-butoxycarbonyl)-L-alanine was substituted for N-(tert-Butoxycarbonyl)glycine. LC/MS [M+H]=m/z 547.3

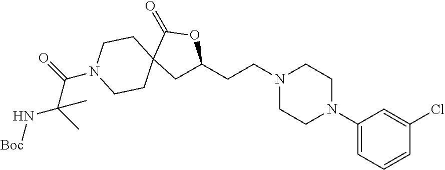 [SMILES: CC(C)(C)OC(=O)NC(C)(C)C(=O)N1CCC2(CC1)C[C@H](CCN1CCN(c3cccc(Cl)c3)CC1)OC2=O]

Preparation of tert-butyl (R)-(1-(3-(2-(4-(3-chlorophenyl)piperazin-1-yl)ethyl)-1-oxo-2-oxa-8-azaspiro[4.5]decan-8-yl)-2-methyl-1-oxopropan-2-yl)carbamate: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except 2-(boc-amino)isobutyric acid was substituted for N-(tert-Butoxycarbonyl)glycine. LC/MS [M+H]=m/z 563.3

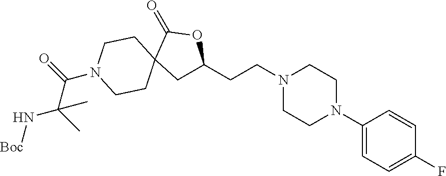 [SMILES: CC(C)(C)OC(=O)NC(C)(C)C(=O)N1CCC2(CC1)C[C@H](CCN1CCN(c3ccc(F)cc3)CC1)OC2=O]

Preparation of tert-butyl (R)-(1-(3-(2-(4-(4-fluorophenyl)piperazin-1-yl)ethyl)-1-oxo-2-oxa-8-azaspiro[4.5]decan-8-yl)-2-methyl-1-oxopropan-2-yl)carbamate: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-2-oxa-8-azaspiro[4.5]decan-1-one and 2-(boc-amino)isobutyric acid was substituted for N-(tert-Butoxycarbonyl)glycine. LC/MS [M+H]=m/z 547.3 Synthesis and Characterization of the 5-HT7 Modulators

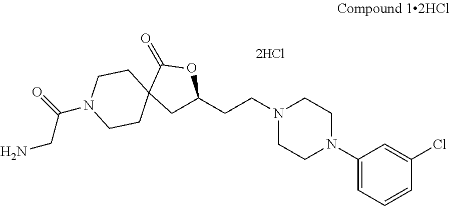 [SMILES: NCC(=O)N1CCC2(CC1)C[C@H](CCN1CCN(c3cccc(Cl)c3)CC1)OC2=O]

Preparation of (R)-3-(2-(4-(3-chlorophenyl)piperazin-1-yl)ethyl)-8-glycyl-2-oxa-8-azaspiro[4.5]decan-1-one dihydrochloride: A 6M HCl in methanol solution was prepared via the addition of acetyl chloride (6 mL) to methanol (15 mL). tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate (0.607 g, 0.119 mmol, 1.0 equiv.) was dissolved in the prepared 6M methanolic HCl solution (13 mL) and let stir at 23° C. for 45 minutes before being diluted with methanol and concentrated in vacuo to produce a crude product as an diHCl salt. LC/MS [M+H]=m/z 435.2

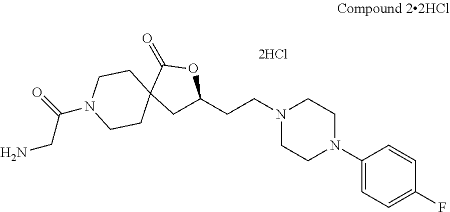 [SMILES: NCC(=O)N1CCC2(CC1)C[C@H](CCN1CCN(c3ccc(F)cc3)CC1)OC2=O]

Preparation of (R)-3-(2-(4-(4-fluorophenyl)piperazin-1-yl)ethyl)-8-glycyl-2-oxa-8-azaspiro[4.5]decan-1-one dihydrochloride: The title compound was prepared according to the procedure for (R)-3-(2-(4-(3-chlorophenyl)piperazin-1-yl)ethyl)-8-glycyl-2-oxa-8-azaspiro[4.5]decan-1-one dihydrochloride, except tert-butyl (R)-(2-(3-(2-(4-(4-fluorophenyl)piperazin-1-yl)ethyl)-1-oxo-2-oxa-8-azaspiro[4.5]decan-8-yl)-2-oxoethyl)carbamate was substituted for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate. LC/MS [M+H]=m/z 419.2

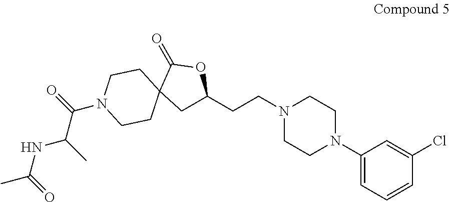 [SMILES: CC(=O)NC(C)C(=O)N1CCC2(CC1)C[C@H](CCN1CCN(c3cccc(Cl)c3)CC1)OC2=O]

Preparation of (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide: A solution of (R)-3-(2-(4-(3-chlorophenyl)piperazin-1-yl)ethyl)-8-glycyl-2-oxa-8-azaspiro[4.5]decan-1-one dihydrochloride (31.6 mg, 0.062 mmol, 1 equiv.), dichloromethane (1.5 mL) and triethylamine (28.5 mg, 0.24 mmol, 4 eq.) was cooled to 0° C. before acetyl chloride (5.0 mg. 0.062 mmol, 1 equiv.) was added to the solution. The reaction solution was allowed to warm to 23° C. and stir for 15 minutes. The reaction was diluted with methanol (˜2 mL), concentrated in vacuo and further purified by column chromatography on a C18 column (ACN/H₂O, 0%˜100%, w/0.1% NH₄OH). LC/MS [M+H]=m/z 477.2

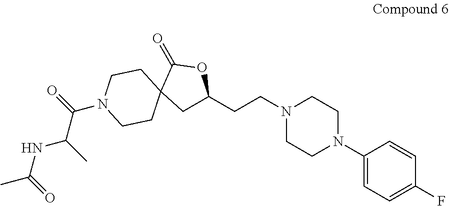 [SMILES: CC(=O)NC(C)C(=O)N1CCC2(CC1)C[C@H](CCN1CCN(c3ccc(F)cc3)CC1)OC2=O]

Preparation of (R)—N-(2-(3-(2-(4-(4-fluorophenyl)piperazin-1-yl)ethyl)-1-oxo-2-oxa-8-azaspiro[4.5]decan-8-yl)-2-oxoethyl)acetamide: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-3-(2-(4-(4-fluorophenyl)piperazin-1-yl)ethyl)-8-glycyl-2-oxa-8-azaspiro[4.5]decan-1-one dihydrochloride was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 461.2

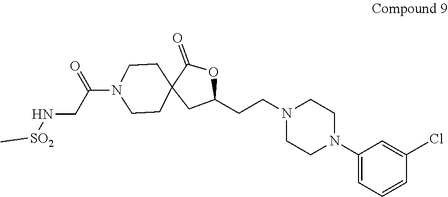 [SMILES: CS(=O)(=O)NCC(=O)N1CCC2(CC1)C[C@H](CCN1CCN(c3cccc(Cl)c3)CC1)OC2=O]

Preparation of (R)—N-(2-(3-(2-(4-(3-chlorophenyl)piperazin-1-yl)ethyl)-1-oxo-2-oxa-8-azaspiro[4.5]decan-8-yl)-2-oxoethyl)methanesulfonamide: A solution of (R)-3-(2-(4-(3-chlorophenyl)piperazin-1-yl)ethyl)-8-glycyl-2-oxa-8-azaspiro[4.5]decan-1-one dihydrochloride (431 mg, 0.845 mmol, 1 equiv.), dichloromethane (17 mL) and triethylamine (516 mg, 5.1 mmol, 6 eq.) was cooled to 0° C. before methanesulfonyl chloride (117 mg. 1.02 mmol, 1.2 equiv.) was added to the solution. The reaction solution was allowed to warm to 23° C. and stir for 15 minutes. The reaction was diluted with methanol (˜10 mL), concentrated in vacuo and further purified by column chromatography on a C18 column (ACN/H₂O, 0% 100%, w/0.1% NH₄OH). LC/MS [M+H]=m/z 513.2

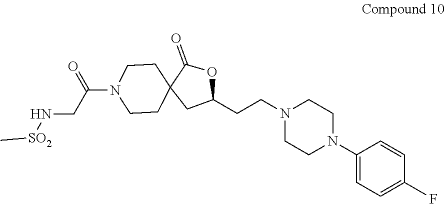 [SMILES: CS(=O)(=O)NCC(=O)N1CCC2(CC1)C[C@H](CCN1CCN(c3ccc(F)cc3)CC1)OC2=O]

Preparation of (R)—N-(2-(3-(2-(4-(4-fluorophenyl)piperazin-1-yl)ethyl)-1-oxo-2-oxa-8-azaspiro[4.5]decan-8-yl)-2-oxoethyl)methanesulfonamide: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)methanesulfonamide, except (R)-3-(2-(4-(4-fluorophenyl)piperazin-1-yl)ethyl)-8-glycyl-2-oxa-8-azaspiro[4.5]decan-1-one dihydrochloride was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 497.2

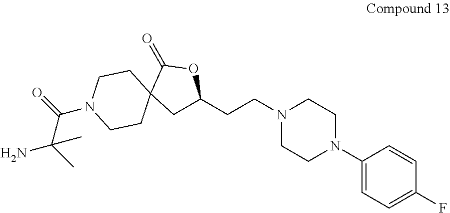 [SMILES: CC(C)(N)C(=O)N1CCC2(CC1)C[C@H](CCN1CCN(c3ccc(F)cc3)CC1)OC2=O]

Preparation of (R)-8-(2-amino-2-methylpropanoyl)-3-(2-(4-(4-fluorophenyl)piperazin-1-yl)ethyl)-2-oxa-8-azaspiro[4.5]decan-1-one: The title compound was prepared according to the procedure for (R)-3-(2-(4-(3-chlorophenyl)piperazin-1-yl)ethyl)-8-glycyl-2-oxa-8-azaspiro[4.5]decan-1-one dihydrochloride, except tert-butyl (R)-(1-(3-(2-(4-(4-fluorophenyl)piperazin-1-yl)ethyl)-1-oxo-2-oxa-8-azaspiro[4.5]decan-8-yl)-2-methyl-1-oxopropan-2-yl)carbamate was substituted for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate. In addition, the product was free based by stirring with Amberlite IRN-78 base resin in methanol for 15 minutes followed by filtration and concentrated in vacuo to produce the product. LC/MS [M+H]=m/z 447.2

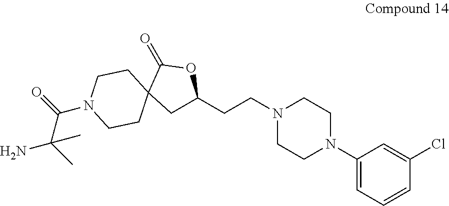 [SMILES: CC(C)(N)C(=O)N1CCC2(CC1)C[C@H](CCN1CCN(c3cccc(Cl)c3)CC1)OC2=O]

Preparation of (R)-8-(2-amino-2-methylpropanoyl)-3-(2-(4-(3-chlorophenyl)piperazin-1-yl)ethyl)-2-oxa-8-azaspiro[4.5]decan-1-one: The title compound was prepared according to the procedure for (R)-3-(2-(4-(3-chlorophenyl)piperazin-1-yl)ethyl)-8-glycyl-2-oxa-8-azaspiro[4.5]decan-1-one dihydrochloride, except tert-butyl (R)-(1-(3-(2-(4-(3-chlorophenyl)piperazin-1-yl)ethyl)-1-oxo-2-oxa-8-azaspiro[4.5]decan-8-yl)-2-methyl-1-oxopropan-2-yl)carbamate was substituted for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate. In addition, the product was free based by stirring with Amberlite IRN-78 base resin in methanol for 15 minutes followed by filtration and concentrated in vacuo to produce the product. LC/MS [M+H]=m/z 463.2

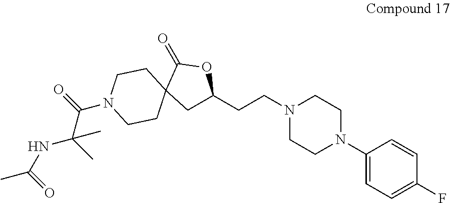 [SMILES: CC(=O)NC(C)(C)C(=O)N1CCC2(CC1)C[C@H](CCN1CCN(c3ccc(F)cc3)CC1)OC2=O]

Preparation of (R)—N-(1-(3-(2-(4-(4-fluorophenyl)piperazin-1-yl)ethyl)-1-oxo-2-oxa-8-azaspiro[4.5]decan-8-yl)-2-methyl-1-oxopropan-2-yl)acetamide: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-8-(2-amino-2-methylpropanoyl)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 488.2

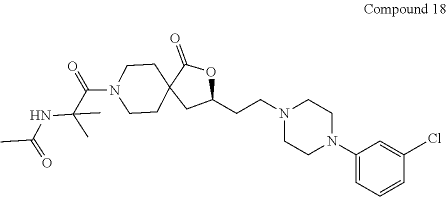 [SMILES: CC(=O)NC(C)(C)C(=O)N1CCC2(CC1)C[C@H](CCN1CCN(c3cccc(Cl)c3)CC1)OC2=O]

Preparation of (R)—N-(1-(3-(2-(4-(3-chlorophenyl)piperazin-1-yl)ethyl)-1-oxo-2-oxa-8-azaspiro[4.5]decan-8-yl)-2-methyl-1-oxopropan-2-yl)acetamide: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-8-(2-amino-2-methylpropanoyl)-3-(2-(4-(3-chlorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 504.2

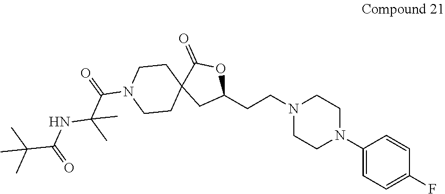 [SMILES: CC(C)(C)C(=O)NC(C)(C)C(=O)N1CCC2(CC1)C[C@H](CCN1CCN(c3ccc(F)cc3)CC1)OC2=O]

Preparation of (R)—N-(1-(3-(2-(4-(4-fluorophenyl)piperazin-1-yl)ethyl)-1-oxo-2-oxa-8-azaspiro[4.5]decan-8-yl)-2-methyl-1-oxopropan-2-yl)pivalamide: The title compound was prepared according to the procedure for (R)—N-(2-(3-(2-(4-(4-fluorophenyl)piperazin-1-yl)ethyl)-1-oxo-2-oxa-8-azaspiro[4.5]decan-8-yl)-2-oxoethyl)pivalamide, except (R)-8-(2-amino-2-methylpropanoyl)-3-(2-(4-(4-fluorophenyl)piperazin-1-yl)ethyl)-2-oxa-8-azaspiro[4.5]decan-1-one was substituted for (R)-3-(2-(4-(4-fluorophenyl)piperazin-1-yl)ethyl)-8-glycyl-2-oxa-8-azaspiro[4.5]decan-1-one dihydrochloride. LC/MS [M+H]=m/z 531.3

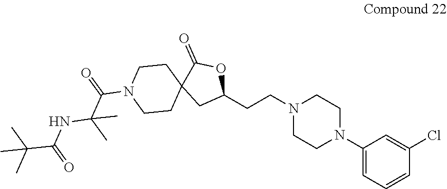 [SMILES: CC(C)(C)C(=O)NC(C)(C)C(=O)N1CCC2(CC1)C[C@H](CCN1CCN(c3cccc(Cl)c3)CC1)OC2=O]

Preparation of (R)—N-(1-(3-(2-(4-(3-chlorophenyl)piperazin-1-yl)ethyl)-1-oxo-2-oxa-8-azaspiro[4.5]decan-8-yl)-2-methyl-1-oxopropan-2-yl)pivalamide: The title compound was prepared according to the procedure for (R)—N-(2-(3-(2-(4-(4-fluorophenyl)piperazin-1-yl)ethyl)-1-oxo-2-oxa-8-azaspiro[4.5]decan-8-yl)-2-oxoethyl)pivalamide, except (R)-8-(2-amino-2-methylpropanoyl)-3-(2-(4-(3-chlorophenyl)piperazin-1-yl)ethyl)-2-oxa-8-azaspiro[4.5]decan-1-one was substituted for (R)-3-(2-(4-(4-fluorophenyl)piperazin-1-yl)ethyl)-8-glycyl-2-oxa-8-azaspiro[4.5]decan-1-one dihydrochloride. LC/MS [M+H]=m/z 547.3

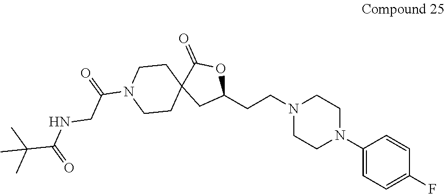 [SMILES: CC(C)(C)C(=O)NCC(=O)N1CCC2(CC1)C[C@H](CCN1CCN(c3ccc(F)cc3)CC1)OC2=O]

Preparation of (R)—N-(2-(3-(2-(4-(4-fluorophenyl)piperazin-1-yl)ethyl)-1-oxo-2-oxa-8-azaspiro[4.5]decan-8-yl)-2-oxoethyl)pivalamide: This reaction was performed in oven-dried glassware under a nitrogen atmosphere. To a small vial was added sodium pivalate (21.8 mg, 0.17 mmol, 1.05 eq.) and dissolved in dimethylformamide (1.5 mL). Following, 1-[Bis(dimethylamino)methylene]-1H-1,2,3-triazolo[4,5-b]pyridinium 3-oxid hexafluorophosphate (70 mg, 0.18 mmol, 1.1 eq.) and N,N-diisopropylethylamine (86.3 mg, 0.67 mmol, 4 eq.) were added and the resulting solution was allowed to stir at 23° C. for 15 minutes. In a separate vial, a solution of (R)-3-(2-(4-(4-fluorophenyl)piperazin-1-yl)ethyl)-8-glycyl-2-oxa-8-azaspiro[4.5]decan-1-one dihydrochloride (81.2 mg, 0.16 mmol, 1.0 eq.), dimethylformamide (400 μL) and N,N-diisopropylethylamine (48 mg, 0.37 mmol, 2.2 eq.) was prepared. Both solutions were combined and allowed to stir at 23° C. for 30 minutes before being diluted with methanol (5 mL) and concentration in vacuo. The resulting residue was suspending in sat. NaHCO₃ (5 mL) and extracted with dichloromethane (3×10 mL). The combined organic layers were dried over Na₂SO₄ and concentrated in vacuo to give a crude product which was further purified by column chromatography on a C18 column (ACN/H₂O, 0%˜100%, w/0.1% NH₄OH). LC/MS [M+H]=m/z 503.3

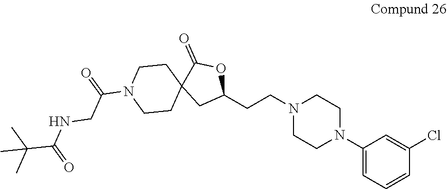 [SMILES: CC(C)(C)C(=O)NCC(=O)N1CCC2(CC1)C[C@H](CCN1CCN(c3cccc(Cl)c3)CC1)OC2=O]

Preparation of (R)—N-(2-(3-(2-(4-(3-chlorophenyl)piperazin-1-yl)ethyl)-1-oxo-2-oxa-8-azaspiro[4.5]decan-8-yl)-2-oxoethyl)pivalamide: The title compound was prepared according to the procedure for (R)—N-(2-(3-(2-(4-(4-fluorophenyl)piperazin-1-yl)ethyl)-1-oxo-2-oxa-8-azaspiro[4.5]decan-8-yl)-2-oxoethyl)pivalamide, except (R)-3-(2-(4-(3-chlorophenyl)piperazin-1-yl)ethyl)-8-glycyl-2-oxa-8-azaspiro[4.5]decan-1-one dihydrochloride was substituted for (R)-3-(2-(4-(4-fluorophenyl)piperazin-1-yl)ethyl)-8-glycyl-2-oxa-8-azaspiro[4.5]decan-1-one dihydrochloride. LC/MS [M+H]=m/z 519.3

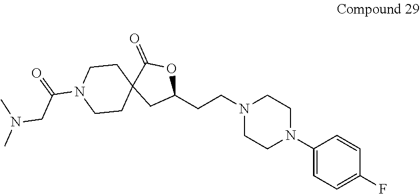 [SMILES: CN(C)CC(=O)N1CCC2(CC1)C[C@H](CCN1CCN(c3ccc(F)cc3)CC1)OC2=O]

Preparation of (R)-8-(dimethylglycyl)-3-(2-(4-(4-fluorophenyl)piperazin-1-yl)ethyl)-2-oxa-8-azaspiro[4.5]decan-1-one: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-2-oxa-8-azaspiro[4.5]decan-1-one dihydrochloride and N,N-dimethylglycine was substituted for N-(tert-Butoxycarbonyl)glycine. In addition, the purification method was replaced by column chromatography on a C18 column. (ACN/H₂O, 0% 100%, w/0.1% NH₄OH). LC/MS [M+H]=m/z 447.3

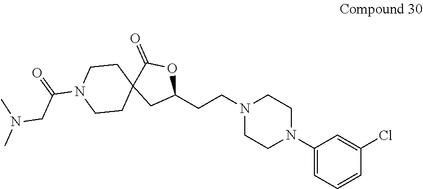 [SMILES: CN(C)CC(=O)N1CCC2(CC1)C[C@H](CCN1CCN(c3cccc(Cl)c3)CC1)OC2=O]

Preparation of (R)-3-(2-(4-(3-chlorophenyl)piperazin-1-yl)ethyl)-8-(dimethylglycyl)-2-oxa-8-azaspiro[4.5]decan-1-one: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except N,N-dimethylglycine was substituted for N-(tert-Butoxycarbonyl)glycine. In addition, the purification method was replaced by column chromatography on a C18 column. (ACN/H₂O, 0%˜100%, w/0.1% NH₄OH). LC/MS [M+H]=m/z 463.3

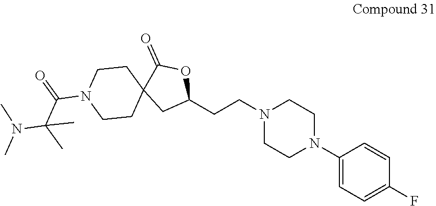 [SMILES: CN(C)C(C)(C)C(=O)N1CCC2(CC1)C[C@H](CCN1CCN(c3ccc(F)cc3)CC1)OC2=O]

Preparation of (R)-8-(2-(dimethylamino)-2-methylpropanoyl)-3-(2-(4-(4-fluorophenyl)piperazin-1-yl)ethyl)-2-oxa-8-azaspiro[4.5]decan-1-one: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-2-oxa-8-azaspiro[4.5]decan-1-one dihydrochloride and 2-(dimethylamino)-2-methylpropanoic acid was substituted for N-(tert-Butoxycarbonyl)glycine. In addition, the purification method was replaced by column chromatography on a C18 column. (ACN/H₂O, 0%˜100%, w/0.1% NH₄OH). LC/MS [M+H]=m/z 475.3

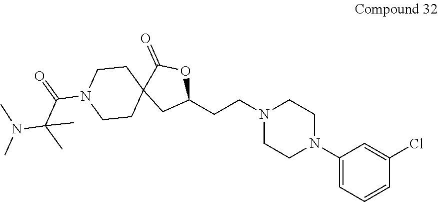 [SMILES: CN(C)C(C)(C)C(=O)N1CCC2(CC1)C[C@H](CCN1CCN(c3cccc(Cl)c3)CC1)OC2=O]

Preparation of (R)-3-(2-(4-(3-chlorophenyl)piperazin-1-yl)ethyl)-8-(2-(dimethylamino)-2-methylpropanoyl)-2-oxa-8-azaspiro[4.5]decan-1-one: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except 2-(dimethylamino)-2-methylpropanoic acid was substituted for N-(tert-Butoxycarbonyl)glycine. In addition, the purification method was replaced by column chromatography on a C18 column. (ACN/H₂O, 0%˜100%, w/0.1% NH₄OH). LC/MS [M+H]=m/z 491.3

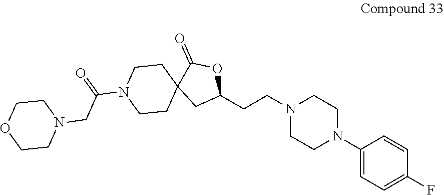 [SMILES: O=C(CN1CCOCC1)N1CCC2(CC1)C[C@H](CCN1CCN(c3ccc(F)cc3)CC1)OC2=O]

Preparation of (R)-3-(2-(4-(4-fluorophenyl)piperazin-1-yl)ethyl)-8-(2-morpholinoacetyl)-2-oxa-8-azaspiro[4.5]decan-1-one: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-2-oxa-8-azaspiro[4.5]decan-1-one dihydrochloride and morpholin-4-yl acetic acid was substituted for N-(tert-Butoxycarbonyl)glycine. In addition, the purification method was replaced by column chromatography on a C18 column. (ACN/H₂O, 0% 100%, w/0.1% NH₄OH). LC/MS [M+H]=m/z 489.3

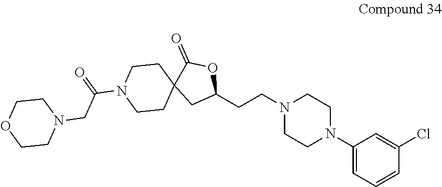 [SMILES: O=C(CN1CCOCC1)N1CCC2(CC1)C[C@H](CCN1CCN(c3cccc(Cl)c3)CC1)OC2=O]

Preparation of (R)-3-(2-(4-(3-chlorophenyl)piperazin-1-yl)ethyl)-8-(2-morpholinoacetyl)-2-oxa-8-azaspiro[4.5]decan-1-one: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except morpholin-4-yl acetic acid was substituted for N-(tert-Butoxycarbonyl)glycine. In addition, the purification method was replaced by column chromatography on a C18 column. (ACN/H₂O, 0%˜100%, w/0.1% NH₄OH). LC/MS [M+H]=m/z 505.3

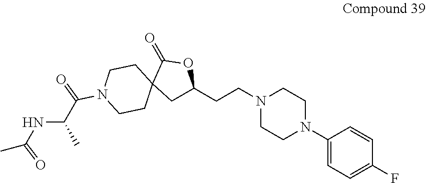 [SMILES: CC(=O)N[C@@H](C)C(=O)N1CCC2(CC1)C[C@H](CCN1CCN(c3ccc(F)cc3)CC1)OC2=O]

Preparation of N—((S)-1-((R)-3-(2-(4-(4-fluorophenyl)piperazin-1-yl)ethyl)-1-oxo-2-oxa-8-azaspiro[4.5]decan-8-yl)-1-oxopropan-2-yl)acetamide: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-8-(L-alanyl)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 475.2

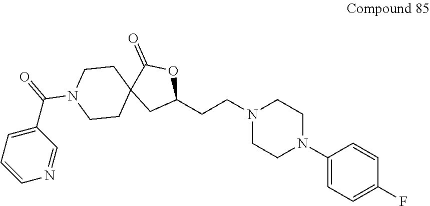 [SMILES: O=C(c1cccnc1)N1CCC2(CC1)C[C@H](CCN1CCN(c3ccc(F)cc3)CC1)OC2=O]

Preparation of (R)-3-(2-(4-(4-fluorophenyl)piperazin-1-yl)ethyl)-8-nicotinoyl-2-oxa-8-azaspiro[4.5]decan-1-one: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)methanesulfonamide, except (R)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride and nicotinoyl chloride hydrochloride was substituted for methanesulfonyl chloride. LC/MS [M+H]=m/z 467.2

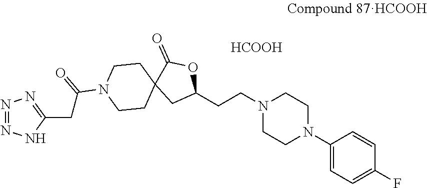 [SMILES: O=C(Cc1nnn[nH]1)N1CCC2(CC1)C[C@H](CCN1CCN(c3ccc(F)cc3)CC1)OC2=O ; "HCOOH"]

Preparation of (R)-8-(2-(1H-tetrazol-5-yl)acetyl)-3-(2-(4-(4-fluorophenyl)piperazin-1-yl)ethyl)-2-oxa-8-azaspiro[4.5]decan-1-one formate: The title compound was prepared according to the procedure for (R)-3-(2-(4-(4-fluorophenyl)piperazin-1-yl)ethyl)-8-(pyridazine-3-carbonyl)-2-oxa-8-azaspiro[4.5]decan-1-one, except 2H-tetrazole-5-acetic acid was substituted for 3-pyridazinecarboxylic acid. In addition, the purification method was replaced by column chromatography on a C18 column. (ACN/H₂O, 0%˜100%, w/0.1% HCOOH). LC/MS [M+H]=m/z 472.2

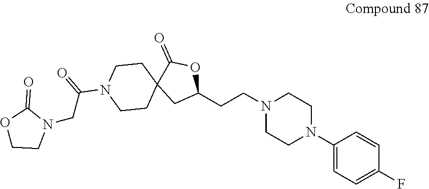 [SMILES: O=C(CN1CCOC1=O)N1CCC2(CC1)C[C@H](CCN1CCN(c3ccc(F)cc3)CC1)OC2=O]

Preparation of (R)-3-(2-(4-(4-fluorophenyl)piperazin-1-yl)ethyl)-8-(2-(2-oxooxazolidin-3-yl)acetyl)-2-oxa-8-azaspiro[4.5]decan-1-one: The title compound was prepared according to the procedure for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate, except (R)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-2-oxa-8-azaspiro[4.5]decan-1-one dihydrochloride and (2-oxo-1,3-oxazolidin-3-yl)acetic acid was substituted for N-(tert-Butoxycarbonyl)glycine. In addition, the purification method was replaced by column chromatography on a C18 column. (ACN/H₂O, 0% 100%, w/0.1% NH₄OH). LC/MS [M+H]=m/z 489.3

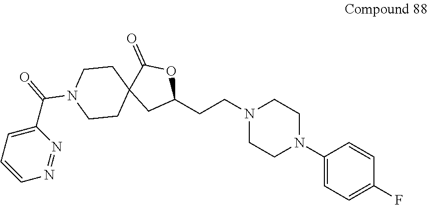 [SMILES: O=C(c1cccnn1)N1CCC2(CC1)C[C@H](CCN1CCN(c3ccc(F)cc3)CC1)OC2=O]

Preparation of (R)-3-(2-(4-(4-fluorophenyl)piperazin-1-yl)ethyl)-8-(pyridazine-3-carbonyl)-2-oxa-8-azaspiro[4.5]decan-1-one: This reaction was performed in oven-dried glassware under a nitrogen atmosphere. To a small vial was added 3-pyridazinecarboxylic acid (14.3 mg, 0.115 mmol, 1.9 eq.) and dissolved in dimethylacetamide (500 μL). Following, N-(3-Dimethylaminopropyl)-N′-ethylcarbodiimide hydrochloride (55.8 mg, 0.291 mmol, 4.8 eq.), 1-benzotriazole (39.3 mg, 0.291 mmol, 4.8 eq.) and N-methylmorpholine (59 mg, 0.584 mmol, 9.6 eq.) were added and the resulting solution was allowed to stir at 23° C. for 15 minutes. Following, (R)-3-(2-(4-(4-fluorophenyl)piperazin-1-yl)ethyl)-2-oxa-8-azaspiro[4.5]decan-1-one (21.9 mg, 0.0607 mmol, 1 eq.) was added using dimethylacetamide (150 μL) to assist transfer. The resulting solution was allowed to stir for 23° C. for 30 minutes before being diluted with methanol (5 mL) and concentration in vacuo. The resulting residue was suspending in sat. NaHCO₃ (5 mL) and extracted with dichloromethane (3×10 mL). The combined organic layers were dried over Na₂SO₄ and concentrated in vacuo to give a crude product which was further purified by column chromatography on a C18 column (ACN/H₂O, 0%˜100%, w/0.1% NH₄OH). LC/MS [M+H]=m/z 468.2

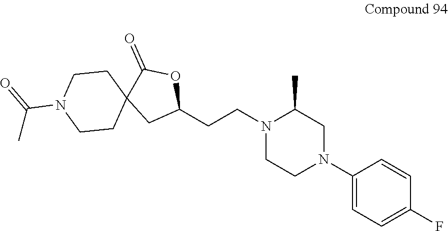 [SMILES: CC(=O)N1CCC2(CC1)C[C@H](CCN1CCN(c3ccc(F)cc3)C[C@@H]1C)OC2=O]

Preparation of (R)-8-acetyl-3-(2-((S)-4-(4-fluorophenyl-2-methylpiperazin-1-yl)ethyl)-2-oxa-8-azaspiro[4.5]decan-1-one: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-3-(2-((S)-4-(4-fluorophenyl)-2-methylpiperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 418.2

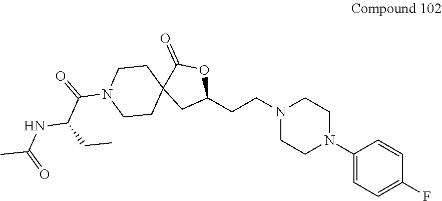 [SMILES: CC[C@H](NC(C)=O)C(=O)N1CCC2(CC1)C[C@H](CCN1CCN(c3ccc(F)cc3)CC1)OC2=O]

Preparation of N—((S)-1-((R)-3-(2-(4-(4-fluorophenyl)piperazin-1-yl)ethyl)-1-oxo-2-oxa-8-azaspiro[4.5]decan-8-yl)-1-oxobutan-2-yl)acetamide: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-8-((S)-2-aminobutanoyl)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 489.2

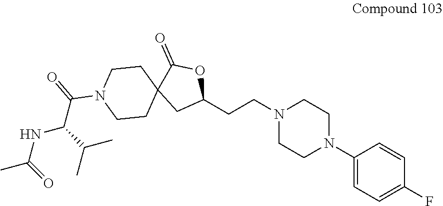 [SMILES: CC(=O)N[C@H](C(=O)N1CCC2(CC1)C[C@H](CCN1CCN(c3ccc(F)cc3)CC1)OC2=O)C(C)C]

Preparation of N—((S)-1-((R)-3-(2-(4-(4-fluorophenyl)piperazin-1-yl)ethyl)-1-oxo-2-oxa-8-azaspiro[4.5]decan-8-yl)-3-methyl-1-oxobutan-2-yl)acetamide: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-8-(L-valyl)-3-(2-(4-(4-fluorophenyl)piperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 502.2

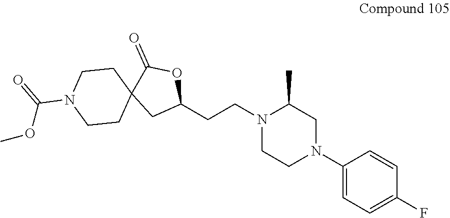 [SMILES: COC(=O)N1CCC2(CC1)C[C@H](CCN1CCN(c3ccc(F)cc3)C[C@@H]1C)OC2=O]

Preparation of methyl (R)-3-(2-((S)-4-(4-fluorophenyl)-2-methylpiperazin-1-yl)ethyl)-1-oxo-2-oxa-8-azaspiro[4.5]decane-8-carboxylate: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-3-(2-((S)-4-(4-fluorophenyl)-2-methylpiperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride and methyl chloroformate was substituted for acetyl chloride. LC/MS [M+H]=m/z 434.2

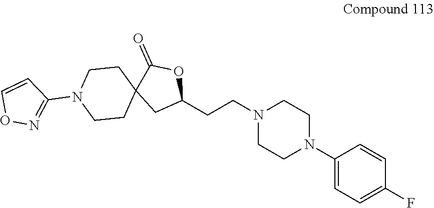 [SMILES: O=C1O[C@@H](CCN2CCN(c3ccc(F)cc3)CC2)CC12CCN(c1ccon1)CC2]

Preparation of (R)-3-(2-(4-(4-fluorophenyl)piperazin-1-yl)ethyl)-8-(isoxazol-3-yl)-2-oxa-8-azaspiro[4.5]decan-1-one: This reaction was performed in oven-dried glassware under a nitrogen atmosphere. To a solution of (R)-3-(2-(4-(4-fluorophenyl)piperazin-1-yl)ethyl)-2-oxa-8-azaspiro[4.5]decan-1-one (1.0 g, 2.76 mmol, 1.0 eq.) and 3-bromoisoxazole (0.614 g, 4.15 mmol, 1.5 eq.) in anhydrous toluene (10.6 mL) was added the following in this order: Pd₂(dba)₃ (0.063 g, 5 mol %), BINAP (0.127 g, 7.5 mol %), triethylamine (0.698 g, 6.6 mmol, 2.5 eq.) and NaOtBu (0.291 g, 3.3 mmol, 1.2 eq.). The resulting mixture was allowed to stir at 80° C. overnight, under a sweep of N₂. The reaction mixture was cooled to RT and then filtered through a plug of Celite. The collected filtrate was concentrated in vacuo to give a crude residue that was further purified by column chromatography (dichloromethane/methanol, 0%˜10%). LC/MS [M+H]=m/z 429.3

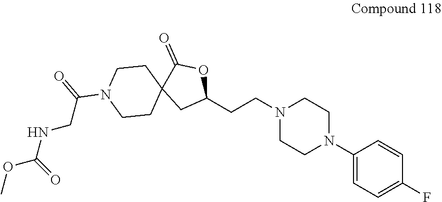 [SMILES: COC(=O)NCC(=O)N1CCC2(CC1)C[C@H](CCN1CCN(c3ccc(F)cc3)CC1)OC2=O]

Preparation of methyl (R)-(2-(3-(2-(4-(4-fluorophenyl)piperazin-1-yl)ethyl)-1-oxo-2-oxa-8-azaspiro[4.5]decan-8-yl)-2-oxoethyl)carbamate: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-3-(2-(4-(4-fluorophenyl)piperazin-1-yl)ethyl)-8-glycyl-2-oxa-8-azaspiro[4.5]decan-1-one dihydrochloride was substituted for (R)-3-(2-(4-(3-chlorophenyl)piperazin-1-yl)ethyl)-8-glycyl-2-oxa-8-azaspiro[4.5]decan-1-one dihydrochloride and methyl chloroformate was substituted for acetyl chloride. LC/MS [M+H]=m/z 477.2

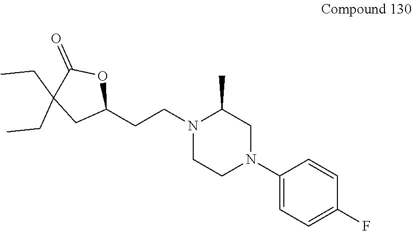 [SMILES: CCC1(CC)C[C@H](CCN2CCN(c3ccc(F)cc3)C[C@@H]2C)OC1=O]

Preparation of (R)-3,3-diethyl-5-(2-((S)-4-(4-fluorophenyl)-2-methylpiperazin-1-yl)ethyl)dihydrofuran-2(3H)-one: The title compound was prepared according to the procedure for tert-butyl (R)-3-(2-((S)-4-(4-fluorophenyl)-2-methylpiperazin-1-yl)ethyl)-1-oxo-2-oxa-8-azaspiro[4.5]decane-8-carboxylate, except (R)-2-(4,4-diethyl-5-oxotetrahydrofuran-2-yl)ethyl 4-methylbenzenesulfonate was substituted for tert-butyl (R)-1-oxo-3-(2-(tosyloxy)ethyl)-2-oxa-8-azaspiro[4.5]decane-8-carboxylate. LC/MS [M+H]=m/z 363.2

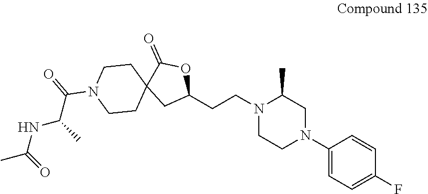 [SMILES: CC(=O)N[C@@H](C)C(=O)N1CCC2(CC1)C[C@H](CCN1CCN(c3ccc(F)cc3)C[C@@H]1C)OC2=O]

Preparation of N—((S)-1-((R)-3-(2-((S)-4-(4-fluorophenyl)-2-methylpiperazin-1-yl)ethyl)-1-oxo-2-oxa-8-azaspiro[4.5]decan-8-yl)-1-oxopropan-2-yl)acetamide: The title compound was prepared according to the procedure for (R)—N-(2-(3-(2-(4-(3-chlorophenyl)piperazin-1-yl)ethyl)-1-oxo-2-oxa-8-azaspiro[4.5]decan-8-yl)-2-oxoethyl)acetamide, except (R)-8-(L-alanyl)-3-(2-((S)-4-(4-fluorophenyl)-2-methylpiperazin-1-yl)ethyl)-2-oxa-8-azaspiro[4.5]decan-1-one was substituted for (R)-3-(2-(4-(3-chlorophenyl)piperazin-1-yl)ethyl)-8-glycyl-2-oxa-8-azaspiro[4.5]decan-1-one dihydrochloride. LC/MS [M+H]=m/z 489.2

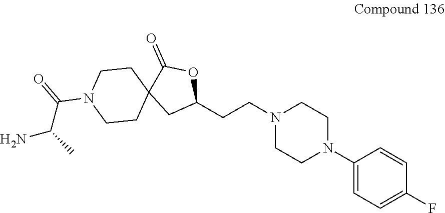 [SMILES: C[C@H](N)C(=O)N1CCC2(CC1)C[C@H](CCN1CCN(c3ccc(F)cc3)CC1)OC2=O]

Preparation of (R)-8-(L-alanyl)-3-(2-(4-(4-fluorophenyl)piperazin-1-yl)ethyl)-2-oxa-8-azaspiro[4.5]decan-1-one: The title compound was prepared according to the procedure for (R)-3-(2-(4-(3-chlorophenyl)piperazin-1-yl)ethyl)-8-glycyl-2-oxa-8-azaspiro[4.5]decan-1-one dihydrochloride, except tert-butyl ((S)-1-((R)-3-(2-(4-(4-fluorophenyl)piperazin-1-yl)ethyl)-1-oxo-2-oxa-8-azaspiro[4.5]decan-8-yl)-1-oxopropan-2-yl)carbamate was substituted for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate. In addition, the product was free based by stirring with Amberlite IRN-78 base resin in methanol for 15 minutes followed by filtration and concentrated in vacuo to produce the product. LC/MS [M+H]=m/z 433.2

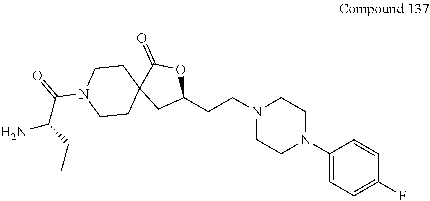 [SMILES: CC[C@H](N)C(=O)N1CCC2(CC1)C[C@H](CCN1CCN(c3ccc(F)cc3)CC1)OC2=O]

Preparation of (R)-8-((S)-2-aminobutanoyl)-3-(2-(4-(4-fluorophenyl)piperazin-1-yl)ethyl)-2-oxa-8-azaspiro[4.5]decan-1-one: The title compound was prepared according to the procedure for (R)-3-(2-(4-(3-chlorophenyl)piperazin-1-yl)ethyl)-8-glycyl-2-oxa-8-azaspiro[4.5]decan-1-one dihydrochloride, except tert-butyl ((S)-1-((R)-3-(2-(4-(4-fluorophenyl)piperazin-1-yl)ethyl)-1-oxo-2-oxa-8-azaspiro[4.5]decan-8-yl)-1-oxobutan-2-yl)carbamate was substituted for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate. In addition, the product was free based by stirring with Amberlite IRN-78 base resin in methanol for 15 minutes followed by filtration and concentrated in vacuo to produce the product LC/MS [M+H]=m/z 447.2

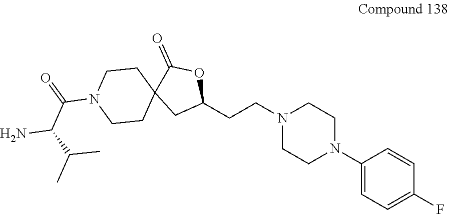 [SMILES: CC(C)[C@H](N)C(=O)N1CCC2(CC1)C[C@H](CCN1CCN(c3ccc(F)cc3)CC1)OC2=O]

Preparation of (R)-8-(L-valyl)-3-(2-(4-(4-fluorophenyl)piperazin-1-yl)ethyl)-2-oxa-8-azaspiro[4.5]decan-1-one: The title compound was prepared according to the procedure for (R)-3-(2-(4-(3-chlorophenyl)piperazin-1-yl)ethyl)-8-glycyl-2-oxa-8-azaspiro[4.5]decan-1-one dihydrochloride, except tert-butyl ((S)-1-((R)-3-(2-(4-(4-fluorophenyl)piperazin-1-yl)ethyl)-1-oxo-2-oxa-8-azaspiro[4.5]decan-8-yl)-3-methyl-1-oxobutan-2-yl)carbamate was substituted for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate. In addition, the product was free based by stirring with Amberlite IRN-78 base resin in methanol for 15 minutes followed by filtration and concentrated in vacuo to produce the product LC/MS [M+H]=m/z 461.2

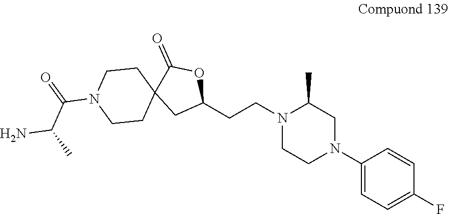 [SMILES: C[C@H](N)C(=O)N1CCC2(CC1)C[C@H](CCN1CCN(c3ccc(F)cc3)C[C@@H]1C)OC2=O]

Preparation of (R)-8-(L-alanyl)-3-(2-((S)-4-(4-fluorophenyl)-2-methylpiperazin-1-yl)ethyl)-2-oxa-8-azaspiro[4.5]decan-1-one: The title compound was prepared according to the procedure for (R)-3-(2-(4-(3-chlorophenyl)piperazin-1-yl)ethyl)-8-glycyl-2-oxa-8-azaspiro[4.5]decan-1-one dihydrochloride, except tert-butyl ((S)-1-((R)-3-(2-((S)-4-(4-fluorophenyl)-2-methylpiperazin-1-yl)ethyl)-1-oxo-2-oxa-8-azaspiro[4.5]decan-8-yl)-1-oxopropan-2-yl)carbamate was substituted for tert-butyl (R)-(2-(3-(2-(4-(3-chlorophenyl)piperazin-1-yl)ethyl)-1-oxo-2-oxa-8-azaspiro[4.5]decan-8-yl)-2-oxoethyl)carbamate. In addition, the product was free based by stirring with Amberlite IRN-78 base resin in methanol for 15 minutes followed by filtration and concentrated in vacuo to produce the product LC/MS [M+H]=m/z 447.2

### Biochemical Studies

Biological activity of the compounds described herein can be determined according to methods known in the art, including as described in International Application No. PCT/US19/31824, which is incorporated herein by reference in its entirety. Exemplary assays are described herein.

Methods

¹Radiolabeled Binding (IC₅₀ and K_{I})

A solution of the compound of the disclosure to be tested is prepared as a 1-mg/ml stock in Assay Buffer or DMSO according to its solubility. A similar stock of the reference compound chlorpromazine is also prepared as a positive control. Eleven dilutions (5× assay concentration) of the compound of the disclosure and chlorpromazine are prepared in the Assay Buffer by serial dilution to yield final corresponding assay concentrations ranging from 10 pM to 10 μM.

A stock concentration of 5 nM [³H]LSD (lysergic acid diethyl amide) is prepared in 50 mM Tris-HCl, 10 mM MgCl₂, 1 mM EDTA, pH 7.4 (Assay Buffer). Aliquots (50 l) of radioligand are dispensed into the wells of a 96-well plate containing 100 μl of Assay Buffer. Duplicate 50-1 aliquots of the compound of the disclosure test and chlorpromazine positive control reference compound serial dilutions are added.

Membrane fractions of cells expressing recombinant 5-HT₇ receptors (50 L) are dispensed into each well. The membranes are prepared from stably transfected cell lines expressing 5-HT₇ receptors cultured on 10-cm plates by harvesting PBS-rinsed monolayers, resuspending and lysing in chilled, hypotonic 50 mM Tris-HCl, pH 7.4, centrifuging at 20,000×g, decanting the supernatant and storing at −80° C.; the membrane preparations are resuspended in 3 ml of chilled Assay Buffer and homogenized by several passages through a 26 gauge needle before using in the assay.

The 250-1 reactions are incubated at room temperature for 1.5 hours, then harvested by rapid filtration onto 0.3% polyethyleneimine-treated, 96-well filter mats using a 96-well Filtermate harvester. Four rapid 500-1 washes are performed with chilled Assay Buffer to reduce non-specific binding. The filter mats are dried, then scintillant is added to the filters and the radioactivity retained on the filters is counted in a Microbeta scintillation counter.

Raw data (dpm) representing total radioligand binding (i.e., specific+non-specific binding) is plotted as a function of the logarithm of the molar concentration of the competitor (i.e., test or reference compound). Non-linear regression of the normalized (i.e., percent radioligand binding compared to that observed in the absence of test or reference compound) raw data is performed in Prism 4.0 (GraphPad Software) using the built-in three parameter logistic model describing ligand competition binding to radioligand-labeled sites:

y=bottom+[(top−bottom)/(1+10×−log IC₅₀)]

where bottom equals the residual radioligand binding measured in the presence of 10 μM reference compound (i.e., non-specific binding) and top equals the total radioligand binding observed in the absence of competitor. The log IC₅₀ (i.e., the log of the ligand concentration that reduces radioligand binding by 50%) is thus estimated from the data and used to obtain the Ki by applying the Cheng-Prusoff approximation:

Ki=IC₅₀/(1+[ligand]/KD)

where [ligand] equals the assay radioligand concentration and KD equals the affinity constant of the radioligand for the target receptor.

Functional Data (K_{b})

Functional efficacy of the compounds of the disclosure on 5-HT₇ serotonin receptors are measured in a cell based cAMP enzyme fragment complementation assay using the HitHunter cAMP assay (DiscoveRx). Cells stably expressing human 5HT₇ receptors are plated in 96-well plates at 4000 cells/well, 16-20 hours prior to assay in growth media (Ultraculture medium, 2 mM GlutaMax and G418 1 mg/mL. Serial dilutions of the agonist, 5-hydroxytryptamine (5-HT), are prepared in a final concentration range of 10 μM to 10 nM. Compounds of the disclosure are prepared in 3-fold serial dilutions to obtain a final concentration range of 10 μM to 0.1 nM. Compounds of the disclosure are tested for agonist activity in the absence of 5-HT and antagonist activity in the presence of 5-HT. For the cAMP assay, the protocol is followed according to the instructions provided by the supplier. Briefly, cells are incubated with a compound of the disclosure for 30 minutes at 37° C. prior to addition of EC₇₀ concentration of 5-HT. After an additional 30 minutes, cAMP antibody/cell lysis solution is added (20 μL/well) and incubated for 60 minutes at room temperature. cAMP XS+EA reagent is added (20 μL/well) and incubated for 2 hours at room temperature. Luminescence is read on the Envision Multilabel plate reader.

Exemplary Kᵢ and K_{b} data for certain compounds according to formulas described (e.g., according to Formulas (I)-(II) such as any of Formulas (A)-(AAA)) herein is provided in Table 1.

While a number of embodiments of this invention have been described, it is apparent that the basic examples may be altered to provide other embodiments that utilize the compounds, methods, and processes of this invention. Therefore, it will be appreciated that the scope of this invention is to be defined by the appended claims rather than by the specific embodiments that have been represented by way of example herein.

What is claimed is:

1. A compound having a structure according to Formula (I*), 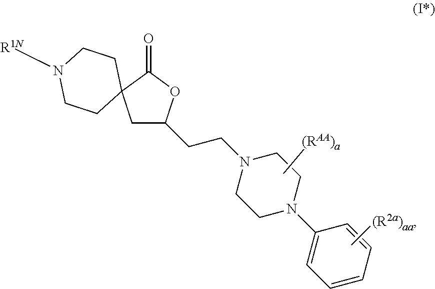
including enantiomers, diastereomers, hydrates, solvates, pharmaceutically acceptable salts, prodrugs and complexes thereof, wherein:
R^{1N} is selected from the group consisting of imidazole, oxazole, isoxazole, 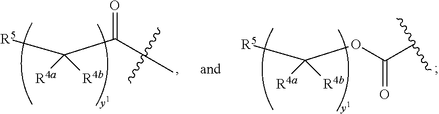
wherein
each R^{4a} and R^{4b} is hydrogen or C₁-C₇ alkyl; or R^{4a} and R^{4b} optionally are taken together with the atoms to which they are bound to form a ring containing 3 to 7 atoms, optionally containing oxygen;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR″COOR^{8j}, NHCONR^{8f}, NR^{8g}COR^{8h} and 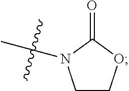 [SMILES: *N1CCOC1=O]
each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R⁸¹ and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
each R^{AA} is independently C₁-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
a is 0, 1, or 2;
aa is 0, 1, or 2;
y¹ is 0, 1 or 2; and
wherein when R⁵ is unsubstituted C₁-C₇ alkyl or unsubstituted C₃-C₇ cycloalkyl, then a is 1 or 2.

2. A compound having a structure according to Formula (I*-N), 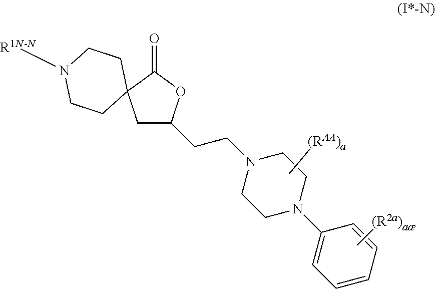
including enantiomers, diastereomers, hydrates, solvates, pharmaceutically acceptable salts, prodrugs and complexes thereof, wherein:
R^{1N-N} is selected from the group consisting of C₆-C₁₀ heteroaryl, five- to ten-membered heteroaryl, 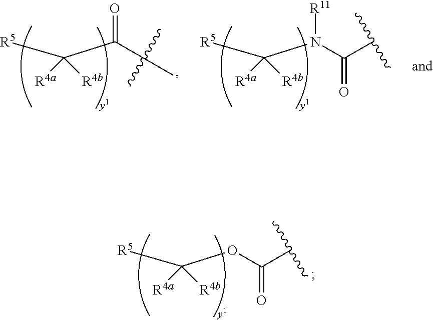
wherein
each R^{4a} and R^{4b} is hydrogen or C₁-C₇ alkyl; or R^{4a} and R^{4b} optionally are taken together with the atoms to which they are bound to form a ring containing 3 to 7 atoms, optionally containing oxygen;
R⁵ is selected from the group consisting of C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}COR^{8h} and 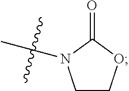 [SMILES: *N1CCOC1=O]
each R^{8a}, R^{8b}, R^{8d}, R^{8g}, and R⁸ⁱ is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
each R^{AA} is independently C₁-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
a is 0, 1, or 2;
aa is 0, 1, or 2;
y¹ is 0, 1 or 2; and
wherein when R⁵ is unsubstituted C₁-C₇ alkyl or unsubstituted C₃-C₇ cycloalkyl, then a is 1 or 2.

3. The compound of claim 1 or 2, having a structure according to Formula (I*-1), 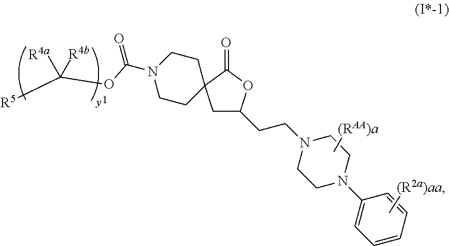
including enantiomers, diastereomers, hydrates, solvates, pharmaceutically acceptable salts, prodrugs and complexes thereof.

4. The compound of claim 1 or 2, having a structure according to Formula (I*-2), 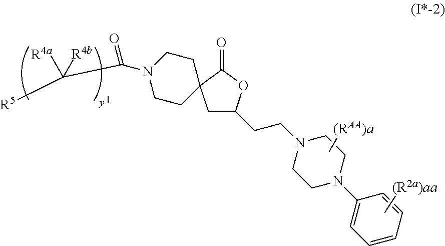
including enantiomers, diastereomers, hydrates, solvates, pharmaceutically acceptable salts, prodrugs and complexes thereof.

5. The compound of claim 2, having a structure according to Formula (I*-3), 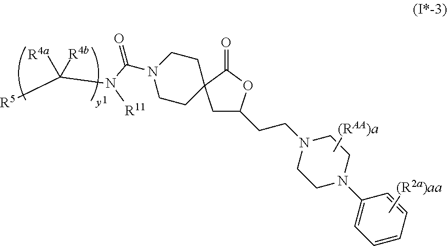
including enantiomers, diastereomers, hydrates, solvates, pharmaceutically acceptable salts, prodrugs and complexes thereof.

6. The compound of claim 1, wherein R^{1N} is: 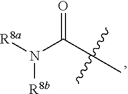
wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl;

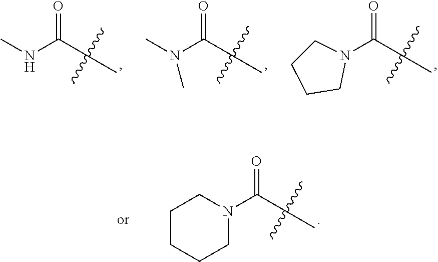

7. The compound of claim 1, wherein R^{1N} is: 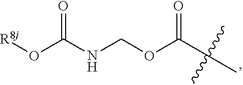
wherein R^{8j} is selected from the group consisting of C₁-C₇ alkyl, C₃-C₇ cycloalkyl, C₆-C₁₀ aryl, and 5- to 10-membered heteroaryl; 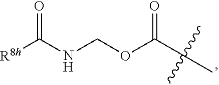
wherein R^{8h} is unsubstituted C₁-C₇ alkyl;
or

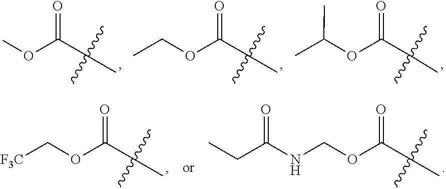

8. The compound of claim 1, wherein R^{1N} is: 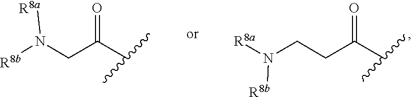
wherein each R^{8a} and R^{8b} is independently H or unsubstituted C₁-C₇ alkyl; 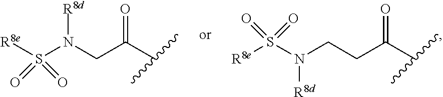
wherein R^{8d} is independently H or unsubstituted C₁-C₇ alkyl, and R^{8e} is unsubstituted C₁-C₇ alkyl; 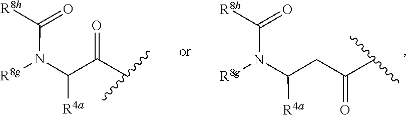
or wherein each of R^{4a} and R^{8g} is independently H or unsubstituted C₁-C₇ alkyl; and R^{8h} is unsubstituted C₁-C₇ alkyl; 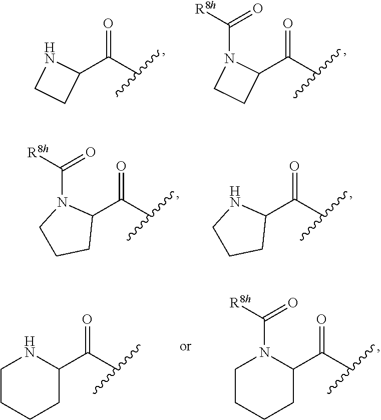
wherein R^{8h} is unsubstituted C₁₋₇ alkyl 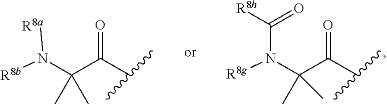
wherein each R^{a}, R^{8b} and R^{8g} is independently H or unsubstituted C₁-C₇ alkyl, and R^{8h} is unsubstituted C₁-C₇ alkyl; 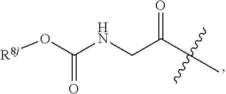
wherein R^{8j} is selected from the group consisting of C₁-C₇ alkyl, C₃-C₇ cycloalkyl, C₆-C₁₀ aryl, and 5- to 10-membered heteroaryl; 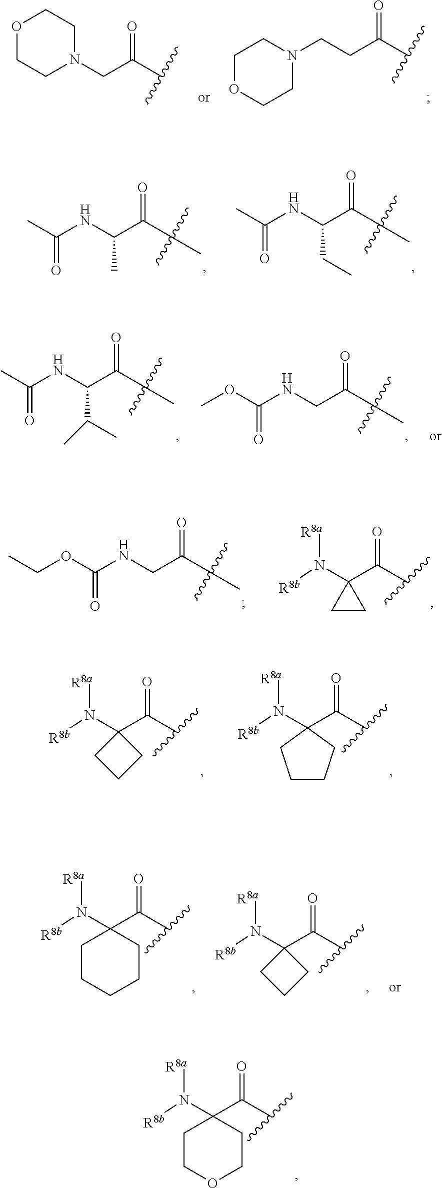
wherein each R^{8a} and R^{8b} is independently H or unsubstituted C₁-C₇ alkyl; 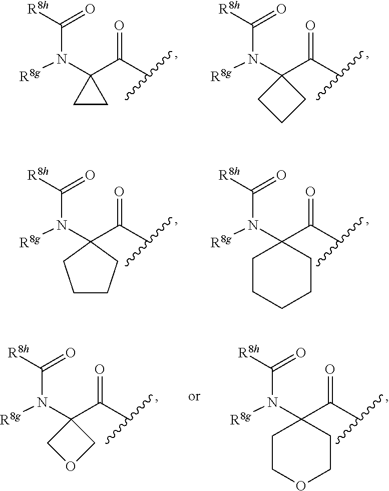
wherein R¹ is independently H or unsubstituted C₁-C₇ alkyl, and R^{8h} is independently unsubstituted C₁-C₇ alkyl;
or

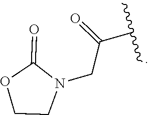 [SMILES: *C(=O)CN1CCOC1=O]

9. The compound of claim 1, wherein R^{1N} is:

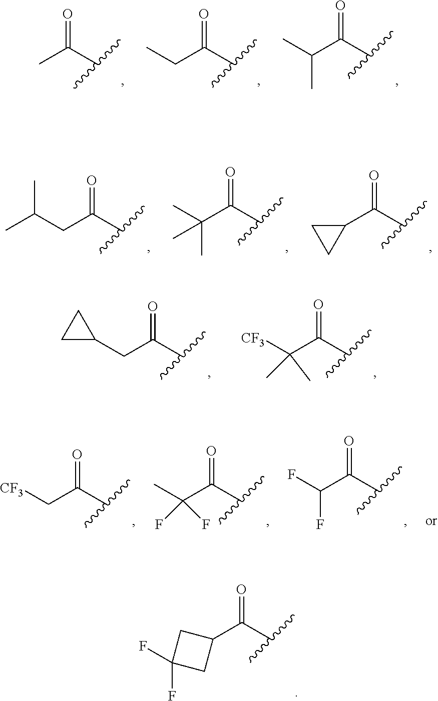

10. The compound of claim 1, wherein R^{1N} is:

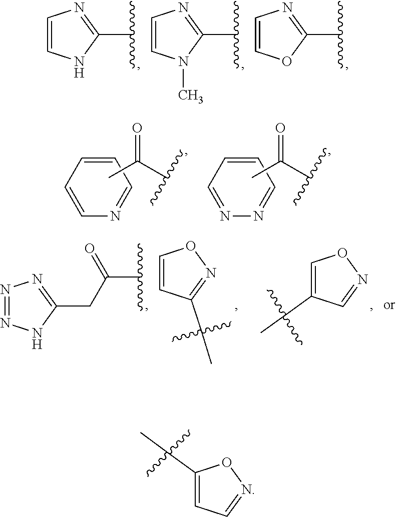

11. A compound having a structure according to Formula (I**), 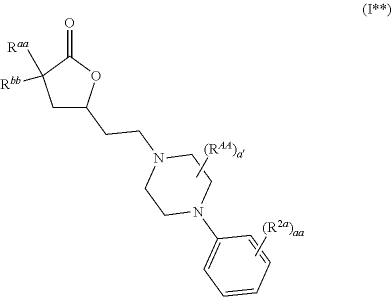
including enantiomers, diastereomers, hydrates, solvates, pharmaceutically acceptable salts, prodrugs and complexes thereof, wherein:
each R^{aa} and R^{bb} is selected from the group consisting of hydrogen, C₁-C₇ alkyl and C₃-C₇ branched alkyl;
each R^{AA} is independently C₁-C₇ linear alkyl;
each R^{2a} is independently halogen, unsubstituted C₁-C₇ alkyl, C₁-C₇ perhaloalkyl, unsubstituted C₁-C₇ alkoxy, C₁-C₇ perhaloalkoxy, or CN;
aa is 0, 1, or 2; and
a′ is 1 or 2.

12. The compound of claim 11, wherein R^{aa} and R^{bb} are each ethyl.

13. The compound of any one of claims 1-10, wherein a is 0 or 1.

14. The compound of any one of claims 1-10, wherein a is 1 or 2, and each R^{AA} is methyl.

15. The compound of claim 11 or 12, wherein a′ is 1 or 2, and each R^{AA} is methyl.

16. The compound of any one of claims 1-15, wherein aa is 1 or 2.

17. The compound of claim 16, wherein each R^{2a} is independently halogen.

18. The compound of claim 17, wherein each R^{2a} is independently —F or —Cl.

19. The compound of any one of claims 1-18, wherein the C5 carbon of the 2-dihydrofuranone has the (R)-configuration.

20. The compound of any one of claims 1-18, wherein the C5 carbon of the 2-dihydrofuranone has the (S)-configuration.

21. A compound having a structure according to Formula (I) 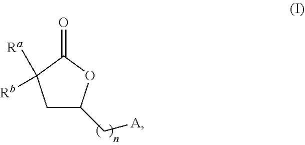
including enantiomers, diastereomers, hydrates, solvates, pharmaceutically acceptable salts, prodrugs and complexes thereof, wherein:
each R^{a} and R^{b} is selected from the group consisting hydrogen, C₁-C₇ alkyl, and C₃-C₇ branched alkyl; or R^{a} and R^{b} are taken together with the atoms to which they are bound to form a carbocyclic ring having from 5 to 7 ring atoms, optionally containing a double bond; or R^{a} and R^{b} are taken together with the atoms to which they are bound to form a ring having from 6 to 8 ring atoms comprising a moiety selected from the group consisting of O, S, SO, SO₂, and NR¹;
A is an N-linked, five- to twelve-membered nitrogen-containing heterocyclyl, wherein said nitrogen-containing heterocyclyl is bicyclic or polycyclic and optionally includes further heteroatoms selected from O, N, and S, and wherein a non-aromatic, nitrogen-containing heterocyclyl further comprises a group R²;
R¹ is a H, C₁-C₇ alkyl, C₃-C₇ cycloalkyl, phenyl, benzyl, five- to six-membered heteroaryl ring, a polar acyl group, or a polar sulfonyl group;
R² is selected from the group consisting of 6- to 10-membered aryl, 5- to 10-membered nitrogen-containing heteroaryl, and 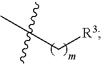
R³ is a 6- to 10-membered aryl or 5- to 10-membered nitrogen-containing heteroaryl;
m is 1, 2, or 3; and
n is 1, 2, 3, or 4; and
wherein when A is 1,2,3,4-tetrahydroquinol-1-yl, 1,2,3,4-tetrahydroisoquinol-2-yl, octahydropyrrolo[3,4-c]pyrrol-1-yl, or 2,6-diazaspiro[3.3]heptan-1-yl, then R^{a} and R^{b} cannot both be methyl, both be ethyl, or both be phenyl nor can R^{a} and R^{b} combine to form unsubstituted C₃-C₆ cycloalkyl.

22. The compound of claim 21, having one of the following structures,

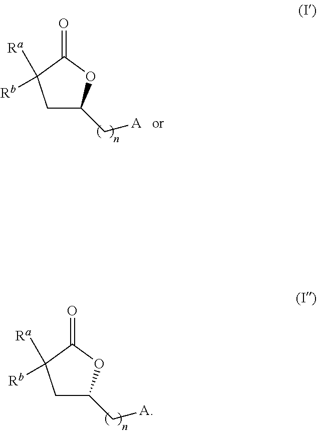

23. The compound of claim 21 or 22, wherein R^{a} and R^{b} are both methyl or ethyl, or R^{a} and R^{b} combine to form unsubstituted cyclopropyl, cyclobutyl, cyclopentyl, or cyclohexyl.

24. The compound of claim 23, having one of the following structures,

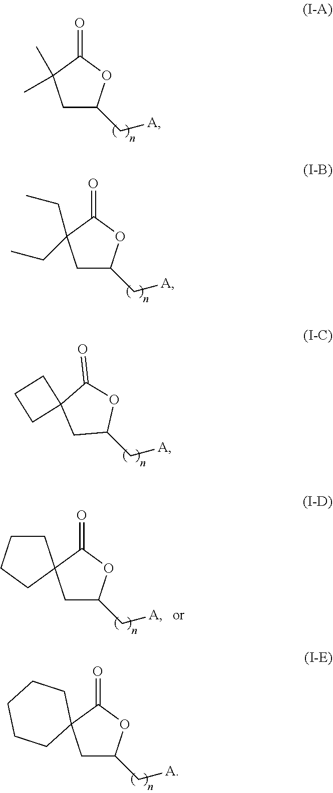

25. The compound of claim 24, having one of the following structures,

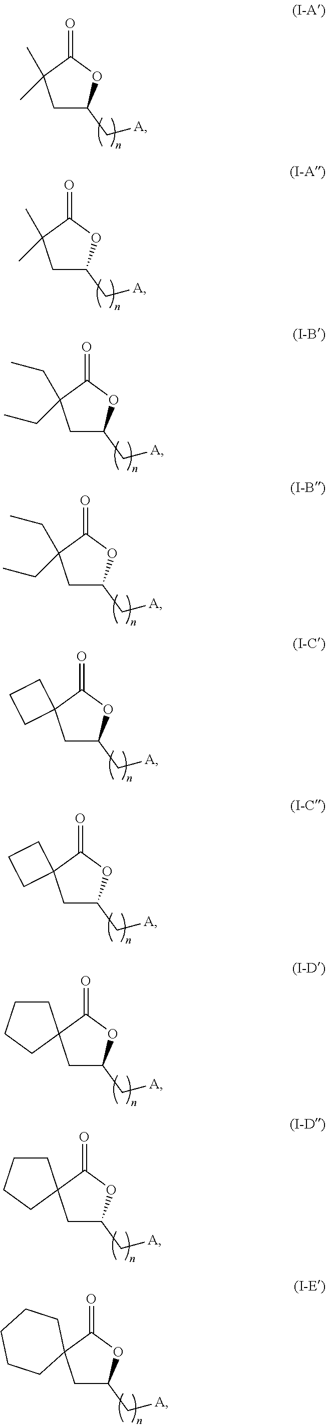

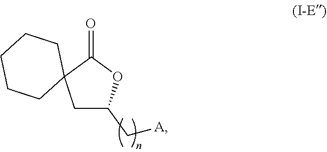

26. The compound of claim 21 or 22, wherein R^{a} and R^{b} are taken together with the atoms to which they are bound to form a ring having from 6 to 8 ring atoms.

27. The compound of claim 26, having a structure according to Formula (I-F),

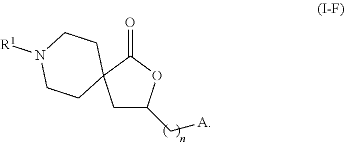

28. The compound of claim 26, having a structure according to one of the following formulas,

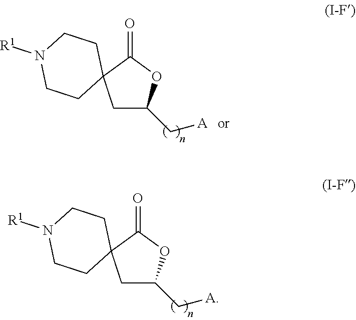

29. The compound of any one of claims 21-28, wherein A is selected from the group consisting of 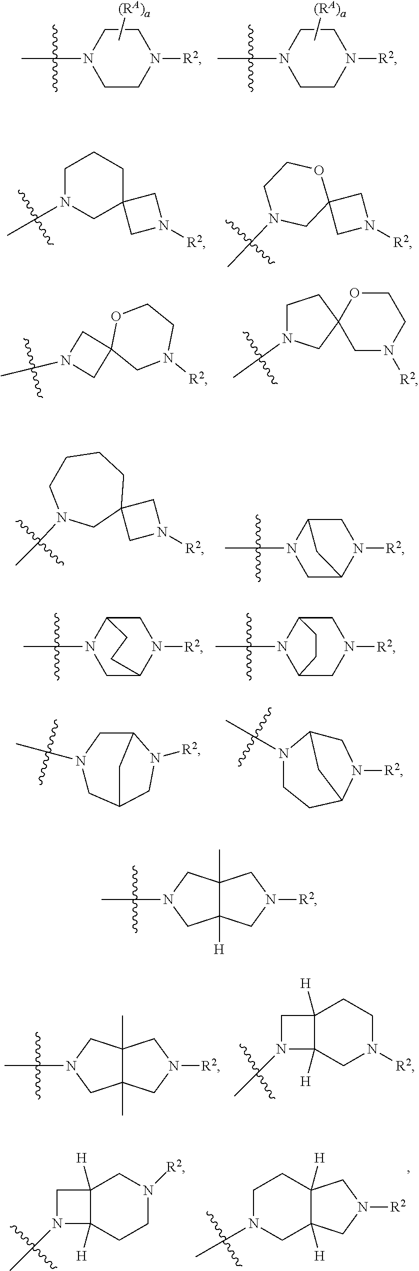 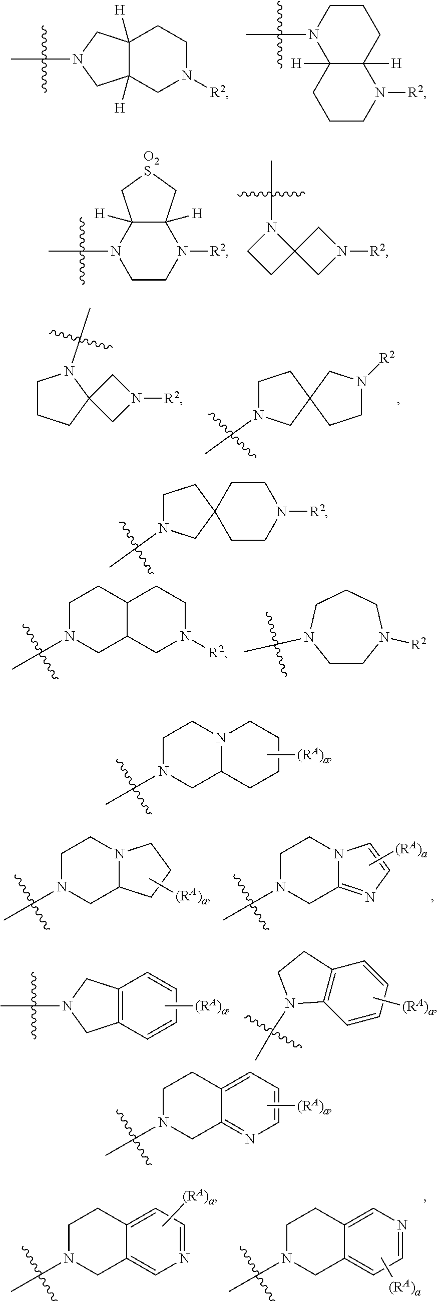 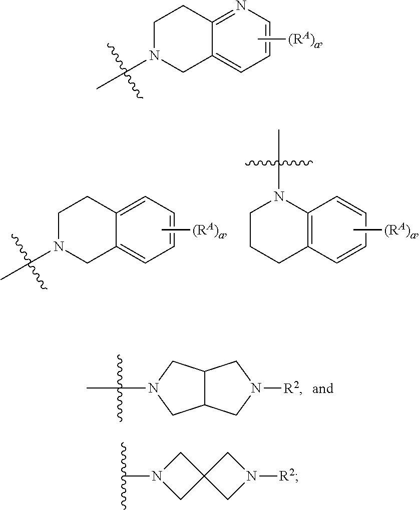
wherein
R² is selected from the group consisting of phenyl, naphthyl, pyridyl, indolyl and 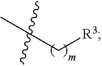
R³ is selected from the group consisting of phenyl, naphthyl, pyridyl and indolyl;
R^{A} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkylamino, arylamino, C₂-C₇ acyl, arylcarbonyl and five- to six-membered heterocyclic group each containing 1 to 4 heteroatoms selected from oxygen, sulfur and nitrogen; and
a is independently 0, 1, or 2.

30. The compound of claim 29, wherein A is selected from the group consisting of

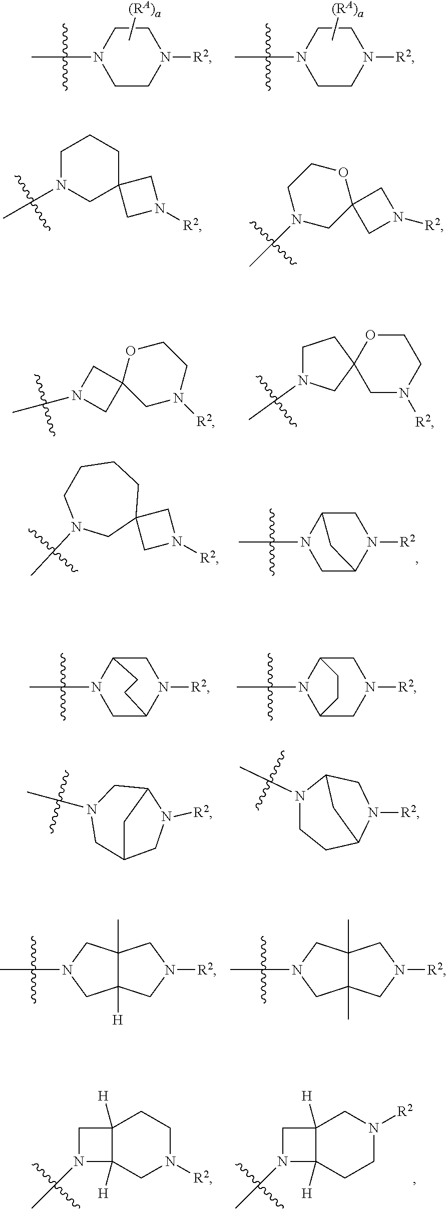

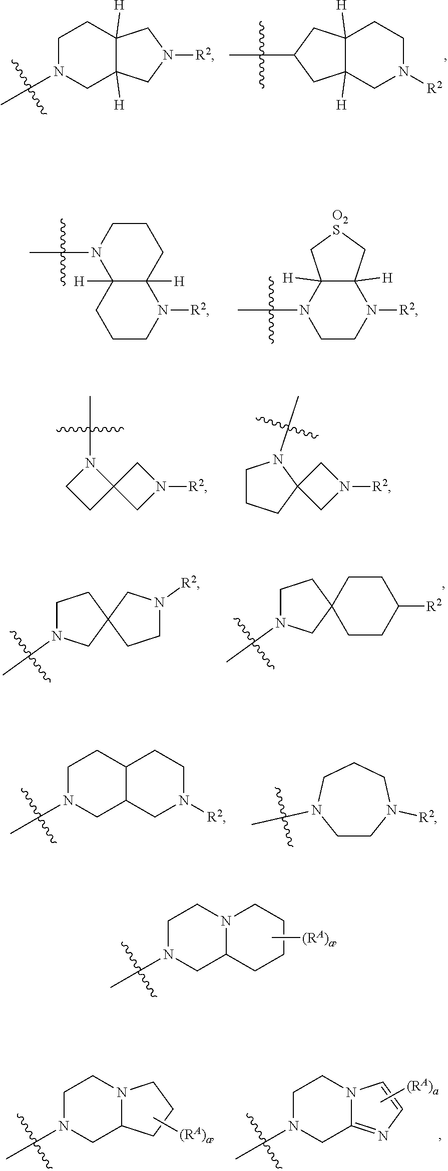

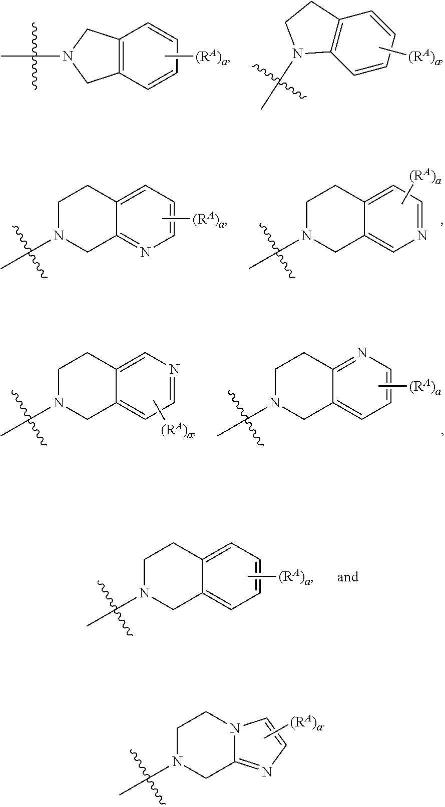

31. The compound of claim 29, wherein A is selected from the group consisting of

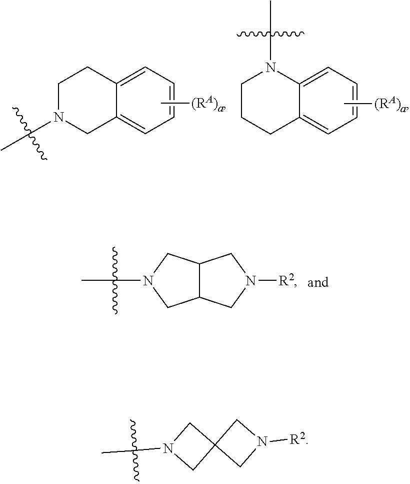

32. The compound of any one of claims 21-31, wherein.
R¹ is selected from the group consisting of H, C₁-C₇ alkyl, C₃-C₇ cycloalkyl, phenyl, benzyl, imidazole, oxazole, isoxazole, 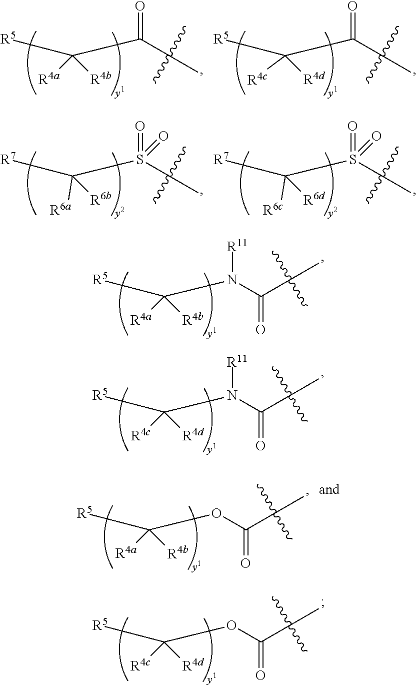
each R^{4a}, R^{4b}, R^{4c}, R^{6a}, R^{6b} and R^{6c} is selected from the group consisting of hydrogen, C₁-C₇ alkyl and C₃-C₇ cycloalkyl;
R^{4a} and R^{4b} optionally are taken together with the atoms to which they are bound to form a ring containing 3 to 7 atoms, optionally containing oxygen;
R^{6a} and R^{6b} optionally are taken together with the atoms to which they are bound to form a ring containing 3 to 7 atoms, optionally containing oxygen;
each R^{4d} and R^{6d} is selected from the group consisting of phenyl, benzyl, pyridyl, —CH₂(pyridyl), imidazole, and —CH₂(imidazole);
R⁵ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NR⁸ⁱCOOR^{8j}, NHCONR^{8f}, NR^{8g}CR^{8h} and 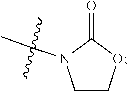 [SMILES: *N1CCOC1=O]
R⁷ is selected from the group consisting of hydrogen, (C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8e}, NHCONR^{8f};
each R^{8a}, R^{8b}, R^{8d}, R^{8g}, and R⁸ⁱ is selected from the group consisting of hydrogen, C₁-C₇ alkyl, and C₃-C₇ cycloalkyl;
R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl;
R^{8j} is selected from the group consisting of C₁-C₇ alkyl, C₃-C₇ cycloalkyl, C₆-C₁₀ aryl, and 5- to 10-membered heteroaryl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
R¹¹ is selected from the group consisting of hydrogen, C₁-C₇ alkyl, and C₃-C₇ cycloalkyl;
y¹ is 0, 1 or 2; and
y² is 0, 1, or 2.

33. The compound of claim 32, wherein R¹ is selected from the group consisting of.

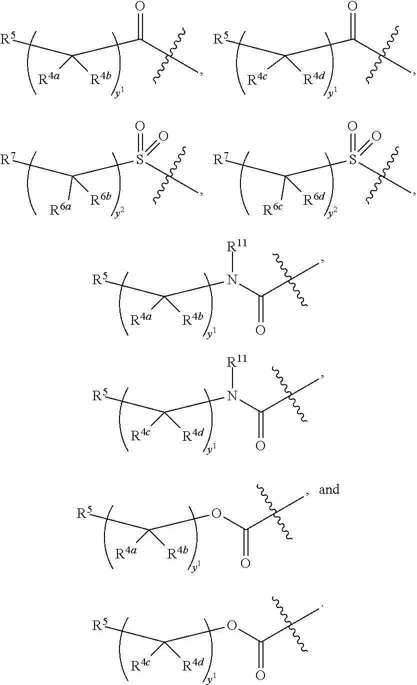

34. The compound of claim 32 or 33, wherein R¹ is COOR⁵, wherein R⁵ is C₆-C₁₀ aryl or 5- to 10-membered heteroaryl; 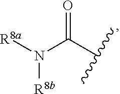
wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl; 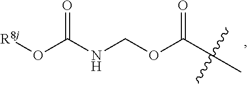
wherein R^{8j} is selected from the group consisting of C₁-C₇ alkyl, C₃-C₇ cycloalkyl, C₆-C₁₀ aryl, and 5- to 10-membered heteroaryl; 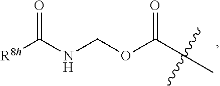
wherein R^{8h} is unsubstituted C₁-C₇ alkyl; 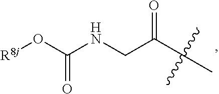
wherein R^{8j} is selected from the group consisting of C₁-C₇ alkyl, C₃-C₇ cycloalkyl, C₆-C₁₀ aryl, and 5- to 10-membered heteroaryl; 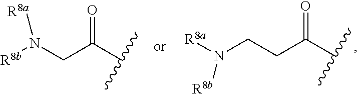
wherein each R^{8a} and R^{8b} is independently H or unsubstituted C₁-C₇ alkyl; 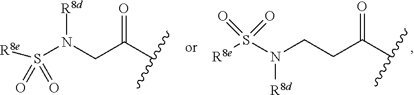
wherein R^{8d} is independently H or unsubstituted C₁-C₇ alkyl, and R⁸¹ is unsubstituted C₁-C₇ alkyl 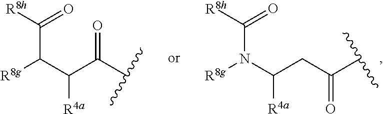
wherein each of R^{4a} and R^{8g} is independently H or unsubstituted C₁-C₇ alkyl; and R^{8h} is unsubstituted C₁-C₇ alkyl; 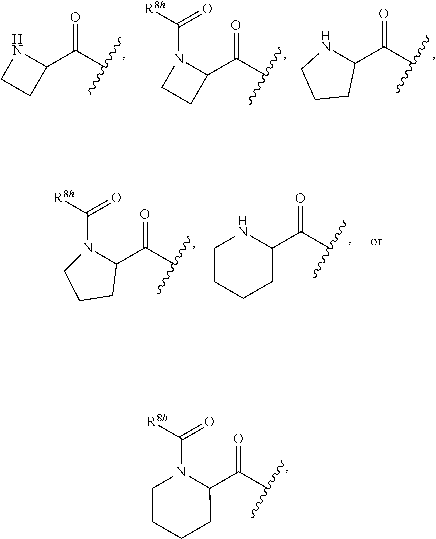
wherein R^{8h} is unsubstituted C₁-C₇ alkyl; 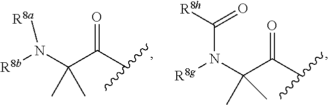
wherein each R^{8a}, R^{8b}, and R^{8g} is independently H or unsubstituted C₁-C₇ alkyl, and R^{8h} is unsubstituted C₁-C₇ alkyl; 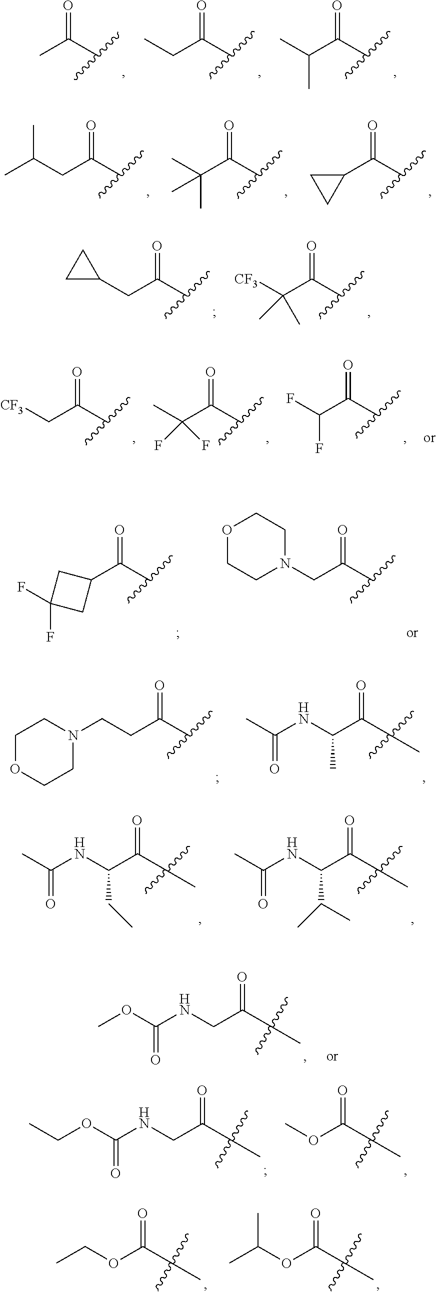 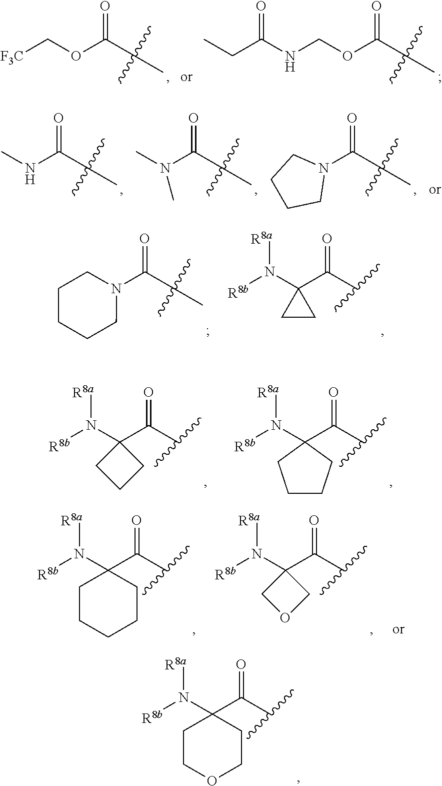
wherein each R^{8a} and R^{8b} is independently H or unsubstituted C₁-C₇ alkyl; 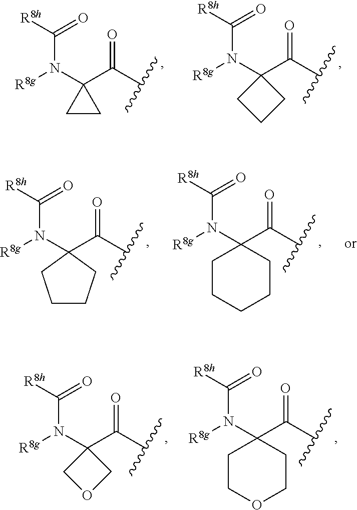
wherein R^{8d} is independently H or unsubstituted C₁-C₇ alkyl, and R^{8h} is independently unsubstituted C₁-C₇ alkyl; or

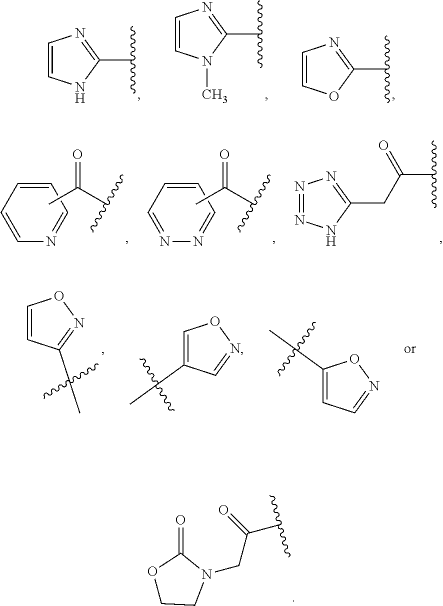

35. A compound having a structure according to Formula (II) 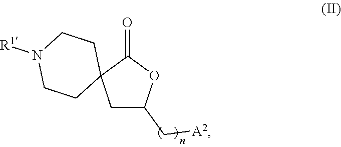
including enantiomers, diastereomers, hydrates, solvates, pharmaceutically acceptable salts, prodrugs and complexes thereof, wherein:
A² is 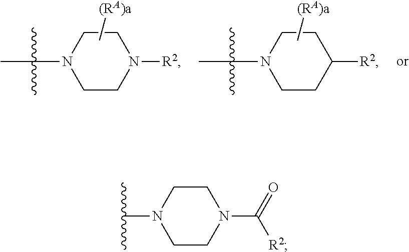
R² is selected from the group consisting of 6- to 10-membered aryl, 5- to 10-membered nitrogen-containing heteroaryl, and 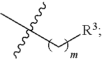
R³ is a 6- to 10-membered aryl or 5- to 10-membered nitrogen-containing heteroaryl;
R^{A} is selected from the group consisting of C₁-C₇ linear alkyl, C₃-C₇ branched alkyl, C₃-C₇ cycloalkyl, C₁-C₇ linear alkoxy, C₃-C₇ branched alkoxy, C₃-C₇ cycloalkoxy, aryloxy, C₁-C₇ linear haloalkyl, C₃-C₇ branched haloalkyl, C₃-C₇ cyclohaloalkyl, C₂-C₇ alkenyl, C₂-C₇ cycloalkenyl, C₂-C₇ alkynyl, aryl, arylalkyl, nitro, hydroxy, mercapto, oxo, thioxo, cyano, carbamoyl, carboxyl, C₁-C₇ alkoxycarbonyl, sulfo, halogen, C₁-C₇ alkylthio, arylthio, C₁-C₇ alkylsulfinyl, arylsulfinyl, C₁-C₇ alkylsulfonyl, arylsulfonyl, amino, C₁-C₇ acylamino, mono- or di-C₁-C₇ alkylamino, C₃-C₇ cycloalkyl amino, arylamino, C₂-C₇ acyl, arylcarbonyl and five- to six-membered heterocyclic group each containing 1 to 4 heteroatoms selected from oxygen, sulfur and nitrogen;
a is 0, 1, or 2; m is 1, 2, or 3;
n is 1, 2, 3, or 4;
R^{1′} is selected from the group consisting of a C₆-C₁₀ aryl, a five- to six-membered heteroaryl ring, 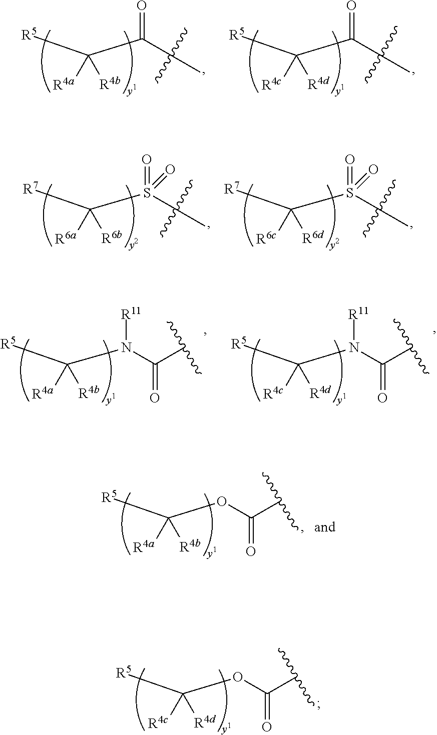
each R^{4a}, R^{4b}, R^{4c}, R^{6a}, R^{6b} and R^{6c} is selected from the group consisting of hydrogen, C₁-C₇ alkyl and C₃-C₇ cycloalkyl;
R^{4a} and R^{4b} optionally are taken together with the atoms to which they are bound to form a ring containing 3 to 7 atoms, optionally containing oxygen;
R^{6a} and R^{6b} optionally are taken together with the atoms to which they are bound to form a ring containing 3 to 7 atoms, optionally containing oxygen;
each R^{4d} and R^{6d} is selected from the group consisting of phenyl, benzyl, pyridyl, —CH₂(pyridyl), imidazole, and —CH₂(imidazole);
R⁵ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8c}, NR⁸ⁱCOOR^{8j}, NHCONR^{8f}, NR^{8g}COR^{8h} and 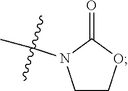 [SMILES: *N1CCOC1=O]
R⁷ is selected from the group consisting of hydrogen, C₁-C₇ alkyl, C₃-C₇ cycloalkyl, C₁-C₇ alkoxy, C₃-C₇ cycloalkoxy, C₁-C₇ haloalkyl, C₃-C₇ cyclohaloalkyl, C₁-C₇ haloalkoxy, C₃-C₇ cyclo haloalkoxy, C₆-C₁₀ aryl, 5- to 10-membered heteroaryl, CN, NR^{8a}R^{8b}, SO₂R^{8c}, NR^{8d}SO₂R^{8c}, NHCONR^{8f};
each R^{8a}, R^{8b}, R^{8d}, R^{8g}, and R⁸ⁱ is selected from the group consisting of hydrogen, C₁-C₇ alkyl, and C₃-C₇ cycloalkyl; or
R^{8j} is selected from the group consisting of C₁-C₇ alkyl, C₃-C₇ cycloalkyl, C₆-C₁₀ aryl, and 5- to 10-membered heteroaryl; or
R^{8a} and R^{8b} optionally are taken together with the atoms to which they are bound to form a heterocyle containing 3 to 7 atoms, optionally containing a group selected from oxygen, sulfur, and NR⁹;
each R^{8c}, R^{8e}, R^{8f} and R^{8h} is C₃-C₇ alkyl or C₃-C₇ cycloalkyl; or
when R^{4a} and R^{8a} both present, or R^{4a} and R^{8g} both present, these groups are optionally taken together with the atoms to which they are bound to form a ring containing 4 to 7 atoms;
R⁹ is selected from the group consisting of hydrogen, C₁-C₇ alkyl, and C₃-C₇ cycloalkyl;
R¹¹ is selected from the group consisting of hydrogen, C₁-C₇ alkyl, and C₃-C₇ cycloalkyl;
y¹ is 0, 1 or 2; and
y² is 0, 1, or 2; and
wherein when A² is 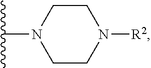
R² is phenyl, R^{1′} is 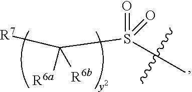
y² is 0, and n is 2, then R⁷ is not methyl, CH₂SO₂CH₃, CH₂CN, tetrahydropyranyl, phenyl, 4-substituted phenyl, or 5- to 8-membered heteroaryl.

36. The compound of claim 35, having one of the following structures,

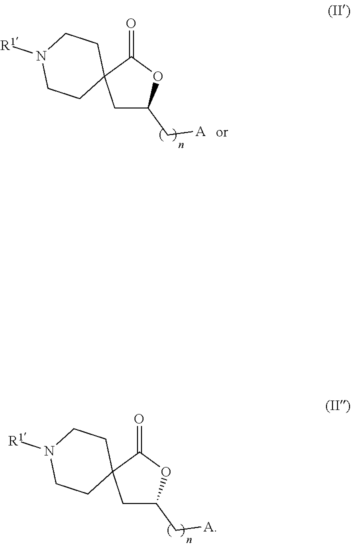

37. The compound of claim 35 or 36, wherein R^{1′} is COOR⁵, wherein R⁵ is C₆-C₁₀ aryl or 5- to 10-membered heteroaryl; 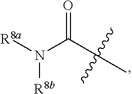
wherein each R^{8a} and R^{8b} is selected from the group consisting of hydrogen, C₁-C₇ alkyl, and C₃-C₇ cycloalkyl; or R^{8a} and R^{8b} optionally are taken together with the atoms to which they are bound to form a heterocyle containing 3 to 7 atoms, optionally containing a group selected from oxygen, sulfur, and NR⁹; and R⁹ is selected from the group consisting of hydrogen, C₁-C₇ alkyl, and C₃-C₇ cycloalkyl; 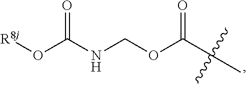
wherein R^{8j} is selected from the group consisting of C₁-C₇ alkyl, C₃-C₇ cycloalkyl, C₆-C₁₀ aryl, and 5- to 10-membered heteroaryl; 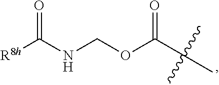
wherein R^{8f} is unsubstituted C₁-C₇ alkyl; 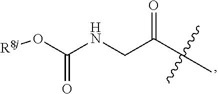
wherein R^{8j} is selected from the group consisting of C₁-C₇ alkyl, C₃-C₇ cycloalkyl, C₆-C₁₀ aryl, and 5- to 10-membered heteroaryl; 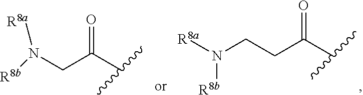
wherein each R^{8a} and R^{8b} is independently H or unsubstituted C₁-C₇ alkyl; 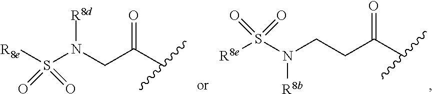
wherein R^{8d} is independently H or unsubstituted C₁-C₇ alkyl, and R^{8f} is unsubstituted C₁-C₇ alkyl 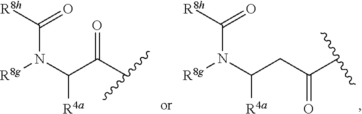
wherein each of R^{4a} and R^{8g} is independently H or unsubstituted C₁-C₇ alkyl; and R^{8h} is unsubstituted C₁-C₇ alkyl; 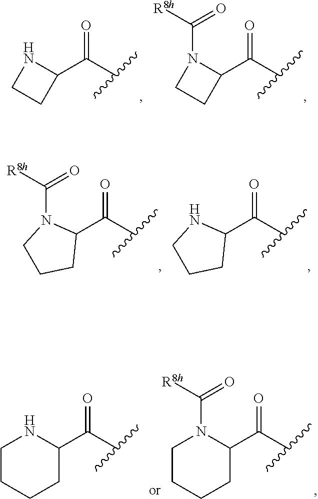
wherein R^{8h} is unsubstituted C₁-C₇ alkyl; 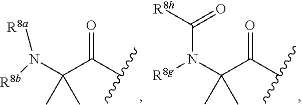
wherein each R^{8a}, R^{8b} and R^{8g} is independently H or unsubstituted C₁-C₇ alkyl, and R^{8h} is unsubstituted C₁-C₇ alkyl; 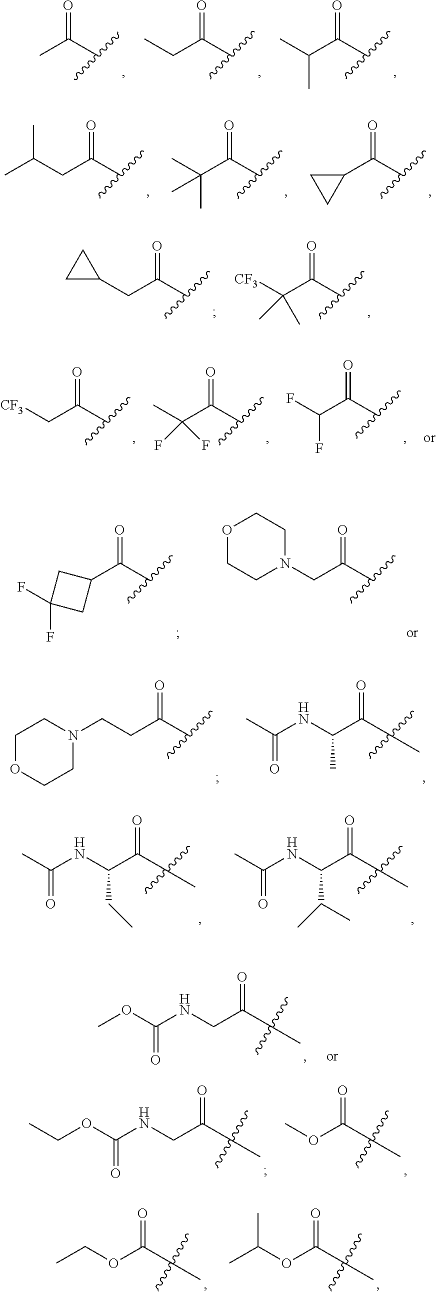 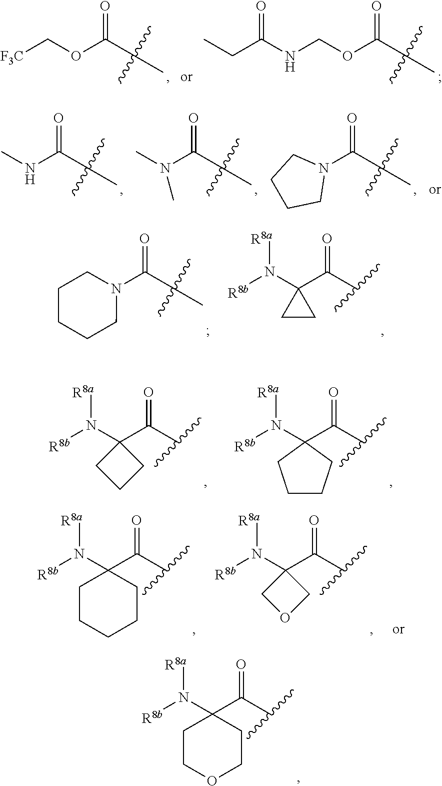
wherein each R^{8a} and R^{8b} is independently H or unsubstituted C₁-C₇ alkyl; 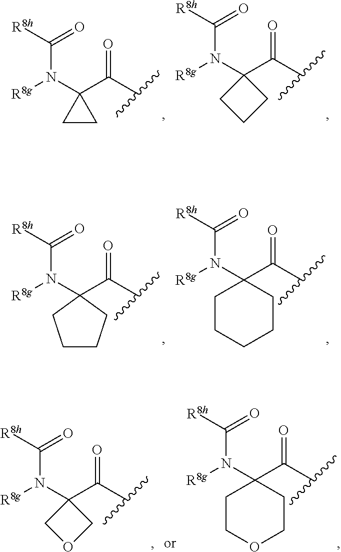
wherein R^{8g} is independently H or unsubstituted C₁-C₇ alkyl, and R^{8h} is independently unsubstituted C₁-C₇ alkyl; or

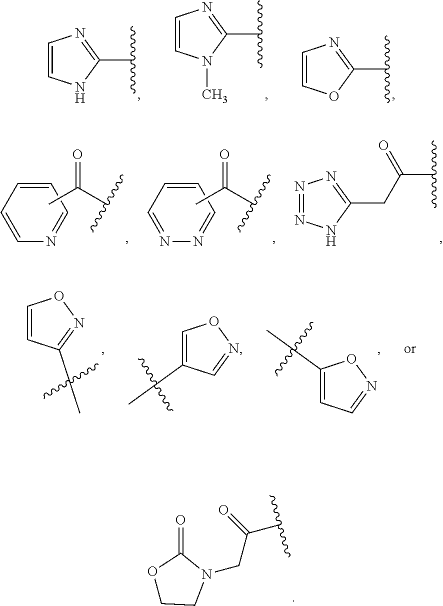

38. The compound of any one of claims 35-37, wherein A² is

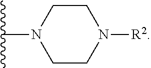

39. The compound of any one of claims 35-37, wherein A² is

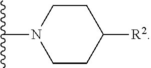

40. The compound of any one of claims 35-37, wherein A² is

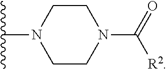

41. A compound selected from the group consisting of Compounds 1-145, or a pharmaceutically acceptable salt thereof.

42. A pharmaceutical composition comprising a compound according to any one of claims 1-41, or a pharmaceutically acceptable salt thereof.

43. A pharmaceutical composition according to claim 42, further comprising at least one pharmaceutically acceptable excipient.

44. A method of treating a disease associated with dysregulation of 5-hydroxytryptamine receptor 7 activity, said method comprising administering to a subject an effective amount of at least one compound according to any one of claims 1-41, or a pharmaceutically acceptable salt thereof.

45. The method of claim 44, wherein the at least one compound, or a pharmaceutically acceptable salt thereof, is administered in a composition further comprising at least one excipient.

46. The method of claim 44 or 45, wherein the disease associated with dysregulation of 5-hydroxytryptamine receptor 7 activity is selected from the group consisting of peripherally selective diseases, nervous system diseases, circadian rhythm disorder, depression, schizophrenia, neurogenic inflammation, hypertension, peripheral, vascular diseases, migraine, neuropathic pain, peripheral pain, allodynia, thermoregulation disorder, learning disorder, memory disorder, hippocampal signaling disorder, sleep disorder, attention deficit/hyperactivity disorder, anxiety, avoidant personality disorder, premature ejaculation, eating disorder, premenstrual syndrome, premenstrual dysphonic disorder, seasonal affective disorder, bipolar disorder, inflammatory bowel disease (IBD), intestinal inflammation, epilepsy, seizure disorders, drug addiction, alcohol addiction, breast cancer, liver fibrosis, chronic liver injury, hepatocellular carcinoma, small intestine neuroendocrine tumors, and lung injury.

47. The method of claim 44 or 45, wherein the disease associated with dysregulation of 5-hydroxytryptamine receptor 7 activity is inflammatory bowel disease (IBD) or intestinal inflammation.
